(19) United States
(12) Patent Application Publication
(10) Pub. No.: US 2023/0047159 A1
(43) Pub. Date: Feb. 16, 2023

(54) REGULATORY T CELL (TREG) COMPOSITIONS AND METHODS FOR TREATING NEURODEGENERATIVE DISEASE

(71) Applicant: THE METHODIST HOSPITAL, Houston, TX (US)

(72) Inventors: Stanley Hersh APPEL, Houston, TX (US); Jason Robert THONHOFF, Missouri City, TX (US); David Robert BEERS, Houston, TX (US); Aaron Drew THOME, Houston, TX (US)

(73) Assignee: THE METHODIST HOSPITAL, Houston, TX (US)

(21) Appl. No.: 17/292,423

(22) PCT Filed: Dec. 4, 2020
(86) PCT No.: PCT/US2020/063378

§ 371 Date: May 7, 2021

Related U.S. Application Data
(60) Provisional application No. 62/944,346, filed on Dec. 5, 2019.

(51) Int. Cl. A61K 35/17; C12N 5/0783; A01N 1/02; A61P 25/28

(52) U.S. Cl. CPC A61K 35/17; C12N 5/0637; A01N 1/0221; A01N 1/0284; A61P 25/28; C12N 2501/998; C12N 2501/2302; C12N 2501/04

(57) ABSTRACT

Disclosed are improved methods for manufacturing large-scale populations of robust, highly pure, and functional T regulatory cells (Tregs). Also disclosed are expanded Treg populations, cryopreserved Treg populations and methods and uses of these cells in compositions formulated for treating one or more mammalian diseases, including, for example, treatment, prophylaxis, and/or amelioration of one or more symptoms of a human neurodegenerative disorder. In particular, the compositions and methods provided herein find clinical use in the treatment and amelioration of one or more symptoms of amyotrophic lateral sclerosis (ALS), Alzheimer's disease, and other neurological diseases and disorders.

### CROSS-REFERENCE TO RELATED APPLICATIONS

This application claims the benefit of U.S. Provisional Application No. 62/944,346, filed Dec. 5, 2019, which is incorporated by reference herein in its entirety.

### 1. FIELD

The present disclosure relates to the fields of medicine, molecular biology, and specifically to the manufacture of medicaments suitable for use in the treatment of mammalian neurodegenerative diseases. In particular, the disclosure provides improved methods for manufacturing robust, highly pure, and functional T regulatory cells useful in the treatment of diseases such as amyotrophic lateral sclerosis (ALS), Alzheimer's disease, and other neurological, as well as inflammatory and autoimmune diseases or dysfunctions.

### 2. BACKGROUND

The clinical manufacturing of expanded autologous CD4⁺CD25^{high}FOXP3⁺ T regulatory cells (Tregs) presents several logistical and cost-related challenges that impede the availability of these therapies to patients who may benefit from their use. Current Treg manufacturing conditions require activation and expansion protocols that are complicated and labor-intensive. Further challenges include the time required to expand the Tregs to the necessary doses and the formulation of a final cryopreserved product after expansion that maintains cellular viability, integrity and function. Despite these challenges, autologous Treg therapies are currently being tested in Phase I clinical studies for Graft-Versus-Host Disease (GvHD) and several autoimmune diseases including type 1 diabetes and Crohn's disease. The therapeutic value of increasing Treg activity is supported by the fact that the efficacy of many immunosuppressive drugs is contingent upon their abilities to stimulate Tregs. Treg therapies may be more advantageous than immunosuppressant drugs as they may limit off target effects, thus improving efficacy and minimizing adverse effects. Therefore, the development of a manufacturing process for the robust production of highly pure and functional Tregs is crucial for future applications of Treg therapies.

### 3. SUMMARY

Treg adoptive cell therapies hold great promise for treating patients with a wide variety of disorders. A challenge to fulfilling the potential of such therapies, however, is to be able to efficiently and quickly produce large numbers of Tregs that exhibit high purity, viability and suppressive potency that can be stored and ready for administration to patients. The methods presented herein address this challenge, by providing methods of producing ex vivo-expanded Treg cell populations that exhibit exemplary viability and purity and potency. Remarkably, the methods presented herein also yield cryopreserved therapeutic populations of expanded Tregs that following thawing and without further expansion maintain the desirable purity, viability and potency characteristics of the expanded Tregs prior to cryopreservation. Thus, the methods presented herein provide the ability to produce potent ex vivo-expanded Treg cell populations that may be utilized as off-the-shelf therapeutics.

The methods presented herein yield unique ex vivo-expanded Treg cell populations. The Tregs described and produced herein exhibit high viability and purity as well as potent suppressive activities, characterized by both an ability to suppress T responder cells as well as a surprising ability to suppress inflammatory cell, e.g., macrophage, activity. As demonstrated herein, the ability to inhibit inflammatory cell, e.g., macrophage, activity is absent in freshly isolated, non-expanded Tregs obtained from either healthy or disease, e.g., ALS donors. Thus, the ex vivo-expanded Tregs presented herein are distinct from freshly isolated Tregs and, in fact, may be characterized as “super-supressor” Tregs. Moreover, as also demonstrated herein, the Treg cell populations described herein exhibit unique gene products signatures.

Thus, also presented herein are ex vivo-expanded Treg cell populations, pharmaceutical compositions comprising such Treg cell populations, cryopreserved ex vivo-expanded therapeutic Treg populations, and pharmaceutical compositions comprising such cryopreserved Tregs following their thawing and without further expansion.

Also presented herein are methods of treatment that utilize the Treg cell populations produced and described herein, including, for example, treatment of neurodegenerative disorders such as amyotrophic lateral sclerosis (ALS), Alzheimer's disease, Parkinson's disease and frontotemporal dementia

In one aspect, provided herein is a method of producing a cryopreserved therapeutic population of regulatory T cells (Tregs), said method comprising the steps of (1) enriching Tregs from a cell sample suspected of containing Tregs, to produce a baseline Treg cell population; (2) expanding the baseline Treg cell population to produce an expanded Treg cell population, wherein the baseline Treg cell population is not cryopreserved prior to the initiation of step (b); and (3) cryopreserving the expanded Treg cell population to produce a cryopreserved therapeutic population of Tregs. In this context, the term “baseline,” or “baseline Treg cell population denotes a population of Tregs that has been enriched from a patient sample but has not yet been expanded. In some embodiments, the expanded Treg cell population and the cryopreserved therapeutic population of Tregs, following thawing and without additional expansion, exhibit an ability to suppress inflammatory cells, as measured by pro-inflammatory cytokine production by the inflammatory cells, wherein the inflammatory cells are macrophages or monocytes from human donors or generated from induced pluripotent stem cells. In some embodiments, the ability of the cryopreserved therapeutic population of Tregs, following thawing and without additional expansion, to suppress inflammatory cells is at least 70% that of the expanded Treg cell population.

In some embodiments, the ability to suppress inflammatory cells is measured by IL-6, TNFα, IL1β, IL8, and/or Interferon-7 production by the inflammatory cells. In some embodiments, the ability to suppress inflammatory cells is measured by IL-6 production by the inflammatory cells. In some embodiments, the cryopreserved therapeutic population of Tregs, following thawing and without additional expansion, exhibits a suppressive function, wherein the suppressive function is greater than that of the baseline Treg cell population, as determined by suppression of proliferation of responder T cells. In some embodiments, the suppressive function of the cryopreserved therapeutic population of Tregs, following thawing and without additional expansion, is at least 25%, at least 50%, at least 75%, at least 100% at least 150%, or at least 300% greater than the suppressive function of the baseline Treg cell population as determined by suppression of proliferation of responder T cells. In some embodiments, the cryopreserved therapeutic population of Tregs, following thawing and without additional expansion, exhibits a suppressive function, wherein the suppressive function is at least 50%, at least 60%, at least 70%, at least 75%, at least 80%, at least 85%, at least 90% or at least 95%, as determined by suppression of proliferation of responder T cells. In some embodiments, the suppressive function of the cryopreserved therapeutic population of Tregs, following thawing and without additional expansion, is at least 50%, at least 60%, at least 70%, at least 75%, at least 80%, at least 85%, at least 90% or at least 95% of the suppressive function of the expanded Treg cell population before cryopreservation. In some embodiments, the proliferation of responder T cells is determined by flow cytometry or thymidine incorporation.

In some embodiments, the viability of the cryopreserved therapeutic population of Tregs, following thawing and without additional expansion, is at least 70%, at least 75%, at least 80%, at least 85%, at least 90% or at least 95%, as determined by trypan blue staining. In some embodiments, the viability of the cryopreserved therapeutic population of Tregs, following thawing and without additional expansion, is at least 70%, at least 75%, at least 80%, at least 85%, at least 90% or at least 95% of the viability of the expanded Treg cell population prior to the expanded Treg cell population being cryopreserved in step (c), as determined by trypan blue staining.

In some embodiments, the cryopreserved therapeutic population of Tregs comprises FoxP3+ Tregs wherein the proportion of FoxP3+ Tregs is increased relative to the proportion of FoxP3+ Tregs in the Tregs in the baseline Treg cell population. In some embodiments, the cryopreserved therapeutic population of Tregs comprises at least 40%, at least 50%, at least 60%, at least 70%, or at least 80% or at least 90% FoxP3+ Tregs, as determined by flow cytometry. In some embodiments, the cryopreserved therapeutic population of Tregs comprise FoxP3-expressing Tregs wherein the expression of FoxP3 is increased in the Tregs relative to expression of FoxP3 in the Tregs in the baseline Treg cell population.

In some embodiments, the cryopreserved therapeutic population of Tregs comprises at least 70%, at least 75%, at least 80%, at least 85%, at least 90% or at least 95% CD4⁺CD25⁺ cells, as determined by flow cytometry. In some embodiments, the cryopreserved therapeutic population of Tregs comprises fewer than 20% CD8⁺ cells, as determined by flow cytometry. In some embodiments, the cryopreserved therapeutic population of Tregs comprises at least 70%, at least 80%, or at least 90% CD4⁺CD25^{high}CD127^{low} Tregs, as determined by flow cytometry.

In some embodiments, the cell sample is a leukapheresis cell sample. In some embodiments, the method further comprises obtaining the cell sample from a donor by leukapheresis. In some embodiments, the cell sample is not stored overnight or frozen before carrying out the enriching step (a). In some embodiments, the cell sample is obtained within 30 minutes before initiation of enriching step (a). In some embodiments, step (a) comprises depleting CD8⁺/CD19⁺ cells then enriching for CD25⁺ cells. In some embodiments, step (b) is carried out within 30 minutes after step (a).

In some embodiments, step (b) comprises culturing the Tregs in a culture medium that comprises beads coated with anti-CD3 antibodies and anti-CD28 antibodies. In some embodiments, the beads are first added to the culture medium within about 24 hours of the initiation of the culturing. In some embodiments, beads coated with anti-CD3 antibodies and anti-CD28 antibodies are added to the culture medium about 14 days after beads coated with anti-CD3 antibodies and anti-CD28 antibodies were first added to the culture medium.

In some embodiments, step (b) further comprises adding IL-2 to the culture medium within about 6 days of the initiation of culturing. In some embodiments, step (b) further comprises replenishing the culture medium with IL-2 about every 2-3 days after IL-2 is first added to the culture medium.

In some embodiments, step (b) further comprises adding rapamycin to the culture medium within about 24 hours of the initiation of the culturing. In some embodiments, step (b) further comprises replenishing the culture medium with rapamycin every 2-3 days after the rapamycin is first added to the culture medium.

In some embodiments, the cryopreserving step (c) is carried out at least 6 days following IL-2 addition to or replenishment of the culture medium in step (b). In some embodiments, the cryopreserving step (c) is carried out about 8-25 days after the initiation of the culturing step (b).

In some embodiments, step(c) comprises cryopreserving the Tregs in a cryoprotectant comprising DMSO. In some embodiments, the cryopreservation step (c) comprises changing the temperature of the population of Tregs in the following increments: 1° C./min to 4° C., 25° C./min to −40° C., 10° C./min to −12° C., 1° C./min to −40° C., and 10° C./min to −80° C.-−90° C. In some embodiments, the cryopreserved therapeutic population of Tregs is frozen at a Treg density of at least 50 million cells/mL. In some embodiments, the cryopreserved therapeutic population of Tregs is frozen in a total volume of 1-1.5 mL. In some embodiments, the method further comprises thawing the cryopreserved therapeutic population of Tregs after cryopreservation for about 1 week, 1 month, about 3 months, about 6 months, about 9 months, about 12 months, about 18 months or about 24 months.

In some embodiments, the cell sample is from a human donor. In some embodiments, the human donor is a healthy donor. In other embodiments, the human donor is diagnosed with or suspected of having a neurodegenerative disorder. In some embodiments, the neurodegenerative disorder is amyotrophic lateral sclerosis, Alzheimer's disease, Parkinson's disease or frontotemporal dementia.

In some embodiments, the population of Tregs is subjected to genetic engineering at any point of the method prior to cryopreserving step (c).

In some embodiments, step (b) is automated. In some embodiments, step (b) takes place in a bioreactor. In some embodiments, In some embodiments, step (b) takes place in a G-REX culture system. In some embodiments, the method is performed in a closed system.

In some embodiments, the method further comprises thawing the cryopreserved therapeutic population of Tregs and, without further expansion, placing the population into a pharmaceutical composition comprising a pharmaceutically acceptable carrier, to produce a Treg pharmaceutical composition. In some embodiments, the Treg pharmaceutical composition comprises normal saline and 5% human serum albumin.

In some embodiments, the method further comprises administering the Treg pharmaceutical composition to a human subject. In some embodiments, the Tregs in the pharmaceutical composition are autologous to the human subject. In some embodiments, the human subject has been diagnosed with or is suspected of having a neurodegenerative disorder. In some embodiments, the neurodegenerative disorder is amyotrophic lateral sclerosis (ALS), Alzheimer's disease, Parkinson's disease or frontotemporal dementia.

In another aspect, provided herein is a cryopreserved therapeutic population of Tregs produced by the method provided herein.

In another aspect, provided herein is a pharmaceutical composition comprising a cryopreserved therapeutic population of Tregs produced by a method provided herein, following thawing and without further expansion, and a pharmaceutically acceptable carrier.

In another aspect, provided herein is an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells. In some embodiments, the ability to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells. In some embodiments, the Treg cell population is autologous to a human subject with ALS. In some embodiments, the Treg cell population has been expanded from a cell sample from a human subject with ALS.

In another aspect, provided herein is a pharmaceutical composition comprising an ex vivo-expanded Treg cell population provided herein and a pharmaceutically acceptable carrier.

In another aspect, provided herein is a cryopreserved therapeutic population of ex vivo-expanded Tregs that, following thawing and without additional expansion, exhibits an ability to suppress inflammatory cells, as measured by pro-inflammatory cytokine production by the inflammatory cells, wherein the inflammatory cells are macrophages or monocytes from human donors or generated from induced pluripotent stem cells. In some embodiments, the ability of the cryopreserved therapeutic population of ex vivo-expanded Tregs to suppress inflammatory cells, following expansion and without additional expansion, is at least 70%, that of the ex vivo-expanded Tregs prior to cryopreservation. In some embodiments, the ability to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells.

In some embodiments, the cryopreserved therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% as determined by suppression of proliferation of responder T cells by flow cytometry or thymidine incorporation. In some embodiments, the cryopreserved therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% of the suppressive function of the ex vivo-expanded Tregs prior to cryopreservation, as determined by suppression of proliferation of responder T cells by flow cytometry or thymidine incorporation. In some embodiments, the cryopreserved therapeutic population of ex vivo-expanded Tregs, following thawing and without additional expansion, exhibits at least 70%, at least 75%, at least 80%, at least 85%, at least 90% or at least 95% viability, as determined by trypan blue staining. In some embodiments, the cryopreserved therapeutic population of ex vivo-expanded Tregs, following thawing and without additional expansion, exhibits a viability that is at least 70%, at least 75%, at least 80%, at least 85%, at least 90% or at least 95% of the viability of the Tregs before cryopreservation, as determined by trypan blue staining.

In some embodiments, the cryopreserved therapeutic population of ex vivo-expanded Tregs, following thawing and without additional expansion, comprises at least 40%, at least 50%, at least 60%, at least 70%, or at least 80% or at least 90% FoxP3+ Tregs, as determined by flow cytometry. In some embodiments, the cryopreserved therapeutic population of ex vivo-expanded Tregs, following thawing and without additional expansion, comprises at least 70%, at least 75%, at least 80%, at least 85%, at least 90% or at least 95% CD4⁺CD25⁺ cells, as determined by flow cytometry. In some embodiments, the cryopreserved therapeutic population of ex vivo-expanded Tregs, following thawing and without additional expansion, comprises fewer than 20% CD8+ cells, as determined by flow cytometry. In some embodiments, the cryopreserved therapeutic population of ex vivo-expanded Tregs, following thawing and without additional expansion, comprises at least 70%, at least 80%, or at least 90% CD4⁺CD25^{high}CD127^{low} Tregs, as determined by flow cytometry. In some embodiments, the ex vivo-expanded Tregs are autologous to a human subject with ALS. In some embodiments, the ex vivo-expanded Tregs have been expanded from a cell sample from a human subject with ALS.

In another aspect, provided herein is a pharmaceutical composition comprising the cryopreserved therapeutic population of Tregs provided herein, following thawing and without further expansion, and a pharmaceutically acceptable carrier.

In another aspect, provided herein is an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population: (a) expression of one or more dysfunctional baseline signature gene products listed in Table 12 and/or Table 13 is decreased relative to the expression of the one or more gene products in baseline Tregs; (b) expression of one or more dysfunctional baseline signature gene products listed in Table 14 is decreased relative to the expression of the one or more gene products in baseline Tregs; (c) expression of one or more Treg-associated signature gene products listed in Table 15 is increased relative to the expression of the one or more gene products in baseline Tregs; (d) expression of one or more mitochondria signature gene products listed in Table 16 is increased relative to the expression of the one or more gene products in baseline Tregs; (e) expression of one or more cell proliferation signature gene products listed in Table 17 is increased relative to the expression of the one or more gene products in baseline Tregs; or (f) expression of one or more highest protein expression signature gene products listed in Table 18 is increased relative to the expression of the one or more gene products in baseline Tregs.

In some embodiments, the ability to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells.

In some embodiments, in the ex vivo-expanded Treg cell population, expression of one or more of the following gene products is increased relative to expression of the one or more gene products in baseline Tregs: ADAM10, AIMP1, AIMP2, ARG2, BCL2L1, BSG, CD2, CD28, CD38, CD74, CD84, CTLA4, FAS, FOXP3, GCLC, HAT1, HLA-DQA1, HLA-DQB1, HLA-DRA, HLA-DRB1, HPGD, ICOS, IL1RN, IRF4, KPNA2, LGALS1, LGMN, PCNA, POFUT1, SATB1, SELPLG, STAT1, TFRC and TNFRSF18.

In some embodiments, in the ex vivo-expanded Treg cell population, expression of one or more of the following gene products is increased relative to expression of the one or more gene products in baseline Tregs: ACAA2, ACADM, ACADVL, ACOT7, ACSL1, ACSL4, ACSL5, AGK, AGMAT, AK4, ARG2, ARL2, AUH, BCL2L1, BDH1, BNIP1, CDK1, CHDH, CIAPIN1, CISD2, COX17, CPOX, CPT1A, CPT2, CYB5B, DAP3, DHRS2, DNM1L, DUT, DYNLL1, ECI1, FDXR, FEN1, FKBP8, GK, GRSF1, HTRA2, L2HGDH, LACTB2, LRPPRC, MAIP1, MAOA, MPST, MRPL1, MRPL12, MRPL13, MRPL14, MRPL17, MRPL22, MRPL37, MRPL39, MRPL4, MRPL43, MRPL44, MRPL46, MRPL48, MRPS11, MRPS14, MRPS2, MRPS27, MRPS31, MRPS35, MRPS9, MTHFD2, MTX1, MYCBP, NDUFA8, NUDT1, OAT, PITRM1, PLSCR3, PMPCA, PPIF, PTRH2, PYCR2, REXO2, RMND1, SFXN2, SLC25A10, SLC25A19, SLC25A4, TIGAR, TIMM13, TIMM23, TMEM14C, TOMM22, TOMM34, TOMM40, and TST.

In some embodiments, in the ex vivo-expanded Treg cell population, expression of one or more of the following gene products is increased relative to expression of the one or more gene products in baseline Tregs: ARL2, ARL3, BCCIP, CCDC124, CDK1, CDK2, CDK5, CDK6, CUL4B, DCTN3, FEN1, HELLS, LIG1, MAD2L1, MAEA, MCM2, MCM2, MCM3, MCM4, MCM5, MCM6, MCM7, MCMBP, NUDC, PCNA, POLD1, POLD2, RALB, RBM38, RFC2, RFC3, RFC4, RFC5, RNASEH2A, RNASEH2B, and SMC2.

In some embodiments, in the ex vivo-expanded Treg cell population, expression of one or more of the following gene products is increased relative to expression of the one or more gene products in baseline Tregs: ACAA2, ACADM, ACADVL, ACOT7, BSG, CACYBP, CD74, CDK1, CPOX, DUT, ECI1, ENO3, FEN1, FKBP3, HIST1H2BJ, HLA-DQA1, HLA-DRA, HLA-DRB1, LGALS1, LGALS3, MCM5, MCM6, MCM7, MTHFD1, NAMPT, NME1, NQO1, PCNA, RAB1A, RALB, SLC25A4, STAT1, STMN1, STMN2, TUBAIB, TUBB4A, TUBB8, TXN, TXNRD1, and WARS.

In some embodiments, in the ex vivo-expanded Treg cell population, expression of one or more dysfunctional baseline signature gene products listed in Table 12 and/or Table 13 is decreased relative to the expression of the one or more gene products in baseline Tregs.

In another aspect, provided herein is an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population, expression of one or more Treg-associated signature gene products listed in Table 14 is increased relative to the expression of the one or more gene products in baseline Tregs. In some embodiments, in the ex vivo-expanded Treg cell population expression of one or more dysfunctional baseline signature gene products listed in Table 12 and/or Table 13 is decreased relative to the expression of the one or more gene products in baseline Tregs.

In some embodiments, the ex vivo-expanded Treg cell population exhibits a suppressive function, wherein the suppressive function is at least 50%, at least 60%, at least 70%, at least 75%, at least 80%, at least 85%, at least 90% or at least 95% as determined by suppression of proliferation of responder T cells by flow cytometry or thymidine incorporation. In some embodiments, the ex vivo-expanded Treg cell population exhibits a suppressive function, wherein the suppressive function is at least 50%, at least 75%, at least 100%, or at least 150% that of baseline Tregs, as determined by suppression of proliferation of responder T cells by flow cytometry or thymidine incorporation. In some embodiments, the ex vivo-expanded Tregs are autologous to a human subject with ALS. In some embodiments, the ex vivo-expanded Tregs have been expanded from a cell sample from a human subject with ALS.

In some embodiments, the expression of the one or more gene products is changed by a log 2 change of at least about 4-fold, at least about 5-fold, at least about 6-fold, at least about 7-fold, at least about 8-fold, at least about 9-fold, or at least about 10-fold. In some embodiments, expression is determined by single-shot proteomic analysis.

In another aspect, provided herein is a pharmaceutical composition comprising the ex vivo-expanded Treg cell population provided herein, following thawing and without t further expansion, and a pharmaceutically acceptable carrier.

In another aspect, provided herein is a cryopreserved composition comprising a therapeutic population of ex vivo-expanded Tregs, wherein following thawing and without t further expansion, the expression of one or more of gene products listed in Table 12-Table 18 is substantially the same in therapeutic population of Tregs as the expression of the one or more gene products in the ex vivo-expanded Tregs prior to cryopreservation. In some embodiments, the one or more gene products is not also listed in Table 19. In some embodiments, the one or more gene products is a gene product associated with a dysfunctional Treg phenotype, a methylation- or epigenetics-associated gene product, a mitochondria-related gene product, or a gene product associated with the cell cycle, cell division, DNA replication or DNA repair. In some embodiments, the one or more gene products is known to be important for the proliferation, health, identification, and/or mechanism of Treg cells. In some embodiments, the therapeutic population of ex vivo-expanded Tregs, following thawing and without additional expansion, exhibits an ability to suppress inflammatory cells, as measured by pro-inflammatory cytokine production by the inflammatory cells, wherein the inflammatory cells are macrophages or monocytes from human donors or generated from induced pluripotent stem cells.

In some embodiments, the ability of the ex vivo-expanded Tregs to suppress inflammatory cells, following expansion and without additional expansion, is at least 70%, that of the ex vivo-expanded Tregs prior to cryopreservation. In some embodiments, the ability of the ex vivo-expanded Tregs to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells.

In some embodiments, the therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% as determined by suppression of proliferation of responder T cells by flow cytometry or thymidine incorporation. In some embodiments, the therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% of the suppressive function of the ex vivo-expanded Tregs prior to cryopreservation, as determined by suppression of proliferation of responder T cells by flow cytometry or thymidine incorporation. In some embodiments, the therapeutic population of ex vivo-expanded Tregs, following thawing and without additional expansion, exhibits at least 70%, at least 75%, at least 80%, at least 85%, at least 90% or at least 95% viability, as determined by trypan blue staining.

In some embodiments, the therapeutic population of ex vivo-expanded Tregs, following thawing and without additional expansion, exhibits a viability that is at least 70%, at least 75%, at least 80%, at least 85%, at least 90% or at least 95% of the viability of the Tregs before cryopreservation, as determined by trypan blue staining.

In some embodiments, the therapeutic population of ex vivo-expanded Tregs, following thawing and without additional expansion, comprises at least 40%, at least 50%, at least 60%, at least 70%, or at least 80% or at least 90% FoxP3+ Tregs, as determined by flow cytometry. In some embodiments, the therapeutic population of ex vivo-expanded Tregs, following thawing and without additional expansion, comprises at least 70%, at least 75%, at least 80%, at least 85%, at least 90% or at least 95% CD4⁺CD25⁺ cells, as determined by flow cytometry. In some embodiments, the therapeutic population of ex vivo-expanded Tregs, following thawing and without additional expansion, comprises fewer than 20% CD8+ cells, as determined by flow cytometry. In some embodiments, the therapeutic population of ex vivo-expanded Tregs, following thawing and without additional expansion, comprises at least 70%, at least 80%, or at least 90% CD4⁺CD25^{high}CD127^{low} Tregs, as determined by flow cytometry. In some embodiments, the ex vivo-expanded Tregs are autologous to a human subject with ALS. In some embodiments, the ex vivo-expanded Tregs have been expanded from a cell sample from a human subject with ALS. In some embodiments, gene product expression is determined by single-shot proteomic analysis.

In another aspect, provided herein is a pharmaceutical composition comprising the cryopreserved composition comprising a therapeutic population of ex vivo-expanded Tregs provided herein following thawing and without further expansion, and a pharmaceutically acceptable carrier.

In another aspect, provided herein is a method of treating a disorder associated with Treg dysfunction, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating a disorder associated with Treg deficiency, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating a disorder associated with overactivation of the immune system, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating an inflammatory condition driven by a T cell response, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating an inflammatory condition driven by a myeloid cell response, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering. In some embodiments, the myeloid cell is a monocyte, macrophage or microglia.

In another aspect, provided herein is a method of treating a neurodegenerative disorder in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the neurodegenerative disease is Amyotrophic Lateral Sclerosis (ALS), Alzheimer's disease, Parkinson's disease, frontotemporal dementia or Huntington's disease.

In another aspect, provided herein is a method of treating an autoimmune disorder in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, systemic lupus erythematosus, cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

In another aspect, provided herein is a method of treating graft-versus-host disease in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the subject has received a bone marrow transplant, kidney transplant or liver transplant.

In another aspect, provided herein is a method of improving islet graft survival in a subject in need thereof, comprising: combining islet transplantation with administering to the subject a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating cardio-inflammation in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the cardio-inflammation is associated with atherosclerosis, myocardial infarction, ischemic cardiomyopathy or heart failure.

In another aspect, provided herein is a method of treating neuroinflammation in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

In another aspect, provided herein is a method of treating a Tregopathy in a subject in need thereof, comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments of the methods of treatment provided herein, the Tregs are autologous to the subject. In other embodiments of the methods of treatment provided herein, the Tregs are allogeneic to the subject.

In some embodiments of the methods of treatment provided herein, the composition is a pharmaceutical composition provided herein.

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

### 4. BRIEF DESCRIPTION OF THE FIGURES

FIG. 1A-1D: Treg percentage and suppressive function increased during each round of Treg infusions. Arrows and vertical dotted lines represent Treg infusions. The 1^{st} Treg infusion was administered on week 0 and then every 2 weeks for a total of 4 infusions. The 5^{th} Treg infusion was administered in each participant on weeks 48, 27 and 33, respectively, and then every 4 weeks for a total of 4 infusions. (FIG. 1A—Participant #1, FIG. 1B—Participant #2 and FIG. 1C—Participant #3) The percentage of CD4⁺CD25⁺FOXP3⁺ Tregs within the total CD4⁺ cell population is shown. Treg percentages are shown at baseline (weeks −4.6, −3.0 and −4.9 in each participant, respectively), the days of the 1^{st} and 5^{th} Treg infusions, the day after each Treg infusion, every 2 weeks during each round of infusions, and 1 month after each round. The data point collected the day after the 4^{th} Treg infusion (week 6) in participant #3 was not determined due to a flow staining error. (FIG. 1D—Participant #1, FIG. 1E—Participant #2 and FIG. 1F—Participant #3) Treg suppressive function is shown on simultaneous days as the Treg percentage.

FIGS. 2A-2F: Disease progression slowed during each round of Treg infusions and correlated with increased Treg suppressive function. Arrows and vertical dotted lines represent Treg infusions. (FIG. 2A—Participant #1, FIG. 2B—Participant #2 and FIG. 2C—Participant #3) Clinical progression is depicted by the ALSFRS-R (white points) and AALS (black points). Clinical progression lines during each round of Treg infusions are enlarged in side panels for the early (1) and later (2) stages of disease. (FIG. 2D—Participant #1, FIG. 2E—Participant #2 and FIG. 2F—Participant #3) Correlation between changes in the AALS and Treg suppressive function is shown (Participant #1; ρ=−0.60, p=0.003, Participant #2; ρ=−0.71, p=0.0026, and Participant #3; ρ=−0.54, p=0.016). Lines represent the best fit as determined by linear regression analysis. Data were analyzed by Spearman's correlation and p values less than 0.05 were considered significant.

FIGS. 3A-3F: Maximal inspiratory pressures stabilized during Treg infusions. Arrows and vertical dotted lines represent Treg infusions. (FIG. 3A—Participant #1, FIG. 3B—Participant #2 and FIG. 3C—Participant #3) The forced vital capacity (FVC) measurements are represented as % predicted values. Measurements are shown at baseline (weeks −4.6, −3.0 and −4.9 in each participant, respectively), immediately prior to each Treg infusion, every 2 weeks during each round of infusions, and 1 month after each round. A solid gray line connects the points between each round of infusions. (FIG. 3D—Participant #1, FIG. 3E—Participant #2 and FIG. 3F—Participant #3) Measurements of maximal inspiratory pressure (MIP) are shown in cm H₂O. MIPs are shown at the same time points as FVC measurements. The MIP values were erroneously not determined for Participant #1 immediately prior to the 5^{th} Treg infusion and for Participant #3 one month after the second round of infusions.

FIGS. 4A-4F show suppressor T cells immunophenotype in Alzheimer disease. FIG. 4A: Gating strategy to identify CD4 and CD8 suppressor T cells; Lymphocytes were delineated by forward/side scatter gating. CD3 was used to identify T cells among the previously selected viable lymphocytes. CD4 T cells and CD8 T cells were identified as uniquely expressing CD4 or CD8 antigens. Tregs were defined as CD4 T cells co-expressing CD25 and FOXP3. The expressions of CD25 and FoxP3 were then determined on CD8 T cells. FIG. 4B: CD4⁺FOXP3⁺CD25^{high} T cell percentage (% of total CD4), did not differ among HC, MCI and Alzheimer groups. FIG. 4C: FoxP3 mean fluorescent intensity in CD4⁺FOXP3⁺CD25^{high} cell population was comparable among the three groups. FIG. 4D) CD25 mean fluorescent intensity in the CD4⁺FOXP3⁺CD25^{high} cell population was reduced in Alzheimer dementia stage. FIG. 4E and FIG. 4F) The percentages of CD8⁺CD25^{high} and CD8⁺FOXP⁺ suppressor T cells (% of total CD8) were increased in MCI patients, compared to HCs. CD8⁺CD25⁺ T cell population differs significantly between MCI and Alzheimer. P-values are *p<0.05 and **p<0.01.

FIGS. 5A and 5B show the Treg suppressive function on Tresp proliferation. CD4⁺CD25^{high} Tregs were cocultured with CD4⁺CD25^{neg} Tresps and proliferation was determined by ³H-thymidine incorporation. FIG. 5A: No correlation between age and suppressive activity of Tregs on Tresp proliferation (1:1 ratio) was noted. FIG. 5B: Suppression (%) of Tregs on Tresp proliferation in MCI vs. HC in 1:1 and 2:1 Tresps:Tregs ratios were comparable. Suppression of Tregs on Tresps proliferation in Alzheimer disease in both 1:1 and 2:1 Tresps:Tregs ratios were reduced compared to both MCI and HC. P-values are *p<0.05, **p<0.01, and ***p<0.001.

FIGS. 6A-6C show the Treg immunophenotype and suppressive function following ex vivo expansion. FIG. 6A: In a second group of individuals, suppression (%) of CD4⁺CD25^{high} Tregs on Tresp proliferation (1:1 ratio) at baseline (base) was compromised in Alzheimer patients (n=10), compared with HC (n=10). Following 10 days of ex-vivo expansion, amplified suppressive function of expanded (exp) Tregs on Tresp proliferation was noted in all groups. CD25 (FIG. 6B) and FoxP3 (FIG. 6C) MFIs in CD4⁺CD25^{high}Treg population were elevated following ex vivo expansion. P-values are *p<0.05, **p<0.01, and ***p<0.001.

FIGS. 7A-7E show the suppression of Tregs on iPSC-derived pro-inflammatory macrophages. Baseline and ex vivo expanded CD4⁺CD25^{high} Tregs were added to iPSC-derived pro-inflammatory macrophages (M1) and relative change (% of M1-only) of pro-inflammatory cytokines were measured. FIG. 7A and FIG. 7B: At baseline, a trend toward decreased macrophage IL6 transcript expression was noted following co-culture with baseline HC Tregs, however this trend was not observed from IL-6 protein levels. Expanded Tregs of HC, MCI and Alzheimer's suppressed M1-IL6 transcript and protein expressions. FIG. 7C and FIG. 7D: show the co-culture of baseline Tregs with M1 did not suppress TNFα transcript and protein levels. Reduction of M1-TNFα, transcript and protein expressions were noted following co-culture with expanded Tregs in all three groups. FIG. 7E: Baseline Tregs did not attenuate IL1B transcript level. Expanded Tregs of Alzheimer disease, MCI and HC displayed an enhanced capacity to suppress M1-derived IL1B transcript. *p<0.05, **p<0.01, ***p<0.001 vs. their corresponding baseline Tregs-M1 co-cultures; ##p<0.01, ###p<0.001 vs. M1-alone cultures.

FIG. 8A and FIG. 8B show the transcript expression profile of immunoregulatory genes in Tregs. Expression profiles (FIG. 8A) of anti-inflammatory cytokines (TGF-β, IL-4, IL-10, IL-13), CD25 and (FIG. 8B) Proximity or contact mediated immunoregulatory markers (Granzyme (GZM) A, CD39, PDL1, PDL2, CTLA4, CD73, GZMB, PD1) were evaluated in expanded and baseline Tregs of HC (n=10), MCI (n=10) and Alzheimer patients (n=10). The data were obtained from qPCR analysis when normalized using β-actin. The transcript levels of IL-13, CD25 and proximity or contact mediated immunoregulatory markers (PD1, GZMB and CD73) were up regulated following ex vivo expansion. P-values are *p<0.05, **p<0.01, and ***p<0.001 vs. their corresponding baseline Tregs.

FIGS. 9A-9H show protein expression of immunoregulatory genes in Tregs. Using flow cytometer, protein expression of immunoregulatory genes (CD73, PD1, IL-13 and GZMB) were analyzed in baseline and corresponding ex vivo expanded Tregs of Alzheimer patients (n=5) and HCs (n=5). Increase in the percentage of (FIG. 9A) CD73⁺, (FIG. 9B) PD1⁺ and (FIG. 9C) IL13⁺ Tregs (% of total CD4⁺CD25^{high} Tregs) were noted following ex vivo expansion in both Alzheimer patients and HCs. The change in the percentage of (FIG. 9D) GZMB⁺ Tregs following expansion were not statistically significant. Mean fluorescence intensities (MFIs) of (FIG. 9E) CD73, (FIG. 9F) PD1 and (FIG. 9H) GZMB were amplified in expanded Tregs. The increase in (FIG. 9G) IL13 MFI in Tregs following expansion was not statistically significant. P-values are *p<0.05, **p<0.01, and ***p<0.001;

FIG. 10A and FIG. 10B show the suppressive mechanism of ex vivo expanded Tregs. Expanded Tregs of Alzheimer patients (n=4) (FIG. 10A) and HC (n=4) (FIG. 10B) were co-cultured with pro-inflammatory macrophages (M1) and relative fold change of IL-6 protein (% of M1-only) were assayed in the presence of IL-13 or CD25 neutralizing antibodies (IL-13 NA and IL-25 NA) or transwells. The absence of direct cell-cell contact by using transwells, blocked suppressive function of ex vivo expanded Tregs. P-values are **p<0.01;

FIGS. 11A and 11B show the purity of expanded and freshly isolated Tregs as measured by the percentage of CD4⁺CD25^{high} (FIG. 11A) or CD4⁺CD25^{high}CD127^{low} (FIG. 11B) cells in a population of CD4+ cells as determined by flow cytometry. N=3, ** indicates p-value of 0.01 or less.

FIGS. 12A and 12B show the expression of CD25 protein (FIG. 12A) and CD127 protein (FIG. 12B) in expanded and freshly isolated Tregs as measured by flow cytometry. N=3, ** indicates p-value of 0.05 or less.

FIGS. 12A and 12B show the granularity and size, respectively, of expanded and freshly isolated Tregs. These data indicate that expanded Tregs are larger than freshly isolated ones and that expanded Tregs have more granularity than freshly isolated ones.

FIG. 14: Process flow diagram for the process of Treg isolation and expansion for patients with Amyotrophic Lateral Sclerosis (ALS).

FIG. 15: Assembling GE Healthcare-Biosafe CS-490.1 kit (PeriCell).

FIG. 16: GE Healthcare Biosafe CS-900.2 kit.

FIG. 17: CliniMACS Tubing Set LS (162-01).

FIG. 18: CliniMACS® Plus Instrument.

FIG. 19: Expansion curves for 12 populations of Tregs.

FIG. 20 shows Viability of Cryopreserved Treg Products. Graph shows results for three validation runs.

FIG. 21 shows Purity of Final Treg Products from FDA Validation Runs. Graph shows results for three validation runs.

FIG. 22 shows Potency of Cryopreserved Treg Products. Graph shows results for three validation runs. Missing bars indicate no data could be collected.

FIG. 23 shows Viability of Final Treg Products from Phase 2a Clinical Trial. Groups of bars show, starting from the, results for Subject Nos. 701-115, 701-114, 701-103, 702-206, 702-205, 702-204, 702-203, 702-202 and 702-201.

FIG. 24 shows Purity of Final Treg Products from Phase 2a Clinical Trial. Groups of bars show, starting from the, results for Subject Nos. 701-115, 701-114, 701-103, 702-206, 702-205, 702-204, 702-203, 702-202 and 702-201.

FIG. 25 shows the potency of Final Treg Products from Phase 2a Clinical Trial. Graph shows, starting from the, results for Subject Nos. 701-115, 701-114, 701-103, 702-206, 702-205, 702-204, 702-203, 702-202 and 702-201.

FIG. 26 shows lack of suppression of proinflammatory macrophages freshly isolated Tregs in vitro. Conditions listed are depicted in the graph from left to right for the Strong M1 (left group of bars) and Weak M1 (right group of bars) experiments.

FIG. 27 shows suppression of proinflammatory macrophages by expanded/cryopreserved Tregs in vitro. Numbers show percentage decrease compared to No Treg control; * indicates a p-value of 0.05 or less, ** indicates a p value of 0.01 or less, indicates a p-value of 0.001 or less.

FIG. 28 shows a heat map of the results of a proteomics analysis of ALS baseline Tregs, a cryopreserved therapeutic population of Tregs from ALS patients following thawing, and of the Tregs following expansion but prior to cryopreservation.

FIG. 29 shows a schematic representation of the methods employed in Example 10.

FIG. 30 shows Nucleofection of GFP-mRNA in T-cells.

FIGS. 31A-31C show Bioinformatics analysis and in-vitro validation of stable mRNA transcripts in T-cells.

FIGS. 32A and 32B show increased cell number and telomere length with hTERT expression in non-transduced and CAR-transduced Tcells. Bars in FIG. 32A and Dots in FIG. 32B show results for Day 0, Day 1 and Day 2 from left to right within each group of three.

FIGS. 33A and 33B show nucleofection of GFP and X-mRNA in T regulatory cells (Tregs).

FIG. 34 shows a summary of the mRNA therapy for improved adoptive T-cell transfer described in Example 10.

FIG. 35 shows a flow chart of an exemplary process of producing a therapeutic population of Tregs in a bioreactor.

### 5. ILLUSTRATIVE EMBODIMENTS

Illustrative embodiments of the invention are included in the text below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and/or time-consuming, but would be a routine undertaking for those of ordinary skill in the art having the benefit of this disclosure.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, and wherein the expansion step begins within about 30-90 minutes of completion of the enrichment step.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, wherein the expansion step begins within about 30-90 minutes of completion of the enrichment step, and wherein the cryopreservation step is initiated after about 15-25 days of expansion.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises culturing the Tregs in a culture medium that comprises beads coated with anti-CD3 antibodies and anti-CD28 antibodies, and (iii) comprises the addition of an expansion agent to the culture medium every 2-3 days.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within 24 h of initiating the culturing and (iii) comprises the addition of an expansion agent to the culture medium every 2-3 days.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing and (iii) comprises the addition of an expansion agent to the culture medium every 2-3 days, beginning within about 6 days of initiating the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing and (iii) comprises the addition of an expansion agent to the culture medium every 2-3 days, beginning within about 6 days of initiating the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing and (iii) comprises the addition of IL-2 to the culture medium every 2-3 days, beginning within about 6 days of initiating the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing, (iii) comprises the addition of an expansion agent to the culture medium every 2-3 days, beginning within about 6 days of initiating the culturing, and (iv) adding rapamycin to the culture medium every 2-3 days, beginning within about 24 hours of the initiation of the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b); and (c) cryopreserving the expanded Treg cell population to produce a cryopreserved therapeutic population of Tregs, wherein the enriching step begins within about 30 to 90 minutes of obtaining the leukapheresis sample, wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing, (iii) comprises the addition of an expansion agent to the culture medium every 2-3 days, beginning within about 6 days of initiating the culturing, and (iv) adding rapamycin to the culture medium every 2-3 days, beginning within about 24 hours of the initiation of the culturing, and wherein the cryopreservation step is initiated after about 15-25 days of expansion.

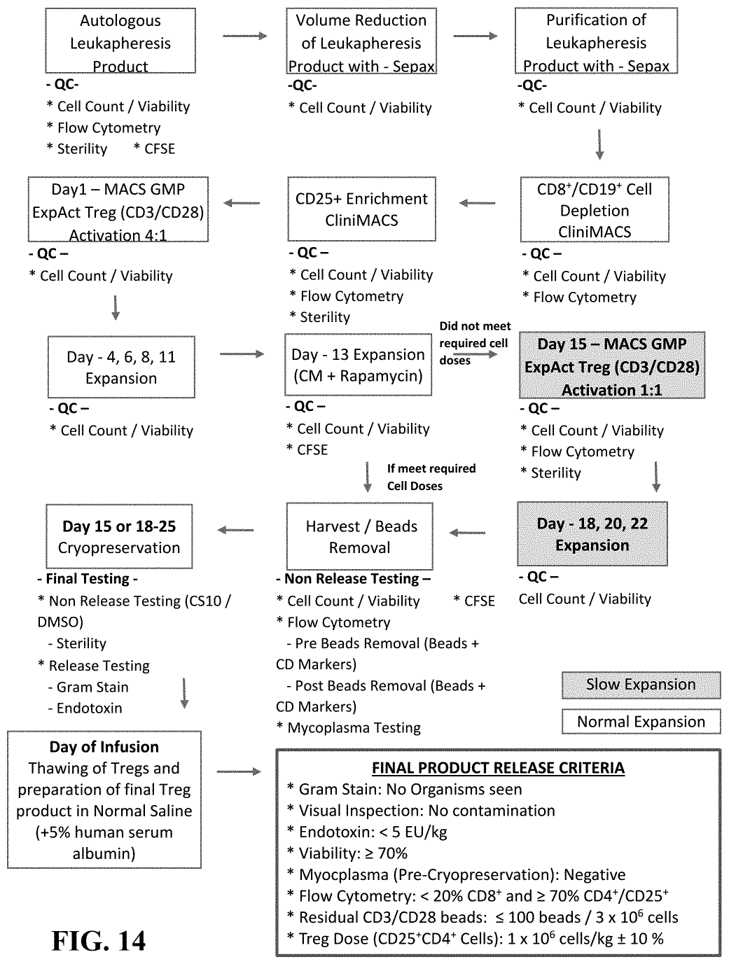

A detailed overview of an exemplary manufacturing process is depicted in FIG. 14. When isolating Tregs for clinical therapies, a CD8/CD19 depletion step followed by a CD25 enrichment step is generally employed and subsequent ex vivo expansion in the presence of IL-2, which supports the survival and proliferation of Tregs, and rapamycin, which stabilizes the Treg population. This isolation and expansion strategy reduces the amount of pro-inflammatory populations (i. e., cytotoxic CDS+ T cells and T effector cells), and increases the purity of the final Treg product. The robust manufacturing of Tregs is limited by the low number of circulating Tregs that can be isolated through leukapheresis and ex vivo separation. Hence, extensive ex vivo expansion is essential in order to obtain sufficient numbers of Tregs to treat patients for a longer period of time. The expansion of functionally compromised Tregs, for example, autologous ALS-derived Tregs, is even more challenging.

Amyotrophic lateral sclerosis, also known as Lou Gehrig's disease, is a rapidly progressive and fatal neurodegenerative disease characterized by the relentless degeneration of upper and lower motor neurons. Increasing evidence shows that dysregulation of the immune system can hasten ALS disease progression. In particular, Tregs are reduced in patients with ALS and more marked reduction is associated with more rapid disease progression. Tregs are a subpopulation of T-lymphocytes consisting of CD4⁺CD25^{hi} hFOXP3⁺ cells that suppress neuroinflammatory responses. This work is the first to identify Treg compositions as a therapy for patients with neurological disorders such as ALS. The safety and therapeutic potential of the adoptive transfer of autologous Tregs as a treatment for ALS has been demonstrated in the Phase I clinical study described in Example 1, below (see Section 8.1). In order to complete the phase 2 trial, in which a larger number of ALS patients will be treated with monthly doses of Tregs over one year, the manufacturing of at least 2 billion Tregs from each study participant is required to meet the dosing demands. Similar demands will be required of an approved therapeutic regimen.

Further, as presented herein, adoptive transfer of autologous Tregs as a treatment for Alzheimer's patients shows great promise, but Tregs from obtained from Alzheimer's patients are also functionally compromised. See Section 8.2. Thus, the expansion of autologous Alzheimer's derived Tregs for therapeutic use face similar challenges as ALS-derived Tregs.

The Treg manufacturing processes described herein overcome challenges of robust Treg expansion and cryopreservation while maintaining phenotypic characteristics and functionality. Current Treg manufacturing protocols are complicated and labor-intensive, which drive up manufacturing costs and are not sustainable as a therapy for large numbers of patients, for example patients with neurodegenerative diseases such as ALS or Alzheimer's disease. The methods described herein address manufacturing challenges at a lower cost through optimization of the manufacturing process, e.g., cGMP manufacturing processes. Moreover, the methods described herein produce an enhanced Treg product with superior suppressive functions that are maintained even when the Tregs are cryopreserved and thawed without further expansion, wherein the Tregs could be more efficacious when infused back into the patients.

For example, the improved Treg manufacturing processes described herein are critical to the advancement of developing a therapy that drastically slows disease progression in ALS because it provides a platform for current and future clinical studies in ALS, and for therapeutic ALS regimens, allows for the effective cryopreservation of ALS-derived autologous Tregs for extended treatment times with successive doses, generates functionally superior Treg products, and is potentially an “off-the-shelf immune-privileged Treg therapy that can be used for treatment of diseases, for example, neurodegenerative diseases including ALS and Alzheimer's disease, autoimmune diseases including Type 1 diabetes and rheumatoid arthritis, and graft versus host disease (GVHD) including GVHD following bone marrow transplantation.

Regulatory T cells (Tregs) account for 5-10% of CD4+ T cells in the peripheral circulation. Their dysfunction contributes to the rapid progression of amyotrophic lateral sclerosis (ALS). The suppressive functions of Tregs isolated from patients with ALS normalize following ex vivo expansion with interleukin (IL)-2 and rapamycin. Thus, expanded functional Tregs represent a potential treatment for ALS that could slow the rate of progression. However, the particular susceptibility of the Treg population to the ongoing disease process and the autologous nature of the proposed treatment pose significant challenges to the development of a Treg therapy that could combat ALS in potentially thousands of patients. A sensible Treg manufacturing process for ALS patients requires an expansion phase that yields sufficient numbers of highly suppressive Tregs to avoid exposing the patients to frequent leukapheresis procedures for Treg isolation. Frequent infusions of optimized Treg doses would be required to continually suppress the progressive neuroinflammatory environment that ensues as ALS progresses. As it would be impractical to expand Tregs from each ALS patient prior to each infusion, the development of a cryopreservation process is crucial in order to limit the per-patient costs of cell manufacturing as well as the man power that would be needed to develop Treg therapies for potentially thousands of patients with ALS. The Treg manufacturing processes described herein have been optimized to produce and cryopreserve large numbers of highly suppressive Tregs for their application in treatment regimens, for example, for their application in methods of treating disorders such as, e.g., neurodegenerative disorders such as ALS and Alzheimer's disease, autoimmune disorders such as Type 1 diabetes and rheumatoid arthritis, and graft versus host disease (GVHD) such as following a bone marrow transplantation, as well as for their application in future clinical trials for ALS patients and potentially other neurodegenerative diseases such as Alzheimer's disease. An optimized Treg therapy minimizes the number of expansion phases and infusions, which makes the therapy more cost effective and sustainable.

Further Illustrative embodiments are as follows;
1. A method of producing a cryopreserved therapeutic population of regulatory T cells (Tregs), said method comprising the steps of:
   (a) enriching Tregs from a cell sample suspected of containing Tregs, to produce a Treg-enriched cell population (baseline Treg cell population);
   (b) expanding the Treg-enriched cell population to produce an expanded Treg cell population, wherein the Treg-enriched cell population is not cryopreserved prior to the initiation of step (b); and
   (c) cryopreserving the expanded Treg cell population to produce a cryopreserved therapeutic population of Tregs.
2. The method of embodiment 1, wherein step (a) comprises enriching for CD25⁺ cells.
3. The method of embodiment 1 or 2, wherein step (a) comprises depleting CD8⁺/CD19⁺ cells.
4. The method of embodiment 1, wherein step (a) comprises depleting CD8+/CD19+ cells then enriching for CD25+ cells.
5. The method of any one of embodiments 1-4, wherein step (b) is carried out within 30 minutes after step (a).
6. The method of any one of embodiments 1-5, wherein step (b) comprises culturing the Tregs in a culture medium that comprises at least one expansion agent.
7. The method of embodiment 6, wherein the expansion agent is a CD3-activating agent.
8. The method of embodiment 7, wherein the CD3-activating agent is an anti-CD3 antibody.
9. The method of embodiment 7 or 8, wherein the CD3-activating agent is first added to the culture medium within about 24 hours of the initiation of the culturing.
10. The method of embodiment 9, wherein the CD3-activating agent is again added to the culture medium about 14 days after the CD3-activating agent was first added to the culture medium.
11. The method of embodiment 6, wherein in the expansion agent is a CD28-activating agent.
12. The method of embodiment 11, wherein the CD28-activating agent is an anti-CD28 antibody.
13. The method of embodiment 11 or 12, wherein the CD28-activating agent is first added to the culture medium within about 24 hours of the initiation of the culturing.
14. The method of embodiment 13, wherein the CD28-activating agent is again added to the culture medium about 14 days after the CD28-activating agent was first added to the culture medium.
15. The method of any one of embodiments 11-14, wherein step (b) further comprises culturing in a culture medium that comprises a CD3-activating agent.
16. The method of embodiment 15, wherein the CD3-activating agent is an anti-CD3 antibody.
17. The method of embodiment 15 or 16, wherein the CD3-activating agent is first added to the culture medium within about 24 hours of the initiation of the culturing.
18. The method of embodiment 17, wherein the CD3-activating agent is again added to the culture medium about 14 days after the CD3-activating agent was first added to the culture medium.
19. The method of embodiment 6, wherein step (b) comprises culturing the Tregs in a culture medium that comprises beads present in a bead:cell ratio of 4:1, wherein the beads are coated with anti-CD3 antibodies and anti-CD28 antibodies.
20. The method of embodiment 19, wherein the beads are first added to the culture medium within about 24 hours of the initiation of the culturing.
21. The method of embodiment 19 or 20, wherein beads coated with anti-CD3 antibodies and anti-CD28 antibodies are added to the culture medium about 14 days after beads coated with anti-CD3 antibodies and anti-CD28 antibodies were first added to the culture medium.
22. The method of embodiment 21, wherein the beads are added to the culture medium in a bead:cell ratio of 1:1.
23. The method of embodiment 6 wherein the expansion agent is IL-2.
24. The method of embodiment 23, wherein the TL-2 is present in the culture medium at a concentration of 500 IU/ml.
25. The method of embodiment 23 or 24, wherein the IL-2 is first added to the culture medium within about 6 days of the initiation of culturing.
26. The method of embodiment 25, wherein step (b) further comprises replenishing the culture medium with TL-2 about every 2-3 days after TL-2 was first added to the culture medium.
27. The method of any one of embodiments 23-26, wherein step (b) further comprises culturing in a culture medium that comprises a CD3-activating agent.
28. The method of embodiment 27, wherein the CD3-activating agent is an anti-CD3 antibody.
29. The method of any one of embodiments 23-26, wherein step (b) further comprises culturing in a culture medium that comprises a CD28-activating agent.
30. The method of embodiment 29, wherein the CD28-activating agent is an anti-CD28 antibody.
31. The method of embodiment 29 or 30, wherein step (b) further comprises culturing in a culture medium that comprises a CD3-activating agent.
32. The method of embodiment 31, wherein the CD3-activating agent is an anti-CD3 antibody.
33. The method of embodiment 23 or 24, wherein step (b) comprises adding beads coated with anti-CD3 antibodies and anti-CD28 antibodies to the culture medium, in a bead:cell ratio of 4:1, within about 24 hours of the initiation of culturing; adding the IL-2 to the culture medium within about 6 days of the initiation of culturing; and replenishing the culture medium with IL-2 every 2-3 days after the IL-2 is first added to the culture medium.
34. The method of embodiment 33, wherein step (b) further comprises adding beads coated with anti-CD3 antibodies and anti-CD28 antibodies, in a bead:cell ratio of 1:1, wherein the adding occurs about 14 days after beads coated with anti-CD3 antibodies and anti-CD28 antibodies were first added to the culture medium.
35. The method of any one of embodiments 1-34, wherein step (b) comprises culturing in a culture medium that comprises a mammalian target of rapamycin (mTor) inhibitor.
36. The method of embodiment 35, wherein the mTor inhibitor is rapamycin.
37. The method of embodiment 36, wherein the rapamycin is first added to the culture medium within about 24 hours of the initiation of the culturing.
38. The method of embodiment 36 or embodiment 37, further comprises replenishing the culture medium with rapamycin every 2-3 days after the rapamycin is first added to the culture medium.
39. The method of any one of embodiments 23-38, wherein the cryopreserving step (c) is carried out at least 6 days following an addition of IL-2 to the culture medium in step (b).
40. The method of any one embodiments 1-39, wherein the cryopreserving step (c) is carried out about 12-25 days after the initiation of the culturing step (b).
41. The method of any one embodiments 1-40, wherein step(c) comprises cryopreserving the Tregs in a cryoprotectant comprising DMSO.
42. The method of embodiment 41, wherein the cryoprotectant comprises 10% DMSO.
43. The method of any one of embodiments 1-42, wherein the cryopreservation step (c) comprises changing the temperature of the population of Tregs in the following increments: 1° C./min to 4° C., 25° C./min to −40° C., 10° C./min to −12° C., 1° C./min to −40° C., and 10° C./min to −80° C.-−90° C.
44. The method of any one of embodiments 1-43 wherein the cryopreserved therapeutic population of Tregs is stored in liquid nitrogen vapor phase.
45. The method of any one of embodiments 1-44, wherein the cryopreserved therapeutic population of Tregs is frozen at a Treg density of at least 50 million cells.mL.
46. The method of any one of embodiments 1-45, wherein the cryopreserved therapeutic population of Tregs is frozen at a concentration of 1×10⁶ cells per kg of body weight of an intended recipient of the Tregs per mL.
47. The method of any one of embodiments 1-46, wherein the cryopreserved therapeutic population of Tregs is cryopreserved in a single cryovial.
48. The method of any one of embodiments 1-47, wherein the cryopreserved therapeutic population of Tregs is frozen at a concentration of 1×10⁶ cells per kg of body weight of an intended recipient of the Tregs per mL.
49. The method of any one of embodiments 1-48, wherein the cryopreserved therapeutic population of Tregs is frozen in a total volume of 1-1.5 mL.
50. The method of any one of embodiment 1-49, wherein the method further comprises thawing the population of Tregs after cryopreservation for about 1 week, 1 month, about 3 months, about 6 months, about 9 months, about 12 months, 18 months or about 24 months.
51. The method of embodiment 50, wherein the cryopreserved therapeutic population of Tregs is thawed in 50 mL of a solution comprising 0.9% sodium chloride and about 5% human serum albumin.
52. The method of embodiment 50, wherein the therapeutic population of Tregs was cryopreserved in a single cryovial containing 1×10⁶ Tregs per kg of body weight of an intended recipient of the Tregs per mL and is thawed in 50 mL of a solution containing 0.9% sodium chloride and about 5% human serum albumin.
53. The method of embodiment 52, wherein the therapeutic population of Tregs in the single cryovial is contained in a volume of 1-1.5 mL.
54. The method of any one of embodiments 1-53, wherein the method further comprises obtaining the cell sample from a donor by leukapheresis.
55. The method of any one of embodiments 1-54, wherein the cell sample is not stored overnight or frozen before carrying out the enriching step (a).
56. The method of any one of embodiments 1-55, wherein the cell sample is obtained within 30 minutes before the enriching step(a).
57. The method of any one of embodiments 1-56, wherein the cell sample is from a human donor.
58. The method of embodiment 57, wherein the human donor is a healthy donor.
59. The method of embodiment 57, wherein the human donor is diagnosed with or is suspected of having a disorder associated with Treg dysfunction.
60. The method of embodiment 57, wherein the human donor is diagnosed with or is suspected of having a disorder associated with Treg deficiency.
61. The method of embodiment 57, wherein the human donor is diagnosed with or is suspected of having a neurodegenerative disorder.
62. The method of embodiment 57, wherein the neurodegenerative disorder is Amyotrophic Lateral Sclerosis (ALS), Alzheimer's disease, Parkinson's disease, Huntington's disease or frontotemporal dementia.
63. The method of embodiment 57, wherein the cell sample is from a donor diagnosed with or suspected of having an autoimmune disease.
64. The method of embodiment 63, wherein the autoimmune disease is systemic sclerosis (scleroderma), polymyosititis, ulcerative colitis, inflammatory bowel disease, Crohn's disease, celiac disease, multiple sclerosis (MS), rheumatoid arthritis (RA), Type I diabetes, psoriasis, dermatomyostitis, systemic lupus erythematosus, cutaneous lupus, myasthenia gravis, autoimmune nephropathy, autoimmune hemolytic anemia, autoimmune cytopenia autoimmune hepatitis, autoimmune uveititis, alopecia, thyroiditis or pemhigus.
65. The method of embodiment 57, wherein the human donor is diagnosed with or suspected of having heart failure or ischemic cardiomyopathy.
66. The method of any one of embodiments 1-63, wherein the population of Tregs is subjected to genetic engineering at any point of the method prior to cryopreserving step (c).
67. The method of embodiment 66, wherein the genetic engineering comprises the introduction of a transgene into the Tregs.
68. The method of embodiment 66, wherein the genetic engineering comprises the introduction of an mRNA into the Tregs.
69. The method of embodiment 66, wherein the genetic engineering comprises the gene editing through CRISPR-Cas9.
70. The method of embodiment 66, wherein the genetic engineering comprises the reduction of gene expression through siRNA or antisense oligonucleotides.
71. The method of any one of embodiments 1-65, wherein step (b) is automated.
72. The method of any one of embodiments 1-71, wherein step (b) takes place in a bioreactor.
73. The method of any one of embodiments 1-71, wherein step (b) takes place in a G-REX culture system.
74. The method of any one of embodiments 1-73, wherein the method is performed in a closed system.
75. The method of embodiment 1, wherein the Treg-enriched cell population comprises dysfunctional Tregs.
76. The method of any one of embodiments 1-75, wherein the cryopreserved therapeutic population of Tregs comprises an increased proportion of CD4⁺CD25^{high} Tregs relative to the proportion of CD4⁺CD25^{high} Tregs in the Treg-enriched cell population, as determined by flow cytometry.
77. The method of any one of embodiments 1-76, wherein the cryopreserved therapeutic population of Tregs comprises an increased proportion of CD4⁺CD25^{high}CD127^{low}Tregs relative to the proportion of CD4⁺CD25^{high}CD127^{low} Tregs in the Treg-enriched cell population, as determined by flow cytometry.
78. The method of any one of embodiments 1-76, wherein the cryopreserved therapeutic population of Tregs comprises CD25⁺ Tregs wherein the expression of CD25 in the Tregs is increased relative to the expression of CD25 in the Tregs in the Treg-enriched cell population, as determined by flow cytometry.
79. The method of embodiment 78, wherein the expression of CD25 is increased by at least about 10-fold, at least about 20-fold, or at least about 30-fold by flow cytometry.
80. The method of any one of embodiments 1-79, wherein the cryopreserved population of Tregs comprises CD127⁺ Tregs wherein the expression of CD127 in the Tregs is not increased greater than 3-fold relative to the expression of CD127 in the Tregs in the Treg-enriched cell population, as determined by flow cytometry.
81. The method of any one of embodiments 1-80, wherein the expression of CD127 is not increased relative to the expression of CD127 in the Tregs in the Treg-enriched cell population, as determined by flow cytometry.
82. The method of any one of embodiments 1-81, wherein the granularity of the Tregs in the cryopreserved therapeutic population of Tregs is increased relative to the granularity of the Tregs in the Treg-enriched cell population, as determined by flow cytometry.
83. The method of embodiment 82 wherein the granularity of the Tregs increased by at least about 1.5-fold, at least about 2-fold, or at least about 2.5-fold.
84. The method of any one of embodiments 1-83, wherein the size of the Tregs in the cryopreserved therapeutic population of Tregs is increased relative to the size of the Tregs in the Treg-enriched cell population, as determined by flow cytometry.
85. The method of embodiment 84, wherein the size of the Tregs increased by at least about 1.2-fold, at least about 1.5-fold, or at least about 2-fold.
86. The method of any one of embodiments 1-85, wherein the Tregs in the cryopreserved therapeutic population of Tregs comprise CTLA4-expressing Tregs wherein the expression of CTLA4 is increased in the Tregs relative to expression of CTLA4 in the Tregs in the Treg-enriched cell population.
87. The method of any one of embodiments 1-86, wherein the cryopreserved therapeutic population of Tregs comprises CTLA4+ Tregs wherein the proportion of CTLA4+ Tregs is increased relative to the proportion of CTLA4+ Tregs in the Tregs in the Treg-enriched cell population.
88. The method of any one of embodiments 1-87, wherein the cryopreserved therapeutic population of Tregs comprises at least 20%, at least 30%, at least 40%, or at least 50% CTLA4+ Tregs, as determined by flow cytometry.
89. The method of any one of embodiments 1-88, wherein the cryopreserved therapeutic population of Tregs comprises FoxP3+ Tregs wherein the proportion of FoxP3+ Tregs is increased relative to the proportion of FoxP3+ Tregs in the Tregs in the Treg-enriched cell population.
90. The method of any one of embodiments 1-89, wherein the cryopreserved therapeutic population of Tregs comprises at least 40%, at least 50%, at least 60%, at least 70%, or at least 80% or at least 90% FoxP3+ Tregs, as determined by flow cytometry.
91. The method of any one of embodiments 1-90, wherein the Tregs in the cryopreserved therapeutic population of Tregs comprise FoxP3-expressing Tregs wherein the expression of FoxP3 is increased in the Tregs relative to expression of FoxP3 in the Tregs in the Treg-enriched cell population.
92. The method of any one of embodiments 1-91, wherein the cryopreserved therapeutic population of Tregs comprises at least 70%, at least 80%, or at least 90% CD4⁺CD25^{high}CD127^{low} Tregs in the Treg-enriched cell population, as determined by flow cytometry.
93. The method of any one of embodiments 1-92 wherein the cryopreserved therapeutic population of Tregs comprises at least 70%, at least 75%, at least 80%, at least 85%, at least 90% or at least 95% CD4⁺CD25⁺ cells, as determined by flow cytometry.
94. The method of embodiment 93, wherein the cryopreserved population of Tregs comprise fewer than 20% CD8+ cells, determined by flow cytometry.
95. The method of any one of embodiments 1-94, wherein the viability of the cryopreserved therapeutic population of Tregs is at least 70%, at least 75%, at least 80%, at least 85%, at least 90% or at least 95%, as determined by trypan blue staining performed following thawing of the cryopreserved therapeutic population.
96. The method of any one of embodiments 1-95, wherein the viability of the cryopreserved therapeutic population of Tregs, as determined by trypan blue staining performed following thawing of the cryopreserved therapeutic population, is at least 70%, at least 75%, at least 80%, at least 85%, at least 90% or at least 95% of the viability of the expanded Treg cell population prior to the expanded Treg cell population being cryopreserved in step (c).
97. The method of any one of embodiments 1-96, wherein the suppressive function of the cryopreserved population of Tregs is at least 50%, at least 60%, at least 70%, at least 75%, at least 80%, at least 85%, at least 90% or at least 95%, as determined by flow cytometry performed following thawing of the therapeutic population.
98. The method of any one of embodiments 1-97, wherein the cryopreserved population of Tregs exhibits a suppressive function that is greater than the suppressive function of the enriched Treg cell population, as measured prior to step (b)), as measured by a Treg suppression assay.
99. The method of any one of embodiments 1-98, wherein the suppressive function of the cryopreserved therapeutic population of Tregs, as determined by flow cytometry following thawing of the therapeutic population, is at least 50%, at least 60%, at least 70%, at least 75%, at least 80%, at least 85%, at least 90% or at least 95% of the suppressive function of the expanded Treg cell population prior to the Tregs being cryopreserved in step (c), as determined by flow cytometry.
100. The method of any one of embodiments 1-99, wherein the cryopreserved therapeutic population of Tregs, following thawing, exhibits an ability to suppress inflammatory cells, as measured by IL-6 production by the inflammatory, wherein the inflammatory cells are macrophages or monocytes from human donors or generated from induced pluripotent stem cells.
101. A composition comprising a cryopreserved therapeutic population of Tregs produced by the method of any one of embodiments 1-100.
102. A cryopreserved composition comprising a therapeutic population of Tregs, wherein upon thawing the therapeutic population of Tregs exhibits greater 50% suppressive function in the absence of need for additional expansion.
103. A cryopreserved composition comprising a therapeutic population of ex vivo-expanded Tregs, wherein upon thawing the therapeutic population of Tregs exhibits greater than 50% suppressive function in the absence of need for additional expansion.
104. The cryopreserved composition of embodiment 102 or 103, wherein the suppressive function of the therapeutic population of Tregs is at least 50%, at least 60%, at least 70%, at least 75%, at least 80%, at least 85%, at least 90% or at least 95% as determined by suppression of proliferation of responder T cells by flow cytometry or thymidine incorporation performed following thawing of the cryopreserved composition.
105. The cryopreserved composition of any one of embodiments 102-104, wherein the suppressive function of the therapeutic population of Tregs following thawing of the cryopreserved composition is at least 50%, at least 60%, at least 70%, at least 75%, at least 80%, at least 85%, at least 90% or at least 95% of the suppressive function of the Tregs before undergoing cryopreservation, as determined by suppression of proliferation of responder T cells by flow cytometry or thymidine incorporation.
106. The composition of any one of embodiments 102-105, wherein upon thawing, the therapeutic population of Tregs exhibits at least 70%, at least 75%, at least 80%, at least 85%, at least 90% or at least 95% viability, as determined by trypan blue staining.
107. The composition of any one of embodiments 102-106, wherein upon thawing, the therapeutic population of Tregs exhibits a viability that is at least 70%, at least 75%, at least 80%, at least 85%, at least 90% or at least 95% of the viability of the Tregs before undergoing cryopreservation, as determined by trypan blue staining.
108. The cryopreserved composition of any one of embodiments 102-107, wherein the composition has been cryopreserved for about 1 months, about 3 months, about 6 months, about 9 months, about 12 months, about 18 months or about 24 months.
109. The cryopreserved composition of any one of embodiments 102-108, wherein the therapeutic population of Tregs comprises at least 70%, at least 75%, at least 80%, at least 85%, at least 90% or at least 95% CD4⁺CD25⁺ cells, as determined by flow cytometry.
110. The cryopreserved composition of any one of embodiments 102-109, wherein the therapeutic population of Tregs comprises at least 70%, at least 75%, at least 80%, at least 85%, at least 90% or at least 95% CD4⁺CD25^{high} cells, as determined by flow cytometry.
111. The cryopreserved composition of any one of embodiments 102-110, wherein the therapeutic population of Tregs comprises at least 70%, at least 75%, at least 80%, at least 85%, at least 90% or at least 95% CD4⁺CD25^{high}CD127^{low} cells, as determined by flow cytometry.
112. The cryopreserved composition of any one of embodiments 102-111, wherein the therapeutic population of Tregs comprises at least 20%, at least 30%, at least 40%, or at least 50% CTLA4+ Tregs, as determined by flow cytometry.
113. The cryopreserved composition of any one of embodiments 102-112, wherein the therapeutic population of Tregs comprises at least 50%, at least 60%, at least 70%, at least 75%, at least 80%, at least 85%, at least 90% or at least 95% FoxP3+ cells, as determined by flow cytometry.
114. The cryopreserved composition of any one of embodiments 102-113, wherein the therapeutic population of Tregs comprises less than 20% CD8+ cells, determined by flow cytometry.
115. The cryopreserved composition of any one of embodiments 102-114, wherein upon thawing, the expression of one or more of the gene products listed in Table 12 though Table 18 is substantially the same in the therapeutic population of Tregs as the expression of the one or more gene products in the Tregs prior to cryopreservation.
116. The cryopreserved composition of any one of embodiments 103-114, wherein upon thawing, the expression of one or more gene products listed in Table 12 and/or Table 13 is decreased relative to the expression of the one or more gene products in baseline Tregs prior to expansion.
117. The cryopreserved composition of any one of embodiments 103-114, wherein upon thawing, the expression of one or more gene products listed in Table 14 is increased relative to the expression of the one or more gene products in baseline Tregs prior to expansion.
118. The cryopreserved composition of any one of embodiments 103-114, wherein upon thawing, the expression of one or more gene products listed in Table 15 is increased relative to the expression of the one or more gene products in baseline Tregs prior to expansion.
119. The cryopreserved composition of embodiment 118, wherein the expression of one or more of the following gene products is increased in the therapeutic population of Tregs relative to the expression of the one or more gene products in baseline Tregs prior to expansion: ADAM10, AIMIP1, AIMP2, ARG2, BCL2L1, BSG, CD2, CD28, CD38, CD74, CD84, CTLA4, FAS, FOXP3, GCLC, HAT1, HLA-DQA1, HLA-DQB1, HLA-DRA, HLA-DRB1, HPGD, ICOS, IL1RN, IRF4, KPNA2, LGALS1, LGMN, PCNA, POFUT1, SATB1, SELPLG, STAT1, TFRC, TNFRSF18,
120. The cryopreserved composition of any one of embodiments 103-114, wherein upon thawing, the expression of one or more gene products listed in Table 16 is increased relative to the expression of the one or more gene products in baseline Tregs prior to expansion.
121. The cryopreserved composition of embodiment 120, wherein the expression of one or more of the following gene products is increased in the therapeutic population of Tregs relative to the expression of the one or more gene products in baseline Tregs prior to expansion: CAA2, ACADM, ACADVL, ACOT7, ACSL1, ACSL4, ACSL5, AGK, AGMAT, AK4, ARG2, ARL2, AUH, BCL2L1, BDH1, BNIP1, CDK1, CHDH, CIAPIN1, CISD2, COX17, CPOX, CPT1A, CPT2, CYB5B, DAP3, DHRS2, DNM1L, DUT, DYNLL1, ECI1, FDXR, FEN1, FKBP8, GK, GRSF1, HTRA2, L2HGDH, LACTB2, LRPPRC, MAIP1, MAOA, MPST, MRPL1, MRPL12, MRPL13, MRPL14, MRPL17, MRPL22, MRPL37, MRPL39, MRPL4, MRPL43, MRPL44, MRPL46, MRPL48, MRPS11, MRPS14, MRPS2, MRPS27, MRPS31, MRPS35, MRPS9, MTHFD2, MTX1, MYCBP, NDUFA8, NUDT1, OAT, PITRM1, PLSCR3, PMPCA, PPIF, PTRH2, PYCR2, REXO2, RMND1, SFXN2, SLC25A10, SLC25A19, SLC25A4, TIGAR, TIMM13, TIMM23, TMEM14C, TOMM22, TOMM34, TOMM40, and TST.
122. The cryopreserved composition of any one of embodiments 103-114, wherein upon thawing, the expression of one or more gene products listed in Table 17 is increased relative to the expression of the one or more gene products in baseline Tregs prior to expansion.
123. The cryopreserved composition of embodiment 122, wherein the expression of one or more of the following gene products is increased in the therapeutic population of Tregs relative to the expression of the one or more gene products in baseline Tregs prior to expansion: ARL2, ARL3, BCCIP, CCDC124, CDK1, CDK2, CDK5, CDK6, CUL4B, DCTN3, FEN1, HELLS, LIG1, MAD2L1, MAEA, MCM2, MCM2, MCM3, MCM4, MCM5, MCM6, MCM7, MCMBP, NUDC, PCNA, POLD1, POLD2, RALB, RBM38, RFC2, RFC3, RFC4, RFC5, RNASEH2A, RNASEH2B, and SMC2.
124. The cryopreserved composition of any one of embodiments 103-114, wherein upon thawing, the expression of one or more gene products listed in Table 18 is increased relative to the expression of the one or more gene products in baseline Tregs prior to expansion.
125. The cryopreserved composition of embodiment 124, wherein the expression of one or more of the following gene products is increased in the therapeutic population of Tregs relative to the expression of the one or more gene products in baseline Tregs prior to expansion: ACAA2, ACADM, ACADVL, ACOT7, BSG, CACYBP, CD74, CDK1, CPOX, DUT, ECI1, ENO3, FEN1, FKBP3, HIST1H2BJ, HLA-DQA1, HLA-DRA, HLA-DRB1, LGALS1, LGALS3, MCM5, MCM6, MCM7, MTHFD1, NAMPT, NME1, NQO1, PCNA, RAB1A, RALB, SLC25A4, STAT1, STMN1, STMN2, TUBAIB, TUBB4A, TUBB8, TXN, TXNRD1, and WARS.
126. The cryopreserved composition of any one of embodiments 116-125, wherein the expression is changed at least about 4-fold, at least about 5-fold, at least about 6-fold, at least about 7-fold, at least about 8-fold, at least about 9-fold, or at least about 10-fold.
127. The cryopreserved composition of any one of embodiments 115-126, wherein expression is determined by single-shot proteomic analysis.
128. A cryopreserved composition comprising a therapeutic population of ex vivo-expanded Tregs, wherein upon thawing, the expression of one or more of the geneproducts listed in Tables 6-11 is substantially the same in the therapeutic population of Tregs as the expression of the one or more gene products in the Tregs prior to cryopreservation.
129. A cryopreserved composition comprising a therapeutic population of ex vivo-expanded Tregs, wherein upon thawing, the expression of one or more gene products listed in Table 12 and/or Table 13 is decreased relative to the expression of the one or more gene products in baseline Tregs prior to expansion.
130. A cryopreserved composition comprising a therapeutic population of ex vivo-expanded Tregs, wherein upon thawing, the expression of one or more gene products listed in Table 14 is increased relative to the expression of the one or more gene products in baseline Tregs prior to expansion.
131. A cryopreserved composition comprising a therapeutic population of ex vivo-expanded Tregs, wherein upon thawing, the expression of one or more gene products listed in Table 15 is increased relative to the expression of the one or more gene products in baseline Tregs prior to expansion.
132. The cryopreserved composition of embodiment 131, wherein the expression of one or more of the following gene products is increased in the therapeutic population of ex vivo-expanded Tregs relative to the expression of the one or more gene products in baseline Tregs prior to expansion: ADAM10, AIMP1, AIMP2, ARG2, BCL2L1, BSG, CD2, CD28, CD38, CD74, CD84, CTLA4, FAS, FOXP3, GCLC, HAT1, HLA-DQA1, HLA-DQB1, HLA-DRA, HLA-DRB1, HPGD, ICOS, IL1RN, IRF4, KPNA2, LGALS1, LGMN, PCNA, POFUT1, SATB1, SELPLG, STAT1, TFRC, TNFRSF18,
133. A cryopreserved composition comprising a therapeutic population of ex vivo-expanded Tregs, wherein upon thawing, the expression of one or more gene products listed in Table 16 is increased relative to the expression of the one or more gene products in baseline Tregs prior to expansion.
134. The cryopreserved composition of embodiment 133, wherein the expression of one or more of the following gene products is increased in the therapeutic population of Tregs relative to the expression of the one or more gene products in baseline Tregs prior to expansion: CAA2, ACADM, ACADVL, ACOT7, ACSL1, ACSL4, ACSL5, AGK, AGMAT, AK4, ARG2, ARL2, AUH, BCL2L1, BDH1, BNIP1, CDK1, CHDH, CIAPIN1, CISD2, COX17, CPOX, CPT1A, CPT2, CYB5B, DAP3, DHRS2, DNM1L, DUT, DYNLL1, ECI1, FDXR, FEN1, FKBP8, GK, GRSF1, HTRA2, L2HGDH, LACTB2, LRPPRC, MAIP1, MAOA, MPST, MRPL1, MRPL12, MRPL13, MRPL14, MRPL17, MRPL22, MRPL37, MRPL39, MRPL4, MRPL43, MRPL44, MRPL46, MRPL48, MRPS11, MRPS14, MRPS2, MRPS27, MRPS31, MRPS35, MRPS9, MTHFD2, MTX1, MYCBP, NDUFA8, NUDT1, OAT, PITRM1, PLSCR3, PMPCA, PPIF, PTRH2, PYCR2, REXO2, RMND1, SFXN2, SLC25A10, SLC25A19, SLC25A4, TIGAR, TIMM13, TIMM23, TMEM14C, TOMM22, TOMM34, TOMM40, and TST.
135. A cryopreserved composition comprising a therapeutic population of ex vivo-expanded Tregs, wherein upon thawing, the expression of one or more gene products listed in Table 17 is increased relative to the expression of the one or more gene products in baseline Tregs prior to expansion.
136. The cryopreserved composition of embodiment 135, wherein the expression of one or more of the following gene products is increased in the therapeutic population of Tregs relative to the expression of the one or more gene products in baseline Tregs prior to expansion: ARL2, ARL3, BCCIP, CCDC124, CDK1, CDK2, CDK5, CDK6, CUL4B, DCTN3, FEN1, HELLS, LIG1, MAD2L1, MAEA, MCM2, MCM2, MCM3, MCM4, MCM5, MCM6, MCM7, MCMBP, NUDC, PCNA, POLD1, POLD2, RALB, RBM38, RFC2, RFC3, RFC4, RFC5, RNASEH2A, RNASEH2B, and SMC2.
137. A cryopreserved composition comprising a therapeutic population of ex vivo-expanded Tregs, wherein upon thawing, the expression of one or more gene products listed in Table 18 is increased relative to the expression of the one or more gene products in baseline Tregs prior to expansion.
138. The cryopreserved composition of embodiment 137, wherein the expression of one or more of the following gene products is increased in the therapeutic population of Tregs relative to the expression of the one or more gene products in baseline Tregs prior to expansion: ACAA2, ACADM, ACADVL, ACOT7, BSG, CACYBP, CD74, CDK1, CPOX, DUT, ECI1, ENO3, FEN1, FKBP3, HIST1H2BJ, HLA-DQA1, HLA-DRA, HLA-DRB1, LGALS1, LGALS3, MCM5, MCM6, MCM7, MTHFD1, NAMPT, NME1, NQO1, PCNA, RAB1A, RALB, SLC25A4, STAT1, STMN1, STMN2, TUBAIB, TUBB4A, TUBB8, TXN, TXNRD1, and WARS. The cryopreserved composition of any one of embodiments 129-137, wherein the expression is changed at least about 4-fold, at least about 5-fold, at least about 6-fold, at least about 7-fold, at least about 8-fold, at least about 9-fold, or at least about 10-fold.
139. The cryopreserved composition of any one of embodiments 128-136, wherein expression is determined by single-shot proteomic analysis.
140. The cryopreserved composition of any one of embodiments 102-140, wherein the cryopreserved therapeutic population of Tregs, following thawing, exhibits an ability to suppress inflammatory cells, as measured by IL-6 production by the inflammatory cells, wherein the inflammatory cells are macrophages or monocytes from human donors or generated from induced pluripotent stem cells.
141. The cryopreserved composition of any one of embodiments 102-140, wherein the cryopreserved composition comprises DMSO.
142. The cryopreserved composition of embodiment 141, wherein the cryopreserved composition comprises 10% DMSO.
143. The cryopreserved composition any one of embodiments 102-142, wherein the therapeutic population of Tregs is present at a concentration of 1×10⁶ cells per kg of body weight of an intended recipient of the Treg population per ml.
144. The cryopreserved composition of embodiment 143, wherein the therapeutic population of Tregs is autologous to an intended recipient of the Treg population.
145. The cryopreserved composition any one of embodiments 102-144, wherein the cryopreserved composition is contained a single cryovial.
146. The cryopreserved composition any one of embodiments 102-145, wherein the total volume of the cryopreserved composition is 1-1.5 mL.
147. The cryopreserved composition of any one of embodiments 102-146, wherein the composition is provided at a concentration of ((1×10⁶ cells)*((patient weight in kg)+(0.3)*(patient weight in kg))/mL).
148. The cryopreserved composition of embodiment 147, wherein the cryopreserved composition is present in a total of 1.2 mL.
149. A method of treating a disorder associated with Treg dysfunction, the method comprising: administering to a subject in need of said treatment a therapeutic population of Tregs, wherein the Tregs had been ex vivo expanded and cryopreserved, and wherein the Tregs are not further expanded prior to the administering.
150. A method of treating a disorder associated with Treg deficiency, the method comprising: administering to a subject in need of said treatment a therapeutic population of Tregs, wherein the Tregs had been ex vivo expanded and cryopreserved, and wherein the Tregs are not further expanded prior to the administering.
151. A method of treating a disorder associated with overstimulation of the immune system, the method comprising: administering to a subject in need of said treatment a therapeutic population of Tregs, wherein the Tregs had been ex vivo expanded and cryopreserved, and wherein the Tregs are not further expanded prior to the administering.
152. A method of treating an inflammatory condition driven by a T cell response, the method comprising: administering to a subject in need of said treatment a therapeutic population of Tregs, wherein the Tregs had been ex vivo expanded and cryopreserved, and wherein the Tregs are not further expanded prior to the administering.
153. A method of treating a neurodegenerative disorder in a subject in need thereof, the method comprising: administering to the subject a therapeutic population of Tregs, wherein the Tregs had been ex vivo expanded and cryopreserved, and wherein the Tregs are not further expanded prior to the administering.
154. The method of embodiment 153, wherein the neurodegenerative disease is Amyotrophic Lateral Sclerosis (ALS), Alzheimer's disease, Parkinson's disease, frontotemporal dementia or Huntington's disease.
155. A method of treating an autoimmune disorder in a subject in need thereof, the method comprising: administering to the subject a therapeutic population of Tregs, wherein the Tregs had been ex vivo expanded and cryopreserved, and wherein the Tregs are not further expanded prior to the administering.
156. The method of embodiment 155, wherein the autoimmune disorder is polymyosititis, ulcerative colitis, inflammatory bowel disease, Crohn's disease, celiac disease, multiple sclerosis (MS), rheumatoid arthritis (RA), Type I diabetes, psoriasis, dermatomyostitis, systemic lupus erythematosus, cutaneous lupus, myasthenia gravis, autoimmune nephropathy, autoimmune hemolytic anemia, autoimmune cytopenia autoimmune hepatitis, autoimmune uveititis, alopecia, thyroiditis or pemhigus.
157. A method of treating graft-versus-host disease in a subject in need thereof, the method comprising: administering to the subject a therapeutic population of Tregs, wherein the Tregs had been ex vivo expanded and cryopreserved, and wherein the Tregs are not further expanded prior to the administering.
158. The method of embodiment 157, wherein the subject has received a bone marrow transplant, kidney transplant or liver transplant.
159. A method of improving islet graft survival in a subject in need thereof, comprising: combining islet transplantation with administering to the subject a therapeutic population of Tregs, wherein the Tregs had been ex vivo expanded and cryopreserved, and wherein the Tregs are not further expanded prior to the administering.
160. A method of treating cardio-inflammation in a subject in need thereof, the method comprising: administering to the subject a therapeutic population of Tregs, wherein the Tregs had been ex vivo expanded and cryopreserved, and wherein the Tregs are not further expanded prior to the administering.
161. The method of embodiment 160, wherein the cardio-inflammation is associated with a atherosclerosis, myocardial infarction, ischemic cardiomyopathy or heart failure. A method of treating neuroinflammation in a subject in need thereof, the method comprising: administering to the subject a therapeutic population of Tregs, wherein the Tregs had been ex vivo expanded and cryopreserved, and wherein the Tregs are not further expanded prior to the administering.
162. The method of embodiment 161, wherein the neuroinflammation is associated with stroke, acute disseminated encephalitis, acute optic neuritis, transverse myelitis, neuromyelitis optica, epilepsy, traumatic brain injury, spinal cord injury, encephalititis or meningitissubject had had a stroke.
163. A method of treating a Tregopathy in a subject in need thereof, comprising: administering to the subject a therapeutic population of Tregs, wherein the Tregs had been ex vivo expanded and cryopreserved, and wherein the Tregs are not further expanded prior to the administering.
164. The method of embodiment 163, wherein the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.
165. The method of any one of embodiments 149-164, wherein the Tregs are autologous to the subject.
166. The method of any one of embodiments 149-164, wherein the Tregs are allogeneic to the subject.
167. The method of any one of embodiments 149-167, wherein, the therapeutic population of Tregs is administered to the subject in an aqueous solution comprising 0.9% sodium chloride and 5% human serum albumin.
168. A method of treating a neurodegenerative disorder in a subject in need thereof, the method comprising: administering to the subject a pharmaceutical composition comprising a therapeutic population of Tregs, wherein the pharmaceutical composition comprises the cryopreserved composition of any one of embodiments 102-148, thawed and suspended in an aqueous solution suitable for administration to the subject.
169. The method of embodiment 168, wherein the neurodegenerative disease is Amyotrophic Lateral Sclerosis (ALS), Alzheimer's disease or Parkinson's disease.
170. The method of embodiment 168 or 169, wherein the aqueous solution comprises 0.9% sodium chloride and 5% human serum albumin.
171. The method of any one of embodiments 168-170, wherein the therapeutic population of Tregs are administered within 6 hours of thawing.
172. The method of any one of embodiments 138-171, wherein about 1×10⁶ Tregs per kg of body weight of the subject are administered.
173. The method of any one of embodiments 138-172, wherein about 1×10⁶ Tregs per kg of body weight of the subject are administered per month for at least two months.
174. The method of any one of embodiments 138-171, wherein about 1×10⁶ Tregs per kg of body weight of the subject are administered per month for the first and second month, and about 2×10⁶ Tregs per kg of body weight of the subject are administered per month for at least a third and fourth month.
175. The method of any one of embodiments 138-171, wherein about 1×10⁶ cells per kg of body weight of the subject are administered per month for the first and second month, about 2×10⁶ cells per kg of body weight of the subject are administered per month for the third and fourth month, and about 3×10⁶ to about 1×10⁹ cells per kg of body weight of the subject are administered per month for at least a fifth and sixth month.
176. The method of any one of embodiments 138-175, wherein the Tregs are administered intravenously.
177. The method of any one of embodiments 138-176, wherein the subject is additionally administered IL-2.
178. The method of embodiment 177, wherein the subject is administered 2×10⁵ IU/m² of IL-2.
179. The method of embodiment 177 or 178, wherein the IL-2 is administered three times a week.
180. The method of any one of embodiments 177-179, wherein the IL-2 is administered subcutaneously.
181. The method of any one of embodiments 177-180, wherein the IL-2 is administered at least 2 weeks, at least 3 weeks, or at least 4 weeks prior to the first Treg infusion.
182. The method of any one of embodiments 138-181, wherein the method results in an increase in the Treg suppressive function in the blood.
183. The method of embodiment 182, wherein the method results in an increase in the Treg suppressive function in the blood from baseline to week 24.
184. The method of any one of embodiments 138-182 wherein the method results in an increase in the number of Tregs in the blood.
185. The method of embodiment 184, wherein the method results in an increase in the number of Tregs in the blood from baseline to week 24.
186. The method of any one of embodiments 138-185 wherein the subject is human.

### 6. EXEMPLARY DEFINITIONS

In accordance with the present disclosure, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and I P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used throughout this application and in the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

“Biocompatible” refers to a material that, when exposed to living cells, will support an appropriate cellular activity of the cells without causing an undesirable effect in the cells, such as a change in a living cycle of the cells, a change in a proliferation rate of the cells, or a cytotoxic effect.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g, does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

The term “for example” or “e.g.” as used herein, is used merely by way of example, without limitation intended, and should not be construed as referring only those items explicitly enumerated in the specification.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “implantable” or “suitable for implantation” means surgically appropriate for insertion into the body of a host, e.g., biocompatible, or having the desired design and physical properties.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without t limitation, recombinant fusion, covalent bonding, disulfide bonding, ionic bonding, hydrogen bonding, electrostatic bonding, and the like.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The terms “operably linked” and operatively linked”, as used herein, refers to that union of the nucleic acid sequences that are linked in such a way, such that the coding regions are contiguous and in correct reading frame. Such sequences are typically contiguous, or substantially contiguous. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In particular embodiments, the mammalian patient is preferably human.

The phrase “pharmaceutically-acceptable” refers to molecular entities and compositions that preferably do not produce an allergic or similar untoward reaction when administered to a mammal, and in particular, when administered to a human.

As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from A/A′-dibenzylethylenediamine or ethylenediamine; and combinations thereof.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gin), Glutamic Acid (E; Glu), Glycine (G; Gly), Histidine (H; His), Isoleucine (I; lie), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Vai), and Lysine (K; Lys). Amino acid residues described herein are preferred to be in the “1” isomeric form. However, residues in the “d” isomeric form may be substituted for any 1-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 pg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 pg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

“Targeting moiety” is any factor that may facilitate targeting of a specific site by a particle. For example, the targeting moiety may be a chemical targeting moiety, a physical targeting moiety, a geometrical targeting moiety, or a combination thereof. The chemical targeting moiety may be a chemical group or molecule on a surface of the particle; the physical targeting moiety may be a specific physical property of the particle, such as a surface such or hydrophobicity; the geometrical targeting moiety includes a size and a shape of the particle. Further, the chemical targeting moiety may be a dendrimer, an antibody, an aptamer, which may be a thioaptamer, a ligand, an antibody, or a biomolecule that binds a particular receptor on the targeted site. A physical targeting moiety may be a surface charge. The charge may be introduced during the fabrication of the particle by using a chemical treatment such as a specific wash. For example, immersion of porous silica or oxidized silicon surface into water may lead to an acquisition of a negative charge on the surface. The surface charge may be also provided by an additional layer or by chemical chains, such as polymer chains, on the surface of the particle. For example, polyethylene glycol chains may be a source of a negative charge on the surface. Polyethylene glycol chains may be coated or covalently coupled to the surface using methods known to those of ordinary skill in the art.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. An anti-neurodegenerative agent may be a preferred therapeutic agent. For a more detailed description of such agents and other therapeutic agents, those skilled in the art are referred to any number of instructive manuals including, but not limited to, the Physician's Desk Reference and Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cA-acting promoter sequence and optionally linked operably to one or more other cA-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cA-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

### 6.1 Methods of Producing Expanded Therapeutic Populations of Trees

Provided herein are methods for the production of expanded Treg cell populations, cryopreserved therapeutic populations of Tregs, and pharmaceutical compositions comprising cryopreserved expanded Tregs that have been thawed and placed into pharmaceutical compositions without further expansion, particularly for use in connection with treating neurodegenerative diseases, autoimmune diseases and other diseases with an inflammatory component. In some embodiments, the method involves the growth and manipulation of patient Tregs outside of the body.

The methods for the production of therapeutic populations of Tregs provided herein may be useful for treating patients with a pathological disease or condition. Also provided herein are therapeutic populations of Tregs produced by methods described herein and pharmaceutical compositions thereof.

In some embodiments, the therapeutic populations of Tregs produced by a method provided herein have advantageous properties for clinical application. For example, in one embodiment, a therapeutic population of Tregs produced by a method provided herein may be cryopreserved without loss of viability, purity or potency. For example, in one embodiment, a therapeutic population of ex vivo-expanded Tregs produced by a method provided herein may be cryopreserved, thawed and without further expansion, demonstrate maintenance of viability, purity and potency as compared to the expanded Tregs prior to cryopreservation. In another embodiment, a therapeutic population of Tregs produced by a method described herein comprises Tregs with higher suppressive ability than the Tregs enriched from the donor samples, or compared to a healthy donor's Tregs. In yet another embodiment, a therapeutic population of Tregs produced by a method described herein comprises Tregs with a suppressive ability that is absent Tregs enriched from the donor samples, or compared to a healthy donor's Tregs. Thus, in some embodiments, a method of producing a therapeutic population of Tregs provided herein is an improved method compared to methods known in the art.

In some embodiments, the method of producing a therapeutic population of Tregs comprises the steps of (1) enriching a cell population obtained from a subject for Tregs; (2) ex vivo expansion of the cell population enriched for Tregs and/or (3) cryopreservation of the expanded Tregs. A population of cells comprising Tregs may be enriched from a biological sample, e.g., a peripheral blood sample or thymic tissue.

### 6.1.1. Treg Enrichment

In some embodiments, methods of producing a therapeutic population of Tregs provided herein comprise a step of enriching Tregs from in a biological donor sample, e.g., a peripheral blood sample or thymic tissue. In some embodiments, the therapeutic population of Tregs is obtained from a serum sample suspected of containing Tregs. In some embodiments, the therapeutic population of Tregs is obtained from a cell sample suspected of containing Tregs, obtained from a donor via leukapheresis. In some embodiments, the therapeutic population of Tregs is obtained from a biological sample suspected of containing Tregs.

In some embodiments, the therapeutic population of Tregs is enriched from a biological sample from a donor subject, in particular a human donor subject. The biological sample can be any sample suspected of containing Tregs, likely to contain Tregs or know to contain Tregs. Such biological samples may be taken directly from the subject, or may be samples resulting from one or more processing steps, such as separation, e.g. selection or enrichment, centrifugation, washing, and/or incubation. Biological samples include, but are not limited to, body fluids, such as blood, plasma, serum, cerebrospinal fluid, synovial fluid, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, and thymus.

In some embodiments, the biological sample is a blood-derived sample, e.g., a samples derived from whole blood, serum, or plasma. In some embodiments, the biological sample is or includes peripheral blood mononuclear cells. In some embodiments, the biological sample is a peripheral blood or serum sample. In some embodiments, the biological sample is a lymph node sample.

In some embodiments, the donor subject is a human subject. In some embodiments, the human donor is a healthy donor.

In some embodiments, the donor subject is diagnosed with or is suspected of having a disorder associated with Treg dysfunction. In some embodiments, the donor subject is diagnosed with or is suspected of having a disorder associated with Treg deficiency. In some embodiments, the donor subject is diagnosed with or is suspected of having a condition driven by a T cell response.

In some embodiments the donor subject is diagnosed with or is suspected of having a neurodegenerative disease. In some embodiments, the donor subject is diagnosed with or is suspected of having Alzheimer's disease, Amyotrophic Lateral Sclerosis, Huntington's disease or frontotemporal dementia.

In some embodiments, the donor subject is diagnosed with or is suspected of having a disorder that would benefit from downregulation of the immune system.

In some embodiments, the donor subject is diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, systemic lupus erythematosus, cutaneous lupus, myasthenia gravis, autoimmune nephropathy, autoimmune hemolytic anemia, autoimmune cytopenia autoimmune hepatitis, autoimmune uveitis, alopecia, thyroiditis or pemhigus.

In some embodiments, the donor subject is diagnosed with or suspected of having heart failure or ischemic cardiomyopathy. In some embodiments, the donor subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation).

In some embodiments, the donor subject is diagnosed with or suspected of having neuroinflammation. Neuroinflammation may be associated, for example, with stroke, acute disseminated encephalitis, acute optic neuritis, transverse myelitis, neuromyelitis optica, epilepsy, traumatic brain injury, spinal cord injury, encephalitis central nervous system (CNS) vasculitis, neurosarcoidosis, autoimmune or post-infectious encephalitis or chronic meningitis.

In some embodiments, the donor subject is diagnosed with or suspected of having chronic inflammatory demyelinating polyradiculoneuropathy (CIDP). In some embodiments, the donor subject is diagnosed with or suspected of having acute inflammatory demyelinating polyneuropathy (AIDP). In some embodiments, the donor subject is diagnosed with or suspected of having Guillain-Barre syndrome (GBS).

In some embodiments, the donor subject is diagnosed with or suspected of having cardo-inflammation, e.g., cardio-inflammation associated with myocardial infarction, ischemic cardiomyopathy, with heart failure.

In some embodiments, the donor subject has had a stroke.

In some embodiments, the donor subject is diagnosed with or suspected of having cancer, e.g., a blood cancer.

In some embodiments, the donor subject is diagnosed with or suspected of having asthma.

In some embodiments, the donor subject is diagnosed with or suspected of having eczema.

In some embodiments, the donor subject is diagnosed with or suspected of having a disorder associated with overactivation of the immune system.

In some embodiments, the donor subject is diagnosed with or suspected of having Tregopathy. The Tregopathy may be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

Methods of obtaining a population of cells suspected to contain, likely to contain or known to contain Tregs from such biological donor samples are known in the art. For example, lymphocytes may be obtained from a peripheral blood sample by leukapheresis. In some embodiments, Tregs are enriched from a population of lymphocytes. In some embodiments, repeated peripheral blood samples are obtained from a donor for producing Tregs. In some embodiments, two or more peripheral blood samples are obtained from a donor. In some embodiments, insufficient Tregs are obtained from a donor sample after expanding for 25 days and a subsequent sample is obtained. In some embodiments, the donor sample undergoes volume reduction (e.g., volume reduction by a method described herein, such as a method described in Section 8.5.3) during the enrichment process.

In some embodiments, biological samples (e.g., leukapheresis samples) from more than one donor are pooled prior to the enrichment process to generate an allogeneic population of Tregs. In some embodiments, biological samples (e.g., leukapheresis samples from 2, 3, 4, or 5 donors are pooled.

Tregs may be enriched from a biological sample by any method known in the art or described herein (e.g., a method described in section 8). In some embodiments, Tregs are enriched from a sample using magnetic bead separation (e.g., CliniMACS Tubing Set LS (162-01) or CliniMACS® Plus Instrument), fluorescent cell sorting, and disposable closed cartridge based cell sorters.

Enrichment for cells expressing one or more markers refers to increasing the number or percentage of such cells in the population of cells, but does not necessarily result in a complete absence of cells not expressing the marker. Depletion of cells expressing one or more markers refers to decreasing the number or percentage of such cells in the population of cells, but does not necessarily result in a complete removal of all cells expressing such marker or markers.

In some embodiments, the enrichment comprises a step of affinity- or immunoaffinity-based separation of cells expressing one or more markers (e.g., Treg cell surface markers). Such separation steps can be based on positive selection, in which the cells expressing one or more markers are retained, and/or on negative selection (depletion), in which the cells not expressing one or more markers are retained.

The separation may be based on the expression (e.g., positive or negative expression) or expression level (e.g., high or low expression) of one or more markers (e.g., Treg cell surface markers). In this context, “high expression” and “low expression” are generally relative to the whole population of cells. In some embodiments, separation of cells may be based on CD8 expression. In some embodiments, separation of cells may be based on CD19 expression. In some embodiments, separation of cells may be based on high CD25 expression.

Thus, in some embodiments, enrichment of Tregs may comprise incubation with an antibody or binding partner that specifically binds to a marker (e.g., a Treg cell surface marker), followed generally by washing steps and separation of cells having bound the antibody or binding partner from those cells having not bound to the antibody or binding partner.

In some embodiments, the antibody or binding partner is bound to a solid support or matrix, such as a sphere or bead, for example a nanoparticle, microbeads, nanobeads, including agarose, magnetic bead or paramagnetic beads. In some embodiments, the spheres or beads can be packed into a column to effect immunoaffinity chromatography. In some embodiments, the antibody or binding partner is detectably labeled. In some embodiments, the antibody or binding partner is attached to small, magnetically responsive particles or microparticles, such as nanoparticles or paramagnetic beads. Such beads are known and are commercially available (e.g., Dynabeads® (Life Technologies, Carlsbad, Calif.), MACS® beads (Miltenyi Biotec, San Diego, Calif.) or Streptamer® bead reagents (IBA, Germany)). Such particles or microparticles may be incubated with the population of cells to be enriched and then placed in a magnetic field. This results in those cells that are attached to the particles or microparticles via the antibody or binding partner being attracted to the magnet and separated from the unbound cells. This method allows for retention of the cells attached to the magnet (positive selection) or removal of the cells attracted to the magnet (negative selection).

In some embodiments, a method of producing a therapeutic population of Tregs provided herein comprises both positive and negative selection during the enrichment step.

In some embodiments, the biological sample is obtained within about 25-35 min, about 35-45 min, about 45-60 min, about 60-75 min, about 75-90 min, about 90-120 min, about 120-150 min, about 150-180 min, about 2-3 h, about 3-4 h, about 4-5 h or about 5-6 h of the beginning of the enriching step. In some embodiments, the sample is obtained within about 30 min of the beginning of the enriching step. In some embodiments, the biological sample is not stored (e.g., stored at 4° C.) over night.

In some embodiments, enrichment of Tregs from a human sample comprises depleting the sample of CD8+ cells. In some embodiments, enrichment of Tregs from a human sample comprises depleting a sample of CD19+ cells. In some embodiments, enrichment of Tregs from a biological sample comprises depleting the sample of CD8+ cells and CD19+ cells. In some embodiments, enrichment of Tregs from a biological sample comprises enriching the cell population for CD25high cells. In some embodiments, enrichment of Tregs from a biological sample comprises depletion of CD8+ cells and CD19+ cells from the sample and enriching the cell population for CD25high cells.

In some embodiments, the population of cells enriched for Tregs comprises an increased proportion of CD4+CD25high Tregs relative to the proportion of CD4+CD25high Tregs in the Tregs prior to enrichment as determined by flow cytometry. In specific embodiments, the proportion of CD4+CD25high Tregs is increased by about 2-fold to about 4-fold, about 4-fold to about 6-fold, about 6-fold to about 8-fold, about 8-fold to about 10-fold, about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold.

In some embodiments, the population of cells enriched for Tregs comprises an increased proportion of CD4+CD25^{high}CD127^{low} Tregs relative to the proportion of CD4+CD25highCD127low Tregs in the Tregs prior to enrichment as determined by flow cytometry. In specific embodiments, the proportion of CD4⁺CD25^{high}CD127^{low} Tregs is increased by about 2-fold to about 4-fold, about 4-fold to about 6-fold, about 6-fold to about 8-fold, about 8-fold to about 10-fold, about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold.

In some embodiments, the population of cells enriched for Tregs comprises CD25+ Tregs wherein the expression of CD25 in the Tregs is increased relative to the expression of CD25 in the Tregs prior to enrichment, as determined by flow cytometry. In specific embodiments, the expression of CD25 is increased by at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold.

In some embodiments, the population of cells enriched for Tregs comprises CD127+ Tregs wherein the expression of CD127 in the Tregs is increased relative to the expression of CD127 in the Tregs prior to enrichment, as determined by flow cytometry. In specific embodiments, the expression of CD127 is increased by at least about 1.5-fold, at least about 2-fold, at least about 2.5-fold, or at least about 3-fold.

In some embodiments, the granularity of the Tregs in the enriched population of Tregs is increased relative to the granularity of the Tregs prior to enrichment, as determined by flow cytometry. In specific embodiments, the granularity of the Tregs increased by at least about 1.5-fold, at least about 2-fold, at least about 2.5-fold, or at least about 3-fold.

In some embodiments, the size of the Tregs in the enriched population of Tregs is increased relative to the size of the Tregs prior to enrichment, as determined by flow cytometry. In specific embodiments, the size of the Tregs increased by at least about 1.2-fold, at least about 1.5-fold, or at least about 2-fold.

### 6.1.2. Methods of Expanding Tregs Ex Vivo

In another aspect, a method provided herein comprises a step of expanding the therapeutic population of Tregs enriched from a biological sample. This expansion of the therapeutic population of Tregs may comprise culturing the cells that have been enriched from a biological samples in media, for example, in serum-free media (e.g., TexMACS Medium). In some embodiments, the cells enriched from a biological sample are cultured about 37° C. and about 5% CO₂. In some embodiments, the cells enriched from a biological sample are cultured out under good manufacturing practice (GMP) conditions. In some embodiments, the cells enriched from a biological sample are cultured in a closed system.

In some embodiments, the expansion of the therapeutic population of Tregs begins within 25-35 min, within 20-40 min, within 15-45 min or within 10-50 min of the enrichment from a biological sample. In some embodiments, the expansion of the therapeutic population of Tregs begins within about 30 min of the enrichment from a biological sample.

Tregs may be expanded ex vivo by culturing the cells in the presence of one or more expansion agents. In some embodiments, the expansion agent is IL-2. The appropriate concentration of IL-2 in the culture media can be determined by a person of skill in the art. In some embodiments, the concentration of IL-2 in the cell culture media is about 5-10 IU/mL, about 10-20 IU/mL, about 20-30 IU/mL, about 30-40 IU/mL, about 40-50 IU/mL, about 50-100 IU/mL, about 100-200 IU/mL, about 200-300 IU/mL, about 300-400 IU/mL, about 400-500 IU/mL, about 500-600 IU/mL, about 600-700 IU/mL, about 700-800 IU/mL, about 800-900 IU/mL, about 900-1000 IU/mL, about 1000-1500 IU/mL, about 1500-2000 IU/mL, about 2000-2500 IU/mL, about 2500-3000 IU/mL, about 3000-3500 IU/mL, about 3500-4000 IU/mL, about 4000-4500 IU/mL, about 4500-5000 IU/mL, about 5000-6000 IU/mL, about 6000-7000 IU/mL, about 7000-8000 IU/mL, about 8000-9000 IU/mL, or about 9000-10,000 IU/mL. In specific embodiments, the concentration of IL-2 in the cell culture media is 500 IU/mL.

In some embodiments, the expansion agent activates CD3, e.g., the expansion agent is an anti-CD3 antibody. In some embodiments, the expansion agent activates CD28, e.g., the expansion agent is an anti-CD28 antibody.

In some embodiments, the expansion agent is a soluble anti-CD3 antibody. In particular embodiments, the anti-CD3 antibody is OKT3. In some embodiments, the concentration of soluble anti-CD3 antibody in the culture media is about 0.1-0.2 ng/mL, about 0.2-0.3 ng/mL, about 0.3-0.4 ng/mL, about 0.4-0.5 ng/mL about 0.5-1 ng/mL, about 1-5 ng/mL, about 5-10 ng/mL, about 10-15 ng/mL, about 15-20 ng/mL, about 20-25 ng/mL, about 25-30 ng/mL, about 30-35 ng/mL, about 35-40 ng/mL, about 40-45 ng/mL, about 45-50 ng/mL, about 50-60 ng/mL, about 60-70 ng/mL, about 70-80 ng/mL, about 80-90 ng/mL, or about 90-100 ng/mL.

In some embodiments, the expansion agent is a soluble anti-CD28 antibody. Non-limiting examples of anti-CD28 antibodies include NA/LE (e.g. BD Pharmingen), IM1376 (e.g. Beckman Coulter), or 15E8 (e.g. Miltenyi Biotec). In some embodiments, the concentration of soluble anti-CD28 antibody in the culture media is about 1-2 ng/mL, about 2-3 ng/mL, about 3-4 ng/mL, about 4-5 ng/mL, about 5-10 ng/mL, about 10-15 ng/mL, about 15-20 ng/mL, about 20-25 ng/mL, about 25-30 ng/mL, about 30-35 ng/mL, about 35-40 ng/mL, about 40-45 ng/mL, about 45-50 ng/mL, about 50-60 ng/mL, about 60-70 ng/mL, about 70-80 ng/mL, about 80-90 ng/mL, about 90-100 ng/mL, about 100-200 ng/mL, about 200-300 ng/mL, about 300-400 ng/mL, about 400-500 ng/mL, 500-600 ng/mL, 600-700 ng/mL, about 700-800 ng/mL, about 800-900 ng/mL, or about 900-1000 ng/mL.

In some embodiments, both an anti-CD3 antibody and an anti-CD28 antibody are present in the cell culture media. In some embodiments, the anti-CD3 antibody and the anti-CD28 antibody are attached to a solid surface. In some embodiments, the anti-CD3 antibody and the anti-CD28 antibody are attached to beads. In some embodiments, beads (e.g., 3.5 μm particles) loaded with CD28 antibodies, anti-biotin antibodies and CD3-Biotin are present in the cell culture medium Such beads are commercially available (e.g., MACS GMP ExpAct Treg Kit, DYNABEADS© M-450 CD3/CD28 T Cell Expander). In specific embodiments, the ratio of anti-CD3 antibody to anti-CD28 antibody on the beads is about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90 or 1:100. In some embodiments, the population of Tregs is cultured in the presence of both IL-2 and beads loaded with CD28 antibodies, anti-biotin antibodies and CD3-Biotin. In some embodiments, the beads coated with anti-CD3 and anti-CD28 antibody are first added to the culture within about 4-5 days of initiating culture. In some embodiments, the beads coated with anti-CD3 and anti-CD28 antibody are again added to the culture medium about 14 days after the beads coated with anti-CD3 and anti-CD28 antibody were first added to the culture medium. In specific embodiments, the ratio of beads to cells in the culture is 10:1, 9:1, 8:1, 7:1, 6:1, 5:1, 4:1, 3:1, 2:1 or 1:1.

The expansion agent or agents may be added to the culture medium every 1, 2, 3, 4, or 5 days. In specific embodiments, the expansion agent is added to the culture medium every 2-3 days. In other specific embodiments, the expansion agent is added to the culture medium on day 6, 8, and 11, wherein day 0 is the day on which the biological sample is obtained from the subject. In some specific embodiments, the expansion agent is not added to the culture medium on day 13, wherein day 0 is the day on which the biological sample is obtained from the subject.

In some embodiments, the one or more expansion agent is first added to the culture within about 16-18 hours, within 18-24 h, within 24-36 h, within 36-48 h, within about 24 h, within about 24 h, within about 3 days, within about 4 days, within about 5 days, within about 6 days, or within about 7 days of initiating culture. In some embodiments, the one or more expansion agent is first added to the culture within about 4-5 days of initiating culture. In some embodiments, the one or more expansion agent is again added to the culture medium about 14 days after the expansion agent was first added to the culture medium.

If no expansion agent is added to the culture on a given day, that day is considered a “rest day.” In some embodiments, no expansion agent is administered during the day preceding the day on which the therapeutic population of Tregs is harvested. In some embodiments, no expansion agent is administered during the 2 days, 3 days, 4 days, 5 days or 6 days preceding the day on which the therapeutic population of Tregs is harvested.

In some embodiments, the therapeutic population of Tregs may be expanded ex vivo by culturing the cells in the presence of one or more agents that inhibit mammalian target of rapamycin (mTor). In some embodiments, the mTor inhibitor is rapamycin. In some embodiments, the mTor inhibitor is an analog of rapamycin (a “rapalog,” e.g., Temsirolimus, Everolimus, or Ridaforolimus). In some embodiments, the mTor inhibitor is ICSN3250, OSU-53, or AZD8055. In some embodiments, the concentration of rapamycin in the cell culture medium is about 1-20 nmol/L, about 20-30 nmol/L, about 30-40 nmol/L, about 40-50 nmol/L, about 50-60 nmol/L, about 60-70 nmol/L, about 70-80 nmol/L, about 80-90 nmol/L, about 90-100 nmol/L, about 100-150 nmol/L, about 150-200 nmol/L, about 200-250 nmol/L, about 250-300 nmol/L, about 300-350 nmol/L, about 350-400 nmol/L, about 400-450 nmol/L, about 450-500 nmol/L, about 500-600 nmol/L, about 600-700 nmol/L, about 700-800 nmol/L, about 800-900 nmo/L or about 900-1000 nmol/L. In some embodiments, the concentration of rapamycin in the cell culture media is about 100 nmol/L.

In some embodiments, the mTor inhibitor is first added to the culture within about 16-18 hours, within 18-24 h, within 24-36 h, within 36-48 h, within about 2 days, about 3 days, about 4 days, about 5 days, about 6 days, or about 7 days of initiating culture. In some embodiments, the mTor inhibitor is added to the culture medium about every 2-3 days.

The therapeutic population of Tregs may be expanded by culturing them for an appropriate duration of time. The time required for expansion resulting in a sufficiently expanded therapeutic population of Tregs for therapeutic application may be readily determined by a person of skill in the art, e.g., by monitoring the proportion of CD4+CD25+ cells using flow cytometry.

For example, in some embodiments, a sufficiently expanded therapeutic population of Tregs is a population of cells that contains more than 70% CD4+CD25+ cells as determined by flow cytometry. In some embodiments, a sufficiently expanded therapeutic population of Tregs is a population that contains about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4+CD25+ cells per kg of body weight of the intended recipient of the therapeutic population of Tregs as determined by flow cytometry. In some embodiments, a sufficiently expanded therapeutic population of Tregs is a population that contains 1×10⁶ CD4+CD25+ cells (+/−10%) per kg of body weight of the intended recipient of the therapeutic population of Tregs as determined by flow cytometry. In some embodiments, a sufficiently expanded therapeutic population of Tregs is a population of cells that contains about or more than about 70% CD4+CD25+ cells and contains 1×10⁶ CD4+CD25+ cells (+/−10%) per kg of body weight of the intended recipient of the therapeutic population of Tregs as determined by flow cytometry.

The intended recipient of the therapeutic population of Tregs may be the same subject as the donor of the biological sample from which the Tregs were enriched.

The number of CD4+CD25+ cells may be determined every day, or every 2, 3, 4, or 5 days. If the culture does not contain a sufficiently expanded therapeutic population of Tregs on Day 15 (wherein day 0 is the day on which the biological sample is obtained from the subject), the cells may be re-activated with one or more expansion agents. If the culture does contain a sufficiently expanded therapeutic population of Tregs on Day 15 (wherein day 0 is the day on which the biological sample is obtained from the subject), the cells may harvested.

In some embodiments, a therapeutic population of Tregs is expanded by culturing for about 6-30 days, about 10-30 days, about 15-25 days, or about 18-22 days. In some embodiments, a therapeutic population of Tregs is expanded by culturing for about 15, 16, 18, 18, 19, 20, 21, 22, 23, 24, or 25 days. In certain embodiments, for example, embodiments comprising automation, partial automation or at least one automated step, a therapeutic population of Tregs is expanded by culturing for about 6-15 days, about 8-15, about 8-12 days, or about 6, 7, 8, 9, 10, 11 or 12 days.

Viability of the cells being expanded in culture may be determined using any method known in the art. For example, the viability of cells being expanded in culture may be determined using trypan blue exclusion. Trypan blue is a dye which is excluded by cells with an intact membrane (viable cells) but taken up by cells with compromised membrane integrity (non-viable cells). Thus, viable cells appear clear under a light microscope, whereas non-viable cells appear blue. Equal amounts of trypan blue and cell suspension are mixed and counted. Viability is expressed as a percentage of trypan blue excluding cells. In some embodiments, a therapeutic population of Tregs comprises about 60%, 65% or 70% viable cells as determined by trypan blue exclusion. In some embodiments, a therapeutic population of Tregs comprises more than about 70% viable cells as determined by trypan blue exclusion. For example, in certain embodiments a therapeutic population of Tregs comprises about 75%, 80%, 85%, 90%, 95% or greater that 95% viable cells as determined by trypan blue exclusion. In some embodiments, viability of the cells being expanded in culture is determined every 2-3 days. In some embodiments, viability of the cells being expanded in culture is determined every day or every 2, 3, 4, or 5 days.

In some embodiments, the cells are washed one or more times during the culturing to remove agents present during the incubation or culturing and/or to replenish the culture medium with one or more additional agents. In some embodiments, the cells are washed during the incubation or culturing to reduce or remove the expansion agent(s). The culture medium may be replaced about every 2, 3, 4, 5, 6 or 7 days, for example, every 2-3 days. In some embodiments, only part of the culture medium (e.g., about 50% of the culture medium) is replaced. In other embodiments, the entire culture medium is replaced. In some embodiments, the cell culture is not centrifuged during a change of culture medium. In some embodiments, the cell culture is not centrifuged during harvesting.

The therapeutic population of Tregs may be harvested by any means known in the art, for example, by centrifugation. In some embodiments, the therapeutic population of Tregs is harvested on day 15, wherein day 0 is the day on which the biological sample is obtained from the subject. In some embodiments, the therapeutic population of Tregs is harvested on day 19, wherein day 0 is the day on which the biological sample is obtained from the subject In some embodiments, the therapeutic population of Tregs is harvested on day 20, wherein day 0 is the day on which the biological sample is obtained from the subject. In some embodiments, the therapeutic population of Tregs is harvested on day 25, wherein day 0 is the day on which the biological sample is obtained from the subject. In some embodiments, the therapeutic population of Tregs is harvested on day 16, 17 or 18, wherein day 0 is the day on which the biological sample is obtained from the subject. In some embodiments, the therapeutic population of Tregs is harvested on day 21, 22, 23, or 24, wherein day 0 is the day on which the biological sample is obtained from the subject.

In some aspects the population of Tregs is subjected to genetic engineering at any point of the method prior to cryopreservation. In some embodiments, the population of Tregs is subjected to genetic engineering two or more times at any point prior to cryopreservation. In some embodiments, the engineering may comprise the introduction of a transgene into the Tregs or the introduction of an mRNA into the Tregs. In some embodiments, the genetic engineering may also comprise the gene editing through CRISPR-Cas9. In some embodiments, the genetic engineering comprises the reduction of gene expression through siRNA or antisense oligonucleotides. In some embodiments, the genetic engineering may allow for the use of a therapeutic population of Tregs provided herein in an allogeneic setting. In some embodiments, the genetic engineering introduces a chimeric antigen receptor (CAR) into the Tregs.

### 6.2 Methods of Cryopreserving and Thawing Trees

In another aspect, provided herein is a cryopreserved population of Tregs having characteristics as described herein. A cryopreserved therapeutic population of Tregs may be produced by the methods described herein. Also disclosed herein are pharmaceutical compositions comprising a cryopreserved therapeutic population of Tregs that has been thawed and is present in a formulation suitable for administration to a subject, for example a human subject. In certain embodiments, the formulation comprises a pharmaceutically acceptable carrier, e.g., normal saline. In certain embodiments, the formulation comprises human serum albumin. In other embodiments, the formulation comprises normal saline and human serum albumin.

In some embodiments, a therapeutic population of Tregs is cryopreserved after expansion. The therapeutic population of Tregs may be cryopreserved in any suitable medium known in the art. Examples of media suitable for cryopreservation include, e.g., CryoStor® CS10. In some embodiments, a therapeutic population of Tregs is frozen in a composition comprising a cryoprotectant, for example, in a composition comprising DMSO (e.g., 10% DMSO). In some embodiments, the therapeutic population of Tregs is frozen in a comprising glycerol. In some embodiments, the cryoprotectant is or comprises DMSO and/or glycerol.

In some embodiments, a therapeutic population of Tregs may be stored at about −200° C. to −190° C., about −180 to −140° C., or about −90 to −70° C. In some embodiments, a therapeutic population of Tregs may be stored at about −196° C. In some embodiments, a therapeutic population of Tregs may be stored at about −80° C. In some embodiments, a therapeutic population of Tregs may be stored in the liquid nitrogen vapor phase. In some embodiments, a therapeutic population of Tregs may be stored on frozen carbon dioxide (dry ice).

In another aspect, a cryopreserved therapeutic population of Tregs may be stored at a first temperature for a period of time, for example an extended period of time, e.g., about month, about 3 months, about 6 months, about 9 months, about 12 months, about 18 months, or about 24 months, and subsequently stored at a second temperature for a shorter period of time (e.g., about 6 hours, about 12 hours, about 24 hours, about 36 hours, or about 48 hours). In another aspect, a cryopreserved therapeutic population of Tregs may be stored at a first temperature for a period of time, e.g., about 6 hours, about 12 hours, about 24 hours, about 36 hours, or about 48 hours, subsequently stored at a second temperature for a longer period of time e.g., about month, about 3 months, about 6 months, about 9 months, about 12 months, about 18 months, or about 24 months. In some embodiments, the first temperature is lower than the second temperature. In some embodiments, the first temperature is about −200° C. to −190° C., about −180 to −140° C., about −90 to −70° C., about −196° C. or about −80° C. In some embodiments, the second temperature is about −80° C. or about −20° C.

In some embodiments, a therapeutic population of Tregs is stored at about −196° C. for about 1 month, about 3 months, about 6 months, about 9 months, about 12 months, about 18 months, or about 24 months and subsequently stored on frozen carbon dioxide for about 6 hours, about 12 hours, about 24 hours, about 36 hours, or about 48 hours. In some embodiments, a therapeutic population of Tregs is stored in the liquid nitrogen vapor phasefor about 1 month, about 3 months, about 6 months, about 9 months, about 12 months, about 18 months, or about 24 months and subsequently stored on frozen carbon dioxide for about 6 hours, about 12 hours, about 24 hours, about 36 hours, or about 48 hours.

In some embodiments, the therapeutic population of Tregs is cryopreserved at a high Treg density. In specific embodiments, the therapeutic population of Tregs is cryopreserved at a concentration of 1×10⁶ cells (+/−10%) per kg of body weight of the intended recipient of the Tregs. The intended recipient of the Tregs may be the same or different individual as the subject from whom the initial biological sample containing Tregs was obtained (the donor). In some embodiments, the cryopreserved therapeutic population of Tregs is stored at a density of at 10 million, at least 20 million, at least 25 million, at least 30 million, at least 35 million, at least 40 million, at least 45 million, at least 50 million, at least 55 million, at least 60 million, at least 65 million, at least 70 million, at least 75 million, at least 80 million, at least 85 million, at least 90 million, at least 95 million, or at least 100 million cells per mL.

In some embodiments, the therapeutic population of Tregs is cryopreserved in a cryovial. In some embodiments, the therapeutic population of Tregs is frozen in a cryovial in a volume of about 0.5 mL to 1 mL, about 1 mL to 1.5 mL, or about 1.5 mL to 2 mL, about 2-5 mL, about 5-10 mL, or about 15-20 mL. In some embodiments, the therapeutic population of Tregs is frozen in a cryovial in a volume of about 1.0 mL, about 1.1 mL, about 1.2 mL, about 1.3 mL, about 1.4 mL, about 1.5 mL, about 1.6 mL, about 1.7 mL, about 1.8 mL, about 1.8 mL, about 1.9 mL or about 2.0 mL.

In some embodiments, the therapeutic population of Tregs is frozen in a cryopreservation bag, e.g., a gas permeable bag. In some embodiments, the therapeutic population of Tregs is frozen in a cryopreservation bag in a volume of about 1-2 mL, about 2-5 mL, about 5-10 mL, about 10-15 mL, or about 15-20 mL. In some embodiments, the therapeutic population of Tregs is frozen in a cryopreservation bag in a volume of about 1 mL, about 2 mL, about 5 mL, about 10 mL or about 20 mL.

In some embodiments, cryopreservation comprises decreasing the temperature of the therapeutic population of Tregs in the following increments: 1° C./min to 4° C., 25° C./min to −40° C., 10° C./min to −12° C., 1° C./min to −40° C., and 10° C./min to −80° C. to −90° C.

The cryopreserved therapeutic population of Tregs may be thawed, for example, about 1 day, about 2 days, about 3 days, about 4 days, about 5 days, about 6 days, about 1 week, about 1-2 weeks, about 2-4 weeks, about 1 month, about 1-2 months, about 2-3 months, about 3-6 months, about 6-9 months, about 9-12 months, about 12-15 months, about 15-18 months, about 18-24 months, about 1-2 years, about 2-3 years, about 3-4 year or about 4-5 years after cryopreservation. In some embodiments, the cryopreserved therapeutic population of Tregs may be thawed, for example, about 1 week, 1 month, about 3 months, about 6 months, about 9 months, about 12 months or about 18 months after cryopreservation.

In some embodiments, the cryopreserved therapeutic population of Tregs is thawed using a method that results in at least 60%, at least 70%, at least 75%, at least 80%, at least 85%, at least 90%, at least 95% or greater than 95% viability of the Tregs as determined, for example, by Trypan Blue exclusion.

In some embodiments, the cryopreserved therapeutic population of Tregs is thawed and diluted in a solution comprising 0.9% sodium chloride. In some embodiments, the cryopreserved therapeutic population of Tregs is thawed and diluted in a solution comprising 0.9% sodium chloride and about 5% human serum albumin. In some embodiments, the cryopreserved therapeutic population of Tregs is thawed and diluted in a solution wherein the resulting solution comprises 0.9% sodium chloride. In some embodiments, the cryopreserved therapeutic population of Tregs is thawed and diluted in a solution wherein the resulting solution comprises 0.9% sodium chloride and about 5% human serum albumin. In certain embodiments, the cryopreserved therapeutic population of Tregs is thawed and without further expansion is placed into a solution as described herein.

In some embodiments, the cryopreserved therapeutic population of Tregs is thawed and diluted in 50 mL of a solution comprising 0.9% sodium chloride and about 5% human serum albumin. In some embodiments, the cryopreserved therapeutic population of Tregs is thawed and diluted in a solution, wherein the resulting solution is a 50 mL solution comprising 0.9% sodium chloride and about 5% human serum albumin. In certain embodiments, the cryopreserved therapeutic population of Tregs is thawed and without further expansion is placed into a solution as described herein.

In some embodiments, one cryovial containing the cryopreserved therapeutic population of Tregs is thawed and placed in 50 mL of a solution comprising 0.9% sodium chloride and about 5% human serum albumin. In some embodiments, one cryovial containing the cryopreserved therapeutic population of Tregs is thawed and placed in solution, wherein the resulting solution is a 50 mL solution comprising 0.9% sodium chloride and about 5% human serum albumin. In certain embodiments, the cryopreserved therapeutic population of Tregs is thawed and without further expansion is placed into a solution as described herein.

In some embodiments, the cryopreserved therapeutic population of Tregs is thawed using an automated thawing system (e.g., a COOK regentec thawing system). In some embodiments, the cryopreserved therapeutic populations of Tregs is rapidly thawed. In some embodiments, the cryopreserved therapeutic population of Tregs is thawed at a controlled rate. An exemplary thawing protocol is described in Section 8.6 below. In some embodiments, the cryopreserved therapeutic population of Tregs is thawed in a water bath. In some embodiments, the cryopreserved therapeutic population of Tregs is thawed at room temperature.

In some embodiments, the therapeutic population of Tregs is administered to a subject within about 2-10 hours, within about 4-8 hours, or within about 5-7 hours of thawing. In some embodiments, the therapeutic population of Tregs is administered to a subject within about 30 minutes, or about 1, 2, 3, 4, 5, 6, 7, 8, 9, 10, 11 or 12 hours of thawing. In specific embodiments, the therapeutic population of Tregs is administered to the subject within about 6 hours of thawing.

In some embodiments, the cryopreserved therapeutic population of Tregs is thawed and administered to the patient without further dilution. In some embodiments, the cryopreserved therapeutic population of Tregs is thawed and administered to the patient without further dilution or further expansion. In some embodiments, the cryopreserved therapeutic population of Tregs is thawed and administered to the patient without further dilution and in combination with normal saline. In some embodiments, the cryopreserved therapeutic population of Tregs is thawed and administered to the patient without further dilution or further expansion and in combination with normal saline. In some embodiments, the thawed cryopreserved therapeutic population of Tregs and normal saline are administered concurrently and intravenously.

In some embodiments, no further expansion of the therapeutic population of Tregs is required between thawing and administration to the subject. In some embodiments, no further expansion of the therapeutic population of Tregs is performed between thawing and administration to the subject.

### 6.2.1. Automation

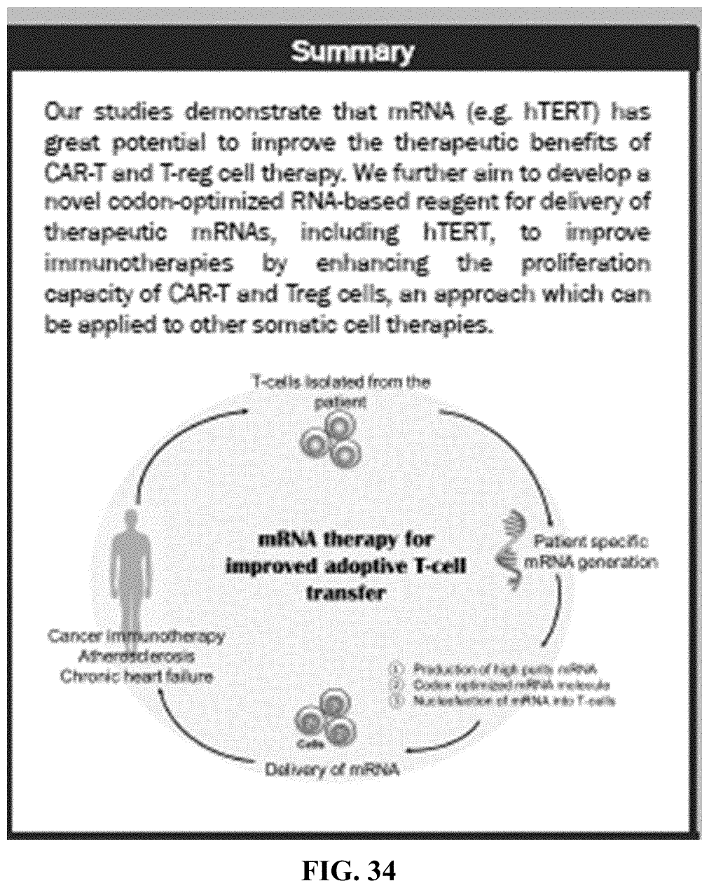

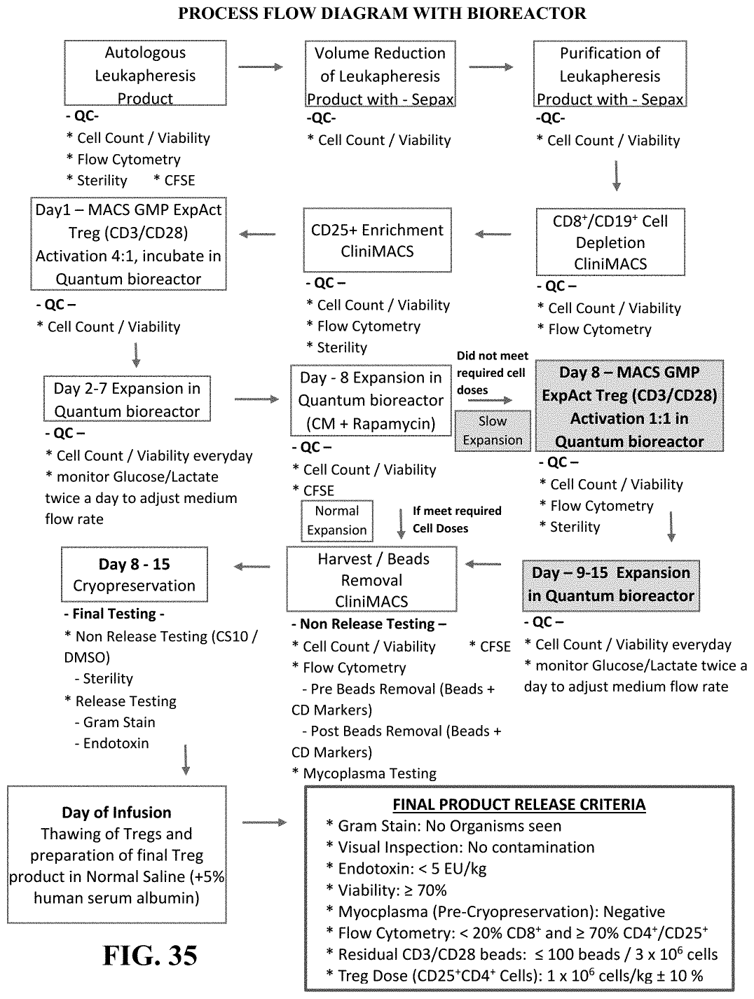

In some embodiments, a method of producing a therapeutic population of Tregs can be carried out in a closed system. The methods in some embodiments are carried out in an automated or partially automated fashion. For example, the method of producing a therapeutic population of Tregs described herein (e.g., the method described in Section 6.1, 8.3 or 8.5 herein) may be carried out in a bioreactor. In some embodiments, the method is carried out in a G-REX® culture system. FIG. 34 shows an exemplary process of producing a therapeutic population of Tregs ins a bioreactor.

In some embodiments, any one or more of the steps of the method of producing a therapeutic population of Tregs can be carried out in a closed system or under GMP conditions. In some embodiments, one or more or all of the steps (e.g., enrichment and/or expansion) is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the steps

In some embodiments, the method of producing a therapeutic population of Tregs comprises a step of expanding the Tregs. In some embodiments, the expansion step is automated. In some embodiments, the Tregs are expanded for 6, 7, 8, 9, 10, 11, or 12 days. In one embodiment, the Tregs are expanded for 8 days. In another embodiment, the Tregs are expanded for 13, 14, or 15 days. In another embodiment, the Tregs are expanded for 15 days.

In some embodiments, the method of producing a therapeutic population of Tregs comprises administering an expansion agent (such as IL-2, a CD3-activating agent and/or a CD28-activating agent) every 1, 2, 3, 4, 5, 6, 7, 8, 9 or 10 days. In some embodiments, the expansion agent (such as IL-2, a CD3-activating agent and/or a CD28-activating agent) is first administered within 24 h of initiating the culture. In some embodiments, the expansion agent (such as IL-2, a CD3-activating agent and/or a CD28-activating agent) is administered daily. In some embodiments, the method of producing a therapeutic population of Tregs comprises replacing the culture medium every 1, 2, 3, 4, 5, 6 or 7 days. In some embodiments, the media may be changed when the level of certain metabolites (e.g., lactacte) reach a predetermined threshold. In some embodiments, the media changes based on the expansion rate of the therapeutic population of Tregs.

In some embodiments, the concentration of cells in the culture is determined on Day 8. In some embodiments, the concentration of cells in the culture is determined on Day 15. In some embodiments, the system remains closed throughout the expansion step.

### 6.3 Compositions

Provided herein are compositions comprising a therapeutic population of Tregs suitable for administration to a subject. In some embodiments, provided herein is a cryopreserved composition comprising a therapeutic population of Tregs having characteristics as described herein. A therapeutic populatin of Tregs, for example, a cryopreserved therapeutic population of Tregs, may be produced by the methods described herein. Also disclosed herein are pharmaceutical compositions comprising a cryopreserved therapeutic population of Tregs that has been thawed and is present in a formulation suitable for administration to a subject, for example a human subject.

In some embodiments, a therapeutic population of Tregs provided herein comprises about 60%, 65%, 70%, 75%, 80%, 85%, 90%, 95% or greater than 95% viable cells as determined by trypan blue exclusion. In some embodiments, a therapeutic population of Tregs provided herein comprises more than about 70% viable cells.

In some embodiments, a therapeutic population of Tregs provided herein comprises about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4+CD25+ cells.

In some embodiments, a therapeutic population of Tregs provided herein comprises about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4+CD25+ cells per mL.

In some embodiments, a therapeutic population of Tregs provided herein comprises about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4+CD25+ cells per kg of body weight of an intended recipient subject as determined by flow cytometry. In some embodiments, a therapeutic population of Tregs provided herein comprises 1×10⁶ CD4+CD25+ cells (+/−10%) per kg of body weight of the subject as determined by flow cytometry. In some embodiments, a therapeutic population of Tregs provided herein comprises about or more than about 70% CD4+CD25+ cells and contains 1×10⁶ CD4+CD25+ cells (+/−10%) per kg of body weight of the subject as determined by flow cytometry. The subject may be the same subject as the donor of the biological sample from which the Tregs were enriched.

A cryopreserved therapeutic population of Tregs provided herein may comprise an increased proportion of CD4⁺CD25^{high} Tregs relative to the proportion of CD4⁺CD25^{high} Tregs in a corresponding baseline Treg cell population, as determined by flow cytometry. In some embodiments, a cryopreserved therapeutic population of Tregs comprises an increased proportion of CD4⁺CD25^{high}CD127^{low} Tregs relative to the proportion of CD4⁺CD25^{high}CD127^{low} Tregs in the baseline Treg cell population, as determined by flow cytometry. In some embodiments, an therapeutic population of Tregs provided herein may comprise an increased proportion of CD4⁺CD25^{high} Tregs relative to the proportion of CD4⁺CD25^{high} Tregs in the baseline Treg cell population, as determined by flow cytometry. In some embodiments, an expanded therapeutic population of Tregs comprises an increased proportion of CD4⁺CD25^{high}CD127^{low} Tregs relative to the proportion of CD4⁺CD25^{high}CD127^{low} Tregs in the baseline Treg cell population, as determined by flow cytometry. In this context, “high” and “low” denote the expression level of a marker (e.g., CD25 or CD127) in a subpopulation of cells relative to the entire population.

In some embodiments, the cryopreserved therapeutic population of Tregs comprises CD25⁺ Tregs wherein the expression of CD25 in the Tregs is increased relative to the expression of CD25 in the Tregs in the baseline Treg cell population, as determined by flow cytometry. The expression of CD25 is increased by at least about 5-fold, at least about 10-fold, at least about 15-fold, at least about 20-fold, at least about 25-fold, at least about 30-fold, at least about 35-fold, at least about 40-fold, at least about 45-fold or at least about 50-fold as determined by flow cytometry.

In some embodiments, the cryopreserved population of Tregs comprises CD127⁺ Tregs wherein the expression of CD127 in the Tregs is not increased greater than 3-fold relative to the expression of CD127 in the Tregs in the baseline Treg cell population, as determined by flow cytometry. In some embodiments, the expression of CD127 is not increased relative to the expression of CD127 in the Tregs in the Treg-enriched cell population, as determined by flow cytometry.

In some embodiments, the cryopreserved therapeutic population of Tregs comprises at least 70%, at least 80%, or at least 90% CD4⁺CD25^{high}CD127^{low} Tregs in the baseline Treg cell population, as determined by flow cytometry. In some embodiments, the cryopreserved therapeutic population of Tregs comprises at least 70%, at least 75%, at least 80%, at least 85%, at least 90% or at least 95% CD4⁺CD25⁺ cells, as determined by flow cytometry. In some embodiments, a composition comprising a therapeutic population of Tregs provided herein comprises less than 20% CD8+ cells and comprises 1×10⁶ CD4+CD25+ cells (+/−10%) per kg of body weight of the subject as determined by flow cytometry. In some embodiments, a composition comprising a therapeutic population of Tregs provided herein comprises less than 20% CD8+ cells, comprises about or more than about 70% CD4+CD25+ cells, and comprises 1×10⁶ CD4+CD25+ cells (+/−10%) per kg of body weight of the subject as determined by flow cytometry.

In some embodiments, the granularity of the Tregs in the cryopreserved therapeutic population of Tregs is increased relative to the granularity of the Tregs in the Treg-enriched cell population, as determined by flow cytometry. In some embodiments, the granularity of the Tregs is increased by at least about 1.5-fold, at least about 2-fold, or at least about 2.5-fold.

In some embodiments, the size of the Tregs in the cryopreserved therapeutic population of Tregs is increased relative to the size of the Tregs in the baseline Treg cell population, as determined by flow cytometry. In some embodiments, the size of the Tregs is increased by at least about 1.2-fold, at least about 1.5-fold, or at least about 2-fold.

In some embodiments, the cryopreserved therapeutic population of Tregs comprises CTLA4+ Tregs wherein the proportion of CTLA4+ Tregs is increased relative to the proportion of CTLA4+ Tregs in the baseline Treg cell population. In some embodiments, an expanded therapeutic population of Tregs comprises CTLA4+ Tregs wherein the proportion of CTLA4+ Tregs is increased relative to the proportion of CTLA4+ Tregs in the baseline Treg cell population. In some embodiments, the cryopreserved therapeutic population of Tregs comprises at least 10%, at least 20%, at least 30%, at least 40%, at least 50%, at least 60%, at least 70%, at least 80%, or at least 90% CTLA4+ Tregs, as determined by flow cytometry.

In some embodiments, the cryopreserved therapeutic population of Tregs comprises FoxP3+ Tregs wherein the proportion of FoxP3+ Tregs is increased relative to the proportion of FoxP3+ Tregs in the baseline Treg cell population. In some embodiments, an expanded therapeutic population of Tregs comprises FoxP3+ Tregs wherein the proportion of FoxP3+ Tregs is increased relative to the proportion of FoxP3+ Tregs in the baseline Treg cell population. In some embodiments, the cryopreserved therapeutic population of Tregs comprises at least 10%, at last 20%, at last 30%, at least 40%, at least 50%, at least 60%, at least 70%, or at least 80% or at least 90% FoxP3+ Tregs, as determined by flow cytometry.

In some embodiments, the Tregs in the cryopreserved therapeutic population of Tregs comprise FoxP3-expressing Tregs wherein the expression of FoxP3 is increased in the Tregs relative to expression of FoxP3 in the Tregs in the baseline Treg cell population prior to expansion. In some embodiments, a therapeutic population of Tregs or a cryopreserved composition comprising a therapeutic population of Tregs provided herein expresses high levels of FoxP3, wherein the one or more regulatory element (e.g., a promoter or an enhancer) of the FOXP3 gene is demethylated. In some embodiments, the Treg specific demethylated region (TSDR) within FOXP3 is demethylated. In some embodiments, the expression of one or more gene products associated with FOXP3 demethylation (e.g. one or more gene products listed in Table 14-Table 19 below) is increased in the therapeutic population of Tregs or in the cryopreserved therapeutic population of Tregs after expansion. In some embodiments, one or more gene products associated with FOXP3 methylation (e.g., one or more gene products listed in Table 12 below) is decreased in the therapeutic population of Tregs or in the cryopreserved therapeutic population of Tregs after expansion.

In some embodiments, a cryopreserved population of Tregs expresses high levels of glucocorticoid-induced tumor necrosis factor receptor (GITR).

In some embodiments, a cryopreserved population of Tregs comprises less than 20% CD8+ cells, determined by flow cytometry.

In some embodiments, the viability of the cryopreserved therapeutic population of Tregs is at least 70%, at least 75%, at least 80%, at least 85%, at least 90% or at least 95%, as determined by trypan blue staining performed following thawing of the cryopreserved therapeutic population. In some embodiments, the viability of the cryopreserved therapeutic population of Tregs, as determined by trypan blue staining performed following thawing of the cryopreserved therapeutic population, is at least 70%, at least 75%, at least 80%, at least 85%, at least 90% or at least 95% of the viability of the expanded Treg cell population prior to the expanded Treg cell population being cryopreserved. In some embodiments, the viability of the therapeutic population of Tregs, as determined by trypan blue staining performed before cryopreserving the therapeutic population of Tregs is increased compared to the viability of the enriched Treg cell population prior to expansion.

In some embodiments, the suppressive function of the cryopreserved population of Tregs is at least 60%, at least 70%, at least 75%, at least 80%, at least 85%, at least 90% or at least 95%, as determined by suppression of proliferation of responder T cells by flow cytometry or thymidine incorporation performed following thawing of the therapeutic population. Suppressive function of Tregs may be assessed, for example by measuring the proliferation of CFSE positive responder T cells by flow cytometry or thymidine incorporation. CFSE is an intracellular marker only present in the responder T cell population. Responder T cells are generally characterized as CD4⁺CD25⁻ T cells and may be isolated using the CD4+CD25+ Regulatory T Cell Isolation Kit (Miltenyi Biotec). For example, a sample maybe separated into a positively selected cell fraction containing CD4⁺CD25⁺ regulatory T cells and the unlabeled CD4+CD25− cell effluent which contains the responder T cell population.

In some embodiments, the cryopreserved population of Tregs exhibits a suppressive function that is greater than the suppressive function of the enriched Treg cell population, as measured prior to expansion, as suppression of proliferation of responder T cells by flow cytometry or thymidine incorporation. In some embodiments, the suppressive function of the cryopreserved therapeutic population of Tregs, as determined by suppression of proliferation of responder T cells by flow cytometry or thymidine incorporation performed following thawing of the therapeutic population, is at least 60%, at least 70%, at least 75%, at least 80%, at least 85%, at least 90% or at least 95% of the suppressive function of the expanded Treg cell population prior to the Tregs being cryopreserved, as determined by flow cytometry. In some embodiments, the suppressive function of the therapeutic population of Tregs, as determined by suppression of proliferation of responder T cells by flow cytometry or thymidine incorporation performed before cryopreserving the therapeutic population of Tregs is increased compared to the suppressive function of the enriched Treg cell population prior to expansion.

In some embodiments, the cryopreserved therapeutic population of Tregs exhibits an ability to suppress inflammatory cells, as measured by IL-6 production by the inflammatory cells (e.g., macrophages or monocytes from human donors or generated from induced pluripotent stem cells). In some embodiments, the cryopreserved therapeutic population of Tregs exhibits an ability to suppress the function of myeloid cells (e.g., macrophages, monocytes, or microglia). In some embodiments, the cryopreserved therapeutic population of Tregs exhibits an ability to suppress the release of inflammatory cytokines (e.g., IL-1β, IL-6, IL-8, TNFα, or INFγ).

In some embodiments, the expanded therapeutic population of Tregs exhibits an ability to suppress inflammatory cells, as measured by IL-6 production by the inflammatory cells (e.g., macrophages or monocytes from human donors or generated from induced pluripotent stem cells). In some embodiments, the expanded therapeutic population of Tregs exhibits an ability to suppress the function of myeloid cells (e.g., macrophages, monocytes, or microglia). In some embodiments, the expanded therapeutic population of Tregs exhibits an ability to suppress the release of inflammatory cytokines (e.g., IL-1β, IL-6, IL-8, TNFα, or INFγ).

In specific embodiments, the cryopreserved population of Tregs exhibits an improved ability to suppress secretion of inflammatory cytokines (e.g., IL-1β, IL-6, IL-8, TNFα, or INFγ) from macrophages relative to the therapeutic population of Tregs prior to expansion. In other specific embodiments, the cryopreserved population of Tregs exhibits an improved ability to suppress secretion of inflammatory cytokines (e.g., IL-1β, IL-6, IL-8, TNFα, or INFγ) from monocytes relative to the therapeutic population of Tregs prior to expansion. In other specific embodiments, the cryopreserved population of Tregs exhibits an improved ability to suppress secretion of inflammatory cytokines (e.g., IL-1β, IL-6, IL-8, TNFα, or INFγ) from microglia relative to the therapeutic population of Tregs prior to expansion.

In specific embodiments, the expanded population of Tregs prior to cryopreservation exhibits an improved ability to suppress secretion of inflammatory cytokines (e.g., IL-1β, IL-6, IL-8, TNFα, or INFγ) from macrophages relative to the therapeutic population of Tregs prior to expansion. In other specific embodiments, the expanded population of Tregs prior to cryopreservation exhibits an improved ability to suppress secretion of inflammatory cytokines (e.g., IL-1β, IL-6, IL-8, TNFα, or INFγ) from monocytes relative to the therapeutic population of Tregs prior to expansion. In other specific embodiments, the expanded population of Tregs prior to cryopreservation exhibits an improved ability to suppress secretion of inflammatory cytokines (e.g., IL-1β, IL-6, IL-8, TNFα, or INFγ) from microglia relative to the therapeutic population of Tregs prior to expansion.

In some embodiments, the cryopreserved population of Tregs is thawed in accordance with a method described herein, e.g. a method described in Section 8.6 below.

### 6.3.1. Gene Product Expression Profile

The therapeutic populations of Tregs and cryopreserved compositions comprising a therapeutic population of Tregs provided herein may be characterized by their gene product expression profiles. For example, the gene product expression profile of a cryopreserved composition comprising a therapeutic population of Tregs provided herein may be compared to the Tregs at baseline. In this context, the term “baseline,” or “baseline Treg cell population denotes a population of Tregs that has been enriched from a patient sample but has not yet been expanded. In some embodiments, the gene product expression profile is substantially the same in a cryopreserved composition comprising a therapeutic population of Tregs compared to the Tregs at baseline. In this context of gene product expression as discussed herein, two values are considered to be substantially the same when the difference between them is not statistically significant (i.e., p>0.05) and/or if the binary log of the fold-difference between the two values is less than about 2-fold (increase or decrease).

Changes in gene product expression may be expressed as “-fold increase” or “-fold decrease” or as the binary logarithm (or “log 2”) of the -fold change. In some embodiments, gene product expression is increased at least about 4-fold. In some embodiments, gene product expression is increased about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold. In some embodiments, gene product expression is decreased at least about 4-fold. In some embodiments, gene product expression is increased about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold.

In some embodiments, the expression level of one or more gene product listed in Table 18 is detectable (i.e., above the level of detection for the method utilized such as single-shot proteomics) in a therapeutic population of Tregs or a cryopreserved composition comprising a therapeutic population of Tregs provided herein. In some embodiments, the expression level of at least 2, at least 5, at least 10, at least 15, at least 20, at least 25, at least 30, at least 35, or at least 40 gene products listed in Table 18 may be detectable (i.e., above the level of detection for the method utilized such as single-shot proteomics) in a therapeutic population of Tregs or a cryopreserved composition comprising a therapeutic population of Tregs provided herein. In some embodiments, the expression level of every gene product listed in Table 18 is detectable (i.e., above the level of detection for the method utilized such as single-shot proteomics) in a therapeutic population of Tregs or a cryopreserved composition comprising a therapeutic population of Tregs provided herein.

In some embodiments, the expression level of one or more gene product listed in Table 18 is among the 50, 60, 70, 80, 90, 100, 110, 120, 130, 140 or 150 most highly-expressed gene products as determined by an unbiased proteomics analysis (e.g., single-shot proteomics) in a therapeutic population of Tregs or a cryopreserved composition comprising a therapeutic population of Tregs provided herein. In some embodiments, the expression level of at least 2, at least 5, at least 10, at least 15, at least 20, at least 25, at least 30, at least 35, or at least 40 gene products listed in Table 18 is among the 50, 60, 70, 80, 90, 100, 110, 120, 130, 140 or 150 most highly-expressed gene products as determined by an unbiased proteomics analysis (e.g., single-shot proteomics) in a therapeutic population of Tregs or a cryopreserved composition comprising a therapeutic population of Tregs provided herein. In some embodiments, the expression level of every gene product listed in Table 18 is among the 50, 60, 70, 80, 90, 100, 110, 120, 130, 140 or 150 most highly-expressed gene products as determined by an unbiased proteomics analysis (e.g., single-shot proteomics) in a therapeutic population of Tregs or a cryopreserved composition comprising a therapeutic population of Tregs provided herein.

The level of gene product expression may be above or below the limit of detection of the method being utilized to measure gene product expression. In some embodiments, the expression level of a gene product listed in any of Table 12-Table 19 may be undetectable (i.e., below the level of detection for the method utilized such as single-shot proteomics) in a therapeutic population of Tregs or a cryopreserved composition comprising a therapeutic population of Tregs provided herein. In some embodiments, the expression level of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in any of Table 12-Table 19 may be undetectable (i.e., below the level of detection for the method utilized such as single-shot proteomics) in a therapeutic population of Tregs or a cryopreserved composition comprising a therapeutic population of Tregs provided herein. In some embodiments, the expression level of every gene product listed in any one of Table 12-Table 19 may be undetectable (i.e., below the level of detection for the method utilized such as single-shot proteomics) in a therapeutic population of Tregs or a cryopreserved composition comprising a therapeutic population of Tregs provided herein.

In some embodiments, the expression level of a gene product listed in any of Table 12-Table 19 may be detectable (i.e., above the level of detection for the method utilized such as single-shot proteomics) in a therapeutic population of Tregs or a cryopreserved composition comprising a therapeutic population of Tregs provided herein. In some embodiments, the expression level of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in any of Table 12-Table 19 may be detectable (i.e., above the level of detection for the method utilized such as single-shot proteomics) in a therapeutic population of Tregs or a cryopreserved composition comprising a therapeutic population of Tregs provided herein. In some embodiments, the expression level of every gene product listed in any one of Table 12-Table 19 may be detectable (i.e., above the level of detection for the method utilized such as single-shot proteomics) in a therapeutic population of Tregs or a cryopreserved composition comprising a therapeutic population of Tregs provided herein.

In some embodiments, the expression level of a gene product listed in any of Table 12-Table 19 may become detectable or undetectable in a therapeutic population of Tregs upon enrichment, expansion or cryopreservation. In some embodiments, the expression level of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in any of Table 12-Table 19 may become detectable or undetectable in a therapeutic population of Tregs upon enrichment, expansion or cryopreservation. In some embodiments, the expression level of every gene product listed in any one of Table 12-Table 19 may become detectable or undetectable in a therapeutic population of Tregs upon enrichment, expansion or cryopreservation.

Gene product expression may be determined by any method known in the art, for example, quantitative real-time PCR, Fluidigm™ Chip assays, RNA sequencing, or proteomics analysis (e.g., single-shot proteomics).

In some embodiments, expression of one or more of the gene products listed in Table 12 and/or Table 13 is decreased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, or at least 90 of the gene products listed in Table 12 and Table 13 is decreased in the Tregs post-expansion compared to the Tregs at baseline. In some embodiments, expression of all the gene products listed in Table 12 and Table 13 is decreased in the Tregs post-expansion compared to the Tregs at baseline.

In some embodiments, expression of one or more of the gene products listed in Table 12 is decreased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, or at least 80 gene products listed in Table 12 is decreased in the Tregs post-expansion compared to the Tregs at baseline. In some embodiments, expression of every gene product listed in Table 12 is decreased in the Tregs post-expansion compared to the Tregs at baseline.

In some embodiments, expression of one or more of the gene products listed in Table 13 is decreased in the Tregs post-expansion compared to the Tregs at baseline. In some embodiments, expression of at least 2, at least 3, at least 4, at least 5, at least 6, at least 7, or at least 8 gene products listed in Table 13 is decreased in the Tregs post-expansion compared to the Tregs at baseline. In some embodiments, expression of every gene product listed in Table 13 is decreased in the Tregs post-expansion compared to the Tregs at baseline.

In some embodiments, expression of one or more of the gene products listed in Table 12 and/or Table 13 that are not also listed in Table 19 is decreased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, or at least 90 of the gene products listed in Table 12 and Table 13 that are not also listed in Table 19 is decreased in the Tregs post-expansion compared to the Tregs at baseline. In some embodiments, expression of all of the gene products listed in Table 12 and Table 13 that are not also listed in Table 19 is decreased in the Tregs post-expansion compared to the Tregs at baseline.

In some embodiments, expression of one or more of the gene products listed in Table 12 that are not also listed in Table 19 is decreased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, or at least 80 gene products listed in Table 12 that are not also listed in Table 19 is decreased in the Tregs post-expansion compared to the Tregs at baseline. In some embodiments, expression of every gene product listed in Table 12 that is not also listed in Table 19 is decreased in the Tregs post-expansion compared to the Tregs at baseline.

In some embodiments, expression of one or more of the gene products listed in Table 13 that are not also listed in Table 19 is decreased in the Tregs post-expansion compared to the Tregs at baseline. In some embodiments, expression of at least 2, at least 3, at least 4, at least 5, at least 6, at least 7, or at least 8 of the gene products listed in Table 13 that are not also listed in Table 19 is decreased in the Tregs post-expansion compared to the Tregs at baseline. In some embodiments, expression of every gene product listed in Table 13 that is not also listed in Table 19 is decreased in the Tregs post-expansion compared to the Tregs at baseline.

In some embodiments, the expression of one or more of the gene products listed in Table 12 and/or Table 13 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80 at least 85, or at least 90 gene products listed in Table 12 and/or Table 13 that are not also listed in Table 19 is substantially the same in the cryopreserved therapeutic population of Tregs upon thawing as the expression of the one or more gene products in the Tregs at baseline. In some embodiments, the expression of every gene product listed in Table 12 and Table 13 that is not also listed in Table 19 is substantially the same in the cryopreserved therapeutic population of Tregs upon thawing as the expression of the one or more gene products in the Tregs at baseline.

In some embodiments, the expression of one or more of the gene products listed in Table 12 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, or at least 80 gene products listed in Table 12 that are not also listed in Table 19 is substantially the same in the cryopreserved therapeutic population of Tregs upon thawing as the expression of the one or more gene products in the Tregs at baseline. In some embodiments, the expression of every gene product listed in Table 12 that is not also listed in Table 19 is substantially the same in the cryopreserved therapeutic population of Tregs upon thawing as the expression of the one or more gene products in the Tregs at baseline.

In some embodiments, the expression of one or more of the gene products listed in Table 13 that are not also listed in Table 19 is substantially the same in the cryopreserved therapeutic population of Tregs upon thawing as the expression of the one or more gene products in the Tregs at baseline. In some embodiments, the expression of at least 2, at least 3, at least 4, at least 5, at least 6, at least 7, or at least 8 gene products listed in Table 13 that are not also listed in Table 19 is substantially the same in the cryopreserved therapeutic population of Tregs upon thawing as the expression of the one or more gene products in the Tregs at baseline. In some embodiments, the expression of every gene product listed in Table 13 that is not also listed in Table 19 is substantially the same in the cryopreserved therapeutic population of Tregs upon thawing as the expression of the one or more gene products in the Tregs at baseline.

In some embodiments, the expression of one or more of the gene products listed in Table 12 and/or Table 13 that are not also listed in Table 19 is substantially the same in the cryopreserved therapeutic population of Tregs upon thawing as the expression of the one or more gene products in the Tregs after expansion. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, or at last 90 gene products listed in Table 12 and/or Table 13 that are not also listed in Table 19 is substantially the same in the cryopreserved therapeutic population of Tregs upon thawing as the expression of the one or more gene products in the Tregs after expansion. In some embodiments, the expression of every gene product listed in Table 12 and/or Table 13 that is not also listed in Table 19 is substantially the same in the cryopreserved therapeutic population of Tregs upon thawing as the expression of the one or more gene products in the Tregs after expansion.

In some embodiments, the expression of one or more of the gene products listed in Table 12 that are not also listed in Table 19 is substantially the same in the cryopreserved therapeutic population of Tregs upon thawing as the expression of the one or more gene products in the Tregs after expansion. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, or at least 80 gene products listed in Table 12 that are not also listed in Table 19 is substantially the same in the cryopreserved therapeutic population of Tregs upon thawing as the expression of the one or more gene products in the Tregs after expansion. In some embodiments, the expression of every gene product listed in Table 12 that is not also listed in Table 19 is substantially the same in the cryopreserved therapeutic population of Tregs upon thawing as the expression of the one or more gene products in the Tregs after expansion.

In some embodiments, the expression of one or more of the gene products listed in Table 13 that are not also listed in Table 19 is substantially the same in the cryopreserved therapeutic population of Tregs upon thawing as the expression of the one or more gene products in the Tregs after expansion. In some embodiments, the expression of at least 2, at least 3, at least 4, at least 5, at least 6, at least 7, or at least 8 gene products listed in Table 13 that are not also listed in Table 19 is substantially the same in the cryopreserved therapeutic population of Tregs upon thawing as the expression of the one or more gene products in the Tregs after expansion. In some embodiments, the expression of every gene product listed in Table 13 that is not also listed in Table 19 is substantially the same in the cryopreserved therapeutic population of Tregs upon thawing as the expression of the one or more gene products in the Tregs after expansion.

In some embodiments, expression of one or more of the gene products listed in Table 14-Table 18 is increased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in Table 14-Table 18 is increased in the Tregs post-expansion compared to the Tregs at baseline. In some embodiments, expression of every gene product listed in Table 14-Table 18 is increased in the Tregs post-expansion compared to the Tregs at baseline.

In some embodiments, expression of one or more of the gene products listed in Table 14 is increased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, or at least 380 gene products listed in Table 14 is increased in the Tregs post-expansion compared to the Tregs at baseline. In some embodiments, expression of every gene product listed in Table 14 is increased in the Tregs post-expansion compared to the Tregs at baseline.

In some embodiments, expression of one or more of the gene products listed in Table 15 is increased in the Tregs post-expansion compared to the Tregs at baseline. In some embodiments, expression of at least 2, at least 5, at least 10, at least 15, at least 20, at least 25, or at least 30 gene products listed in Table 15 is increased in the Tregs post-expansion compared to the Tregs at baseline. In some embodiments, expression of every gene product listed in Table 15 is increased in the Tregs post-expansion compared to the Tregs at baseline.

In some embodiments, expression of one or more of the gene products listed in Table 16 is increased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, or at least 85 gene products listed in Table 16 is increased in the Tregs post-expansion compared to the Tregs at baseline. In some embodiments, expression of every gene product listed in Table 16 is increased in the Tregs post-expansion compared to the Tregs at baseline.

In some embodiments, expression of one or more of the gene products listed in Table 17 is increased in the Tregs post-expansion compared to the Tregs at baseline. In some embodiments, expression of at least 2, at least 5, at least 10, at least 15, at least 20, at least 25, or at least 30 gene products listed in Table 17 is increased in the Tregs post-expansion compared to the Tregs at baseline. In some embodiments, expression of every gene product listed in Table 17 is increased in the Tregs post-expansion compared to the Tregs at baseline.

In some embodiments, expression of one or more of the gene products listed in Table 18 is increased in the Tregs post-expansion compared to the Tregs at baseline. In some embodiments, expression of at least 2, at least 5, at least 10, at least 15, at least 20, at least 25, at least 30, or at least 35 gene products listed in Table 18 is increased in the Tregs post-expansion compared to the Tregs at baseline. In some embodiments, expression of every gene product listed in Table 18 is increased in the Tregs post-expansion compared to the Tregs at baseline.

In some embodiments, expression of one or more of the gene products listed in Table 14-Table 18 that are not also listed in Table 19 is increased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in Table 14-Table 18 that are not also listed in Table 19 is increased in the Tregs post-expansion compared to the Tregs at baseline. In some embodiments, expression of every gene product listed in Table 14-Table 18 that is not also listed in Table 19 is increased in the Tregs post-expansion compared to the Tregs at baseline.

In some embodiments, expression of one or more of the gene products listed in Table 14 that are not also listed in Table 19 is increased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in Table 14 that are not also listed in Table 19 is increased in the Tregs post-expansion compared to the Tregs at baseline. In some embodiments, expression of every gene product listed in Table 14 that is not also listed in Table 19 is increased in the Tregs post-expansion compared to the Tregs at baseline.

In some embodiments, expression of one or more of the gene products listed in Table 15 that are not also listed in Table 19 is increased in the Tregs post-expansion compared to the Tregs at baseline. In some embodiments, expression of at least 2, at least 5, at least 10, at least 15, at least 20, at least 25, or at least 30 gene products listed in Table 15 that are not also listed in Table 19 is increased in the Tregs post-expansion compared to the Tregs at baseline. In some embodiments, expression of every gene product listed in Table 15 that is not also listed in Table 19 is increased in the Tregs post-expansion compared to the Tregs at baseline.

In some embodiments, expression of one or more of the gene products listed in Table 16 that are not also listed in Table 19 is increased in the Tregs post-expansion compared to the Tregs at baseline. In some embodiments, expression of at least 2, at least 5, at least 10, at least 15, at least 20, at least 25, at least 30, at least 35, at least 40, at least 45, at least 50, at least 55, at least 60, at least 65, at least 70, at least 75, at least 80, or at least 85 gene products listed in Table 16 that are not also listed in Table 19 is increased in the Tregs post-expansion compared to the Tregs at baseline. In some embodiments, expression of every gene product listed in Table 16 that is not also listed in Table 19 is increased in the Tregs post-expansion compared to the Tregs at baseline.

In some embodiments, expression of one or more of the gene products listed in Table 17 that are not also listed in Table 19 is increased in the Tregs post-expansion compared to the Tregs at baseline. In some embodiments, expression of at least 2, at least 5, at least 10, at least 15, at least 20, at least 25, or at least 30 gene products listed in Table 17 that are not also listed in Table 19 is increased in the Tregs post-expansion compared to the Tregs at baseline. In some embodiments, expression of every gene product listed in Table 17 that is not also listed in Table 19 is increased in the Tregs post-expansion compared to the Tregs at baseline.

In some embodiments, expression of one or more of the gene products listed in Table 18 that are not also listed in Table 19 is increased in the Tregs post-expansion compared to the Tregs at baseline. In some embodiments, expression of at least 2, at least 5, at least 10, at least 15, at least 20, at least 25, at least 30, or at least 35 products listed in Table 18 that are not also listed in Table 19 is increased in the Tregs post-expansion compared to the Tregs at baseline. In some embodiments, expression of every gene product listed in Table 18 that is not also listed in Table 19 is increased in the Tregs post-expansion compared to the Tregs at baseline.

In some embodiments, the expression of one or more of the gene products listed in Table 14-Table 18 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in Table 14-Table 18 that are not also listed in Table 19 is substantially the same in the cryopreserved therapeutic population of Tregs upon thawing as the expression of the one or more gene products in the Tregs at baseline. In some embodiments, the expression of every gene product listed in Table 14-Table 18 that are not also listed in Table 19 is substantially the same in the cryopreserved therapeutic population of Tregs upon thawing as the expression of the one or more gene products in the Tregs at baseline.

In some embodiments, the expression of one or more of the gene products listed in Table 14 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in Table 14 that are not also listed in Table 19 is substantially the same in the cryopreserved therapeutic population of Tregs upon thawing as the expression of the one or more gene products in the Tregs at baseline. In some embodiments, the expression of every gene product listed in Table 14 that are not also listed in Table 19 is substantially the same in the cryopreserved therapeutic population of Tregs upon thawing as the expression of the one or more gene products in the Tregs at baseline.

In some embodiments, the expression of one or more of the gene products listed in Table 15 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, or at least 30 gene products listed in Table 15 that are not also listed in Table 19 is substantially the same in the cryopreserved therapeutic population of Tregs upon thawing as the expression of the one or more gene products in the Tregs at baseline. In some embodiments, the expression of every gene product listed in Table 15 that are not also listed in Table 19 is substantially the same in the cryopreserved therapeutic population of Tregs upon thawing as the expression of the one or more gene products in the Tregs at baseline.

In some embodiments, the expression of one or more of the gene products listed in Table 16 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, or at least 85 gene products listed in Table 16 that are not also listed in Table 19 is substantially the same in the cryopreserved therapeutic population of Tregs upon thawing as the expression of the one or more gene products in the Tregs at baseline. In some embodiments, the expression of every gene product listed in Table 16 that are not also listed in Table 19 is substantially the same in the cryopreserved therapeutic population of Tregs upon thawing as the expression of the one or more gene products in the Tregs at baseline.

In some embodiments, the expression of one or more of the gene products listed in Table 17 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, or at least 30 gene products listed in Table 17 that are not also listed in Table 19 is substantially the same in the cryopreserved therapeutic population of Tregs upon thawing as the expression of the one or more gene products in the Tregs at baseline. In some embodiments, the expression of every gene product listed in Table 17 that are not also listed in Table 19 is substantially the same in the cryopreserved therapeutic population of Tregs upon thawing as the expression of the one or more gene products in the Tregs at baseline.

In some embodiments, the expression of one or more of the gene products listed in Table 18 that are not also listed in Table 19 is substantially the same in the cryopreserved therapeutic population of Tregs upon thawing as the expression of the one or more gene products in the Tregs at baseline. In some embodiments, the expression of at least 2, at least 5, at least 10, at least 15, at least 20, at least 25, at least 30, or at least 35 gene products listed in Table 18 that are not also listed in Table 19 is substantially the same in the cryopreserved therapeutic population of Tregs upon thawing as the expression of the one or more gene products in the Tregs at baseline. In some embodiments, the expression of every gene product listed in Table 18 that are not also listed in Table 19 is substantially the same in the cryopreserved therapeutic population of Tregs upon thawing as the expression of the one or more gene products in the Tregs at baseline.

In some embodiments, the expression of one or more of the gene products listed in Table 14-Table 18 that are not also listed in Table 19 is substantially the same in the cryopreserved therapeutic population of Tregs upon thawing as the expression of the one or more genes in the Tregs after expansion. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in Table 14-Table 18 that are not also listed in Table 19 is substantially the same in the cryopreserved therapeutic population of Tregs upon thawing as the expression of the one or more genes in the Tregs after expansion. In some embodiments, the expression of every one of the gene products listed in Table 14-Table 18 that are not also listed in Table 19 is substantially the same in the cryopreserved therapeutic population of Tregs upon thawing as the expression of the one or more genes in the Tregs after expansion.

In some embodiments, the expression of one or more of the gene products listed in Table 14 that are not also listed in Table 19 is substantially the same in the cryopreserved therapeutic population of Tregs upon thawing as the expression of the one or more genes in the Tregs after expansion. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products listed in Table 14 that are not also listed in Table 19 is substantially the same in the cryopreserved therapeutic population of Tregs upon thawing as the expression of the one or more genes in the Tregs after expansion. In some embodiments, the expression of every one of the gene products listed in Table 14 that are not also listed in Table 19 is substantially the same in the cryopreserved therapeutic population of Tregs upon thawing as the expression of the one or more genes in the Tregs after expansion.

In some embodiments, the expression of one or more of the gene products listed in Table 15 that are not also listed in Table 19 is substantially the same in the cryopreserved therapeutic population of Tregs upon thawing as the expression of the one or more genes in the Tregs after expansion. In some embodiments, the expression of at least 2, at least 5, at least 10, at least 15, at least 20, at least 25, or at least 30 gene products listed in Table 15 that are not also listed in Table 19 is substantially the same in the cryopreserved therapeutic population of Tregs upon thawing as the expression of the one or more genes in the Tregs after expansion. In some embodiments, the expression of every one of the gene products listed in Table 15 that are not also listed in Table 19 is substantially the same in the cryopreserved therapeutic population of Tregs upon thawing as the expression of the one or more genes in the Tregs after expansion.

In some embodiments, the expression of one or more of the gene products listed in Table 16 that are not also listed in Table 19 is substantially the same in the cryopreserved therapeutic population of Tregs upon thawing as the expression of the one or more genes in the Tregs after expansion. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, or at least 85 gene products listed in Table 16 that are not also listed in Table 19 is substantially the same in the cryopreserved therapeutic population of Tregs upon thawing as the expression of the one or more genes in the Tregs after expansion. In some embodiments, the expression of every one of the gene products listed in Table 16 that are not also listed in Table 19 is substantially the same in the cryopreserved therapeutic population of Tregs upon thawing as the expression of the one or more genes in the Tregs after expansion.

In some embodiments, the expression of one or more of the gene products listed in Table 17 that are not also listed in Table 19 is substantially the same in the cryopreserved therapeutic population of Tregs upon thawing as the expression of the one or more genes in the Tregs after expansion. In some embodiments, the expression of at least 2, at least 5, at least 10, at least 15, at least 20, at least 25, or at least 30 gene products listed in Table 17 that are not also listed in Table 19 is substantially the same in the cryopreserved therapeutic population of Tregs upon thawing as the expression of the one or more genes in the Tregs after expansion. In some embodiments, the expression of every one of the gene products listed in Table 17 that are not also listed in Table 19 is substantially the same in the cryopreserved therapeutic population of Tregs upon thawing as the expression of the one or more genes in the Tregs after expansion.

In some embodiments, the expression of one or more of the gene products listed in Table 18 that are not also listed in Table 19 is substantially the same in the cryopreserved therapeutic population of Tregs upon thawing as the expression of the one or more genes in the Tregs after expansion. In some embodiments, the expression of at least 2, at least 5, at least 10, at least 15, at least 20, at least 25, at least 30, or at least 35 gene products listed in Table 18 that are not also listed in Table 19 is substantially the same in the cryopreserved therapeutic population of Tregs upon thawing as the expression of the one or more genes in the Tregs after expansion. In some embodiments, the expression of every one of the gene products listed in Table 18 that are not also listed in Table 19 is substantially the same in the cryopreserved therapeutic population of Tregs upon thawing as the expression of the one or more genes in the Tregs after expansion.

In some embodiments, one or more gene products listed in Table 12 and/or Table 13 is decreased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 12 and/or Table 13 is decreased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 12 is decreased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 12 is decreased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 13 is decreased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 13 is decreased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 14-Table 18 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 14-Table 18 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 14 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 14 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 15 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 15 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 16 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 16 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 17 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 17 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, one or more gene products listed in Table 18 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline. In some embodiments, one or more gene products listed in Table 18 is increased at least about 4-fold, about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold in cryopreserved Tregs relative to Tregs at baseline but the one or more gene products is substantially similar between cryopreserved Tregs and Tregs after expansion but before cryopreservation.

In some embodiments, the expression of one or more gene products associated with a dysfunctional Treg phenotype (e.g., one or more of the gene products listed in Table 12) is decreased in the Tregs post-expansion compared to the Tregs at baseline. A dysfunctional Treg phenotype includes, for example, dysregulated calcium dynamics, loss of MECP2 binding ability to 5-Hydroxymethylcytosine (5hmC)-DNA, dysregulation of MECP2 expression or activity, and loss of MECP2 regulation, phosphorylation or binding abilities. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with a dysfunctional Treg phenotype (e.g., one or more of the gene products listed in Table 12) is decreased in the Tregs post-expansion compared to the Tregs at baseline.

In some embodiments, the expression of one or more gene products associated with a dysfunctional Treg phenotype (e.g., one or more of the gene products listed in Table 12) is substantially the same in the cryopreserved composition of Tregs relative to the expanded population of Tregs prior to cryopreservation. A dysfunctional Treg phenotype includes, for example, dysregulated calcium dynamics, loss of MECP2 binding ability to 5hmC-DNA, dysregulation of MECP2 expression or activity, and loss of MECP2 regulation, phosphorylation or binding abilities. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with a dysfunctional Treg phenotype (e.g., one or more of the gene products listed in Table 12) is substantially the same in the cryopreserved composition of Tregs relative to the expanded population of Tregs prior to cryopreservation.

In some embodiments, the expression of one or more gene products associated with a dysfunctional Treg phenotype (e.g., one or more of the gene products listed in Table 12) that are not also listed in Table 19 is decreased in the Tregs post-expansion compared to the Tregs at baseline. A dysfunctional Treg phenotype includes, for example, dysregulated calcium dynamics, loss of MECP2 binding ability to 5hmC-DNA, dysregulation of MECP2 expression or activity, and loss of MECP2 regulation, phosphorylation or binding abilities. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with a dysfunctional Treg phenotype (e.g., one or more of the gene products listed in Table 12) that are not also listed in Table 19 is decreased in the Tregs post-expansion compared to the Tregs at baseline.

In some embodiments, the expression of one or more gene products associated with a dysfunctional Treg phenotype (e.g., one or more of the gene products listed in Table 12) that are not also listed in Table 19 is substantially the same in the cryopreserved composition of Tregs relative to the expanded population of Tregs prior to cryopreservation. A dysfunctional Treg phenotype includes, for example, dysregulated calcium dynamics, loss of MECP2 binding ability to 5hmC-DNA, dysregulation of MECP2 expression or activity, and loss of MECP2 regulation, phosphorylation or binding abilities. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with a dysfunctional Treg phenotype (e.g., one or more of the gene products listed in Table 12) that are not also listed in Table 19 is substantially the same in the cryopreserved composition of Tregs relative to the expanded population of Tregs prior to cryopreservation.

In some embodiments, the expression of one or more methylation- or epigenetics-associated gene products (e.g., one or more of the gene products listed in Table 13) is decreased in the Tregs post-expansion compared to the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 methylation- or epigenetics-associated gene products (e.g., one or more of the gene products listed in Table 13) is decreased in the Tregs post-expansion compared to the Tregs at baseline.

In some embodiments, the expression of one or more methylation- or epigenetics-associated gene products (e.g., one or more of the gene products listed in Table 13) is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 methylation- or epigenetics-associated gene products (e.g., one or more of the gene products listed in Table 13) is substantially the same in the cryopreserved composition of Tregs relative to the expanded population of Tregs prior to cryopreservation.

In some embodiments, the expression of one or more methylation- or epigenetics-associated gene products (e.g., one or more of the gene products listed in Table 13) that are not also listed in Table 19 is decreased in the Tregs post-expansion compared to the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 methylation- or epigenetics-associated gene products (e.g., one or more of the gene products listed in Table 13) that are not also listed in Table 19 is decreased in the Tregs post-expansion compared to the Tregs at baseline.

In some embodiments, the expression of one or more methylation- or epigenetics-associated gene products (e.g., one or more of the gene products listed in Table 13) that are not also listed in Table 19 is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 methylation- or epigenetics-associated gene products (e.g., one or more of the gene products listed in Table 13) that are not also listed in Table 19 is substantially the same in the cryopreserved composition of Tregs relative to the expanded population of Tregs prior to cryopreservation.

In some embodiments, the expression of one or more gene products known to be important for the proliferation, health, identification, and/or mechanism of Treg cells (e.g., one or more of the gene products listed in Table 15) is increased in a cryopreserved composition of Tregs relative to the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products known to be important for the proliferation, health, identification, and/or mechanism of Treg cells (e.g., one or more of the gene products listed in Table 15) is increased in a cryopreserved composition of Tregs relative to the expression of the one or more gene products in the Tregs at baseline.

In some embodiments, the expression of one or more gene products known to be important for the proliferation, health, identification, and/or mechanism of Treg cells (e.g., one or more of the gene products listed in Table 15) is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products known to be important for the proliferation, health, identification, and/or mechanism of Treg cells (e.g., one or more of the gene products listed in Table 15) is substantially the same in the cryopreserved composition of Tregs relative to the expanded population of Tregs prior to cryopreservation.

In some embodiments, the expression of one or more gene products known to be important for the proliferation, health, identification, and/or mechanism of Treg cells (e.g., one or more of the gene products listed in Table 15) that are not also listed in Table 19 is increased in a cryopreserved composition of Tregs relative to the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products known to be important for the proliferation, health, identification, and/or mechanism of Treg cells (e.g., one or more of the gene products listed in Table 15) that are not also listed in Table 19 is increased in a cryopreserved composition of Tregs relative to the expression of the one or more gene products in the Tregs at baseline.

In some embodiments, the expression of one or more gene products known to be important for the proliferation, health, identification, and/or mechanism of Treg cells (e.g., one or more of the gene products listed in Table 15) that are not also listed in Table 19 is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products known to be important for the proliferation, health, identification, and/or mechanism of Treg cells (e.g., one or more of the gene products listed in Table 15) that are not also listed in Table 19 is substantially the same in the cryopreserved composition of Tregs relative to the expanded population of Tregs prior to cryopreservation.

In some embodiments, the expression of one or more gene products known to be important for the proliferation, health, identification, and/or mechanism of Treg cells (e.g., one or more of the gene products listed in Table 15) is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products known to be important for the proliferation, health, identification, and/or mechanism of Treg cells (e.g., one or more of the gene products listed in Table 15) is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation.

In some embodiments, the expression of one or more gene products known to be important for the proliferation, health, identification, and/or mechanism of Treg cells (e.g., one or more of the gene products listed in Table 15) that are not also listed in Table 19 is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products known to be important for the proliferation, health, identification, and/or mechanism of Treg cells (e.g., one or more of the gene products listed in Table 15) that are not also listed in Table 19 is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation.

In some embodiments, the expression of one or more mitochondria-related gene products (e.g., one or more of the gene products listed in Table 16) is increased in a cryopreserved composition of Tregs relative to the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 mitochondria-related gene products (e.g., one or more of the gene products listed in Table 16) is increased in a cryopreserved composition of Tregs relative to the expression of the one or more gene products in the Tregs at baseline. In some embodiments, the expression of one or more mitochondria-related gene products (e.g., one or more of the gene products listed in Table 16) is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 mitochondria-related gene products (e.g., one or more of the gene products listed in Table 16) is substantially the same in the cryopreserved composition of Tregs relative to the expanded population of Tregs prior to cryopreservation.

In some embodiments, the expression of one or more mitochondria-related gene products (e.g., one or more of the gene products listed in Table 16) that are not also listed in Table 19 is increased in a cryopreserved composition of Tregs relative to the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 mitochondria-related gene products (e.g., one or more of the gene products listed in Table 16) that are not also listed in Table 19 is increased in a cryopreserved composition of Tregs relative to the expression of the one or more gene products in the Tregs at baseline. In some embodiments, the expression of one or more mitochondria-related gene products (e.g., one or more of the gene products listed in Table 16) that are not also listed in Table 19 is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 mitochondria-related gene products (e.g., one or more of the gene products listed in Table 16) that are not also listed in Table 19 is substantially the same in the cryopreserved composition of Tregs relative to the expanded population of Tregs prior to cryopreservation.

In some embodiments, the expression of one or more mitochondria-related gene products (e.g., one or more of the gene products listed in Table 16) is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 mitochondria-related gene products (e.g., one or more of the gene products listed in Table 16) is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation.

In some embodiments, the expression of one or more mitochondria-related gene products (e.g., one or more of the gene products listed in Table 16) that are not also listed in Table 19 is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 mitochondria-related gene products (e.g., one or more of the gene products listed in Table 16) that are not also listed in Table 19 is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation.

In some embodiments, the expression of one or more gene products associated with the cell cycle (in particular, cell cycle progression), cell division, DNA replication or DNA repair (e.g., one or more of the gene products listed in Table 17) is increased in a cryopreserved composition of Tregs relative to the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with the cell cycle (in particular, cell cycle progression), cell division, DNA replication or DNA repair (e.g., one or more of the gene products listed in Table 17) is increased in a cryopreserved composition of Tregs relative to the expression of the one or more gene products in the Tregs at baseline.

In some embodiments, the expression of one or more gene products associated with the cell cycle (in particular, cell cycle progression), cell division, DNA replication or DNA repair (e.g., one or more of the gene products listed in Table 17) is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with the cell cycle (in particular, cell cycle progression), cell division, DNA replication or DNA repair (e.g., one or more of the gene products listed in Table 17) is substantially the same in the cryopreserved composition of Tregs relative to the expanded population of Tregs prior to cryopreservation.

In some embodiments, the expression of one or more gene products associated with the cell cycle (in particular, cell cycle progression), cell division, DNA replication or DNA repair (e.g., one or more of the gene products listed in Table 17) that are not also listed in Table 19 is increased in a cryopreserved composition of Tregs relative to the expression of the one or more gene products in the Tregs at baseline. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with the cell cycle (in particular, cell cycle progression), cell division, DNA replication or DNA repair (e.g., one or more of the gene products listed in Table 17) that are not also listed in Table 19 is increased in a cryopreserved composition of Tregs relative to the expression of the one or more gene products in the Tregs at baseline. In some embodiments, the expression of one or more gene products associated with the cell cycle (in particular, cell cycle progression), cell division, DNA replication or DNA repair (e.g., one or more of the gene products listed in Table 17) that are not also listed in Table 19 is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with the cell cycle (in particular, cell cycle progression), cell division, DNA replication or DNA repair (e.g., one or more of the gene products listed in Table 17) that are not also listed in Table 19 is substantially the same in the cryopreserved composition of Tregs relative to the expanded population of Tregs prior to cryopreservation.

In some embodiments, the expression of one or more gene products associated with the cell cycle (in particular, cell cycle progression), cell division, DNA replication or DNA repair (e.g., one or more of the gene products listed in Table 17) is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with the cell cycle (in particular, cell cycle progression), cell division, DNA replication or DNA repair (e.g., one or more of the gene products listed in Table 17) is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation.

In some embodiments, the expression of one or more gene products associated with the cell cycle (in particular, cell cycle progression), cell division, DNA replication or DNA repair (e.g., one or more of the gene products listed in Table 17) that are not also listed in Table 19 is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation. In some embodiments, the expression of at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 gene products associated with the cell cycle (in particular, cell cycle progression), cell division, DNA replication or DNA repair (e.g., one or more of the gene products listed in Table 17) that are not also listed in Table 19 is increased in a cryopreserved composition of Tregs relative to the expression of the one or more gene products in the Tregs at baseline and the expression of the same one or more gene products is substantially the same in the cryopreserved composition of Tregs relative to the expanded population of Tregs prior to cryopreservation.

### 6.3.2. Pharmaceutical Compositions

In some embodiments, a composition provided herein is a pharmaceutical composition comprising a therapeutic population of Tregs and a carrier, excipient, or diluent. In some embodiments, a composition provided herein is a pharmaceutical composition comprising an effective amount of a therapeutic population of Tregs and a carrier, excipient, or diluent.

The carrier, excipient, or diluent may be any pharmaceutically acceptable carrier, excipient or diluent, known in the art. Examples of pharmaceutically acceptable carriers include non-toxic solids, semisolids, or liquid fillers, diluents, encapsulating materials, formulation auxiliaries or carriers. In some embodiments, a composition comprising a therapeutic population of Tregs provided herein further comprises a pharmaceutically acceptable carrier. A pharmaceutically acceptable carrier can include all solvents, dispersion media, coatings, antibacterial and antifungal agents, isotonic and absorption delaying agents, and the like, compatible with pharmaceutical administration. Examples of such carriers or diluents include, but are not limited to, water, saline, Ringer's solutions, dextrose solution, and 5% human serum albumin. Liposomes and non-aqueous vehicles such as fixed oils may also be used. In some embodiment, the pharmaceutical composition comprises a therapeutic population of Tregs suspended in a sterile buffer.

In some embodiments, a pharmaceutical composition provided herein comprises no more than 0.1% v/v, no more than 0.2% v/v, no more than 0.3% v/v, no more than 0.4% v/v, no more than 0.5% v/v, no more than 0.6% v/v, no more than 0.7% v/v, no more than 0.8% v/v, no more than 0.9% v/v, no more than 1% v/v, no more than 1.1% v/v, no more than 1.2% v/v, no more than 1.3% v/v, no more than 1.4% v/v or no more than 1.5% v/v DMSO.

In some embodiments, a composition comprising a therapeutic population of Tregs provided herein comprises no contaminants. In some embodiments, a composition comprising a therapeutic population of Tregs provided herein comprises comprises a sufficiently low level of contaminants as to be suitable for administration, e.g., therapeutic administration, to a subject, for example a human subject. Contaminants include, for example, bacteria, fungus, mycoplasma, endotoxins or residual beads from the expansion culture. In some embodiments, a composition comprising a therapeutic population of Tregs provided herein comprises less than about 5 EU/kg endotoxins. In some embodiments, a composition comprising a therapeutic population of Tregs provided herein comprises about or less than about 100 beads per 3×10⁶ cells.

In some embodiments, a composition comprising a therapeutic population of Tregs provided herein is sterile. In some embodiments, isolation or enrichment of the cells is carried out in a closed or sterile environment, for example, to minimize error, user handling and/or contamination. In some embodiments, sterility may be readily accomplished, e.g., by filtration through sterile filtration membranes.

### 6.4 Methods of Treatment

Provided herein are methods of treatment comprising administering an effective amount of an expanded population of Tregs as described herein to a subject in need thereof. For example, provided herein are methods of treatment comprising administering an effective amount of an ex vivo-expanded population of Tregs as described herein, e.g., as produced by the methods presented herein, to a subject in need thereof.

Provided herein are methods of treatment comprising administering an effective amount of an expanded population of Tregs as described herein, wherein the population has been cryopreserved, to a subject in need thereof. For example, provided herein are methods of treatment comprising administering an effective amount of an ex vivo-expanded population of Tregs as described herein, e.g., as produced by the methods presented herein, wherein the population has been cryopreserved, to a subject in need thereof.

Provided herein are methods of treatment comprising administering an effective amount of an expanded population of Tregs as described herein, wherein the population has been cryopreserved, to a subject in need thereof, wherein the cryopreserved population is thawed and administered to the subject without further expansion. For example, provided herein are methods of treatment comprising administering an effective amount of an ex vivo-expanded population of Tregs as described herein, e.g., as produced by the methods presented herein, wherein the population has been cryopreserved, to a subject in need thereof, wherein the cryopreserved population is thawed and administered to the subject without further expansion.

Provided herein are methods of treatment comprising administering an effective amount of a cryopreserved composition comprising a therapeutic population of Tregs to a subject in need thereof. In some embodiments provided herein are methods for treating neurodegenerative disorders in a subject in need thereof comprising administering an effective amount of a cryopreserved composition comprising a therapeutic population of Tregs to a subject in need thereof. In certain embodiments, the cryopreserved population is thawed and administered to the subject without further expansion.

In some embodiments, the subject is diagnosed with or is suspected of having a disorder associated with Treg dysfunction. In some embodiments, the subject is diagnosed with or is suspected of having a disorder associated with Treg deficiency. In some embodiments, the subject is diagnosed with or is suspected of having a condition driven by a T cell response.

In some embodiments the subject is diagnosed with or is suspected of having a neurodegenerative disease. In some embodiments, the subject is diagnosed with or is suspected of having Alzheimer's disease, Amyotrophic Lateral Sclerosis, Huntington's disease, Parkinson's disease, or frontotemporal dementia.

In some embodiments, the subject is diagnosed with or is suspected of having a disorder that would benefit from downregulation of the immune system.

In some embodiments, the subject is diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, systemic lupus erythematosus, cutaneous lupus, myasthenia gravis, autoimmune nephropathy, autoimmune hemolytic anemia, autoimmune cytopenia autoimmune hepatitis, autoimmune uveitis, alopecia, thyroiditis or pemphigus.

In some embodiments, the subject is diagnosed with or suspected of having heart failure or ischemic cardiomyopathy. In some embodiments, the subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation).

In some embodiments, the subject is diagnosed with or suspected of having neuroinflammation. Neuroinflammation may be associated, for example, with stroke, acute disseminated encephalomyelitis (ADEM), acute optic neuritis, transverse myelitis, neuromyelitis optica (NMO), epilepsy, traumatic brain injury, spinal cord injury, encephalitis central nervous system (CNS) vasculitis, neurosarcoidosis, autoimmune or post-infectious encephalitis, or chronic meningitis.

In some embodiments, the subject is diagnosed with or suspected of having cardo-inflammation, e.g., cardio-inflammation associated with atheroscleorosis, myocardial infarction, ischemic cardiomyopathy, with heart failure.

In some embodiments, the subject is diagnosed with or suspected of having chronic inflammatory demyelinating polyradiculoneuropathy (CIDP). In some embodiments, the subject is diagnosed with or suspected of having acute inflammatory demyelinating polyneuropathy (AIDP). In some embodiments, the subject is diagnosed with or suspected of having Guillain-Barre syndrome (GBS).

In some embodiments, the subject has had a stroke.

In some embodiments, the subject is diagnosed with or suspected of having cancer, e.g., a blood cancer.

In some embodiments, the subject is diagnosed with or suspected of having asthma.

In some embodiments, the subject is diagnosed with or suspected of having eczema.

In some embodiments, the subject is diagnosed with or suspected of having a disorder associated with overactivation of the immune system.

In some embodiments, the subject is diagnosed with or suspected of having Tregopathy. The Tregopathy may be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4+CD25+ cells per kg of body weight of the subject are administered. In some embodiments, 1×10⁶ CD4+CD25+ cells (+/−10%) per kg of body weight of the subject are administered.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25⁺ cells are administered to a patient.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25⁺ cells are administered to a patient in one infusion.

In some embodiments, a cryopreserved composition comprising a therapeutic population of Tregs is administered within about 30 minutes, about 1 h, about 2-3 h, about 3-4 h, about 4-5 h, about 5-6, about 6-7 h, about 7-8 h, about 8-9 h, or about 9-10 h of thawing the cryopreserved composition comprising a therapeutic population of Tregs. The cryopreserved composition comprising a therapeutic population of Tregs may be stored at about 2° C. to about 8° C. (e.g., at about 40) between thawing and administration.

In some embodiments, one dose of a therapeutic population of Tregs or a composition comprising a therapeutic population of Tregs is administered to a subject. In some embodiments, a therapeutic population of Tregs or a composition comprising a therapeutic population of Tregs is administered more than once. In some embodiments, a therapeutic population of Tregs or a composition comprising a therapeutic population of Tregs is administered two or more times. In some embodiments, a therapeutic population of Tregs or a composition comprising a therapeutic population of Tregs is administered every 1-2 weeks, 2-3 weeks, 3-4 weeks, 4-5 weeks, 5-6 weeks, 6-7 weeks, 7-8 weeks, 8-9 weeks, 9-10 weeks, 10-11 weeks, 11-12 weeks, every 1-2 months, 2-3 months, 3-4 months, 4-5 months, 5-6 months, 6-7 months, 7-8 months, 8-9 months, 9-10 months, 10-11 months, 11-12 months, 13-14 months, 14-15 months, 15-16 months, 16-17 months, 17-18 months, 18-19 months, 19-20 months, 20-21 months, 21-22 months, 22-23 months, 23-24 months, every 1-2 years, 2-3 years, 3-4 years or 4-5 years.

In some embodiments, about 1×10⁶ Tregs per kg of body weight of the subject are administered in the first administration and the number of Tregs administered is increased in the second third and subsequent administration. In some embodiments, about 1×10⁶ Tregs per kg of body weight of the subject are administered in the first two administrations, and the number of Tregs administered is increased in every other administration thereafter (e.g., the 4^{th}, 6^{th}, 8^{th} and 10^{th} administration). Thus, for example, about 1×10⁶ Tregs per kg of body weight of the subject may be administered per month for the first and second month, and about 2×10⁶ Tregs per kg of body weight of the subject may be administered per month for the third and fourth month, and/or about 3×10⁶ cells per kg of body weight of the subject are administered per month for the fifth and sixth month.

In some embodiments, a method of treatment provide herein comprises administering a therapeutic population of autologous Tregs or a composition comprising a therapeutic population of autologous Tregs to the subject. In other embodiments, a method of treating a neurodegenerative disorder in a subject comprises administering a therapeutic population of allogeneic Tregs or a composition comprising a therapeutic population of allogeneic Tregs to the subject.

### 6.4.1. Additional Therapies

In some embodiments, In some embodiments, a subject treated in accordance with the method of treatment described herein further received one or more additional therapy or additional therapies.

In some embodiments, the subject is additionally administered IL-2. The dose of IL-2 may be about 0.5-1×10⁵ IU/m², about 1-1.5×10⁵IU/m², about 1.5-2×10⁵IU/m², about 2-2.5×10⁵IU/m², about 2.5-3×10⁵IU/m², about 3-3.5×10⁵IU/m², about 3.5-4×10⁵IU/m², about 4-4.5×10⁵IU/m², about 4.5-5×10⁵IU/m², about 5-6×10⁵IU/m², about 6-7×10⁵IU/m², about 7-8×10⁵IU/m², about 8-9×10⁵IU/m², about 9-10×10⁵IU/m², about 10-15×10⁵IU/m², about 15-20×10⁵IU/m², about 20-25×10⁵IU/m², about 25-30×10⁵IU/m², about 30-35×10⁵IU/m², about 35-40×10⁵IU/m², about 40-45×10⁵IU/m², about 45-50×10⁵IU/m², about 50-60×10⁵IU/m², about 60-70×10⁵IU/m², about 70-80×10⁵IU/m², about 80-90×10⁵IU/m², or about 90-100×10⁵ IU/m². In specific embodiments, the subject is administered 2×10⁵IU/m² of IL-2.

The IL-2 may be administered one, two or more times a month. In some embodiments, the IL-2 is administered three times a month.

In some embodiments, the IL-2 is administered subcutaneously.

The IL-2 may be administered at least 2 weeks, at least 3 weeks, or at least 4 weeks prior to the first Treg infusion.

In some embodiments, the subjected treated in accordance with the methods described herein receives one or more additional therapies are for the treatment of Alzheimer's. Addition therapies for the treatment of Alzheimer's may include acetylcholinesterase inhibitors (e.g., donepezil (Aricept®), galantamine (Razadyne®), or rivastigmine (Exelon®)) or NMDA receptor antagonists (e.g., Memantine (Akatinol®, Axura®, Ebixa®/Abixa®, Memox® and Namenda®). Additional therapies may also include anti-inflammatory agents (e.g., nonsteroidal anti-inflammatory drugs (NSAID) such as ibuprofen, indomethacin, and sulindac sulfide)), neuronal death associated protein kinase (DAPK) inhibitors such as derivatives of 3-amino pyridazine, Cyclooxygenases (COX-1 and -2) inhibitors, or antioxidants such as vitamins C and E.

In some embodiments, a subject treated in accordance with the methods described herein receives on or more additional therapies for the treatment of ALS. Additional therapies for the treatment of ALS may include Riluzole (Rilutek®) or Riluzole (Rilutek®)

In some embodiments, the therapeutic population of Tregs or the composition comprising a therapeutic composition of Tregs is administered to a subject by intravenous infusion.

### 6.4.2. Methods of Determining Treatment Effect

In some embodiments, a method of treatment provided herein results in an increase in the Treg suppressive function in the blood from baseline. In some embodiments, a method of treatment provided herein results in an increase in the Treg suppressive function in the blood from baseline to week 4, week 8, week 16, week 24, week 30 or week 36. In some embodiments, a method of treatment provided herein results in an increase in the Treg suppressive function in the blood from baseline to week 24. In some embodiments, a method of treatment provided herein results in an increase in the Treg numbers in the blood from baseline. In some embodiments, a method of treatment provided herein results in an increase in the Treg numbers in the blood from baseline to week 4, week 8, week 16, week 24, week 30 or week 36. In some embodiments, a method of treatment provided herein results in an increase in the Treg numbers in the blood from baseline to week 24.

The effect of a method of treatment provided herein may be assessed by monitoring clinical signs and symptoms of the disease to be treated.

In some embodiments, method of treatment provided herein results in a change in the Appel ALS score compared to baseline. The Appel ALS score measures overall progression of disability or altered function. In some embodiments, the Appel ALS score decreases in a subject treated in accordance with a method provided herein compared to baseline, indicating an improvement of symptoms. In other embodiments, the Appel ALS score remains unchanged ins a subject treated in accordance with a method provided herein compared to baseline.

In some embodiments, a method of treatment provided herein results in a change in the Amyotrophic Lateral Sclerosis Functional Rating Scale-revised (ALSFRS-R) score compared to baseline. The ALSFRS-R score assesses the progression of disability or altered function. In some embodiments, the ALSFRS-R score increases in a subject treated in accordance with a method provided herein compared to baseline, indicating an improvement of symptoms. In other embodiments, the Appel ALSFRS-R score remains unchanged in a subject treated in accordance with a method provided herein compared to baseline.

In some embodiments, a method of treatment provided herein results in a change in forced vital capacity (FVC; strength of muscles used with expiration) compared to baseline, where the highest number is the strongest measurement. In some embodiments, FVC increases in a subject treated in accordance with a method provided herein compared to baseline. In other embodiments, FVC remains unchanged in a subject treated in accordance with a method provided herein compared to baseline.

In some embodiments, a method of treatment provided herein results in a change in Maximum Inspiratory Pressure (MIP; strength of muscles used with inspiration) compared where the highest number is the strongest measurement. In some embodiments, MIP increases in a subject treated in accordance with a method provided herein compared to baseline. In other embodiments, MIP remains unchanged in a subject treated in accordance with a method provided herein compared to baseline.

In some embodiments, a method of treatment provided herein results in a change in Neuropsychiatric Inventory Questionnaire (NPI-Q) compared to baseline. The NPI-Q provides symptom Severity and Distress ratings for each symptom reported, and total Severity and Distress scores reflecting the sum of individual domain scores. In some embodiments, the NPI-Q score decreases in a subject treated in accordance with a method provided herein compared to baseline. In other embodiments, NPI-Q score remains unchanged in a subject treated in accordance with a method provided herein compared to baseline.

In some embodiments, a method of treatment provided herein results in a decrease in the frequency of GI symptoms, anaphylaxis or seizures compared to baseline.

In some embodiments, a method of treatment provided herein results in a change in a change in CSF amyloid and/or CSF tau protein (CSF-tau) compared to baseline. In some embodiments, the levels of CSF amyloid and/or CSF tau protein decreases in a subject treated in accordance with a method provided herein compared to baseline. In other embodiments, the levels of CSF amyloid and/or CSF tau protein remains unchanged in a subject treated in accordance with a method provided herein compared to baseline.

In some embodiments, a method of treatment provided herein results in a change in Clinical Dementia Rating (CDR) compared to baseline. The CDR rates memory, orientation, judgment and problem-solving, community affairs, home and hobbies, and personal care, and a global rating is then generated, ranging from 0-no impairment to 3-severe impairment. In some embodiments, the CDR decreases in a subject treated in accordance with the methods provided herein compared to baseline. In other embodiments, the CDR remains unchanged in a subject treated in accordance with a method provided herein compared to baseline.

In some embodiments, a method of treatment provided herein results in a change in Alzheimer's Disease Assessment Scale (ADAS)-cog13 score compared to baseline. ADAS-cog tests cognitive performance and has an upper limit is 85 (poor performance) and lower limit is zero (best performance). In some embodiments, the ADAS-cog13 score decreases in a subject treated in accordance with a method provided herein compared to baseline. In other embodiments, the ADAS-cog13 score remains unchanged in a subject treated in accordance with a method provided herein.

The efficacy of a method of treatment described herein may be assessed at about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, at about 2-3 months, 3-4 months, 4-5 months, 5-6 months, 6-7 months, 7-8 months, 8-9 months, about 9-10 months, about 10-11 months, about 11-12 months, about 12-18 months, about 18-24 months, about 1-2 years, about 2-3 years, about 3-4 years, about 4-5 years, about 5-6 years, about 6-7 years, about 7-8 years, about 8-9 years, or about 9-10 years after initiation of treatment in accordance with the method described herein.

### 6.5 Kits

Provided herein are kits comprising a therapeutic composition of Tregs or a composition comprising a therapeutic population of Tregs provided herein.

In some embodiments, a kit provided herein comprises instructions for use, additional reagents (e.g., sterilized water or saline solutions for dilution of the compositions), or components, such as tubes, containers or syringes for collection of biological samples, processing of biological samples, and/or reagents for quantitating the amount of one or more surface markers in a sample (e.g., detection reagents, such as antibodies).

In some embodiments, the kits contain one or more containers containing a therapeutic population of Tregs or a composition comprising a therapeutic population of Tregs for use in the methods provided herein. The one or more containers holding the composition may be a single-use vial or a multi-use vial. In some embodiments, the article of manufacture or kit may further comprise a second container comprising a suitable diluent. In some embodiments, the kit contains instruction for use (e.g., dilution and/or administration) of a therapeutic population of Tregs or a composition comprising a therapeutic population of Tregs provided herein.

### 7. BIOLOGICAL FUNCTIONAL EQUIVALENTS

Modification and changes may be made in the structure of the nucleic acids, or to the vectors comprising them, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to the codon table, below.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

Codon Table

| | Amino Acids | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | uuu | | | | |
| Glycine | Gly | G | GGA | GGC | GGG | GGU | | |
| Histidine | His | H | CAC | CAU | | | | |
| Isoleucine | lie | I | AUA | AUC | AUU | | | |
| Lysine | Lys | K | AAA | AAG | | | | |
| Leucine | Leu | L | UUA | UUG | CUA | cue | CUG | CUU |
| Methionine | Met | M | AUG | | | | | |
| Asparagine | Asn | N | AAC | AAU | | | | |
| Proline | Pro | P | CCA | ccc | CCG | ecu | | |
| Glutamine | Gin | Q | CAA | CAG | | | | |
| Arginine | Arg | R | AGA | AGG | CGA | CGC | CGG | CGU |
| Serine | Ser | S | AGC | AGU | UCA | ucc | UCG | UCU |
| Threonine | Thr | T | ACA | ACC | ACG | ACU | | |
| Valine | Vai | V | GUA | GUC | GUG | GUU | | |
| Tryptophan | Tip | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0+1); glutamate (+3.0+1); serine (+0.3); asparagine (+0.2); glutamine (+0.2);

glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within +0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### 8. EXAMPLES

The examples appearing in this section are provided to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the invention.

### 8.1 Example 1: Expanded Autologous Regulatory T Lymphocyte Infusions in ALS: A Phase 1, First-in-Human Study

Tregs suppress the proliferation of responder T lymphocytes and the activation of microglia. In ALS patients, the expression of the Treg master transcription factor FOXP3 is reduced in rapidly progressing patients, with subsequent impairment of Treg suppressive function. FOXP3 expression and Treg suppressive function correlate with the extent and rapidity of disease progression. When expanded ex vivo in the presence of interleukin (IL)-2 and rapamycin, Treg suppressive function is restored. These data all suggested that infusions of expanded autologous Tregs will improve Treg suppressive function in vivo and slow rates of disease progression in ALS patients.

To test these hypotheses, a first-in-human phase 1 study was conducted to determine whether infusions of expanded autologous Tregs into ALS patients were safe and tolerable during early and later stages of disease. IL-2 was administered concomitantly in the participants in an effort to stabilize and possibly enhance the suppressive functions of the infused Tregs. Infusions of Tregs were safe and well-tolerated in all participants. Treg numbers and suppressive function increased after each infusion. The infusions slowed progression rates during early and later stages of disease, and Treg suppressive function correlated with slowing of disease progression per the Appel ALS scale. In addition, respiratory measures of maximal inspiratory pressure also stabilized, particularly in two participants, during Treg infusions. The phase I results demonstrated the safety and potential benefit of expanded autologous Treg infusions, thus providing further impetus for clinical trials employing these compositions in ALS patients. The results of the phase 1 clinical trial testing the safety of Treg infusions into ALS patients are described further below.

### 8.1.1. ABSTRACT

### 8.1.1.1 Objective

To determine whether autologous infusions of expanded regulatory T lymphocytes (Tregs) into ALS patients are safe and tolerable during early and later stages of disease.

### 8.1.1.2 Methods

Three ALS patients, with no family history of ALS, were selected based on their differing sites of disease onset and rates of progression. Participants underwent leukapheresis, and Tregs were subsequently isolated and expanded ex vivo. Tregs (1×10⁶ cells/kg) were administered intravenously at early stages (4 doses over 2 months) and later stages (4 doses over 4 months) of disease. Concomitant interleukin (IL)-2 (2×10⁵ IU/m²/injection) was administered subcutaneously 3 times weekly over the entire study period. Participants were closely monitored for adverse effects and changes in disease progression rates. Treg numbers and suppressive function were assayed during and following each round of Treg infusions.

### 8.1.1.3 Results

Infusions of Tregs were safe and well-tolerated in all participants. Treg numbers and suppressive function increased after each infusion. The infusions slowed progression rates during early and later stages of disease. Spearman's correlation analyses showed that increased Treg suppressive function correlated with slowing of disease progression per the Appel ALS scale for each participant: participant #1; ρ (rho)=−0.60, p=0.003, participant #2; ρ=−0.7l, p=0.0026, and participant #3; ρ=−0.54, p=0.016. Measures of maximal inspiratory pressure also stabilized, particularly in two participants, during Treg infusions.

### 8.1.1.4 Conclusions

These results demonstrate the safety and potential benefit of expanded autologous Treg infusions, warranting further clinical trials in ALS patients. The correlation between Treg suppressive function and disease progression underscores the significance of using Treg suppressive function as an indicator of clinical status.

### 8.1.1.5 Classification of Evidence

This study provides class IV evidence. This is a phase 1 trial with no controls.

### 8.1.2. INTRODUCTION

CD4⁺CD25⁺FOXP3⁺ regulatory T lymphocytes (Tregs) are a subpopulation of T lymphocytes that are immunosuppressive and maintain tolerance to self-antigens, with their dysfunction playing a pivotal role in the development of autoimmune disorders (Refs. 1-4). In ALS mice, infusions of Tregs slow disease progression and prolong survival, and Tregs suppress the proliferation of responder T lymphocytes and the activation of microglia (Refs 5,6). In ALS patients, the expression of the Treg master transcription factor FOXP3 is reduced in rapidly progressing patients (Ref. 7), with subsequent impairment of Treg suppressive functions; FOXP3 expression and Treg suppressive functions correlate with the extent and rapidity of disease progression (Ref. 8). When expanded ex vivo in the presence of interleukin (IL)-2 and rapamycin, Treg suppressive function is restored (Refs. 8,9). These data suggest that infusions of expanded autologous Tregs will improve Treg suppressive function in vivo and slow rates of disease progression in ALS patients. To test these hypotheses, a first-in-human phase 1 study was initiated to determine whether infusions of expanded autologous Tregs into ALS patients were safe and tolerable during early and later stages of disease. IL-2 was administered concomitantly in the participants in an effort to stabilize and possibly enhance the suppressive functions of the infused Tregs. Further, the relationship between ex vivo Treg suppressive functions and participants' clinical statuses was explored.

### 8.1.3. METHODS

### 8.1.3.1 Primary Research Question

Are infusions of expanded autologous Tregs safe and tolerable in ALS patients during both early and later stages of disease? This study is a phase 1 trial with no controls and provides class IV evidence that infusions of expanded autologous Tregs are safe and tolerable during early and later stages of disease.

### 8.1.3.2 Standard Protocol Approvals, Registrations, and Patient Consents

Approvals from the Food and Drug Administration and Institutional Review Board at Houston Methodist Hospital were obtained prior to study initiation. Written informed consent was obtained prior to enrollment. The study was registered on clinicaltrials.gov (NCT03241784).

### 8.1.3.3 Study Design and Participant Selection Criteria

This study was conducted at the Houston Methodist Neurological Institute. Patients with no family history of ALS, and with differing sites of symptom onset and rates of disease progression, were recruited from Houston Methodist Hospital's MDA/ALSA ALS clinic. Three participants with arm, bulbar, and leg-onset ALS, respectively, were enrolled in the trial (Table 1). Participants were recruited, treated and followed between January 2016 and February 2018.

The participants underwent a total of 8 infusions of expanded autologous Tregs with concomitant subcutaneous IL-2 injections. Four Treg infusions were administered every 2 weeks at an early stage of the disease followed by 4 Treg infusions administered every 4 weeks at a later stage. Each Treg dose was 1×10⁶ cells/kg. The Treg dose was empirically determined, but was selected within the range of what has been shown to be safe and tolerable in patients with type 1 diabetes (Ref. 4). IL-2 was administered subcutaneously 3 times weekly at a dose of 2×10⁵ IU/m²/injection beginning the day after the first Treg infusion and continued throughout the study period.

### 8.1.3.4 Infusions of Expanded Autologous Tregs

Leukapheresis was performed one month prior to the first Treg infusion. Tregs were isolated and expanded ex vivo in the Good Manufacturing Practice-compliant facility at M.D. Anderson Cancer Center according to a previously described protocol (Refs. 8,9). Each Treg infusion was administered intravenously through a peripheral line and participants were closely monitored for any infusion-related adverse responses for 4 hours following the infusion.

### 8.1.3.5 Clinical Evaluations

The revised ALS Functional Rating Scale (ALSFRS-R), Appel ALS rating scale (AALS) (Ref. 10) and maximal inspiratory pressure (MIP) measurements were performed immediately prior to each Treg infusion, every 2 weeks during each round of infusions, and monthly after each round. Forced vital capacity (FVC) was monitored at each evaluation as a component of the AALS. Participants were asked about adverse events at each encounter.

### 8.1.3.6 Assessing Treg Percentage and Suppressive Function in the Peripheral Blood

Peripheral blood was drawn one month prior to the first Treg infusion, immediately before each infusion, the day after each infusion, every 2 weeks during each round of infusions, and monthly after each round. The percentage of CD4⁺CD25⁺FOXP3⁺ Tregs within the total CD4⁺ population was assessed by flow cytometry (Ref. 8). Treg suppressive function on the proliferation of autologous responder T lymphocytes was assessed by [³H]-thymidine incorporation (Ref. 8).

### 8.1.3.7 Statistical Analysis

Correlation between changes in the AALS and Treg suppressive function was determined by Spearman's correlation using GraphPad Prism 7 software and depicted by Spearman's rho (ρ). Two-tailed p values less than 0.05 were considered significant. Data collected between the first and fifth Treg infusions were compared with values from the day of the first infusion. Data collected after the fifth Treg infusion were compared with the values from the day of the fifth infusion.

### 8.1.3.8 Data Availability

Individual de-identified participant data not published within the article including clinical evaluations and Treg percentage and suppressive function results will be shared by request from any qualified investigator.

### 8.1.4. RESULTS

### 8.1.4.1 Safety

No infusion-related adverse events or clinically significant changes in safety labs or EKG findings were observed. All participants noted dramatic increases in the frequency, intensity and distribution of fasciculations during each round of infusions. Fasciculations were noted within a few minutes to a few days following each Treg infusion, and lasting from days to more than 1 month following the completion of each round of infusions. Participant #1 experienced increased muscle cramps in his legs from weeks 2 to 6, two falls on weeks 17 and 23, and an episode of pharyngitis on week 10. Participant #2 underwent placement of a percutaneous endoscopic gastrostomy tube on week 9. He developed aspiration pneumonia on week 19 and his IL-2 injections were temporarily suspended until week 23. His progressive dysphagia and episode of aspiration pneumonia were likely due to his bulbar ALS. Participant #2 dropped out of the study on week 50 due to his progressive disease and was placed in hospice care. He expired on week 51 due to respiratory failure secondary to ALS. Participant #3 developed two suspected gastrointestinal infections and an upper respiratory infection between weeks 24 and 29. She reported mild dyspnea on exertion beginning on week 48.

### 8.1.4.2 Treg Percentage and Suppressive Function Increased During Infusions

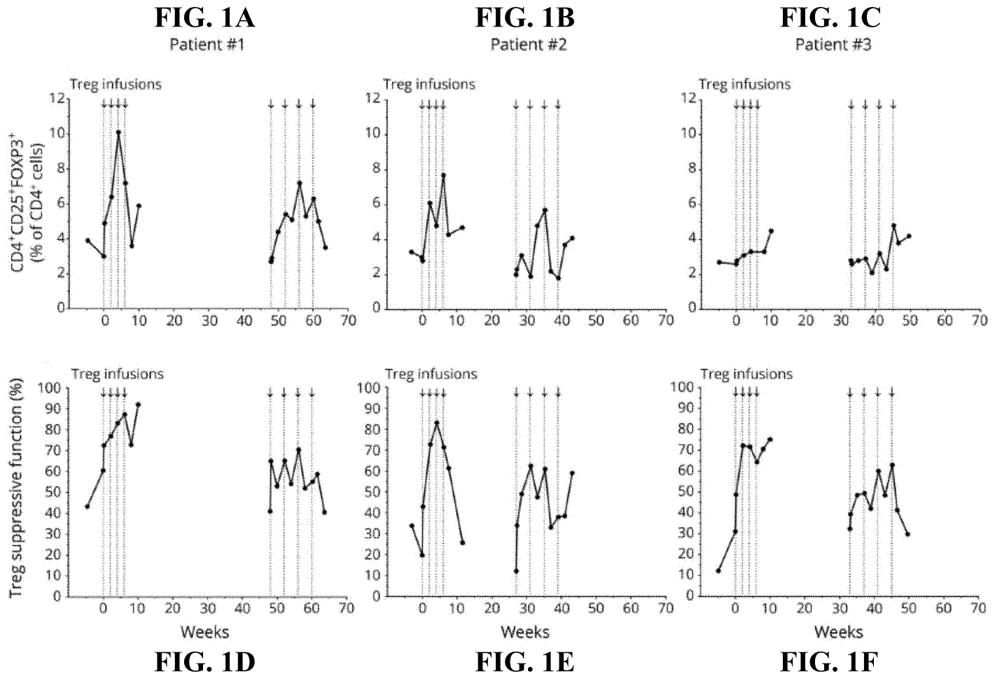

In all participants, Treg percentage (FIGS. 1A-1C) and suppressive function (FIGS. 1D-1F) increased during the first round of infusions, declined between each round of infusions, and increased again during the second round. 8.1.4.3 Enhanced Treg suppressive function correlated with slowing of functional decline

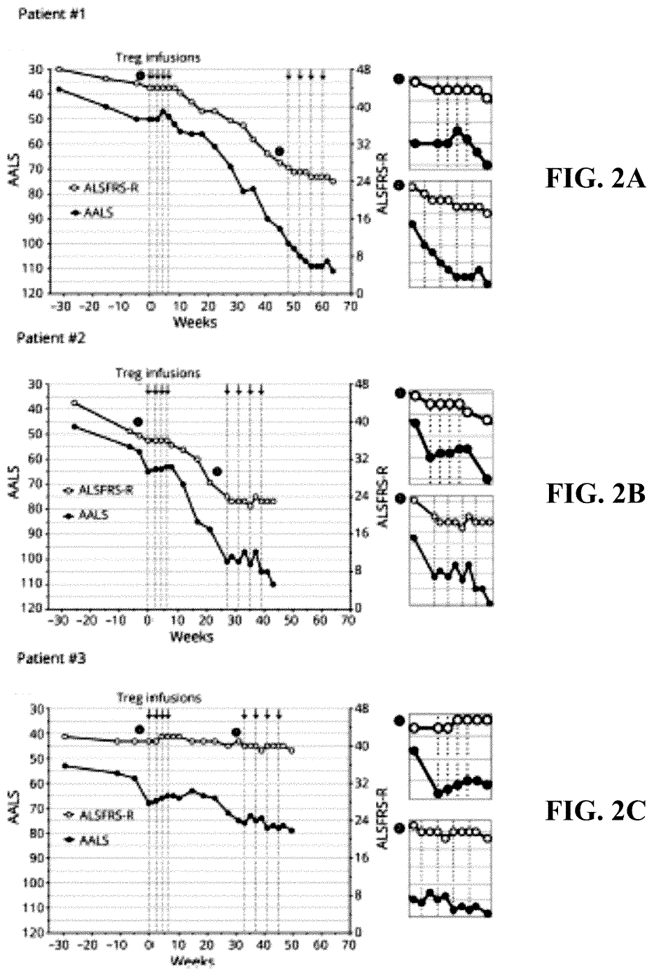

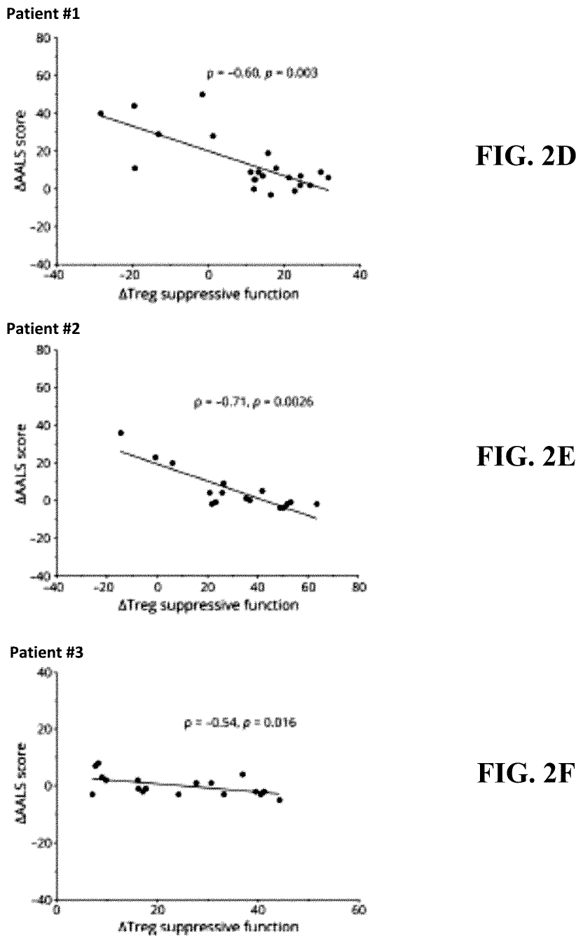

In all participants, the rate of decline of the ALSFRS-R and AALS slowed for 2 months during the first round of infusions, accelerated between each round of infusions, and slowed again over 4 months during the second round (FIG. 2A-2C). Spearman's correlation showed that increased Treg suppressive function correlated with slowing of disease progression per the AALS for each participant (FIG. 2D-2F; ρ=−0.60, p=0.003 in participant #1; ρ=−0.71, p=0.0026 in participant #2; and ρ=−0.54, p=0.016 in participant #3). The larger the increase in Treg suppressive function, the smaller the decline in the AALS at the next clinical evaluation.

### 8.1.4.4 Maximal Inspiratory Pressures Stabilized During Infusions

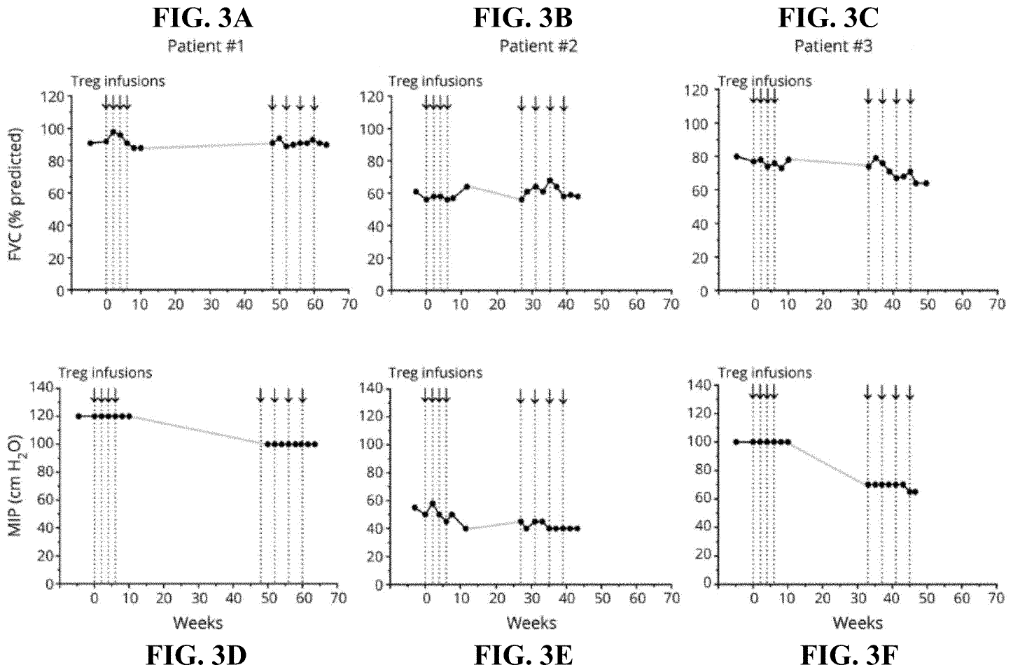

In all participants, the FVC remained relatively unchanged during the Treg infusions and between each round (FIG. 3A-3C). In participants #1 and #3, the MIPs were stable during the first round of infusions and deteriorated between each round. The MIPs again stabilized during the second round. Participant #2 was treated with noninvasive ventilation prior to enrollment and continued treatment throughout the study. Participant #2's MIPs were low, but remained relatively stable during each round of infusions and between each round (FIG. 3D-3F).

### 8.1.5. DISCUSSION

Three ALS patients were infused with autologous expanded Tregs with concomitant subcutaneous IL-2 injections at early and later stages of disease. Tregs were also infused at a later stage of disease to determine whether the infusions remained safe and the beneficial effects on disease progression could be extended by increasing the dosing interval. Treg infusions were safe and well-tolerated regardless of the burden of disease. Treg suppressive function correlated with changes in the AALS; the greater the improvement in Treg suppressive function, the slower the rate of clinical progression. This correlation supports the value of Treg suppressive function as a meaningful indicator of clinical status. In addition, Treg infusions did not adversely affect respiratory function and appeared to stabilize the decline in MIPs in the two participants who were not being treated with noninvasive ventilation.

In a prior pilot study, low dose IL-2 administered subcutaneously for 1 year in 5 patients with ALS was safe and tolerable, but did not appear to alter the clinical course or increase endogenous Treg numbers, likely related to impaired endogenous Treg responsiveness to IL-2 (unpublished results). In the present study, subcutaneous injections of low dose IL-2 were administered to stabilize the infused expanded Tregs. During infusions in all participants, several data points were observed in which IL-2 could have enhanced the proliferation and function of infused Tregs. However, IL-2 was not of critical value in the interim between each round when Treg percentage and suppressive function, and clinical status deteriorated.

Common to all participants was the perceived increase in fasciculations. The rapid onset of fasciculations suggests a peripheral action in the lower motor neuron possibly mediated by immune modulation of ectopic axonal firing. In the earlier IL-2 alone pilot study, increased fasciculations were not observed, suggesting that the infused Tregs directly or indirectly caused the fasciculations in this study. Also, common to all participants, was the occurrence of infections during the study; pharyngitis in participant #1, aspiration pneumonia in participant #2, and gastrointestinal and upper respiratory infections in participant #3. A potential increased risk of infections with Treg and IL-2 treatment is concerning and requires further study in a larger number of ALS patients.

Although this study lacked blinding and placebo controls, slowing of disease progression was observed during the initial infusions at an early stage of disease and the subsequent 4 monthly infusions at a later stage of disease. Administering the ALSFRS-R to the participants during the initial infusions every 2 weeks enhances the likelihood of a placebo effect. However, stabilization of the AALS has not been observed in prior studies of subcutaneous IL-2 injections (unpublished results) or infusions of allogeneic hematopoietic stem cells (Ref. 11). Furthermore, the increased Treg suppressive function, which correlated with the clinical state, and the observed stabilization of MIPs, were not likely to have been influenced by a placebo effect. Increased clinical progression rates were observed between each round of infusions, but it was not clear whether the progression was related to the cessation of Treg infusions or would have occurred spontaneously. More pertinent is the observation that subsequent Treg infusions were beneficial at later stages of disease with increased disease burden and rate of progression. Circulating functional Tregs may slow disease progression by suppressing peripheral proinflammatory monocytes/macrophages and responder T lymphocytes, as well as entering the CNS and suppressing activated microglia. Defining peripheral and central actions of Tregs merits further investigation.

The results from this study support the need for a phase 2, randomized, placebo-controlled trial over a longer period to test the clinical efficacy, safety and tolerability of different doses of Tregs in a larger number of ALS patients. The goal of future studies is to determine whether optimized doses of Tregs infused at regular intervals prolong slowed progression and minimize the more rapid progression associated with cessation of Treg infusions.

As shown above, the Treg dose used in the phase I study, for example, was 1×10⁶/kg/infusion resulting in an average individual dose of 70-100 million cells per infusion. (See section 8.1 below). Eight doses were administered to each patient and each dose required its own manufacturing campaign that involved a 25-day ex vivo expansion, which drastically increased the efforts and costs per patient. Prolonged expansion times also result in Treg products with reduced suppressive capabilities, which would not be optimal for patients, e.g., patients with ALS. A successful manufacturing process would produce large numbers of highly functional Tregs over a shorter expansion time. From one patient exposure to leukapheresis with one expansion campaign, sufficient numbers of Tregs should be produced to accommodate larger phase 11/111 studies that require higher doses of Tregs over longer periods of time. Likewise, from one patient sample, e.g., from a single leukapheresis, with one expansion campaign, sufficient numbers of Tregs should be produced for an entire therapeutic regimen, for example, an entire therapeutic regimen for treatment of a neurodegenerative disease such as ALS or Alzheimer's disease, an autoimmune disease such as Type 1 diabetes or rheumatoid arthritis, or graft versus host disease (GVHD) such as GVHD following a bone marrow transplantation.

To achieve these goals, several aspects of Treg expansion have been optimized and a more controlled manufacturing platform has been developed that yields vast numbers of highly functional Tregs that are cryopreserved, e.g., cryopreserved under cGMP conditions (see Sections 8.3, 8.5, and 8.6 below). Cryopreserved Tregs can be sent to patient locations where they can be thawed and infused into patients at different doses and frequencies under controlled conditions. Exemplary standard operating procedures for the manufacturing process are provided below, see, e.g., Sections 8.3, 8.5, and 8.6.

A Phase II study is planned to begin this year, which will evaluate whether the Treg therapy improves Treg function in ALS patients. During the trial, the safety of monthly Treg infusions and higher doses of Treg infusions will be determined over a year long period.

### 8.1.6. REFERENCES

1. Sakaguchi S. Naturally arising Foxp3-expressing CD25+CD4+ regulatory T cells in immunological tolerance to self and non-self. Nat Immunol 2005; 6:345-352.
2. Viglietta V, Baecher-Allan C, Weiner H L, Hafler D A. Loss of functional suppression by CD4+CD25+ regulatory T cells in patients with multiple sclerosis. J Exp Med 2004; 199:971-979.
3. Dejaco C, Duftner C, Grubeck-Loebenstein B, Schirmer M. Imbalance of regulatory T cells in human autoimmune diseases. Immunology 2006; 117:289-300.
4. Bluestone J A, Buckner J H, Fitch M, et al. Type 1 diabetes immunotherapy using polyclonal regulatory T cells. Sci Transl Med 2015; 7:315ra189.
5. Beers D, Henkel J S, Zhao W, et al. Endogenous regulatory T lymphocytes ameliorate amyotrophic lateral sclerosis in mice and correlate with disease progression in subjects with amyotrophic lateral sclerosis. Brain 2011; 134:1293-1314.
6. Zhao W, Beers D R, Liao B, Henkel J S, Appel S H. Regulatory T lymphocytes from ALS mice suppress microglia and effector T lymphocytes through different cytokine-mediated mechanisms. Neurobiol Dis 2012; 48:418-428.
7. Henkel J S, Beers D R, Wen S, et al. Regulatory T-lymphocytes mediate amyotrophic lateral sclerosis progression and survival. EMBO Mol Med 2013; 5:64-79.
8. Beers D R, Zhao W, Wang J, et al. ALS patients' regulatory T lymphocytes are dysfunctional, and correlate with disease progression rate and severity. J Clin Invest Insight 2017; 2:e89530.
9. Alsuliman A, Appel S H, Beers D R, et al. A robust, good manufacturing practice-compliant, clinical-scale procedure to generate regulatory T cells from patients with amyotrophic lateral sclerosis for adoptive cell therapy. Cytotherapy 2016; 18:1312-1324.
10. Haverkamp L J, Appel V, Appel S H. Natural history of amyotrophic lateral sclerosis in a database population. Validation of a scoring system and a model for survival prediction. Brain 1995; 118: 707-719.
11. Appel S H, Engelhardt J I, Henkel J S, et al. Hematopoietic stem cell transplantation in patients with sporadic amyotrophic lateral sclerosis. Neurology. 2008; 71:1326-1334.

TABLE 1
Participant characteristics.

| Participant No. | 1 | 2 | 3 |
|---|---|---|---|
| Age (yr) | 47 | 46 | 56 |
| Sex | Male | Male | Female |
| Initial weight (kg) | 92 | 77 | 79 |
| Site of symptom onset | Arm | Bulbar | Leg |
| Time from symptom onset to | 7 | 18 | 12 |
| diagnosis (months) | | | |
| Time from symptom onset to 1^{st} Treg | 14 | 24 | 38 |
| infusion (months) | | | |
| Riluzole use at study entry | Yes | Yes | Yes |
| Non-invasive ventilation use at study entry | No | Yes | No |
| ALSFRS-R just prior to 1^{st} Treg infusion | 44 | 36 | 41 |
| AALS just prior to 1^{st} Treg infusion | 50 | 65 | 68 |
| Forced vital capacity (L) just prior to | 5.25 | 2.84 | 2.24 |
| 1^{st} Treg infusion | | | |
| Forced vital capacity (% predicted) just prior | 92 | 56 | 77 |
| to 1^{st} Treg infusion | | | |
| Maximal inspiratory pressure (cm H₂O) just | 120 | 50 | 100 |
| prior to 1^{st} Treg infusion | | | |

### 8.2 Example 2: Restoring Regulatory T Cells Dysfunction in Alzheimer Disease Through Ex Vivo Expansion

As the role of Tregs in Alzheimer's disease patients is poorly understood, the peripheral blood population and function of Tregs was evaluated during the disease course. It was found that Tregs exhibit a failure in suppressive activity at the clinical Alzheimer dementia stage, which could shift the immune system response towards a pro-inflammatory state, both in the periphery and in the brain. Further, the potential of ex vivo expansion of Tregs to restore homeostasis was also evaluated, as described below.

### 8.2.1. MATERIALS AND METHODS

### 8.2.1.1 Patient Recruitment and Alzheimer-Defined Population

Patients met “research criteria for MCI (mild cognitive impairment) and probable Alzheimer dementia, incorporating neurodegeneration biomarkers” (decreased 18-fluorodeoxyglucose (FDG) uptake in temporo-parietal cortex in positron emission tomography (PET); and/or disproportionate atrophy in medial temporal lobe and medial parietal cortex on magnetic resonance imaging), according to 2011 guidelines from the National Institute on Aging-Alzheimer's Association (NIA-AA) (Albert et al., 2011; Jack et al., 2011; McKhann et al., 2011). Written informed consent was obtained from all patients and aged-matched healthy controls (HC) according to the Declaration of Helsinki following ethics approval from the Institutional Review Board (IRB) at the Houston Methodist Research Institute. Staging of dementia severity was based on the Clinical Dementia Rating Scale (CDR) assessment instrument (Morris, 1993; O'Bryant et al., 2008). Participants with a global CDR score of 0.5 were categorized as mild cognitive impairment (MCI) (n: 42, M/F: 18/24, mean Age: 71.4) and patients with a CDR≥1 were considered to have Alzheimer dementia [n: 46 (24 CDR1 and 22 CDR2&3), M/F: 20/26, mean Age: 70.0]. Enrolled healthy controls were required to have a CDR of 0 (n: 41, M/F: 17/24, mean Age: 69.3)ou

### 8.2.1.2 Flow Cytometry

Multicolor flow cytometry was used to assess the immunophenotype of Tregs. Antibodies against the following molecules were provided by: CD3 BV650 (BD Biosciences), CD8 BV450 (BD Biosciences), CD4 APC-H7 (BD Biosciences), CD25 PerCPCy5.5 (BD Biosciences), CD73 eFluor 450 (eBioscience™), PD-1 BV 650 (Biolegend). Dead cells were stained by LIVE/DEAD® Fixable Blue Dead Cell Stain Kit (Life Technology). For intracellular staining, cells were fixed and permeabilized using the FoxP3/Transcription Factor Staining Buffer Set (eBioscience), and then stained with FoxP3 Alexa Fluor 488 (eBioscience), IL13-PE (eBioscience) and Granzyme B APC (BioLegend). Appropriate isotype controls were used to set the quadrants and to evaluate background staining. Cells were analyzed using an LSRII flow cytometer with BD FACSDIVA software.

### 8.2.1.3 Immune Cell Isolation

Mononuclear immune cells were isolated from peripheral blood of participants using Lymphoprep (Stemcell) density gradient centrifugation. Tregs and responders T cells (Tresps) were isolated using the CD4⁺CD25⁺ Regulatory T Cell Isolation Kit (Miltenyi Biotec) according to the manufacturer's instructions. To increase purity, the positively selected cell fraction containing the CD4⁺CD25⁺ regulatory T cells was run twice through the MS column. The unlabeled CD4⁺CD25 cell effluent was collected as the Tresp population.

### 8.2.1.4 Treg Suppression Assays on Tresp Proliferation

Isolated Tresps were placed in a 96-well plate at a density of 50,000 cells per well followed by co-culture of corresponding Tregs at a ratio of 1:1 and 2:1 (Tresps:Tregs) in at least triplicates. A CD3/CD28 T cell stimulation reagent (Miltenyi Biotec) was added to the co-cultures. After 5 days of culture, cells were pulsed with tritiated thymidine (1 Ci/well; Amersham Life Sciences) for 18 hours. The cells were harvested, and thymidine uptake was measured using a gas-operated-plate reader (Packard Instruments). The percent suppression of proliferation was calculated using the following formula: Percentage suppression=100−[(counts per minute of proliferating Tresps in the presence of Tregs/counts per minute of proliferating Tresps in the absence of Tregs)×100].

### 8.2.1.5 Suppression Assays on IPS-Derived Pro-Inflammatory Macrophages

The protocol for generation of mature monocyte cells from induced pluripotent stem cells (iPSCs) consists of 5 sequential steps through which mature monocytes are differentiated from human pluripotent cells in a stepwise manner (Yanagimachi et al., 2013). Mature monocytes were polarized to pro-inflammatory macrophages through treatment with GM-CSF (50 ng/mL) for 7 days, lipopolysaccharide (0.1 ng/mL) and interferon-7 (0.2 ng/mL) for one hour. These iPSC-derived macrophages robustly produced proinflammatory IL-6, TNFα and IL-1B cytokines. Tregs from patients and healthy controls were co-cultured with activated macrophages for short (4 hours) and longer (24 hour) time periods. Cultured media was collected to assess cytokine protein levels via ELISA. Cells were then collected from culture and messenger RNA was isolated for the examination of cytokine transcripts. For IL-13 and CD25 blockade in the co-culture, anti-human IL-13 Ab (MAB2131) and anti-human CD25 Ab (MAB223) were used from R&D Systems. A falcon insert system (Life Science) was utilized to block direct contact between Treg and macrophage populations in the co-culture.

### 8.2.1.6 RNA Purification, RT-PCR Analysis

Using Trizol reagent, followed by Direct-zol RNA MiniPrep Kit (Zymo Research), messenger RNA was extracted from patient immune cell populations and in vitro cell experiments. Quantitative PCR experiments were performed using a One-Step RT-PCR kit with SYBR Green and run on the Bio-Rad iQ5 Multicolor Real-Time PCR Detection Systems. Primers for the study were purchased from BioRad and the relative expression level of each messenger RNA was calculated using the ΔΔCt method with normalization to β-actin and relative to control samples.

### 8.2.1.7 Ex Vivo Tregs Expansion

Bead-selected CD4⁺CD25^{high} T lymphocytes were suspended at a concentration of 1×10⁶ cells/mL in media containing 100 nM of rapamycin (Miltenyi Biotec), 500 IU/mL IL-2 and Dynabeads™ Human Treg Expander (Gibco™) at a 4:1 bead-to-cell ratio. Fresh media containing rapamycin and IL-2 were added to the cells every 2 to 3 days. After 10 days of culture, cells were harvested and washed. The number of expanded Tregs and their suppressive functions were assayed.

### 8.2.1.8 Statistical Analysis

The minimum sample size was calculated to achieve 80% power with 5% significance level to detect the differences of Treg characteristic and function between varying stages of Alzheimer disease. Investigators were blinded to the identity of the groups during outcome assessment. Comparisons were performed using paired or unpaired student's t-test (for two groups) or oneway ANOVA (for more than two groups). Correlations were determined using the linear regression. Data were expressed as Mean±SEM and p values less than 0.05 were considered significant.

### 8.2.2. RESULTS

As discussed below, this study documented significant alteration in the immunophenotype as well as the suppressive activity of Tregs throughout the course of Alzheimer disease.

Also, for the first time in Alzheimer disease, the potential for ex vivo expansion of patients' Tregs was evaluated. Following ex vivo expansion, the immunoregulatory capacity of Tregs was substantially enhanced in both patients and healthy controls through a cell contact-mediated mechanism. These expanded Tregs expressed 20-fold higher levels of CD25 protein on their cell surfaces.

The suppressive activities of these ex vivo expanded Tregs were also examined. Following expansion, the ability of Tregs to suppress Tresp proliferation was significantly increased in both patients and healthy controls. Interestingly, following expansion the immunophenotype and suppressive activity of Tregs from advanced Alzheimer disease patients were comparable to those of healthy controls. Further, while freshly isolated Tregs showed no effects on activated macrophages, ex vivo expanded Tregs displayed substantially enhanced suppressive activity on pro-inflammatory macrophage-derived cytokine production.

The data presented here demonstrate that the ex vivo expansion of Tregs restores their immunophenotype and suppressive function at the Alzheimer disease stage. These results provide a rationale for the autologous transfer of expanded Tregs in Alzheimer disease patients.

### 8.2.2.1 Suppressor T Cells' Immunophenotype in Alzheimer Disease

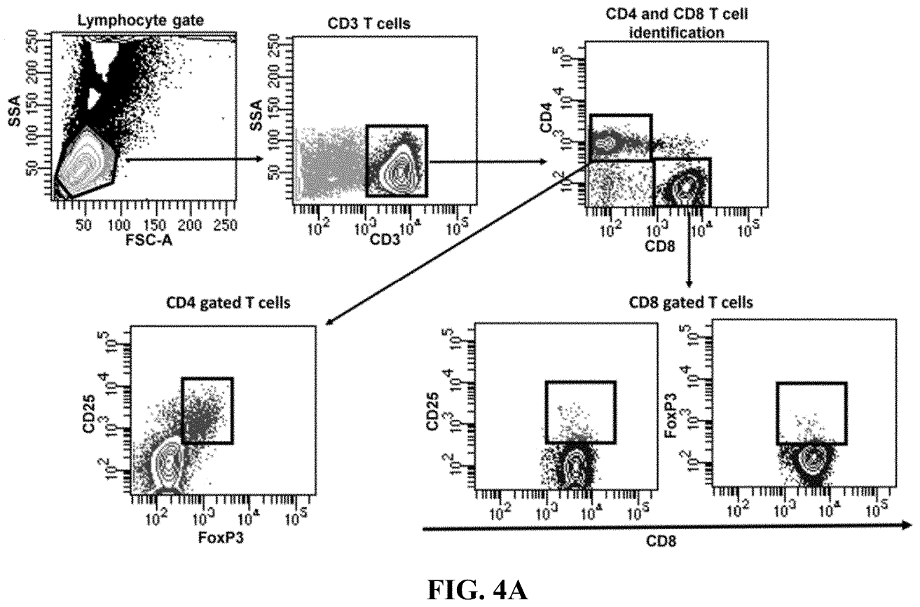

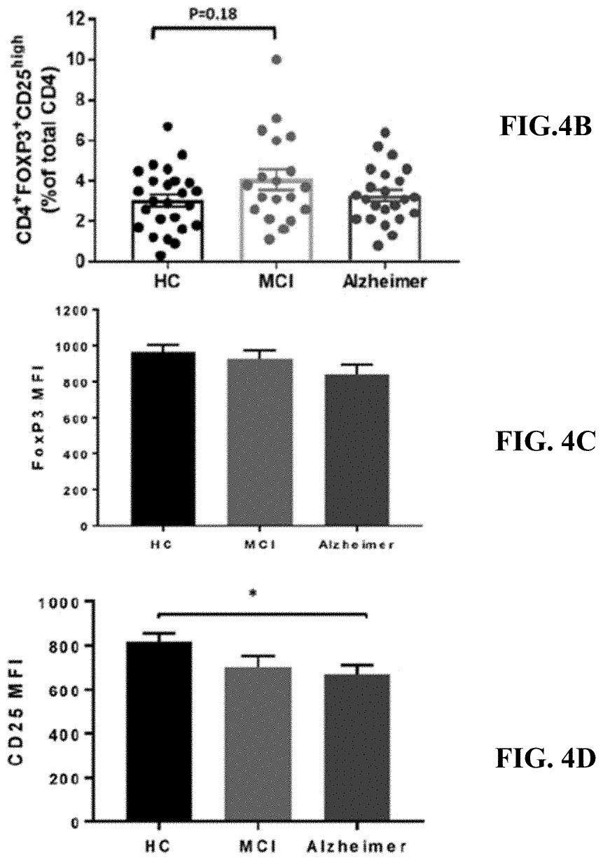

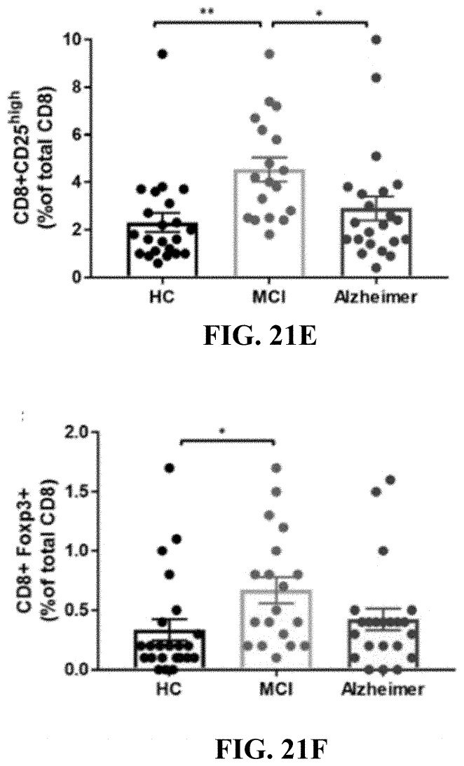

Single blood samples from 20 Alzheimer patients, 18 MCI and 27 HC were examined by flow cytometry. In the present study, about half of the patients in the Alzheimer group had moderate to severe dementia (CDR 2R&3). A gating strategy (FIG. 4A) was applied that allows the identification of the Tregs by an existing established phenotype (CD4⁺FoxP3⁺CD25^{high}) The percentage of CD4⁺FOXP3⁺CD25^{high} Tregs did not differ among HC, MCI and Alzheimer groups (FIG. 4B).

While the mean fluorescent intensity (MFI) of FoxP3 in CD4⁺FoxP3⁺CD25^{high} Tregs was comparable among three groups (FIG. 4C), decline in CD25 MFI was identified in Alzheimer group compared with HC (FIG. 4D). In evaluating CD8 suppressor T cells, the percentages of CD8⁺CD25^{high} and CD8⁺FOXP⁺ cells were increased in MCI compared to healthy controls and returned to baseline level at Alzheimer dementia stage (FIG. 4E, and FIG. 4F).

### 8.2.2.2 Compromised Suppressive Function of Tregs on Corresponding Tresps in Alzheimer Disease

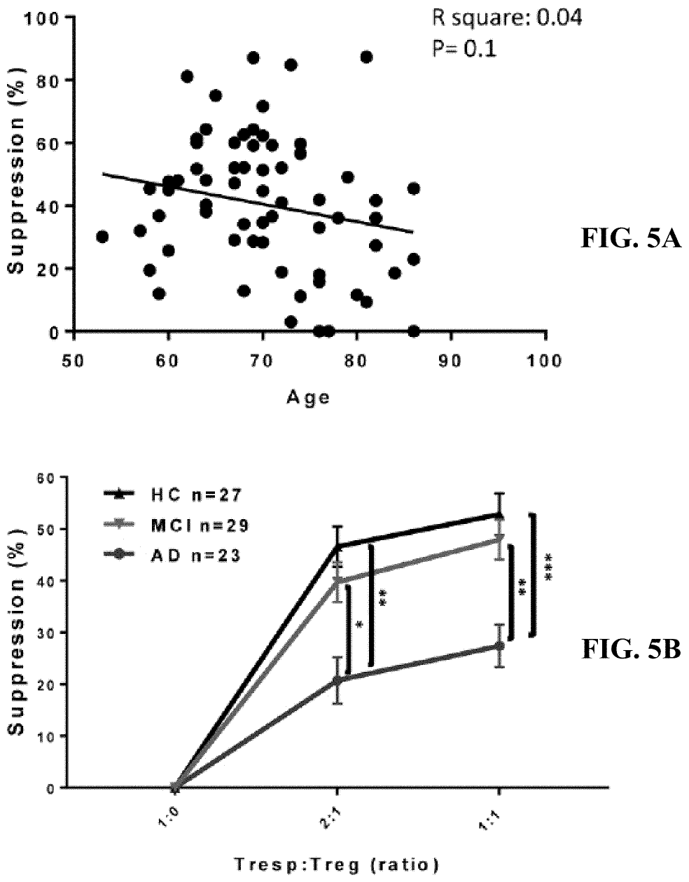

CD4⁺CD25^{high}Tregs were positively selected from whole blood based on CD25 expression. Tregs and Tresps were co-cultured with varying numbers of Tregs against the same number of Tresps. First, the effects of age and gender on the suppressive function of Tregs were evaluated. There was no difference in the suppressive function of Tregs among men and women. No correlation was found between Treg suppressive activity and the age of the subjects (FIG. 5A). Treg suppressive activity in MCI (n=29) was comparable to HC (n=27) at both 1:1 and 2:1 Tresp:Treg ratios (1:1 ratio: HC=52.8%±4.36, MCI=47.68% 4.40, p=0.933; 2:1 ratio, HC=46.55%±3.88, MCI=39.71%±3.84, p=0.890). The suppressive function of Tregs in Alzheimer disease patients (n=23) was reduced compared to both HC (1:1 ratio: Alzheimer disease=27.45%±4.11, Alzheimer disease vs. HC p=0.0001; 2:1 ratio Alzheimer disease=23.3%±4.96, Alzheimer vs. HC p=0.0018) and MCI (1:1 ratio: Alzheimer disease vs. MCI: p=0.0034, 2:1 ratio: p=0.0357) (FIG. 5B).

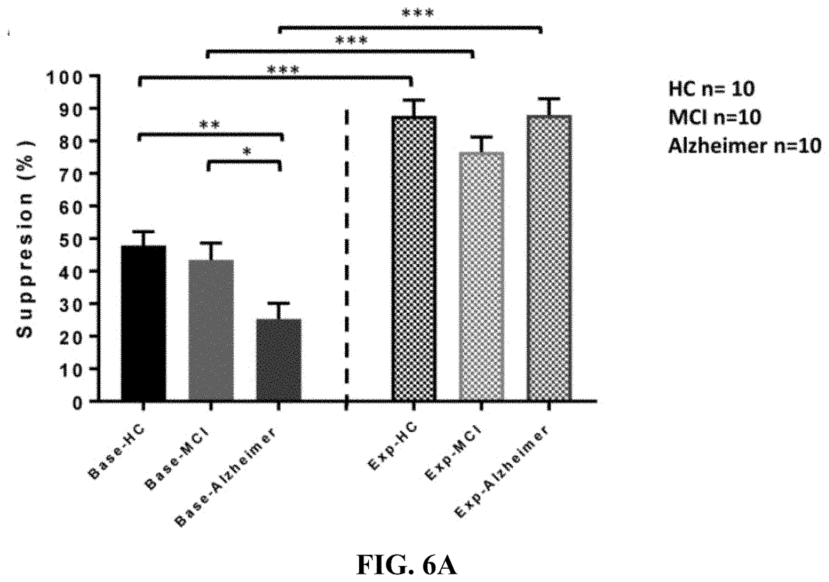

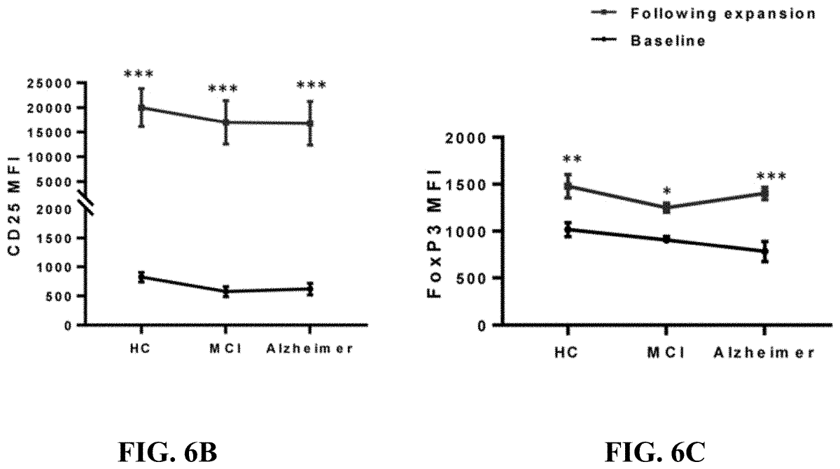

Enhanced immunophenotype and suppressive activity of Tregs on Tresp proliferation following ex vivo expansion. In another experiment, distinct smaller sets of patients and controls were enrolled (10 Alzheimer patients, 10 MCI and 10 HC). CD4⁺CD25^{high} Tregs were isolated from blood and the suppressive function of Tregs on Tresp proliferation at a 1:1 ratio was assayed. Similar to the initial finding (FIG. 5B), the suppressive function of MCI Tregs (43.45%±5.20) was comparable to HC (47.89%±4.23) (p=0.983), but the suppressive function of Tregs in Alzheimer disease (25.27%±4.81) was decreased compared to HC (p=0.0130) (FIG. 6A). Tregs from the same individuals were expanded ex vivo in the presence of IL-2, rapamycin and CD3/CD28 beads for 10 days. The expansion rates of Tregs isolated from the HC, MCI and Alzheimer disease groups were not different. Enhancement in the suppressive function of ex vivo expanded (exp) Tregs on Tresp proliferation was noted in all groups compared to their respective baselines (base) Tregs (exp-HC=87.7%±4.82, exp vs. base-HC p=0.0001, exp-MCI=76.6%±4.61, exp vs. base-MCI p=0.0002, exp-Alzheimer=87.98%±4.97, exp vs. base-Alzheimer p<0.0001). Interestingly, in contrast to baseline Tregs, the suppressive activity of the expanded Tregs became comparable among patients and healthy controls (FIG. 6A). The immunophenotypes of baseline and expanded Tregs were also assayed by flow cytometry. Following expansion, the MFI of CD25 was remarkably (approximately 20-fold) increased in all three groups (FIG. 6B). The MFI of FoxP3 in expanded Tregs was also elevated (approximately 1.5 to 2-fold) (FIG. 6C). CD25 and FoxP3 MFIs in expanded Tregs were comparable between patients and controls. Amplified suppression of Tregs on iPSC-derived pro-inflammatory macrophages following ex vivo expansion. CD4⁺CD25^{high}Tregs were co-cultured with pro-inflammatory iPSC-derived macrophages (M1) and the relative changes of IL-6, TNFα, and IL-1B transcripts (after 4 hrs) and protein levels in the supernatant (after 24 hrs) were assayed.

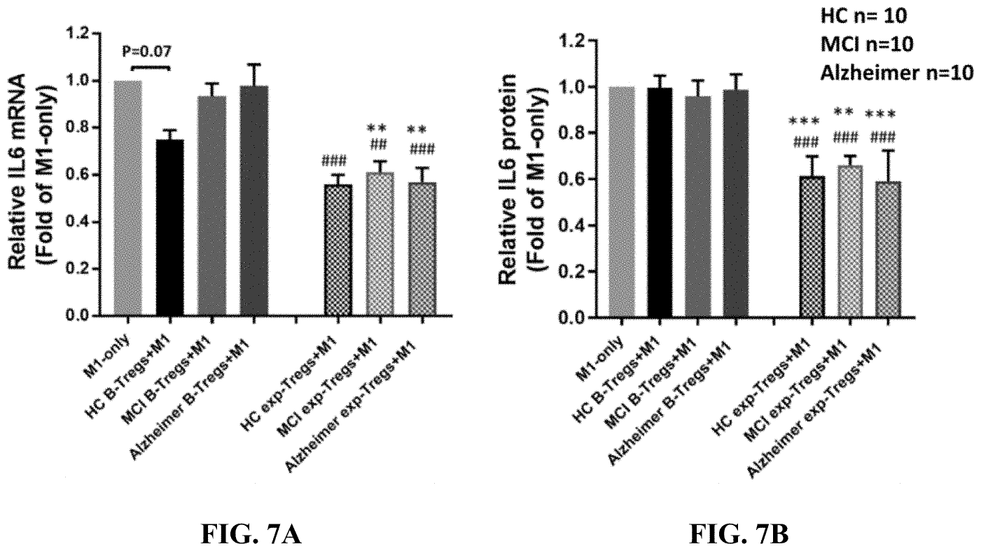

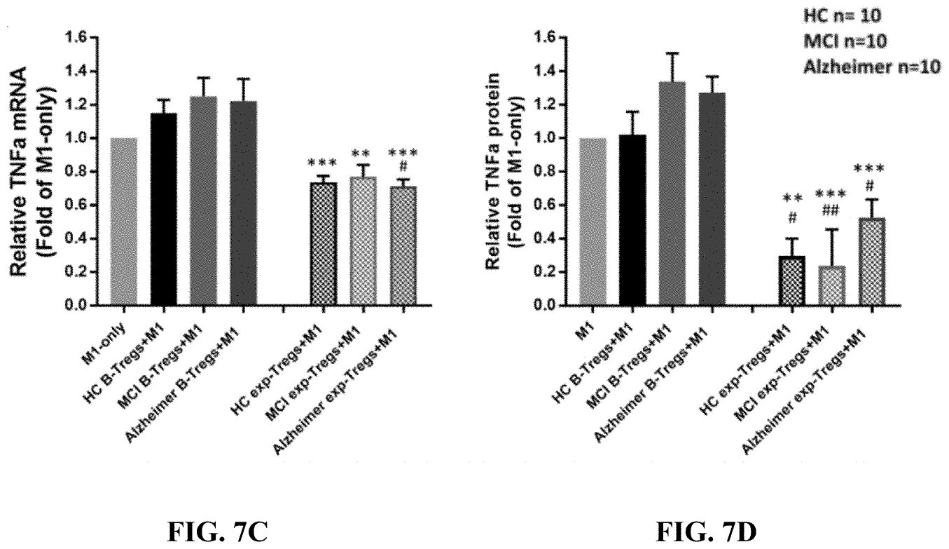

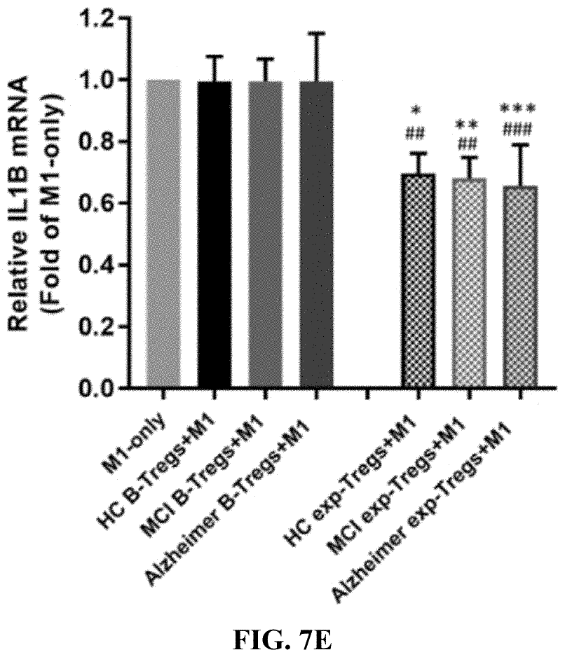

At baseline (B), Tregs from HC attenuated macrophage IL-6 transcript expression by 25% but not significant (p=0.071) (FIG. 7A).

Baseline Tregs of MCI or Alzheimer patients showed no suppression on M1-derived IL-6 transcript or protein (FIG. 7A and FIG. 7B). The Tregs from these same subjects were expanded ex vivo for 10 days and then co-cultured with pro-inflammatory macrophages. A reduction of M1-derived IL-6 transcript and protein expression levels were noted following co-culture with expanded Tregs in all groups (FIG. 7A and FIG. 7B). The co-culture of baseline Tregs with M1 did not attenuate TNFα transcript and protein levels. Expanded Tregs of Alzheimer disease, MCI and HC displayed an enhanced capacity to suppress M1-derived TNFα transcript and protein, compared to corresponding baseline Tregs (FIG. 7C and FIG. 7D). Similar to IL6 and TNFα findings, while baseline Tregs did not alter IL1B transcript levels, expanded Tregs displayed an enhanced capacity to suppress M1-derived IL1B transcript in all three groups (FIG. 7E). However, in evaluating IL1B protein, the protein values were too low to be measured reliably.

### 8.2.2.3 Up-Regulated Immunoregulatory Gene Expressions in Expanded Tregs

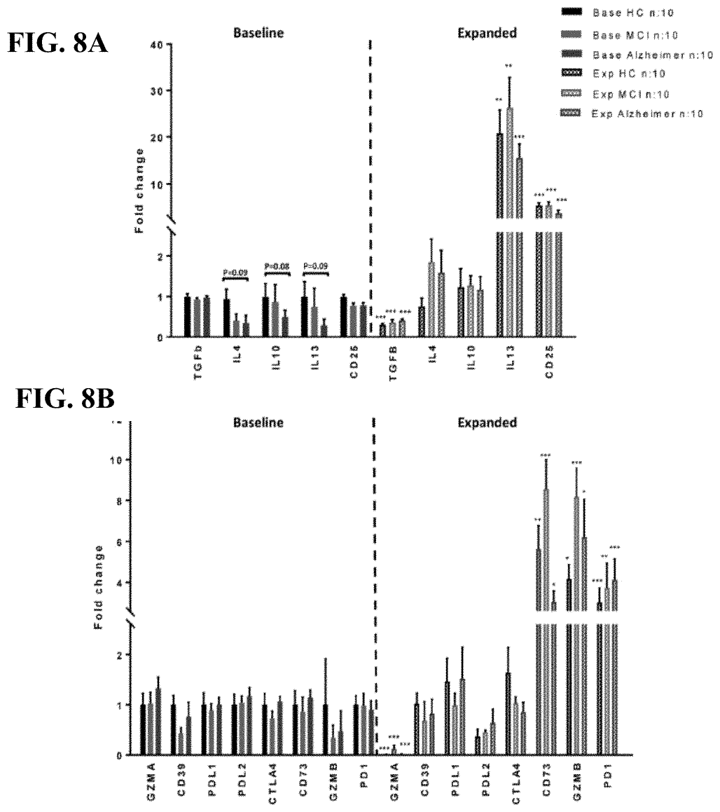

Messenger RNA was extracted from Tregs of 10 HC, 10 MCI and 10 AD subjects at baseline and following their respective expansions. Transcript levels of immunosuppressive cytokines known to be released from Tregs including TGFβ, IL-10, IL-4, and IL-13, CD25, and immunomodulatory markers that require cell-to cell proximity or contact (PD1, CTLA4, Granzyme (GZM) A&B, PDL1, PDL2, CD39 and CD73) were examined by quantitative PCR. At baseline, trends toward reduced IL-4, IL-10 and IL-13 transcript expression levels were noted in the Alzheimer's group. TGFβ expression levels were comparable between Alzheimer, MCI and HC at baseline, but following expansion, the expression levels were down-regulated in all groups. In contrast to IL-4 and IL-10 that did not change following expansion, IL-13 and CD25 transcripts were up-regulated in expanded Tregs (FIG. 8A). The expression levels of PD1, PDL1, PDL2, CTLA4, CD39, CD73 and Granzyme A&B in Tregs were comparable between HC, MCI and AD at baseline. While there were no changes in CD39, PDL1, PDL2 and CTLA4 transcripts following ex vivo expansion, up-regulation of PD1, GZMB and CD73 transcripts in expanded Tregs were observed in all groups. Granzyme A expression levels were down-regulated in expanded Tregs (FIG. 8B).

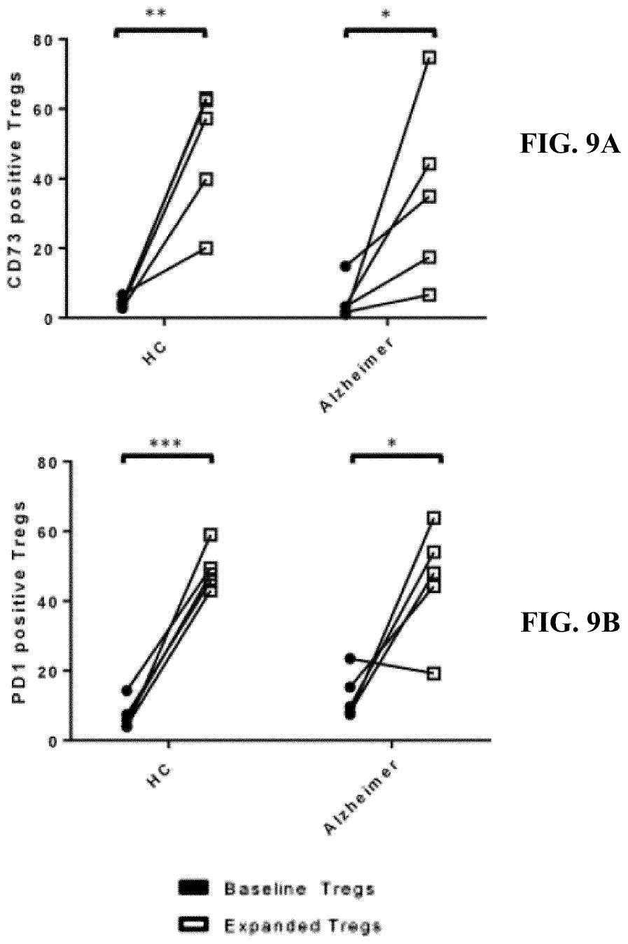

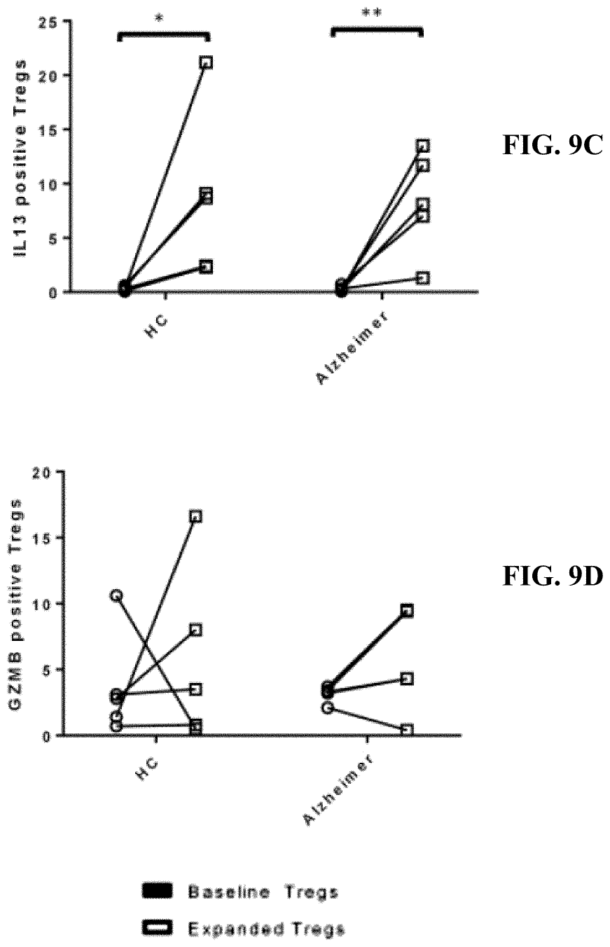

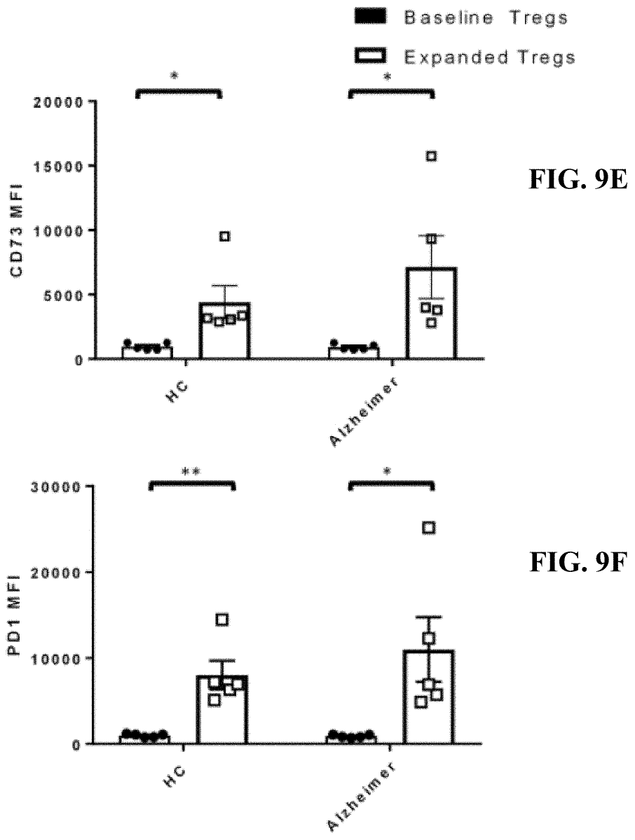

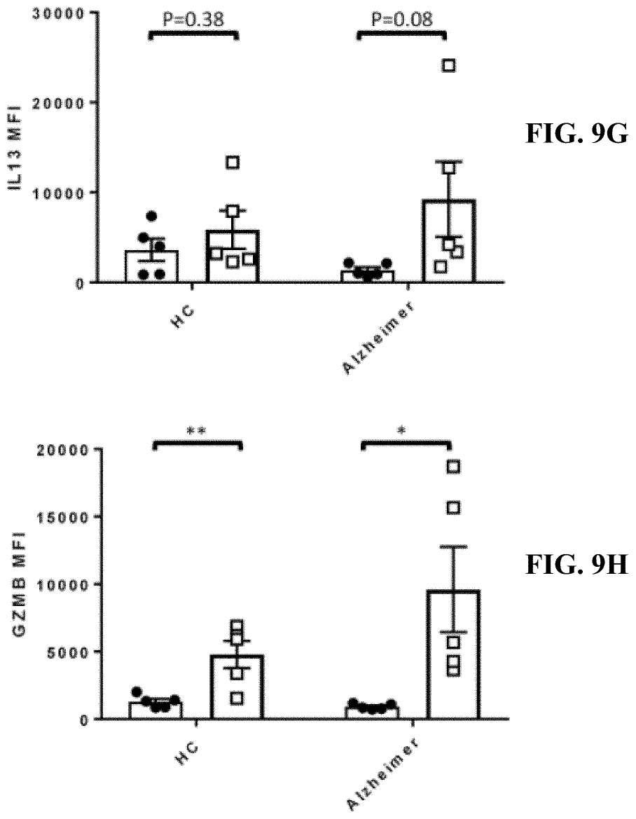

The protein expression of genes with upregulated transcript levels following expansion, were further analyzed in 5 Alzheimer patients and 5 HCs, using flow cytometry. Higher percentage of CD73⁺ (FIG. 9A), PD1⁺ (FIG. 9B) and IL-13⁺ Tregs (FIG. 9C) (% of total CD4⁺CD25^{high} T cells) were observed following ex vivo expansion in both Alzheimer patients and HCs. In addition to increased frequency, significant enhancement in CD73 (FIG. 9E) and PD1 (FIG. 9F) MFIs were noted in expanded Tregs. The difference between percentage of GZMB⁺ Treg subpopulation between baseline and expanded Tregs were not different (FIG. 9D), but GZMB MFIs were significantly amplified following expansion (FIG. 9H).

### 8.2.2.4 Ex Vivo Expanded Tregs Immunomodulation Works Via a Contact-Mediated Mechanism

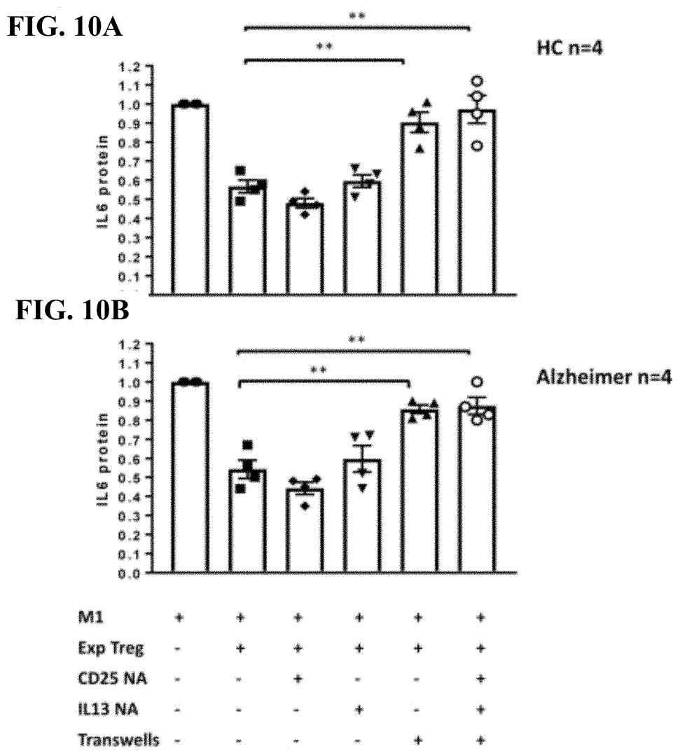

Immunoregulatory genes that up-regulated in ex vivo expanded Tregs were further scrutinized as potential candidates for enhanced Treg suppressive function. To test the direct effects of these gene products, expanded Tregs were co-cultured with pro-inflammatory macrophages and the changes in the suppressive function of Tregs on M1-derived IL-6 protein production were measured in the presence of neutralizing anti-IL-13, anti-CD25 or avoiding cell-cell contact using transwells. This assay was replicated with expanded Tregs of total four Alzheimer and four HC individuals. Expanded Alzheimer disease and HC Tregs attenuated M1-derived IL-6 protein by 44% and 46%, respectively (FIG. 10A and FIG. 10B). The addition of CD25 or IL-13 neutralizing antibodies to the co-culture did not affect the abilities of expanded Tregs to suppress IL-6 protein production. However, when the expanded Tregs were placed in a transwell, their suppressive function was abrogated in both Alzheimer disease and HC groups. As soluble factors, released from expanded Tregs, could still pass through the transwells, these data suggested that expanded Tregs required cell-to-cell contact to exert their suppressive function on pro-inflammatory macrophages.

### 8.2.3. REFERENCES

Albert, M. S., et al., 2011. The diagnosis of mild cognitive impairment due to Alzheimer's disease: recommendations from the National Institute on Aging-Alzheimer's Association workgroups on diagnostic guidelines for Alzheimer's disease. Alzheimers Dement. 7, 270-9.
Jack, C. R., Jr., et al., 2011. Introduction to the recommendations from the National Institute on Aging-Alzheimer's Association workgroups on diagnostic guidelines for Alzheimer's disease. Alzheimers Dement. 7, 257-62.
McKhann, G. M., et al., 2011. The diagnosis of dementia due to Alzheimer's disease: recommendations from the National Institute on Aging-Alzheimer's Association workgroups on diagnostic guidelines for Alzheimer's disease. Alzheimers Dement. 7, 263-9.
Morris, J. C., 1993. The Clinical Dementia Rating (CDR): current version and scoring rules. Neurology. 43, 2412-4.
O'Bryant, S. E., et al., 2008. Staging dementia using Clinical Dementia Rating Scale Sum of Boxes scores: a Texas Alzheimer's research consortium study. Arch Neurol. 65, 1091-5.
Yanagimachi, M. D., et al., 2013. Robust and highly-efficient differentiation of functional monocytic cells from human pluripotent stem cells under serum- and feeder cell-free conditions. PLoS One. 8, e59243.

### 8.3 Example 3: Details of the Improved Tre2 Manufacturing Protocol

Treatment using expanded Tregs is a promising treatment for a wide variety of disoders, for example, neurodegenerative disorders such as ALS and Alzheimer's disease, as described in Examples 1 and 2 above. However, technical challenges still prevent the wide application of Treg therapy. The methods for producing ex vivo-expanded Treg cell populations, included cryopreserved therapeutic Treg cell populations, described in the present application address these challenges.

The following is a list of representative steps illustrating an embodiment of the improved Treg manufacturing protocol. The exemplary protocol allows for the cryopreservation of a therapeutic population of Tregs after expansion, thus providing large amounts of Tregs at short notice, without the need for further expansion before administration. A more detailed summary of a representative Treg manufacturing protocol is presented at FIG. 14. As noted in the description of FIG. 14, the improved Treg manufacturing method may be used to expand Tregs from ALS patients. It is to be understood that the Treg production methods presented herein may also be applied to expand Tregs from other starting materials, including from cell samples from subjects with other disorders, e.g., other neurodegenerative disorders, or from healthy donor subjects.
1) Fresh leukapheresis products are used immediately after isolation on Day 0. They are not stored at 4° C. overnight.
2) Unique 4-Step process to isolate CD25+ cells following leukapheresis—Volume reduction, then purification, then CD8+/CD19+ depletion, and finally CD25+ enrichment.
3) Freshly isolated CD25+ cells are immediately placed into culture media. They are not cryopreserved first.
4) IL-2 is administered every 2-3 days on Days 6, 8 and 11. Cells are not discarded during expansion and thus, the scale is much larger requiring larger volumes of media.
5) During media changes, half the media is removed and replenished on Day 1, Day 4 and Day 6. Cells removed during these media changes are not returned to the culture flasks.
6) Cells are not centrifuged during media changes throughout the process with rare exceptions. They are only centrifuged on the day of cryopreservation in order to resuspend the cells in freezing media and transfer into cryovials.
7) Cells rest on Day 13 rather than Day 11 (no IL-2 is administered during the rest day).
8) Cryopreservation is performed at the end of the process, which could be anywhere from Day 15 to Day 25 depending on when the expansion process reaches the dose needed to treat the patient for 1 year per a clinical protocol.
9) Cryopreservation is performed manually on a large scale immediately following cell harvesting and bead removal. CryoStor® (CS10) freezing media is added at 4° C., and cells are transferred to cryovials within 10 minutes. Each cryovial represents 1 dose that would be thawed and administered back to the patient per the clinical protocol.
10) Cryovials are immediately frozen in the Controlled Rate Freezer per optimized freezing protocol and then transferred to liquid nitrogen vapor for long-term storage. The stability of the cells has been validated in long-term storage up to 18 months.

### 8.4 Example 4: Phenotype of Expanded Trees by Flow Cytometry

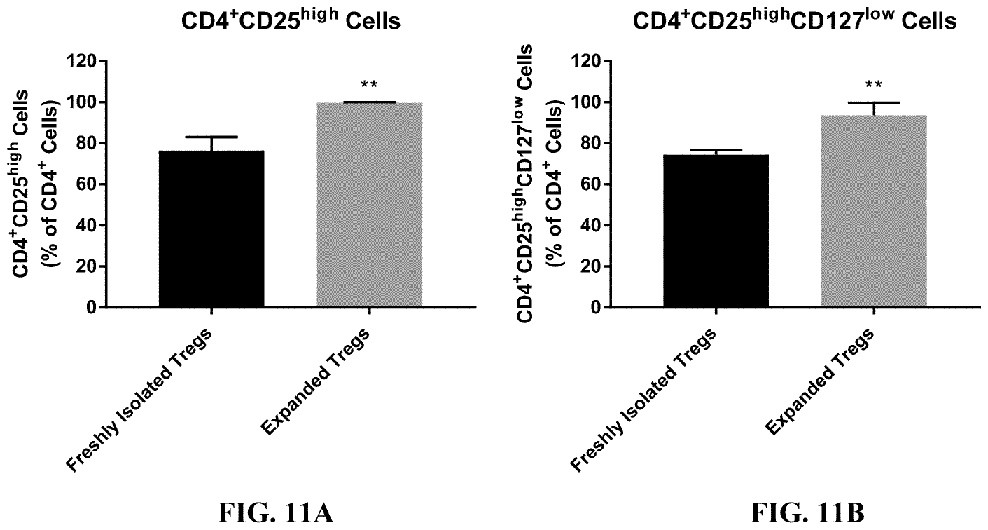

FIGS. 11A and 11B show the purity of expanded and freshly isolated Tregs as measured by the percentage of CD4+CD25high (FIG. 11A) or CD4+CD25highCD127low (FIG. 11B) cells in a population of CD4+ cells as determined by flow cytometry. These data show that the expanded Tregs are purer than freshly isolated Tregs. N=3, ** indicates p-value of 0.01 or less.

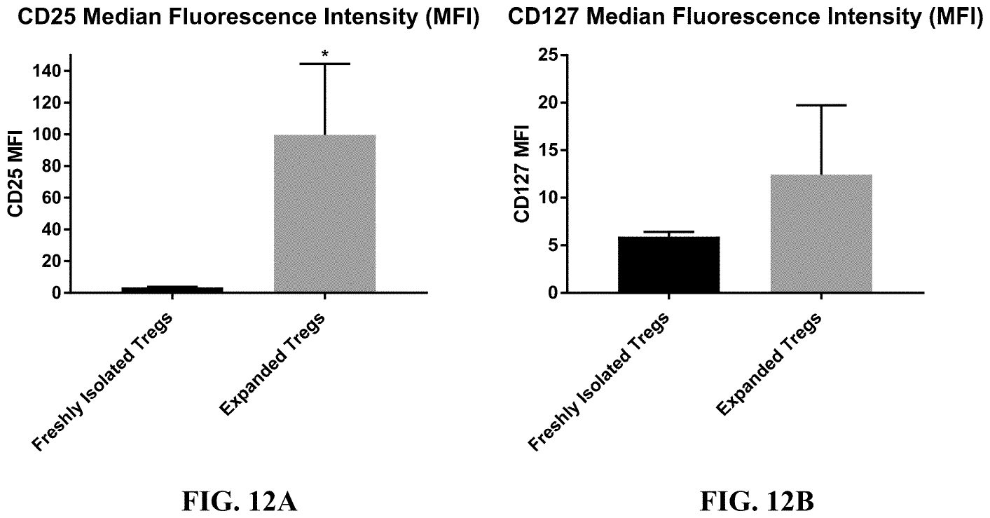

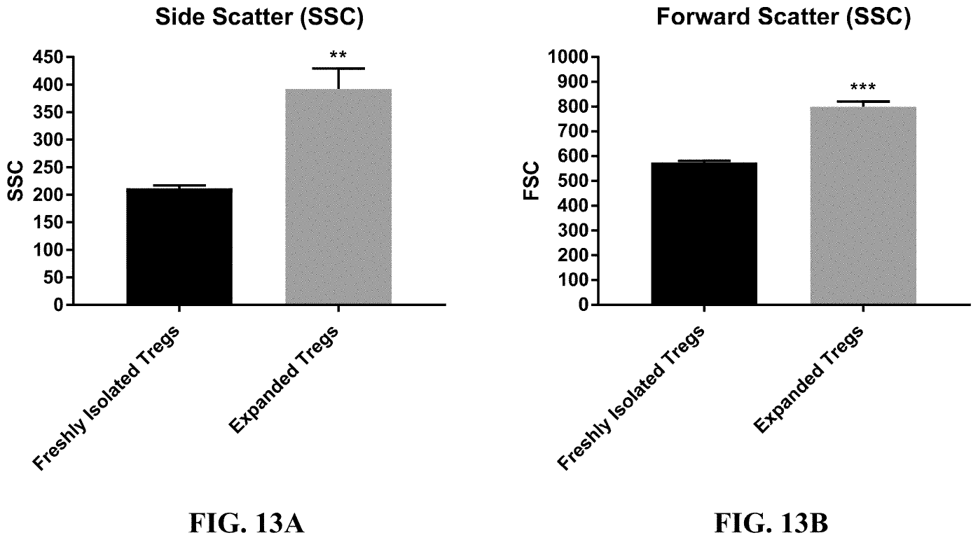

FIGS. 12A and 12B show the expression of CD25 protein (FIG. 12A) and CD127 protein (FIG. 12B) in expanded and freshly isolated Tregs as measured by flow cytometry. These data show that CD25 expression was increased approximately 30-fold in expanded Tregs compared to freshly isolated Tregs, while CD127 protein expression was not significantly increased (approximately 2-fold) in expanded Tregs compared to freshly isolated Tregs. N=3, ** indicates p-value of 0.05 or less.

FIGS. 13A and 13B show the granularity and size, respectively, of expanded and freshly isolated Tregs. These data indicate that expanded Tregs are larger than freshly isolated ones and that expanded Tregs have more granularity than freshly isolated ones.

### 8.5 Example 5: Exemplary Protocol for Isolation and Expansion of Regulatory T Cells from ALS Patient's Leukapheresis Product

Presented herein are exemplary instructions and steps for isolation and expansion of Regulatory T Cells (Tregs) from an ALS patient's leukapheresis product. This protocol may also be applied to isolation and expansion of leukapheresis products from non-ALS subjects, e.g., healthy subjects, for example, as part of allogeneic Treg treatments, or patients exhibiting a different disorder, for example a different neurodegenerative disorder.

A process flow diagram is shown in FIG. 14.

### 8.5.1. Procedure

Processing is preceded by decontaminating the biological safety cabinet (BSC) surface and equipment with Deconquat (Veliek Associates Inc.) followed by 70% ethanol and cleaning all work surfaces with 70% isopropyl alcohol (IPA).

Cell Culture and Harvest Parameter:
Slow=growing expansion: 25 days of cell culture with an estimated cell number<2×10⁹ cells—second leukapheresis may be required.
Normal expansion: 25 days of cell culture with an estimated cell number≥2×10⁹ cells.
Fast-growing expansion: estimated cell number≥2×10⁹ cells in ≤15 days of cell culture.
All cell culture expansions should maintain a purity of 70% CD4+CD25+ cells with viability above 70% prior to sterile vialing and cryopreservation.

### 8.5.2. Step 01: Patient Leukapheresis Product Receiving

Gently mix leukapheresis products on the rockers rotators at room temperature.

IMPORTANT NOTE: Leukapheresis products must be processed within 24 hours.

IMPORTANT NOTE: Do not seal off extra tubing on the leukapheresis product bag. The extra tubing will be needed to sterile connect to Sepax Tubing set in the following step.

Verify that total volume of the leukapheresis product is between 100 mL and 840 mL

Note: If the leukapheresis product is less than 100 mL, add an equal volume of CliniMACS Buffer with 1% HSA.

QC: Obtain the following samples for QC tests.
Cell count and Viability determination: 0.75-1.0 mL (Sysmex and Manual Trypan Blue exclusion method)
Cell Flow Analysis: CD Markers—3 mL
Sterility (14 days aerobic and anaerobic culture): inoculate 0.5 mL of the leukapheresis product in each of the long-term culture bottles.

### 8.5.3. Step 02: Volume Reduction of Leukapheresis Products Using the Ge Healthcare/Biosafe Sepax 2 Rm with the Pericell Protocol and Cs490.1 Kit

### 8.5.3.1 Preparation of Working Buffer—CliniMACS Buffer 1% v/v HSA

Have the following items available inside BSC:
FLEXBUMIN 25%, Albumin (Human), USP—100 mL bag
CliniMACS EDTA/PBS Buffer (1 L Bag)
60 mL and 140 mL syringes and 18G needles
A sampling site coupler

Insert a sampling site coupler to a bag of FLEXBUMIN 25%, Albumin (Human), USP—100 mL.

Using a syringe to draw 40 mL of FLEXBUMIN 25%, Albumin (Human), USP—100 mL to add to 960 mL of CliniMACS EDTA/PBS Buffer (1 L). Mix well by inverting the bag 3-5 times.

### 8.5.3.2 Starting Material Preparation—GE Healthcare-Biosafe CS-490.1 Kit (PeriCell)

### 8.5.3.2.1 Starting Material Cell Density and Input Volume

PeriCell input volume is 100 mL-840 mL.

Cells Density of leukapheresis product should be ≤44×10⁶ cells/mL.

For the high cell density leukapheresis products, adjust the Cell Density to ≤44×10⁶ cells/mL with CliniMACS Buffer 1% HSA.

### 8.5.3.2.2 Assembling GE Healthcare-Biosafe CS-490.1 Kit (PeriCell)

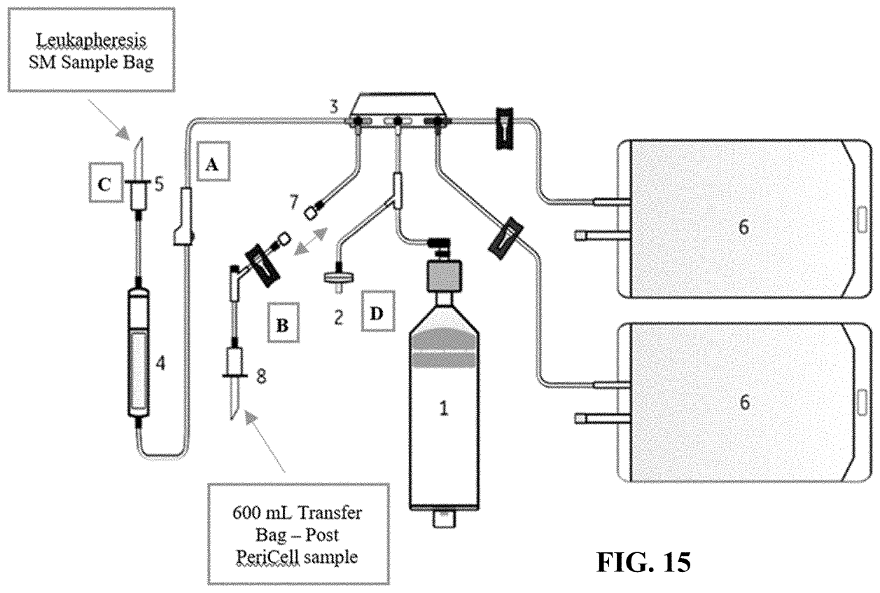

Refer to FIG. 15 for an illustration.

Note: For use with PeriCell protocol. (A) Red roller clamp. (B) Female luer for 600 mL transfer bag. (C) Spike with in-line bubble trap for Starting leukapheresis product. (D) Pressure sensor line.

Place a CS 490.1 kit blister pack in BSC along with the Starting Leukapheresis product bag.

Close the red roller clamp (A).

Spike a 600 mL transfer pack bag to the female luer of the tubing part with the blue stopcock (B). After that connect the tubing part with the transfer pack bag to the main CS 490.1 kit (see diagram above). Affix a label on the transfer pack bag: Post PeriCell product.

Attach the leukapheresis product bag to the kit by spiking one available port of the leukapheresis bag with the free spike (containing the bubble trap) extending from the blue stopcock (C).

Move assembled CS490.1 kit and kit tray (including the Tyvek cover) to the Sepax 2 RM Cell Processing device.

### 8.5.3.3 PeriCell Protocol Selection

Switch on the Sepax 2 RM, record the instrument number used on the batch record and from the Main Menu, select PBSC Applications, then PeriCell

Open the Change Parameters option.

Enter and/or verify the following parameters.
Input Product Volume: Enter the volume of the leukapheresis product.
Note: The minimum and maximum input volume is 100 mL-840 mL
Final Product Volume: Enter 120 mL
Note: Leukapheresis Product cells concentration should be less than 44 million cells/mL
Note: The minimum and maximum output volume is 30 mL-600 mL
Washing Cycles: Default value is 2
Pause Switch Bag: Default value is OFF. Leave on default value

Once the entries have been verified, select the Configure Trace ID option from the Protocol Options menu.

Note: This only needs to be done the first time a machine is used with the Pericell protocol and then reconfigured only after annual PM.

Enter Donation ID, Operator, and Kit ID.

Once traceability item selections have been selected, return to the Protocol Options menu by pressing the back arrow.

Load the assembled CS 490.1 kit on the Sepax 2 device and follow the instructions on the screen.

Load the separation chamber into the chamber, seat completely, and close chamber cover.

Hang product bags on appropriate hooks/supports.

Seat stopcocks and rotary pins with all stopcocks in “T” position.

Connect the pressure sensor line (D)

Ensure all lines are free of twists or crimps.

Select the Start Procedure option from the Protocol Options menu.

Enter the Donation ID and Operator's initial

Kit ID: Use barcode reader to enter kit ID from barcode on Tyvek cover of kit being used.

Press Input, Done, Continue and wait for the kit to validate.

After the kit is successfully validated, follow screen prompts to open the red roller clamp.

Press the Start Procedure button to initiate protocol.

When protocol is complete, end of process message will appear on the screen.

Follow screen prompts and perform actions as directed to remove the disposable kit

Remove bags and pressure sensor line

Strip cell lines and waste lines and close all clamps.

Heat seal to remove final product bag, transfer the bag to BSC.

NOTE: Ensure that all containers are labeled before they are detached from the tubing kits

Remove kits and dispose it in an appropriate waste container

### 8.5.3.4 Post PeriCell Cell Counts and Viability Determination

Obtain 1.0 mL of the Post PeriCell product for QC tests.

Cell count and Viability determination: 0.75-1.0 mL (Sysmex and Manual Trypan Blue exclusion method).

### 8.5.4. Step 03: Leukapheresis Product Purification Using the Ge Healthcare—Biosafe Sepax 2 Rm Neatcell Protocol and Cs900.2 Kit

### 8.5.4.1 Preparation of Working Buffer—CliniMACS Buffer 1% v/v HAS

From 1 L CliniMACS EDTA/PBS Buffer (1 L), remove 40 mL buffer and add 40 mL FLEXBUMIN 25%, Albumin (Human), USP—100 mL bag (HSA).

Mix the bag well by rocking back and forth 3-5 times.

### 8.5.4.2 Starting Material and NeatCell Protocol Information

The NeatCell protocol, used with the CS-900.2 kit, is designed to process 30-120 mL starting material. The final product volume is 45 mL fixed volume.

Note: The NeatCell protocol will only accept a maximum input volume of 120 mL but it will remove and process up to 130 mL of the starting material. For high cell density sample, adjust the density following step below.

NeatCell Specification: Max Volume: 120 mL. Max Cell Density: 160×10⁶ Cells/mL

### 8.5.4.3 Assembling the GE Healthcare Biosafe CS-900.2 Kit

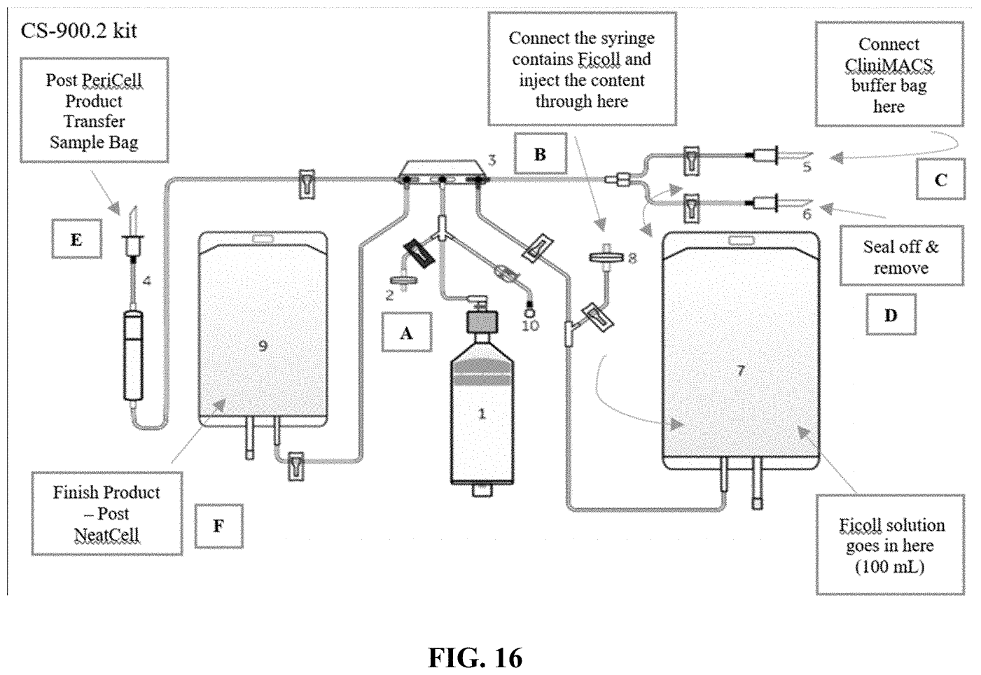

Refer to FIG. 16 for an illustration.

Place the CS-900.2 kit blister pack in the BSC along with the Starting Material Cell Product bag.

Inside the BSC, open and prepare the kit for connection to the Starting Material Cell Product bag.

Note: Refer to the Operator's Manual to identify all kit components and for instructions on preparing the kit

CLOSE all clamps on the kit except for the RED clamp (A).

Add 100 mL of Ficoll Paque Premium to the kit's density gradient medium/waste bag through the microbial filtered port (after opening clamp), then seal off the line. (B)

Obtain a 1 L bag Working Buffer—CliniMACS Buffer 1% v/v HSA.

Connect the Working Buffer bag to the CS-900.2 kit by spiking it with a Y connector spike line connected to the kit's red stopcock. (C).

Seal off the unused Y connector line. (D).

Spike the post-PeriCell Product bag to the kit upstream of the bubble chamber according to SOP EQ:3 and NeatCell Protocol. (E)

### 8.5.4.4 NeatCell Protocol Selection

Switch on the Sepax 2 RM and load the NeatCell protocol.

Verify that pre-stored NeatCell Protocol parameters are correct—i.e. initial volume, FP volume, etc. Note: to change parameters, refer to the Operator's Manual (OM-1773)

Install the assembled kit on the Sepax 2 RM and launch the automated procedure by following all steps in the Sepax 2 RM protocol manual and on the instrument's display.

At the end of the automated procedure, follow the steps IPicated in the protocol and on the display and discard the kit in biohazard trash.

Seal off the remaining CliniMACS Buffer 1% HSA bag leaving at least 6 inches of line.

Seal off Transfer bag containing the Post-NeatCell product. (F)

Remove kit and dispose in biohazard waste container.

### 8.5.4.5 Post NeatCell Cell Counts and Viability Determination

Gently mix the Transfer pack container thoroughly by inverting back and forth 5-6 times.

QC—Attach a 5 mL syringe to the Transfer pack container to draw 0.5 mL for cell count and viability determination.

### 8.5.5. Step 04: Depletion of Cd8+ and Cd19+ Cells (Clinimacs)

### 8.5.5.1 Preparation of Working Buffer—CliniMACS Buffer 1% HSA (Prepare 2×1 L Bags)

Under the BSC, insert a sample site coupler to a FLEXBUMIN 25%, Albumin (Human), USP—100 mL bag (HSA).

From 1 L CliniMACS EDTA/PBS Buffer (1 L), remove 40 mL buffer and add 40 mL FLEXBUMIN 25%, Albumin (Human), USP—100 mL bag (HSA).

Mix the bag well by rocking back and forth 3-5 times.

### 8.5.5.2 Labeling Cells with CD8+ and CD19+ Micro Beads for Depletion

Normal Scale Preparation:
One vial of CliniMACS CD8 Reagent (7.5 mL) is sufficient for labeling up to 4×10⁹ (4,000×10⁶) CD8⁺ positive cells out of up to 40×10⁹ (40,000×10⁶) leukocyte cells.
One vial of CliniMACS CD19 Reagent (7.5 mL) is sufficient for labeling up to 5×10⁹ CD19-positive cells out of up to 40×10⁹ (40,000×10⁶) leukocyte cells.

Optimal combined labeling requires a sample volume of 87.5 mL plus the entire volume of one vial of CliniMACS CD8 Reagent and one vial of CliniMACS CD19 Reagent.

The following information is needed to prepare Cells and Microbeads solution mix: Obtain Post NeatCell cell counts information.

Prepare Cells/Micro-beads solution mix as follow:
Add the required volume of working buffer to the post NeatCell product to obtain 87.5 mL.
Use 5 cc syringe and needle to add the entire vial of CD8 (7.5 mL) and CD19 (7.5 mL) Micro-beads to the cell suspension.

| | | |
|---|---|---|
| One vial of CliniMACS CD8 Reagent: | | 7.5 mL |
| One vial of CliniMACS CD 19 Reagent: | | 7.5 mL |
| | Total: | 15.0 mL |

Total Solution mix volume: Cell Product+Working Buffer+CD8/CD19 Micro-beads
   Total: 102.5 mL

Mix the cell and Micro-beads suspension gently by inverting the bag container back and forth 3-4 times. Incubate for 30 min at room temperature on an orbital shaker at 25 rpm.

### 8.5.5.3 Washing Labeled Cellular Product with Working Buffer—CliniMACS Buffer 1% HAS

After incubation, add Working Buffer—CliniMACS Buffer 1% HSA to a final volume of 600 mL to wash the cells.
Transfer pack container volume: 102.5 mL
CliniMACS Buffer 1% HSA: 497.5 mL
   Total: 600 mL

After adding Working Buffer, clamp the transfer bag to avoid leakage of the content during handling and centrifugation.

Outside the BSC use TCD to sterile weld an empty 1000 mL transfer pack container.

Measure the weight of the cells product bag container.

Place a bag of equal weight to balance the centrifuge.

Centrifuge the cells product to obtain a pellet. Centrifugation conditions: Time: 10 min, speed: 300 g, temperature: room temperature (18-25° C.), medium brake.

After the centrifugation, use plasma expresser to remove as much supernatant as possible.

Seal and discard the supernatant waste bag container.

Continue to the next step with the product transfer pack container containing cell and micro beads.

### 8.5.5.4 Preparing Cells for CD8 and CD19 Depletion

Follow steps below to re-suspend cells in CliniMACS PBS/EDTA Buffer containing 1% HSA. Note: The Cell Density shouldn't exceed 4×10⁸ (400×10⁶) cells/mL.

Perform the following calculation to prepare cell suspension for loading
Target Cell Density: <400×10⁶ cells/mL
Target Volume (mL) for Depletion: 100 mL
Note: Dilute Cells in Working Buffer—CliniMACS EDTA/PBS Buffer 1% HSA

Use a syringe to add 100 mL of CliniMACS PBS/EDTA Buffer—1% HSA to re-suspend the cell pellet.

Gently mix cell suspensions by inverting back and forth 5-6 times.

The cell suspension is ready to load into CliniMACS tubing set.

### 8.5.5.5 Installation of CliniMACS Tubing Set LS (162-01) for CD8 and CD19 Depletion

TABLE 2
Reference Table.

| | | | | Tubing | No. of | |
|---|---|---|---|---|---|---|
| Antigen/ | | Application | No. of | Set | Tubing | |
| REF No. | Application | Capacity | Vials | (REF No.) | Sets | Separation Program |
| D8CD19/ | Depletion | from 80 × 10⁹ | CD8 −1 vial | 200-073- | 1 | Depletion |
| REF 275-01 | Large scale | total Cells | CD19 −1 | 205/162-01 | | 2.1* |
| REF 179-01 | | | vial | | | |

*Depletion 2.1 Parameters: Cell Density: 20-400 × 10⁶/mL, Percentage of label cells: 1-99%, sample Loading Volume: 40-350 mL.

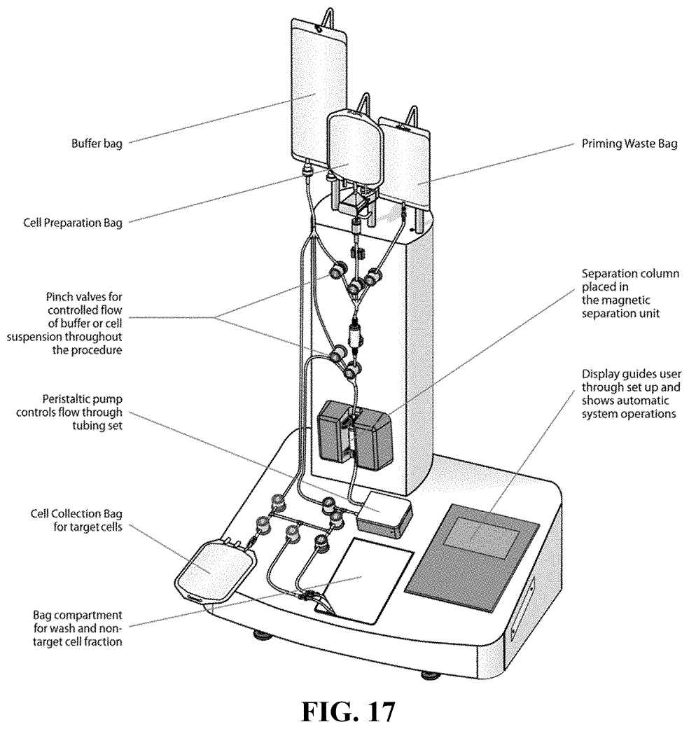

See FIG. 17 for an illustration of the CliniMACS Tubing Set LS (162-01).

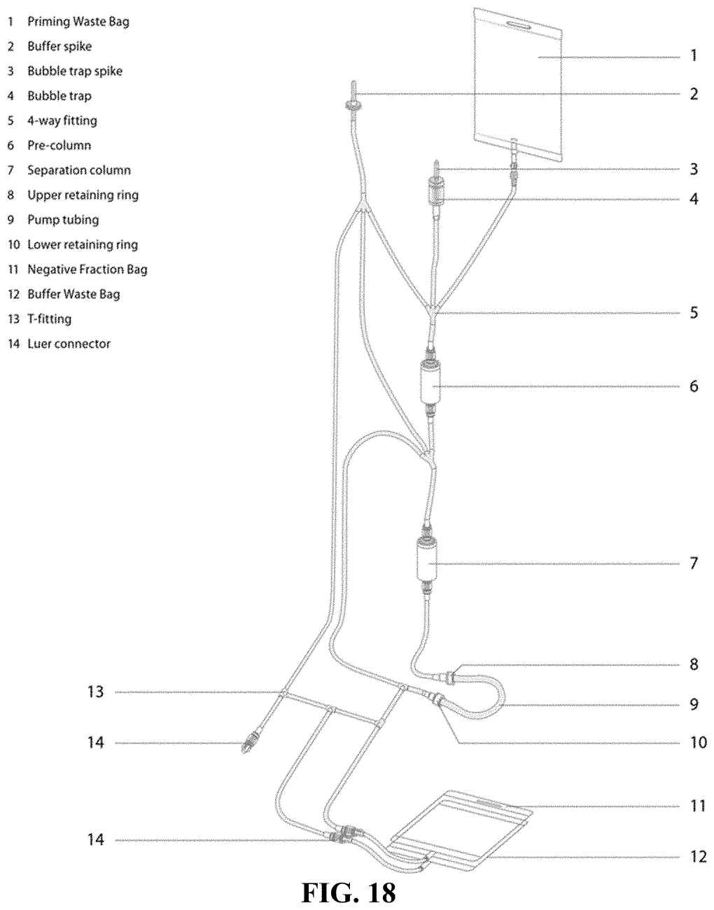

The depletion of CD8+/CD19+ from the cell fraction is performed by automatic cell separation using the CliniMACS® Plus Instrument in combination with CliniMACS PBS/EDTA Buffer in 1% HSA, the CliniMACS Tubing Set LS and software sequence DEPLETION 2.1. The depleted fraction (cells without CD8+/CD19+) is collected in the Cell Collection Bag (not provided with the tubing set). See FIG. 18 for an illustration of the Important Note: Label a 600 mL transfer bag “Cell Collection Bag—Positive Fraction”. Weld the transfer bag to the luer connector (no. 14 of the reference diagram shown in FIG. 18). Make sure the bag line is free with no flow blockage.

Switch on the CliniMACS® Plus Instrument and select DEPLETION 2.1.

Confirm choice by pressing “ENT” and select a tubing set.

Enter the order number of the selected tubing set.

Note: The order number (Ref. no.) can be found on the product label.

Note: If cell number is lower than the minimal concentration input 20×10⁶ cells/mL.

For Percentage of labeled cells—Enter: 40%.

Total volume of the sample ready for loading on the CliniMACS Tubing Set.—Enter: 100 mL.

Note: If the instrument screen refers to output of 1200 mL of target cells, connect a second 600 mL Transfer Bag to the pre-connected Cell Collection Bag in daisy chain configuration.

Follow the instructions given on the CliniMACS Plus Instrument screen and connect appropriate bags to the tubing set using Luer/Spike Interconnectors. Ensure that the slide clamps of the Luer/Spike Interconnectors are open.

If more than 1 L of buffer is needed, connect two buffer bags using a Plasma Transfer Set with two couplers. Use the second port of one of the buffer bags for the connection to the tubing set.

Follow the instructions on the instrument screen for the installation of the tubing set and start the automated separation program.

When the run has completed the CliniMACS will display the “Final Handling” screen.

After the separation has completed,

Record the process code:

Gently mix the cell suspension bag.

### 8.5.5.6 QC—Post CD8/CD19 Depletion

Attach a 5 mL syringe to the Post Depletion Cell Collection Bag to draw 5.0 mL of the cell Product for QC testing:
Cell count and Viability determination: 0.75-1.0 mL (Manual Trypan Blue exclusion method).
Cell Flow Analysis: CD Markers—2 mL.

### 8.5.6. Step 05: Positive Selection of Cd25+ Tregs (Clinimacs)

### 8.5.6.1 Preparation of Working Buffer—CliniMACS Buffer 1% HSA (Prepare 2×1 L Bags)

Have the following items available inside BSC:
2× FLEXBUMIN 25%, Albumin (Human), USP—100 mL bag
2× CliniMACS EDTA/PBS Buffer (1 L Bag)
2×60 mL/140 mL syringes
2×16/18G needles
2× sample site coupler.

Insert a sample site coupler to a FLEXBUMIN 25%, Albumin (Human), USP—100 mL bag (HSA).

From 1 L CliniMACS EDTA/PBS Buffer (1 L), remove 40 mL buffer and add 40 mL FLEXBUMIN 25%, Albumin (Human), USP—100 mL bag (HSA).

Mix the bag well by rocking back and forth 3-5 times.

Repeat the steps to prepare an additional bag of Working Buffer—CliniMACS Buffer 1% HSA. Set them aside in the BSC for later use.

### 8.5.6.2 Wash Cellular Product with Working Buffer—CliniMACS Buffer 1% HAS

Obtain post CD8/CD19 depleted cells product from STEP 04. Follow the steps below to wash cells prior to CD25 labeling

Add Working Buffer—CliniMACS Buffer 1% HSA to a final volume of 600 mL to wash the cells.

After adding Working Buffer, clamp the cell transfer bag to avoid leakage during handling and centrifugation.

Use TCD to sterile weld an empty transfer bag of the same size to the cell product bag.

Measure the weight of the product and empty bags.

Load the bags in upward position into Bio16 Centrifuge. Place bags of equal volume to balance the weight in centrifuge.

Centrifuge to obtain a cell pellet. Centrifugation conditions: time: 10 min, speed: 300 g, temperature: Room temperature (18-25° C.), medium brake.

After the centrifugation, use plasma expresser to remove as much supernatant as possible.

Gently mix the cells by rocking back and forth 5-6 times.

Seal and discard transfer bags containing supernatant.

Set aside the cell pellet for the next step.

### 8.5.6.3 Labeling Cells with CD25 for Enrichment CD25 Micro-Beads Specification

One vial of CliniMACS CD25 Reagent (7.5 mL) is sufficient for labeling up to 0.6×10⁹ (600×10⁶) CD25-positive cells out of up to 40×10⁹ total leukocyte cells.

Optimal labeling requires a sample volume of 190 mL plus the entire volume of one vial of CliniMACS CD25. Use Working Buffer CliniMACS 1% HSA for volume adjustment

Prepare Cells/CD25 solution mix as follows:
Add the required volume of working buffer to the cell pellet.
   Working Buffer—CliniMACS Buffer 1% HSA: 190 mL
Using a syringe, add 3.75 mL of CD25 Micro-beads
   CliniMACS CD25 Reagent: 3.75 mL
      Total: 193.75 mL

Mix the Cell/Beads Suspension gently by inverting the transfer pack container back and forth 3-4 times. Incubate for 30 min at room temperature on an orbital shaker at 25 rpm.

### 8.5.6.4 Wash Cellular Product with Working Buffer—CliniMACS Buffer 1% HAS

Add Working Buffer—CliniMACS Buffer 1% HSA to final volume of 600 mL to wash cells.
Cells/CD25 suspension: 193.75 mL
CliniMACS Buffer 1% HSA: 406.3 mL
   Total: 600 mL

After adding Working Buffer, clamp the cell transfer bag to avoid leakage during handling and centrifugation.

Use TCD to sterile weld a 1000 mL transfer bag to the cell product bag.

Measure the weight of the product and empty bags.

Load the bags in upward position into Bio16 Centrifuge. Place bags of equal volume to balance the weight in centrifuge.

Centrifuge to obtain a pellet. Centrifugation conditions: Time: 10 min, speed: 300 g, temperature: Room temperature (18-25° C.), medium brake.

After the centrifugation, using plasma expresser to remove as much supernatant as possible.

Gently disrupt the cell pellets.

Seal the waste transfer bags containing supernatant and retain the bag until the process is complete.

### 8.5.6.5 Preparation of Cellular Product for CD25 Enrichment

Follow the steps below to re-suspend cells in CliniMACS PBS/EDTA Buffer containing 1% HSA. Note: Cell Density Specification: ≤4×10⁸ (400×10⁶) cells/mL.

CD25+ Enrichment product specification: Cell Density: <400×10⁶ cells/mL, Suspension Volume (mL) for CD25⁺ Enrichment: 100 mL.

Add CliniMACS PBS/EDTA Buffer—1% HSA to the cell product to the final volume 100 mL.

Gently mix to homogenize cell suspensions by inverting back and forth 5-6 times.

The cell suspension product is ready to load into CliniMACS column.

### 8.5.6.6 Installation of CliniMACS Tubing Set LS (162-01) for CD25 Enrichment

Refer to Manufacturer SOP for CliniMACS tubing sets (200-073-205 REF 162-01) installation. See FIG. 17 for an illustration.

TABLE 3
Reference Table

| | | | | | No. of | |
|---|---|---|---|---|---|---|
| Antigen/ | | Application | No. of | Tubing Set | Tubing | |
| REF No. | Application | Capacity | Vials | (REF No.) | Sets | Separation Program |
| CD25/ | Enrichment | from 80 × 10⁹ | 1 vial | 200-073- | 1 | Enrichment 3.2 |
| 274-01 | CD25+ | total Cells | | 205/162-01 | | |

The Enrichment of CD25+ from the cell fraction is performed by automatic cell separation using the CliniMACS® Plus Instrument in combination with CliniMACS PBS/EDTA Buffer in 1% HSA, the CliniMACS Tubing Set LS and software sequence ENRICHMENT 3.2. The Enriched fraction is collected in the Cell Collection Bag (not provided with the tubing set). See FIG. 18 for an illustration.

Important Note: Label a 600 mL transfer bag “Cell Collection Bag: CD25+ enriched”. Weld the transfer bag to the luer connector (no. 14 of the reference diagram in FIG. 18). Make sure the bag line is free with no flow blockage.

Switch on the CliniMACS® Plus Instrument and select ENRICHMENT 3.2.

Confirm choice by pressing “ENT” and select a tubing set.

Enter the order number of the selected tubing set.

Note: The order number (Ref. no.) can be found on the product label.

Follow the instructions given on the CliniMACS Plus Instrument screen and connect appropriate bags to the tubing set using Luer/Spike Interconnectors. Ensure that the slide clamps of the Luer/Spike Interconnectors are open.

If more than 1 L of buffer is needed, connect two buffer bags using a Plasma Transfer Set with two couplers. Use the second port of one of the buffer bags for the connection to the tubing set.

Follow the instructions on the instrument screen for the installation of the tubing set and start the automated separation program.

When the run has completed, the CliniMACS will display the “Final Handling” screen.

After the separation has completed, record the process code.

### 8.5.6.7 QC—Post CD25 Enrichment

Gently mix the cells suspension thoroughly by inverting the Collection Bag: CD25⁺ enriched back and forth 5-6 times.

Attach a 5 mL syringe to the Cell Collection Bag to draw 5.0 mL of the sample.
Cell count and Viability determination: 0.75-1.0 mL (Manual Trypan Blue exclusion and Sysmex).
Cell Flow Analysis: CD Markers—3 mL.
Sterility (14 day aerobic and anaerobic cultures): inoculate 0.5 mL of cell suspension in each of the long-term culture bottles.

### 8.5.7. DAY 0—INITIATION OF TREGS CELL EXPANSION FROM CD25+ ENRICHED LEUKAPHERESIS PRODUCT: CULTURE MEDIUM PREPARATION

Have the following items available inside BSC:
Human AB Serum, USP—100 mL bottle.
TexMACS Medium—0.1 um filtered.
60 mL/140 mL syringes
16/18 G needles
Sample site couplers
QA pre-printed Labels: TexMACS_CM

Prepare 1 L of TexMACS_CM in a sterile Media Bottle.
TexMACS Media: 950 mL
Human AB Serum, USP: 50 mL
   Total: 1000 mL

Mix well by rocking back and forth 3-5 times.

Attached the pre-printed QA released labels TexMACS_CM

### 8.5.7.1 Day 0—Initiation of Tregs Expansion from CD25+ Enriched Leukapheresis Product

Stage a 140 mL syringe, a sterile 250 mL conical centrifuge tube, and the Cell Product Bag (CD25+ enriched cells) inside the BSC.

Using a 140 mL syringe, carefully draw CD25+ enriched cells from the bag into the syringe.

Carefully, inject cells suspension into a 250 mL conical tube.

Centrifuge the tube to obtain a cell pellet. Centrifugation conditions: Time: 10 min, speed: 300 g, room temperature (18-25° C.), medium brake

After centrifugation, carefully decant the supernatant.

Wash the cell pellet with 200 mL of TexMACS Medium+5% Human AB Serum

Centrifuge the tube to obtain a cell pellet. Centrifugation conditions: time: 10 min, speed: 300 g, room temperature (18-25° C.), medium brake.

After centrifugation, carefully decant the supernatant.

Add the required volume of TexMACS_CM to the cells pellets. Refer to the Calculation below.

Obtain Cell Count results from Post CD25 ENRICHMENT Product. □

Calculate the cell culture density at 0.8-1.0×10⁶ cells/mL.

Gently re-suspend cells pellets in CM by pipetting up and down until cell pellet is dispersed.

Transfer cell suspension into flasks.

Incubate the Flasks for 16-18 hours at 37° C. in a humidified mixture of 95% air and 5% CO₂

### 8.5.8. Ex Vivo Expansion Pre-Procedural Consideration—Medium Change, Rapamycin Formulation, IL-2 formulation, Stimulation and Activation

### 8.5.8.1 Medium Change

Ensure cells concentration is between 0.5×10⁶ cells/mL and 1.2×10⁶ cells/mL after each medium change.

Ensure cells concentration is 0.5-0.7×10⁶ cells/mL for MACS GMP ExpAct Treg Kit (CD3/CD28 Beads). Activation is carried out on Day 1 and re-stimulation on Day 15.

Prepare TexMACS GMP Complete Medium supplemented with 5% human AB serum.

Pre-warm TexMACS GMP Medium supplemented to Room Temperature before use.

Prepare working stocks of recombinant human interleukin 2 (rh TL-2) 500 IU/ul ready prior to use.

Prepare working stocks of Rapamycin 0.0001 mg/μL ready prior to use.

All refrigerated and frozen solutions and reagents must be allowed to reach room temperature at least 30 minutes before use (except for the T cell activation reagents and antibodies, which are kept at 2-8° C. until use).

### 8.5.8.2 Cell Count and Viability Determination

Count cells and record the results for assessment of cell viability and rate of expansion.

Record cell counts to one decimal place (i.e. 1.2×10⁶ cell/mL).

Calculate cell viability and total cell number using the Trypan Blue exclusion method and Sysmex automate counter as designated by MF-001 and MF-002.

### 8.5.8.3 Cell Cultures Medium Removal

Stand the flasks upright for at least 20 minutes without disturbing them, then move to the BSC and remove 50% of total medium volume.

If cell viability is over 90%, expand cells by changing the cell culture media to obtain 0.5×10⁶ cells/mL-1.2×10⁶ cells/mL.

Gently mixing the cell culture by swirling cell suspension. If cell clusters are detected, pipette until single cell suspension is obtained.

### 8.5.8.4 Formulating Working Buffer—TexMACS/5% Human AB Serum

Add 50 mL of Human AB Serum to 950 mL TexMACS Medium.

### 8.5.8.5 Formulating Working Stock—Rapamycin (0.0001 mg/L)

Stock Solution comes diluted in acetyl nitrile at a concentration of 1 mg/mL.

Rapamycin Molecular Weight (MW)=914.17 g/mol (1000 g/mol)

Make 1:10 dilution by diluting 1 mL of stock solution in 9 mL of culture medium

To obtain final concentration 100 nmol/L of Rapamycin, add 1 □l of the working stock to 1 mL of culture medium (1:1000). (For example: add 1000 μl (1 mL) of Rapamycin in 1 L of culture medium)

Make 1.2 mL aliquots of Rapamycin in 1.5 mL tubes. Attach labels and store at −20° C.

### 8.5.8.6 Preparation of rhIL-2 (Stock Concentration: 500 IU/Ul)

Note: Adjust final concentration of rhIL-2 to 500 IU/ml in culture medium

Preparation of 0.2% Acetic Acid fresh for each patient.

### 49.9 mL of Water (USP grade)+0.1 mL of Acetic Acid

Reconstitute Lyophilized rhIL2 according to manufacturer's instructions:

Reconstitute the rhIL-2 lyophilized product in 200 μl 0.2% acetic acid to a final concentration of 250 μg/ml

Target Working Stock Concentration: 500 IU/μl

Store rhIL-2 in the original container for 4 weeks at 2° C. to 8° C. under sterile conditions after reconstitution.

### 8.5.9. Step 06: Day 1—Stimulation of Tregs (Cd25+) with Macs Gmp Expact Treg Beads @ 4:1

Expand cells in TexMACS_CM+ Rapamycin

Addition of MACS GMP ExpAct Treg Kit (CD3/CD28 Beads) @ 4:1

### 8.5.9.1 Day 1—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.9.2 Day 1—Media Change (TexMACS Medium+5% Human AB Serum+ Rapamycin) and Addition of MACS ExpAct Treg Beads for Stimulation (4:1)

Pool cells together in a sterile bottle.

Remove 0.2 mL sample for cell count and viability assessment.

Adjust cell suspension volume to obtain cell density 0.5-0.7×10⁶ cells/ml.

Prepare TexMACS_CM (5% Human Serum AB).

Prepare Rapamycin (100 nmol/L).

Calculate amount of MACS GMP ExpAct Treg Beads for stimulation (4:1).

Note: MACs GMP ExpAct Kit contain 3.5 μm particles, which are preloaded with CD28 antibodies, anti-biotin antibodies and CD3-Biotin. Each vial contains 1×10⁹ ExpAct Treg Beads (2×10⁵/μL). MACS GMP ExpAct Treg Beads and Treg cells should be at a bead-to-cell ratio of 4:1 for initial stimulation.

Calculate volume of Rapamycin (100 nmol/L).

Add the required volume of Rapamycin to the cells cultures.

Add the number of MACS GMP ExpAct Treg Beads to cell culture—MIX WELL.

### 8.5.9.3 Day 1—Expansion and Incubation

Aliquot cell suspension into flasks.

Transfer the flask(s) to 37° C./CO₂ incubator

### 8.5.10. Step 07: Day 4—Texmacs_Cm+ Rapamycin Replenishment Only

### 8.5.10.1 Change Media with TexMACS_CM+ Rapamycin

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.10.2 Day 4—Media Change (TexMACS Medium+5% Human AB Serum+ Rapamycin)

Stand the flasks upright for at least 20 minutes and then remove approximately ⅔ volume of the media.

Pool the remaining cells together in a sterile bottle.

Remove 0.2 mL for cell count and viability assessment.

Calculate total cells count.

Adjust cell suspension volume to obtain Cell Density 0.5-1.2×10⁶ cells/mL.

Prepare TexMACS_CM (5% Human Serum AB). Note: Expires 14 days after preparation when stored at 2-8° C.

Prepare Rapamycin (100 nmol/L) Calculate volume of Rapamycin (100 nmol/L).

Add the required volume of Rapamycin to the cells cultures pool.

### 8.5.10.3 Day 4—Expansion and Incubation

Aliquot cell suspension into flasks.

Transfer the flask(s) to 37° C./CO₂ incubator.

### 8.5.11. Step 08: Day 6—Texmacs_Cm+ Rapamycin+Il-2 Replenishment

Change cell culture media with TexMACS_CM+ Rapamycin+IL-2.

Decontaminate BSC, work surfaces and equipment with Deconquat followed by 70% Ethanol.

### 8.5.11.1 Day 6—Culture Inspection

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.11.2 Day 6—Media Change with TexMACS_CM+ Rapamycin+TL-2

Stand the flasks upright for at least 20 minutes and then remove approximately 50% of the media.

Pool the remaining cells together in a sterile bottle.

Remove 0.2 mL for cell count and viability assessment.

Calculate total cells count.

Adjust cell suspension volume to obtain Cell Density 0.8-1.2×10⁶ cells/mL.

Prepare TexMACS_CM (5% Human Serum AB). Note: Expires 14 days after preparation when stored at 2-8° C.

Prepare Rapamycin (100 nmol/L).

Calculate volume of Rapamycin (100 nmol/L).

Add the required volume of Rapamycin to the cells cultures.

Prepare IL-2 (500 IU/mL): Calculate volume of IL-2 (500 IU/ml). Add the required volume of IL-2 to the cells cultures.

### 8.5.11.3 Day 6—Expansion and Incubation

Aliquot cell suspension into flasks.

Transfer the flask(s) to 37° C./CO₂ incubator.

### 8.5.12. Step 09: Day 8—Texmacs_Cm+ Rapamycin+Il-2 Replenishment

Expand cells in TexMACS_CM+ Rapamycin+IL-2

### 8.5.12.1 Day 8—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.12.2 Day 8—Media Change (TexMACS Medium+5% Human AB Serum+ Rapamycin+IL-2)

Stand the flasks upright for at least 20 minutes and then remove approximately 50% of the media.

Pool cells together in a sterile bottle.

Remove 0.2 mL for cell count and viability assessment.

Calculate total cells count.

Adjust cell suspension volume to obtain Cell Density 0.8-1.2×10⁶ cells/mL.

Prepare TexMACS_CM (5% Human Serum AB). Note: Expires 14 days after preparation when stored at 2-8° C.

Prepare Rapamycin (100 nmol/L) Calculate volume of Rapamycin (100 nmol/L). Add the required volume of Rapamycin to the cells cultures.

Prepare IL-2 (500 IU/mL) Calculate volume of IL-2 (500 IU/ml). Add the required volume of IL-2 to the cells cultures.

### 8.5.12.3 Day 8—Expansion and Incubation

Aliquot cell suspension into flasks.

Transfer the flask(s) to 37° C./CO₂ incubator.

### 8.5.13. Step 10: Day 11—Texmacs_Cm+ Rapamycin+Il-2 Replenishment

Expand cells in TexMACS_CM+ Rapamycin+IL-2 Replenishment.

### 8.5.13.1 Day 11—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.13.2 Day 11—Media Change (TexMACS_CM+ Rapamycin+IL-2)

Stand the flasks upright for at least 20 minutes and then remove approximately 50% of the media.

Pool cells together in a sterile bottle.

Remove 0.2 mL for cell count and viability assessment.

Calculate total cells count.

Adjust cell suspension volume to obtain Cell Density 0.8-1.2×10⁶ cells/mL.

Prepare TexMACS_CM (5% Human Serum AB). Note: Expires 14 days after preparation when stored at 2-8° C.

Prepare Rapamycin (100 nmol/L) Calculate volume of Rapamycin (100 nmol/L) Add the required volume of Rapamycin to the cells cultures.

Prepare IL-2 (500 IU/mL) Calculate volume of IL-2 (500 IU/ml) Add the required volume of IL-2 to the cells cultures.

### 8.5.13.3 Day 11—Expansion and Incubation

Aliquot cell suspension into flasks.

Transfer the flask(s) to 37° C./CO₂ incubator

### 1.1 Step11: Day 13—TexMACS_CM+ Rapamycin Replenishment ONLY

Expand cells in TexMACS_CM+ Rapamycin.

### 8.5.13.4 Day 13—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.13.5 Day 13—Media Change (TexMACS Medium+5% Human AB Serum+ Rapamycin)

Stand the flasks upright for at least 20 minutes and then remove approximately 50% of the media. Note: DO NOT discard medium.

Remove 0.2 mL of supernatant for cell count and viability assessment.

Pool the remaining cells together in a sterile bottle.

Remove 0.2 mL sample for cell count and viability assessment.

Calculate total cells count.

Adjust cell suspension volume to obtain Cell Density 0.8-1.2×10⁶ cells/mL.

Prepare TexMACS_CM (5% Human Serum AB) Note: Expires 14 days after preparation when stored at 2-8° C.

Prepare Rapamycin (100 nmol/L) Calculate volume of Rapamycin (100 nmol/L)

Add the required volume of Rapamycin to the cells cultures.

### 8.5.13.6 Day 13—Expansion and Incubation

Aliquot cell suspension into flasks

Transfer the flask(s) to 37° C./CO₂ incubator.

### 8.5.14. Step12: Day 15—Re-Activating Tregs with Macs Gmp Expact Treg Beads and Cm Replenishment+Il-2 Feed (if Required)

### 8.5.14.1 Day 15—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.14.2 Day 15—Media Change (TexMACS Medium+5% Human AB Serum+ Rapamycin+IL-2)

Stand the flasks upright for at least 20 minutes and then remove approximately 50% of the media. Centrifuge to obtain a cell pellet.

Pool the remaining cells together in a sterile bottle.

Remove 0.2 mL sample for cell count and viability assessment

Calculate total cells count.

Adjust cell density to 0.5-0.7×10⁶ cells/mL.

Prepare TexMACS_CM (5% Human Serum AB). Note: Expires 14 days after preparation when stored at 2-8° C.

Prepare Rapamycin (100 nmol/L) Calculate volume of Rapamycin (100 nmol/L).

Add the calculated volume to the cells cultures pool.

Prepare IL-2 (500 IU/mL) Calculate volume of IL-2 (500 IU/ml). Add the calculated volume to the cell cultures pool;

Calculate amount of MACS GMP ExpAct Treg Beads for stimulation (1:1);

Note: MACs GMP ExpAct Kit contain 3.5 μm particles, which are preloaded with CD28 antibodies, anti-biotin antibodies and CD3-Biotin. Each vial contains 1×10⁹ ExpAct Treg Beads (2×10⁵/μL). MACS GMP ExpAct Treg Beads and Treg cells should be at a bead-to-cell ratio of 1:1 for secondary stimulation.

Add required amount of MACS GMP ExpAct Treg Beads to cell culture volume—MIX WELL.

### 8.5.14.3 Day 15—Expansion and Incubation

Aliquot cell suspension into flasks.

QC:
1) Cell count and Viability determination: 0.75-1.0 mL (Manual Trypan Blue exclusion method)
2) Cell Flow Analysis: CD Markers—2 mL
3) Sterility (14 day aerobic and anaerobic cultures): inoculate 0.5 mL of cell suspension in each of the long-term culture bottles for FP sterility

Transfer the flask(s) to 37° C./CO₂ incubator.

### 8.5.15. Step 13: Day 18—Complete Medium Replenishment and Il 2 Feed (if Required)

Expand cells in TexMACS_CM+ Rapamycin+IL-2.

### 8.5.15.1 Day 18—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.15.2 Day 18—Media Change (TexMACS Medium+5% Human AB Serum+ Rapamycin+IL-2)

Stand the flasks upright for at least 20 minutes and then remove approximately 50% of the media. Centrifuge to obtain a cell pellet.

Pool the remaining cells in flasks together in a sterile bottle.

Remove 0.2 mL sample for cell count and viability assessment.

Calculate total cells count.

Adjust cell suspension volume to obtain Cell Density 0.8-1.2×10⁶ cells/mL.

Prepare TexMACS_CM (5% Human Serum AB). Note: Expires 14 days after preparation when stored at 2-8° C.

Prepare Rapamycin (100 nmol/L). Calculate volume of Rapamycin (100 nmol/L). Add the calculated volume to the cell cultures pool.

Prepare IL-2 (500 IU/mL). Calculate volume of IL-2 (500 IU/ml). Add the calculated volume to the cell cultures pool.

### 8.5.15.3 Day 18—Expansion and Incubation

Aliquot cell suspension into flasks.

Transfer the flask(s) to 37° C./CO₂ incubator.

### 8.5.16. Step 14: Day 20—Complete Medium Replenishment and Il 2 Feed (if Required)

Expand cells in TexMACS_CM+ Rapamycin+IL-2.

### 8.5.16.1 Day 20—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.16.2 Day 20—Media Change (TexMACS Medium+5% Human AB Serum+ Rapamycin+IL-2)

Stand the flasks upright for at least 20 minutes and then remove approximately 50% of the media.

Centrifuge to obtain a cell pellet.

Pool the remaining cells in flasks together in a sterile bottle.

Remove 0.2 mL sample for cell count and viability assessment.

Calculate total cells count.

Adjust cell suspension volume to obtain Cell Density 0.8-1.2×10⁶ cells/mL.

Prepare TexMACS_CM (5% Human Serum AB). Note: Expires 14 days after preparation when stored at 2-8° C.

Prepare Rapamycin (100 nmol/L). Calculate volume of Rapamycin (100 nmol/L). Add the calculated volume to the cell cultures pool.

Prepare IL-2 (500 IU/mL). Calculate volume of IL-2 (500 IU/ml). Add the calculated volume to the cell cultures pool.

### 8.5.16.3 Day 20—Expansion and Incubation

Aliquot cell suspension into flasks.

Transfer the flask(s) to 37° C./CO₂ incubator.

### 8.5.17. STEP 15: DAY 22: COMPLETE MEDIUM REPLENISHMENT ONLY (IF REQUIRED)

### 8.5.17.1 Day 22—Complete Medium Replenishment Only

Expand cells in TexMACS_CM+ Rapamycin

### 8.5.17.2 Day 22—Culture Inspection

Decontaminate BSC, work surfaces and equipment with Deconquat followed by 70% Ethanol.

Retrieve culture flasks from the incubator and visually inspect each flask for integrity (no cracks/leaks) and no turbidity (potential contamination). In case of loss of integrity or turbidity, record flask number and problem, place the flask in quarantine and immediately contact PI for further instructions.

### 8.5.17.3 Day 22—Media Change (TexMACS Medium+5% Human AB Serum+ Rapamycin)

Stand the flasks upright for at least 20 minutes and then remove approximately 50% of the media.

Centrifuge to obtain a cell pellet.

Pool the remaining cells in flasks together in a sterile bottle.

Remove 0.2 mL sample for cell count and viability assessment.

Calculate total cells count.

Adjust cell suspension volume to obtain Cell Density 0.8-1.2×10⁶ cells/mL.

Prepare TexMACS_CM (5% Human Serum AB). Note: Expires 14 days after preparation when stored at 2-8° C.

Prepare Rapamycin (100 nmol/L). Calculate volume of Rapamycin (100 nmol/L). Add the calculated volume to the cells cultures pool

### 8.5.17.4 Day 22—Expansion and Incubation

Aliquot cell suspension into flasks.

Transfer the flask(s) to 37° C./CO₂ incubator.

### 8.5.18. Step 16: Cells Harvest and Removal of Macs Gmp Activation Beads Using Clinimacs

### 8.5.18.1 Preparation of Working Buffer—CliniMACS Buffer 1% HSA (Prepare 2×1 L Bags)

Have the following items available inside BSC:
2× FLEXBUMIN 25%, Albumin (Human), USP—100 mL bag
2× CliniMACS EDTA/PBS Buffer (1 L Bag)
2×60 mL/140 mL syringes
2×16/18G needles
2× sample site coupler

Insert a sample site coupler to a FLEXBUMIN 25%, Albumin (Human), USP—100 mL bag (HSA).

From 1 L CliniMACS EDTA/PBS Buffer (1 L), remove 40 mL buffer and add 40 mL FLEXBUMIN 25%, Albumin (Human), USP—100 mL bag (HSA).

Mix the bag well by rocking back and forth 3-5 times.

Repeat the steps to prepare an additional bag of Working Buffer—CliniMACS Buffer 1% HSA. Set them aside in the BSC for later use.

### 8.5.18.2 Cellular Flasks Inspection

Retrieve culture flasks from the incubator and visually inspect each flask for integrity (no cracks/leaks) and no turbidity (potential contamination). In case of loss of integrity or turbidity, record flask number and problem in the Comments section below, place the flask in quarantine and immediately contact PI for further instructions.

### 8.5.18.3 Cellular Product Wash and Preparation for Beads Depletion

Place the following items in the BSC:
4-6 Sterile bottles (1 L)
8×500 mL sterile conical tubes and adapters
2-4 1.8 mL cryovials for QC samples
600 mL Transfer bag

Stage 4-12×500 mL sterile conical centrifuge tubes in BSC. Then carefully and evenly distribute the cell suspension among the centrifuge tubes.

Centrifuge the tubes—Time: 10 min/Speed: 300 g/Room temperature (18-25° C.)/low brake.

Gently decant the supernatant into sterile waste containers. Note: Careful not to lose cells pellets.

Repeat the steps above for the remaining cell suspension.

Once the cells pellets are obtained, add 20 mL CliniMACS PBS/EDTA (1% HSA) to each of the conical tubes. Re-suspend cell pellets thoroughly by gently pipetting up and down.

Pool the remaining cells together in one tube—MIX WELL.

Wash the tubes with 20 mL of CliniMACS PBS/EDTA (1% HSA). Add the washing volume to the pooled cells.

Remove 0.2 mL of the cell sample for cell count and viability assessment.

Calculate total cells count.

Calculate Total Cells+Beads number.

Adjust Cells and Beads concentration to obtain 4×10⁷ (40×10⁶)/mL Note: Adjust the volume with CliniMACS PBS/EDTA Buffer+1% HSA.

IMPORTANT: Follow the steps above to re-suspend cells and beads at a concentration of 4×10⁷ (40×10⁶) cells/mL in CliniMACS PBS/EDTA Buffer containing 1% HSA. The final volume should not exceed 350 mL.

Using syringe to transfer the cell suspension to a 600 mL sample transfer bag before connect to the CliniMACS Tubing Set LS (168-01).

QC: Flow Analysis: Prepare QC samples for flow analysis:
Tregs CD makers panel—10×10⁶ cells
Pre-Bead Removal sample—10×10⁶ cells×3

### 8.5.18.4 Installation of CliniMACS Depletion Tubing Set LS (168-01)

Refer to Manufacturer SOP for CliniMACS tubing sets (200-073-205 REF 162-01) installation. See FIG. 17 for an illustration.

The depletion of MACS GMP ExpAct Treg Beads from the original fraction is performed by automatic cell separation using the CliniMACS® Plus Instrument in combination with CliniMACS PBS/EDTA Buffer in 1% HSA, the CliniMACS Tubing Set LS and software DEPLETION 2.1. The depleted fraction (cells without MACS GMP ExpAct Treg Beads) is collected in the Cell Collection Bag (not included with the tubing set). See FIG. 18 for an illustration

Important Note: Label a 600 mL transfer bag “Cell Collection Bag—Positive Fraction”. Make sure the bag is free with no flow blockage.

Switch on the CliniMACS® Plus Instrument and select DEPLETION 2.1. for depletion of MACS GMP ExpAct Treg Beads.

Confirm choice by pressing “ENT” and select a tubing set. Enter the order number of the selected tubing set. The order number (Ref no.) can be found on the product label. Be aware that the separation program DEPLETION 2.1 is a “staged loading” program. The program includes a query for the following parameters in order to adjust the separation sequence for each IPividual sample and to provide important information on the required buffer and bag volumes.

WBC concentration (in this application: Cell number plus MACS GMP ExpAct Treg Beads number/mL). Note: If cell number plus MACS GMP ExpAct Treg Beads number/mL is lower than minimal concentration, choose 20×10⁶ cells/mL.

Percentage of labeled cells (in this application: Percentage of MACS GMP ExpAct Treg Beads among total cells (cells+MACS GMP ExpAct Treg Beads))—Enter: 45%.

Total volume of the sample ready for loading on the CliniMACS Tubing Set. Note: If the instrument screen refers to output of 1200 mL of target cells, connect a second 600 mL Transfer Bag to the pre-connected Cell Collection Bag in daisy chain configuration.

Follow the instructions given on the CliniMACS Plus Instrument screen and connect appropriate bags to the tubing set using Luer/Spike Interconnectors. Ensure that the slide clamps of the Luer/Spike Interconnectors are open.

If more than 1 L of buffer is needed, connect two buffer bags using a Plasma Transfer Set with two couplers. Use the second port of one of the buffer bags for the connection to the tubing set.

Follow the instructions on the instrument screen for the installation of the tubing set and start the automated separation program.

When the run has completed the CliniMACS will display the “Final Handling” screen.

After the separation has been completed, Record the process code.

### 8.5.18.5 Final Cellular Product Preparation

Clamp or heat seal the tubing above the luer lock connecting the Cell Collection Bag to the CliniMACS tubing set. Make three hermetic seals in the tubing directly below valve No. 9. Carefully sever the distal seal to disconnect the Cell Collection bag from the tubing set and transfer the cells to the BSC.

Use a syringe to measure the cell suspension volume before transferring to 250 mL conical centrifuge tubes for cell dose preparation.

IMPORTANT: re-suspend the cellular product thoroughly.

Remove 0.2±0.1 mL for cell count and viability.

Calculate total cells count

QC: Draw QC samples from Cell Collection bag (Positive Fraction—Target cells)
Flow Analysis: Post-Bead Removal counts—10×10⁶ cells×3
Flow Analysis: CDs markers panel—10×10⁶ cells
Mycoplasma Testing: 0.2 mL

### 8.5.19. Step 18: Sterile Fill/Finish and Cryopreservation

### 8.5.19.1 Cell Dose Calculation

Cell Dose Calculation from Patient's weight:

Patient's weight: ______ kg.

Target dose: 1.0×10⁶ viable cells/kg×Patient weight (kg)=Target dose 10⁶ cells.

Compensation dose=30% of Target dose 10⁶ cells.

Dose volume: 1 mL

Final Dose Formulation (Target dose+Compensation Dose)×10⁶ cells/1 mL./dose

### 8.5.19.2 Cell Dose Preparation

Equip 2 sterile 250 mL conical tubes in BSC.

Using a 50 mL transfer pipettes, aliquot the cell suspension evenly among the 2 conical centrifuge tubes.

Centrifuge—Time: 10 min/Speed: 300 g/Room temperature (18-25° C.)/medium brake.

After centrifugation, carefully remove supernatant into a waste container. Note: DO NOT discard medium until processing is complete.

Add 5 mL CryoStor CS10 (2-8° C.) to cell pellet. Re-suspend cell pellet by gently pipetting up and down.

Add the remaining volume of CryoStor CS10 (2-8° C.) to the cell suspension volume to meet to the Cell Dose Calculation.

Calculate viable cell count.

Final dose/vial calculation:

Total Viable Cells×10⁶ cells÷Final Dose Formulation×10⁶ cells=Total Doses Vol. mL+1.2 mL/Vial=Number of Vials

### 8.5.19.3 Fill-Finish and Cryopreservation

IMPORTANT: Make sure to precool Controlled Rate Freezer (CRF) to 4° C. prior to beginning the fill-finish procedure:
Clean the CRF chamber with approved disinfectant followed by 70% IPA.
Insert the temperature probe into a cryovial with CS10, placed in a cryovial rack.
Proceed to start up CRF following instrument SOP.
Obtain cold beads bath (˜4° C.) and stage in BSC.

### 8.5.19.4 Fill-Finish Parameters

Total Fill Volume/Vial: 1.2 mL
Vial type and size: CellSeal Closed-System Cryogenic Vial (2 mL)
Total No. of Vials=Total Fill Volume/1.2 mL

### 8.5.19.5 Procedure

Place LABELED CellSeal Cryogenic (2 mL) Vials ready in a rack inside the BSC.

Swab the luer lock port with an alcohol wipe. Allow the port to air dry up to 1 minute.

Use a 30 mL syringe connected to a 3 mL syringe by an adapter to draw up the sample.

It is recommended to draw a small volume of air into the syringe first and then draw the sample. The air will be used to level the column of liquid in the tubing and it also help in pushing the entire sample into the vial.

Once connected, push the sample into the vial.

Seal the air vent tubing above the filter, making this a closed system. Use clean scissors to cut off additional tubing.

Perform a visual inspection and verify labels.

Transport the final product vials in cold rack to the Controlled Rate Freezer

Place the vials inside the CRF and close the door.

Freeze cells using the following protocol:
1° C./min to 4° C.
25° C./min to −40° C.
10° C./min to −12° C.
1° C./min to −40° C.
10° C./min to −90° C.

Once the cycle is complete, transfer the vials to liquid nitrogen (LN₂) vapor tank for storage.

### 8.5.19.6 Fill/Finish QC

Table 4, below, presents the release criteria the final cell product must satisfy. Some release criteria may be determined after thawing, e.g., visual inspection, viability, endotoxin levels, gram stain and sterility testing. Some release criteria may be determined either after thawing or right before cryopreservation, e.g., flow analysis for CD8⁺ cells, flow analysis for CD4⁺CD25⁺ cells and testing for residual beads.

TABLE 4
Release Criteria

| Test | Specification |
|---|---|
| Visual Inspection | No evidence of contamination |
| Viability | ≥70% |
| Endotoxin (LAL) | <5 EU/kg |
| Gram Stain | Negative |
| Flow Analysis: CD8+ | <20% |
| Flow Analysis: CD4+ CD25+ | ≥70% |
| Residual CD3/CD28 beads | ≤100 beads/3 × 10⁶ cells |
| Non-Release Testing | Aerobic: No growth Anaerobic: |
| Sterility - 14 days | No growth |
| (Aerobic and Anaerobic cultures) | |

### 8.6 Example 6: Exemplary Protocol for Thawing Cryopreserved Trees

The following is a description of an illustrative protocol providing instructions and steps for thawing cryopreserved Tregs in 5% HSA Saline for Infusion. This protocol is not intended to limit the scope of the disclosure.

### 8.6.1. Procedure

Remove a vial of Cryopreserved Tregs from LN2 storage and put it on dry ice in a transport container.

Transfer the cryovial to cGMP laboratory. Verify patient, cryovial, and final product labels information.

Decontaminate BSC surface and equipment with Deconquat solution for 10 minutes. After 10 minutes, wipe clean with sterile 70% IPA wipes.

Insert sampling site couplers into outlet ports of the following items:
1. FLEXBUMIN 25%, Albumin (Human), USP—100 mL bag (HSA)
2. Saline Buffer (250 mL Bag)—cold (4° C.)
3. 2 sterile transfer packs (150 mL)

Follow steps below to prepare two 50 mL normal saline (+5% HSA) solution in sterile transfer packs.
1. Using 60 mL syringe, draw 40 mL of Saline Buffer to add to each of sterile transfer packs.
2. Using 20 mL syringe, draw 10 mL of FLEXBUMIN 25%, Albumin (Human), USP to add to each of the 40 mL of Saline Buffer above.
3. Mix the bags by inverting 3-5 times.
4. Affix the QA issued product ID label to one of the bag. Label the other bag as normal saline−5% HSA.

Follow the steps below to thaw a vial of cryopreserved Tregs using COOK regentec thawing system.
1. Connect the supplied main adaptor to the thawing system and apply power.
2. Press the Up/Down selector button to select Rapid Thaw rate. Press Enter.
3. The indicator ring will glow orange, then green when ready.
4. Press the down selector button until the Enter Volume profile appears (the third option).
5. Use the Up/Down button to enter 1.2 mL. Press Enter.
6. Insert the vial. Holding by the top, press the vial down and hold until the device clamps. The indicator will glow purple when the vial is at the correct depth.

Thawing begins right away after a short period of analysis; progress is indicated on the display. When the thaw is complete, the vial is ejected and the indicator ring will glow green if successful. If unsuccessful, the indicator will glow orange.

Once the thawing cycle is complete, bring the vial to the BSC. Follow steps below to re-suspend Tregs in 50 mL normal saline (+5% HSA).
1. Cut open the vent tube with a pair of clean scissors. Remove the foil on the bottom of the retrieval port.
2. Thoroughly swab the retrieval port septum with a sterile alcohol wipe. Allow to air dry before accessing.
3. Obtain the sample transfer pack bag with the product ID label.
4. Connect a 3 mL syringe to the bag through the sampling coupler to draw 2 ml of normal saline (+5% HSA) into the syringe. Disconnect the syringe from the bag.
5. Attach 18G needle to the syringe to puncture the retrieval port septum and insert the needle into the septum at a 45-600 angle.
6. Increase the angle of the needle gradually as the needle enters the vial.
7. Draw the entire Tregs product into the syringe with 2 ml of normal saline (+5% HSA).
8. Carefully remove the needle from the syringe to discard in a sharp container.
9. Re-connect the syringe to the sample site coupler on the sample transfer pack bag with the product ID label.
10. Slowly inject the cells suspension into the transfer pack bag. Note: Mix the bag by inverting 3-5 times. Make sure to wash off the remaining cells from the syringe by drawing the cells suspensions and dispensing back into the bag (repeat 3-5 times).
11. Obtain 0.25 mL of the Tregs suspension for cell count with Sysmex Automated Cell Counter.
12. Assess cells viability by hemocytometer Trypan Blue manual cell count.
13. Calculate total viable cells in 50 mL normal saline (+5% HSA):
   Cell Concentration×10⁶/mL×Cell Suspension Vol.=Total Cell No.×10⁶ Cells;
   Total Cell No.×10⁶ cells×Viability %=Total Viable Cells×10⁶ Cells
14. Calculate Treg dose:
   (Required Dose Cell No.×10⁶ cells/Total Viable Cells×10⁶ cells)×50 mL (Initial Suspension Vol.)=Required Thawed Cells Vol. mL;
   50 mL—Required Thawed Cell Vol. mL=Excess Volume To Be Removed
15. Using a 60 mL syringe, remove the excess volume of Tregs suspension from the sample. Note: Transfer the excess volume to a sterile container. Do not discard until the procedure is complete.
16. Add an equal volume of normal saline (+5% HSA) to the sample so that the final volume is 50 mL. Mix the cells suspension by inverting the bag 3-5 times.
17. Repeat cell count with sysmex and record the result.

### 8.6.2. Release Testing QC

Sysmex Cell Count: require 0.25 ml of the sample for the cell count.

Viability Assessment (Manual Hemocytometer and TB): require 0.25 ml of the sample.

IMPORTANT NOTE: Always use normal saline at +4° C. to make cell dilution for viability assessment. Cells and Tryphan blue ratio should be: 20 μl of Tregs Cells: 4 μl of TB (0.4%).

Endotoxin Test (in-laboratory test): requires 0.2 ml of the sample

Gram Stain (STAT test—Memorial Herman Micro Laboratory): Requires 0.5 mL of the sample.

### 8.6.3. Special Consideration—Placebo Preparation

Perform Steps above steps to prepare placebos.

Add 1 ml of CS10 in place of Tregs to prepare placebo samples.

Sterility (14 days aerobic and anaerobic culture): inoculate 0.5 mL of the final product in each of the long-term culture bottles.

### 8.6.4. Release Criteria

Release criteria are shown in Table 5.

TABLE 5
Release Criteria.

| Test | Specification |
|---|---|
| Visual Inspection | No Evidence of Contamination |
| Gram Stain | No Organism Seen |
| Endotoxin | <5 EU/kg |
| Viability | ≥70% |
| Mycoplasma (Pre-Freeze) | Negative |
| Immunophenotyping (Pre-Freeze) | <20% CD8⁺ cells |
| | ≥70% CD4⁺ CD25⁺ cells |
| Residual Beads (Pre-Freeze) | ≤100 beads/3 × 10⁶ cells |
| Treg Dose (CD4⁺ CD25⁺ Cells) | 1 × 10⁶ cells/kg ± 10% |

### 8.7 Example 7: Characterization of Expanded Treg Cell Populations

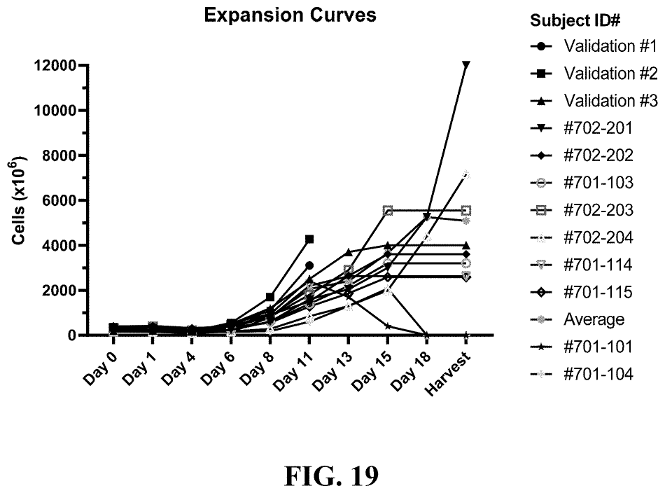

This example describes the characteristics (e.g., viability, purity, and potency) of expanded Treg cell populations produced by the methods described herein. FIG. 19 shows the expansion curce for all samples tested using the described method. Validation runs #1-3 were completed prior to initiating the phase 2 trial. Subject ID #'s are listed for expansion curves from each subject enrolled in the phase 2 trial. Two subjects experienced expansion failures (#701-101 and #701-104). The mechanism of Treg expansion failure in these cases is unknown. There is donor-dependent variability in the expansion rates. Whether ALS contributed to these subjects' Treg expansion failures is unknown. The average expansion curve for all successful expansions is also shown. All successful expansions reached the target dose for the phase 2 trial of 2000×10⁶ cells within 25 days. The average number of Tregs collected at harvest (occurring between day 11 and day 25) was 5090×10⁶ cells.

### 8.7.1. Expanded Treg Cell Populations from Validation Runs

Expanded Treg cell populations were produced following the methods described herein, in particular, the protocol described in Sections 8.3 and 8.5, above. The expanded Treg cell populations were cryopreserved following the methods described herein, in particular the protocol described in Section 8.6. The cryopreserved Treg cell populations were thawed after approximately 1 week or after 1, 3, 6, 9, 12, or 18 months of cryopreservation, following the methods described herein, in particular, the protocol described in Section 8.5, above. The thawed Treg cell population were resuspended in a total of 50 mL of normal saline with 5% human serum albumin (HSA) as described herein, and stored at 4° C. Three samples of populations of cryopreserved Tregs were characterized in these experiments.

Tregs expanded via the methods presented herein produced populations of Tregs that possess exemplary suppressive function, viability and purity. Remarkably, the Treg cell populations maintain this high potency, viability, purity and potency even after cryopreservation and thawing, without additional expansion, even after the Treg cell populations have been cryopreserved for up to 18 months (the latest time point assessed).

### 8.7.1.1 Viability of Cryopreserved Treg Products

Cell viability was assessed by trypan blue staining. Trypan blue is a dye which is excluded by cells with an intact membrane (viable cells) but taken up by cells with compromised membrane integrity (non-viable cells). Thus, viable cells appear clear under a light microscope, whereas non-viable cells appear blue. Equal amounts of trypan blue and cell suspension are mixed and counted. Viability is expressed as a percentage of trypan blue excluding cells.

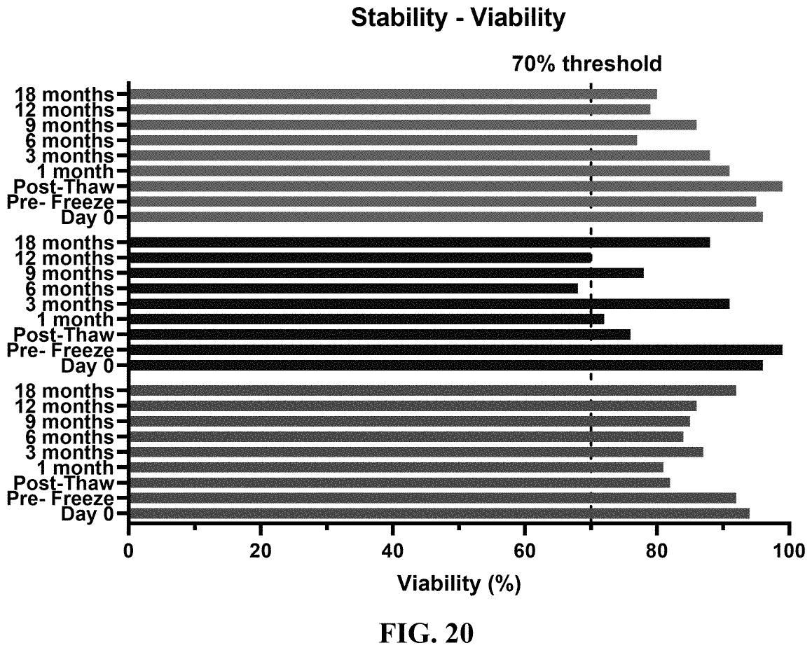

The viability was determined immediately after isolating fresh CD4⁺CD25⁺ cells (Day 0), immediately prior to cryopreservation following expansion (Pre-Freeze), and then 1 hour after thawing (Post-Thaw) the cryopreserved product (completed within a week following cryopreservation). Stability studies on the cryopreserved products were then performed at 1 month, 3 months, 6 months, 9 months, 12 months and 18 months following cryopreservation. Cell viability was assessed in the same manner for the stability study. Results of three validation runs are shown in FIG. 20. For all 3 validation runs, the cell viability after thawing and resuspension remained above the threshold of ≥70% even after 18 months in cryopreservation. See Table 6 for the average viability at baseline, after expansion and after cryopreservation.

TABLE 6
Viability

| Time | Mean | SD | N |
|---|---|---|---|
| Day 0 | 95.3 | 1.2 | 3 |
| Pre-Freeze | 95.3 | 3.5 | 3 |
| Post-Thaw | 85.7 | 11.9 | 3 |
| 1 month | 81.3 | 9.5 | 3 |
| 3 months | 88.7 | 2.1 | 3 |
| 6 months | 76.3 | 8.0 | 3 |
| 9 months | 83.0 | 4.4 | 3 |
| 12 months | 78.3 | 8.0 | 3 |
| 18 months | 86.7 | 6.1 | 3 |

### 8.7.1.2 Purity of Cryopreserved Treg Products

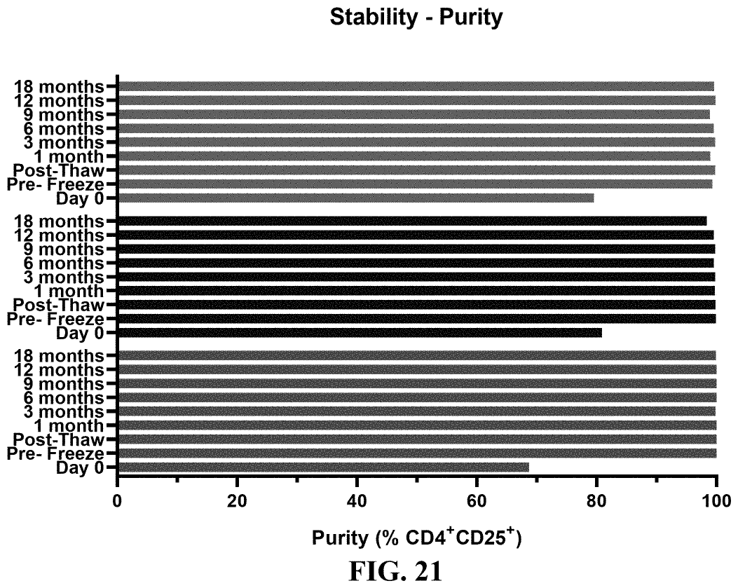

The purity of the expanded Treg cell populations was assessed by flow cytometry. The percentage of CD4⁺CD25⁺ cells was determined at baseline following the CD25⁺ cell enrichment/CD8+/CD19+ depletion step (Day 0), immediately prior to cryopreservation following expansion (Pre-Freeze), and after thawing and preparation of the final Treg product. Stability studies were also completed at 1 month, 3 months, 6 months, 9 months, 12 months and 18 months following cryopreservation. Results for three validation runs are shown in FIG. 21. On Day 0, the purity was 68.75%, 80.85% and 79.56% for each validation run, respectively. Immediately prior to cryopreservation, the purity increased to 99.97%, 99.87% and 99.30%, respectively. Post-thaw purity was maintained above 99% in all three validation runs. Stability studies also showed maintenance of purity above 98% even after 18 months in cryopreservation. See Table 7 for the average purity at baseline, after expansion and post cryopreservation.

TABLE 7
Purity

| Time | Mean | SD | N |
|---|---|---|---|
| Day 0 | 76.4 | 6.6 | 3 |
| Pre-Freeze | 99.7 | 0.4 | 3 |
| Post-Thaw | 99.9 | 0.1 | 3 |
| 1 month | 99.6 | 0.5 | 3 |
| 3 months | 99.8 | 0.0 | 3 |
| 6 months | 99.7 | 0.3 | 3 |
| 9 months | 99.5 | 0.6 | 3 |
| 12 months | 99.8 | 0.2 | 3 |
| 18 months | 99.3 | 0.8 | 3 |

### 8.7.1.3 Potency of Cryopreserved Treg Products

Potency of the expanded Treg cell populations was determined during the validation runs. The suppressive function of the expanded Treg cell populations was assessed by the proliferation of T responder cells (Tresp), which were freshly isolated from the same healthy control donor for each respective validation run per an established Treg Suppression Assay protocol.

Briefly, Carboxyfluorescein succinimidyl ester (CFSE) is an effective method to monitor cell proliferation. CFSE covalently labels long-lived intracellular molecules with the fluorescent dye, Carboxyfluorescein. Due to the ability to differentiate by flow cytometry analysis between labeled and unlabeled cells using CFSE, it is possible to analyze the proliferation of labeled target cells separate from unlabeled Tregs in co-culture experiments. CFSE readily crosses intact cell membranes. Once inside the cells, intracellular esterases cleave the acetate groups to yield the fluorescent Carboxyfluorescein molecule. The succinimidyl ester groups react with primary amines, crosslinking the dye to intracellular proteins. CD4⁺CD25⁺ cells are selected from the freshly isolated mononucleated cell fraction. Selection is performed using the matrix of the columns composed of ferromagnetic spheres that amplify the magnetic field by 10,000-fold. Cells are stained with magnetic bead-conjugated antibodies and separated by the high magnetic gradient within the column. Then the freshly isolated Tregs and responder T cells are co-cultured in a defined ratio in the presence of an optimized polyclonal stimulus, the Treg Suppression Inspector, while the proliferative response is measured. The Treg Suppression Inspector is an optimized T cell stimulation reagent for a Treg suppression assay, which is based on Anti-Biotin MACS iBead™ Particles that are loaded with biotinylated CD2, CD3, and CD28 antibodies. Activation of responder T cells (CD4⁺CD25⁻ or CD4⁺ T cells) with MACSiBead™ Particles leads to proliferation, whereas Tregs in the presence of the stimulus are not activated. Co-culture of responder T cells, MACSiBead™ Particles and Tregs results in reduced proliferation of responder T cells due to suppression by Tregs.

The Treg suppressive function was determined for expanded Treg cell populatins pre- and post-cryopreservation. For comparision, Treg suppressive function was also determined for baseline Tregs (freshly isolated, Day 0 Tregs following enrichment/depletion but prior to expansion).

In comparison to expanded Treg populations expanded from ALS patients known in the art, which exhibit less than 45% suppressive ability (see, e.g., Beers et al., JCI Insight. 2017; 2(5):e89530), cells produced following the methods described herein, in particular, the protocol described in Sections 8.3 and 8.5, above, exhibited suppressive abilities of over 80%. This suppressive ability remained high (above 65%) even after the Tregs were cryopreserved for up to 18 months and thawed following the methods described herein, in particular the protocol described in Section 8.6.

The Tresp were pre-incubated with carboxyfluorescein succinimidyl ester (CFSE) and incubated with or without the final Treg product at a 1:1 ratio (Tregs:Tresp). The Treg suppressive function was assayed by flow cytometry through which the percentage of total Tresp proliferation was determined.

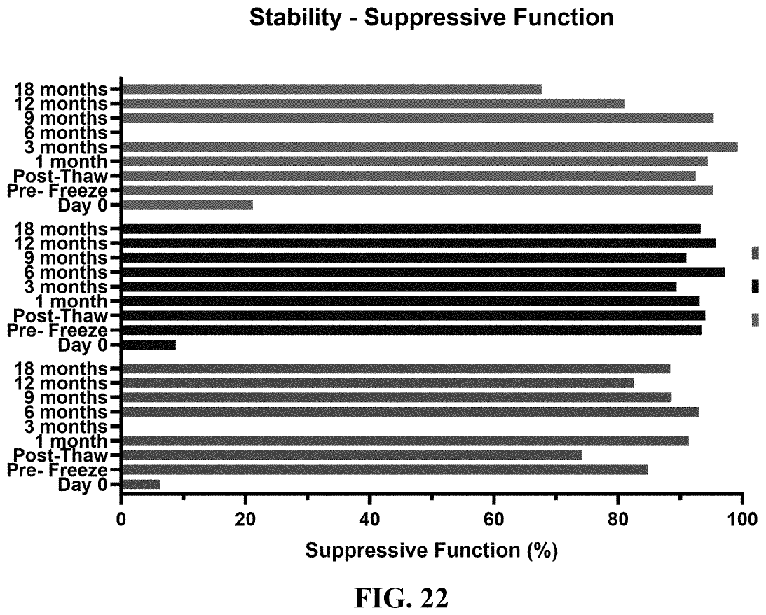

Treg suppressive function at baseline prior to expansion (Day 0) was only 6.3%, 8.8% and 21.2% for each validation run, respectively. Following expansion but immediately prior to cryopreservation (Pre-Freeze), the Treg suppressive function had increased to 84.8%, 93.4% and 95.3%, respectively. The Treg suppressive function was relatively maintained following thawing (post-thaw) in each validation run. Stability studies were completed at 1 month, 3 months, 6 months, 9 months, 12 months and 18 months following cryopreservation. Results for three validation runs are shown in FIG. 22. Maintenance of a Treg suppressive function well above baseline was demonstrated all the way out to 18 months in cryopreservation (88.4%, 93.3% and 67.7%, respectively). See Table 8 for the average potency at baseline, after expansion and after cryopreservation.

TABLE 8
Potency

| Time | Mean | SD | N |
|---|---|---|---|
| Day 0 | 12.1 | 8.0 | 3 |
| Pre-Freeze | 91.2 | 5.6 | 3 |
| Post-Thaw | 86.9 | 11.1 | 3 |
| 1 month | 93.0 | 1.5 | 3 |
| 3 months | 94.4 | 7.0 | 2 |
| 6 months | 95.1 | 3.0 | 2 |
| 9 months | 91.7 | 3.4 | 3 |
| 12 months | 86.4 | 8.1 | 3 |
| 18 months | 83.1 | 13.6 | 3 |

### 8.7.2. Expanded Treg Cell Populations from Phase 2a Clinical Trial

Populations of cryopreserved Tregs from an additional nine subjects (enrolled in a randomized, placebo-controlled, phase 2a trial to study the biological activity, safety, and tolerability of autologous Tregs in ALS were produced following the methods described herein, in particular, the protocol described in Sections 8.3 and 8.5, above, and thawed following the methods described herein, in particular the protocol described in Section 8.6, above, and were resuspended in a total of 50 mL of normal saline with 5% human serum albumin (HSA) and stored at 4° C.

Tregs expanded via the methods presented herein produced populations of Tregs that possess exemplary suppressive function, viability and purity. Remarkably, the Treg cell populations maintain this high potency, viability, purity and potency even after cryopreservation and thawing, without additional expansion, even after the Treg cell populations have been cryopreserved for up to 18 months (the latest time point assessed). Furthermore, cryopreserved Tregs were able to potently suppress proinflammatory macrophages, while freshly isolated ones were not.

### 8.7.2.1 Viability of Cryopreserved Treg Populations

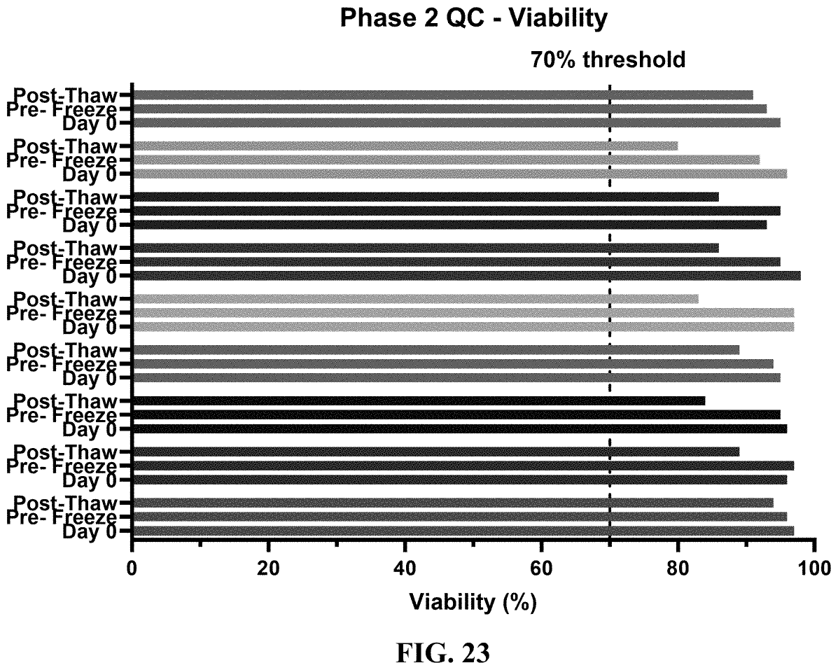

Cell viability was assessed by trypan blue staining. The viability was determined immediately after isolating fresh CD4⁺CD25⁺ cells (Day 0), immediately prior to cryopreservation following expansion (Pre-Freeze), and then 1 hour after thawing (Post-Thaw) the cryopreserved product. Results are shown in FIG. 23. Groups of bars show, starting from the, results for subject Nos. 701-115, 701-114, 701-103, 702-206, 702-205, 702-204, 702-203, 702-202 and 702-201. The validated manufacturing and cryopreservation/thawing processes resulted in cell viability after thawing well above the threshold of ≥70% meeting the release criteria for infusion into all subjects. See Table 9 for the average viability at baseline, after expansion and after cryopreservation.

TABLE 9
Phase 2 Trial Samples - Viability

| Time | Mean | SD | N |
|---|---|---|---|
| Day 0 | 95.9 | 1.5 | 9 |
| Pre-Freeze | 94.9 | 1.7 | 9 |
| Post-Thaw | 86.9 | 4.3 | 9 |

### 8.7.2.2 Purity of Cryopreserved Treg Populations

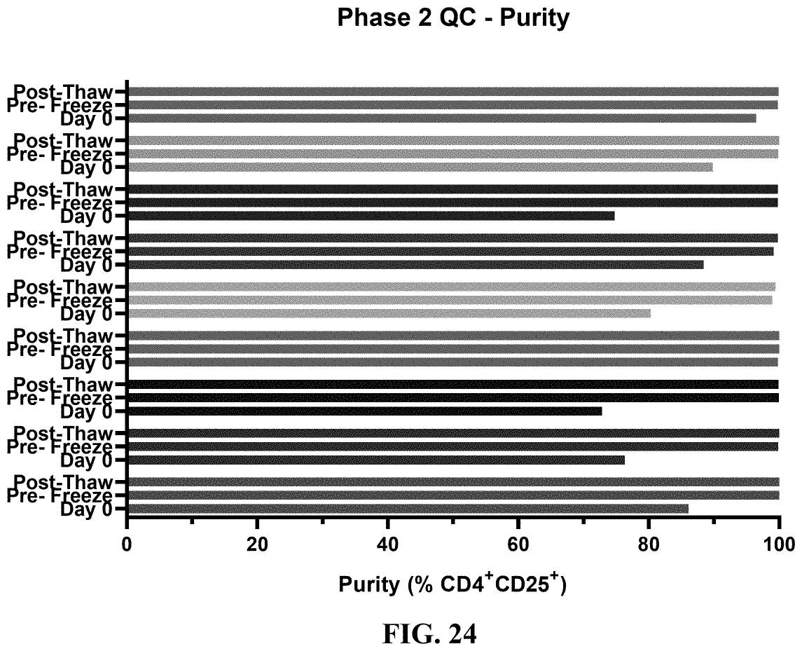

The percentage of CD4⁺CD25⁺ cells was determined at baseline following the CD25⁺ cell enrichment step (Day 0), immediately prior to cryopreservation following expansion (Pre-Freeze), and after thawing (Post-Thaw). Results for Treg populations expanded from nine subjects enrolled in the phase 2a clinical trial are shown in FIG. 24. Groups of bars show, starting from the, results for Subject Nos. 701-115, 701-114, 701-103, 702-206, 702-205, 702-204, 702-203, 702-202 and 702-201. The manufacturing process yielded Treg populations with purities ranging from 98.92 to 99.98% following expansion (Pre-Freeze). Further, the Treg purity was maintained above 99% in each subject following cryopreservation and thawing (Post-Thaw). See Table 10) for average purity at baseline, after expansion and after cryopreservation.

TABLE 10
Phase 2 Trial Samples - Purity

| Time | Mean | SD | N |
|---|---|---|---|
| Day 0 | 85.0 | 9.6 | 9 |
| Pre-Freeze | 99.7 | 0.4 | 9 |
| Post-Thaw | 99.9 | 0.2 | 9 |

### 8.7.2.3 Potency of Cryopreserved Treg Populations

The suppressive function of the Trege populations was assessed by measuring the effect on the proliferation of T responder cells (Tresp), which were freshly isolated from the same healthy control donor for each respective validation, run per an established Treg Suppression Assay SOP. The Treg suppressive function was not determined on freshly isolated Tregs from each patient prior to expansion, but all subjects were rapidly-progressing patients and expected to have a low Treg suppressive function at baseline. The Tresp were pre-incubated with carboxyfluorescein succinimidyl ester (CFSE) and incubated with or without the Treg population at a 1:1 ratio (Tregs:Tresp). The Treg suppressive function was assayed by flow cytometry through which the percentage of total Tresp proliferation was determined.

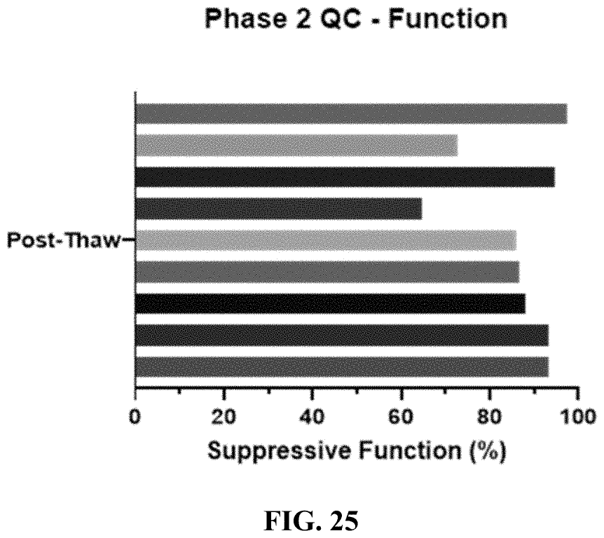

Results are shown in FIG. 25. Graph shows, starting from the, results for Subject Nos. 701-115, 701-114, 701-103, 702-206, 702-205, 702-204, 702-203, 702-202 and 702-201. Treg suppressive functions ranged from 64.8% to 97.6% (Mean 86.3%±10.9%) following expansion, cryopreservation and thawing. Of note, the Treg suppressive functions observed in the final phase 2a Treg products utilizing the manufacturing process were much higher than the Treg suppressive functions observed utilizing the manufacturing process for the phase 1 trial (range 27.8%-44.2%; mean 37.3%±8.5%) as measured by the same technique (n=3, see, e.g., Beers et al., JCI Insight. 2017; 2(5):e89530). See Table 11 for average potency after cryopreservation.

TABLE 11
Phase 2 Trial Samples - Potency

| Time | Mean | SD | N |
|---|---|---|---|
| Post-Thaw | 86.3 | 10.9 | 9 |

### 8.7.3. The Ex Vivo-Expanded Treg Cell Populations Suppress Proinflammatory Macrophages

Ex vivo-expanded Treg cell populations were produced from three patients with ALS, then cryopreserved and thawed following the methods described herein, in particular, the protocols described in Sections 8.3, 8.5, and 8.6, above. Following thawing and without t additional expansion, the Tregs were resuspended in a total of 50 mL of normal saline with 5% human serum albumin (HSA) and stored at 4° C. The ability to suppress macrophages was determined by measuring IL-6 production using ELISA.

The data presented in this example show that a therapeutic population of Tregs produced by a method here, in particular, the protocols described in Sections 8.3, 8.5, and 8.6, above, exhibits excellent purity, viability, and potency. Potency includes the ability to suppress inflammatory cell activity as shown, for example, by the ability to suppress secretion of IL-6 from proinflammatory macrophages. This ability to suppress inflammatory macrophages was seen even after cryopreservation and thawing, and without additional expansion after cryopreservation. Conversely, freshly isolated Tregs enriched from either ALS patients or from healthy volunteers failed to suppress proinflammatory macrophages. Thus, the results demonstrate that the Tregs produced using the method presented herein substantially differ from Tregs as they naturally exist in a subject (e.g., a healthy subject or a subject having a neurodegenerative disorder such as ALS).

### 8.7.3.1 Freshly Isolated Tregs do not Suppress Proinflammatory Macrophages

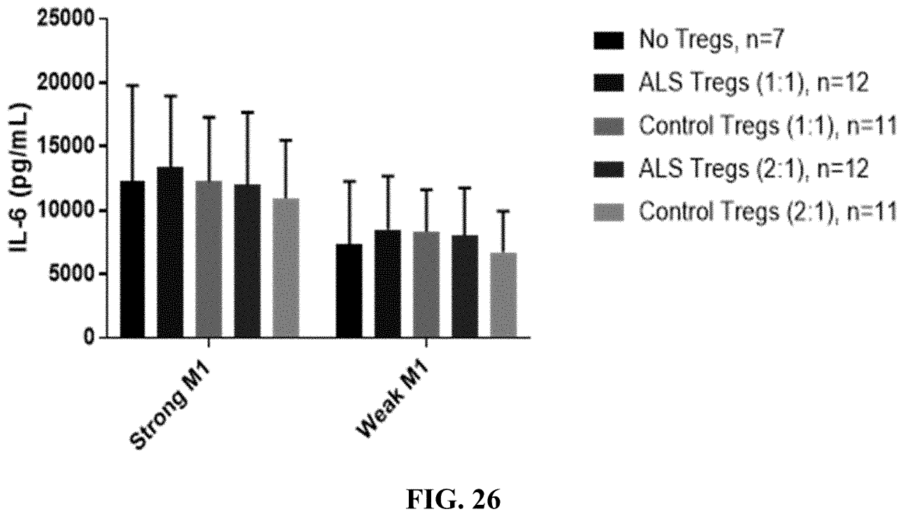

Neither freshly isolated Tregs from ALS patients (n=12) nor healthy control volunteers (n=11) suppressed proinflammatory macrophages, as measured by macrophage IL-6 production in vitro. To determine whether Tregs directly suppressed activated macrophages, freshly isolated Tregs from slow/fast-progressing ALS patients and healthy controls were cocultured at 1:1 and 2:1 ratios with induced pluripotent stem cell-derived proinflammatory macrophages (iPSC-M1s). IL-6 production by the macrophages was measured by ELISA. Results are shown in FIG. 26 (conditions listed are depicted in the graph from left to right for the Strong M1 (left group of bars) and Weak M1 (right group of bars) experiments). The data showed that neither Tregs from ALS patients nor from healthy controls suppressed IL-6 production from iPSC-M1s by ELISA. This was observed in both strong and weak M1 macrophages (strong M1s were stimulated with 10-fold higher concentrations of LPS and IFNγ compared to weak M1s).

### 8.7.3.2 Cryopreserved Therapeutic Populations of Tregs Suppress Proinflammatory Macrophages

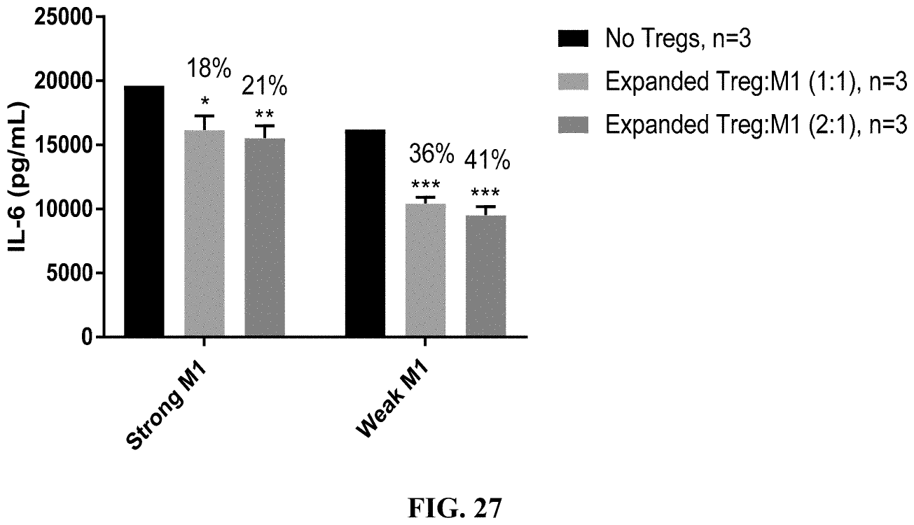

Cryopreserved therapeutic Treg populations produced via the methods presented herein, upon thawing and testing with no additional expansion, exhibit an ability todramatically suppress myeloid cells, indicating that the expanded Treg cell populations, included the cryopreserved therapeutic Treg populations described herein, exhibit a major characteristic likely indicative of benefit for treatment of patients, including ALS patients. Results are shown in FIG. 27, demonstrating suppression of proinflammatory macrophages, as measured by macrophage IL-6 production in vitro. This is in contrast to Tregs freshly isolated either from ALS patients (n=3) or healthy donors (n=3); see previous section. Numbers show percentage decrease compared to No Treg control; * indicates a p-value of 0.05 or less, ** indicates a p value of 0.01 or les, *** indicates a p-value of 0.001 or less. Of note, the weaker the proinflammatory state of the MIs macrophages, the stronger the suppressive activity of the expanded Treg cell populations.

### 8.8 Example 8: Characterization of Freshly Isolated, Ex Vivo-Expanded and Cryopreserved Tre2 Cell Populations by Proteomics

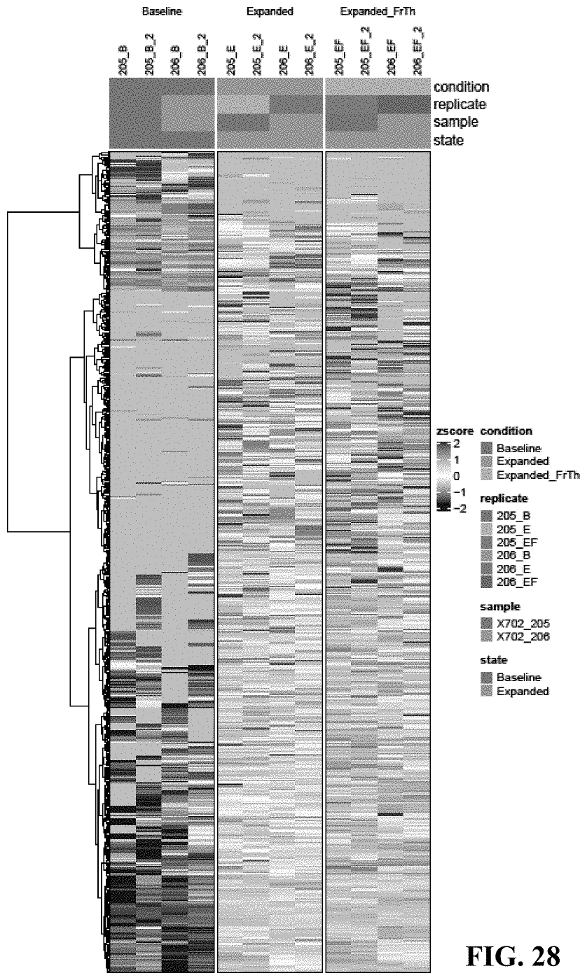

The results presented herein, for example, the results presented in the previous example, demonstrate the exemplary purity, viability and potency of the Treg populations produced via the methods presented herein. The experiments presented in this example describe a proteomic analysis of baseline Tregs, expanded Tregs and a cryopreserved therapeutic population of Tregs following thawing, without additional expansion. The results of these experiments further demonstrate that the Tregs produced via the methods presented herein constitute a unique Treg population that not only exhibits superior functional characteristics but also exhibits unique gene product signatures that are remarkably conserved among the Treg populations produced from both patient samples (see FIG. 28). As discussed below, these signatures differ substantially from the baseline Treg gene product signature and are indicative of the health and potency of the expanded Tregs. Surprisingly, the signatures are virtually unchanged when comparing the Tregs pre- and post-cryopreservation.

### 8.8.1. Groups Analyzed in the Experiment

Baseline Tregs—Tregs at baseline levels derived from ALS patients (patient 205 with two independent inputs/runs and patient 206 with two independent inputs/runs).

Expanded Tregs—The same patients' Tregs following the expansion described herein, in particular, the protocols described in Sections 8.3, 8.5, and 8.6, above (expanded patient 205 with two independent inputs/runs and expanded patient 206 with two independent inputs/runs).

Cryopreserved therapeutic population of Tregs—The same patients' expanded Tregs following a freeze/thaw cycle (cryopreservation then thawing and testing without t additional expansion) from storage (expanded patient 205 Tregs after freeze/thaw with two independent inputs/runs and expanded patient 206 Tregs after freeze/thaw with two independent inputs/runs).

### 8.8.2. Proteomic Profiling Methods

Proteomic profiling via single-shot proteomic analysis was performed on Treg Baseline, Treg Expanded, and Treg Expanded Post Thaw cells. Cell pellets were lysed by RIPA buffer. For each sample, 5 μg of protein supernatant was mixed with NuPAGE LDS Sample Buffer (Thermo, NP0007) and boiled at 90° C. for 10 minutes. The proteins were separated on pre-cast NuPAGE Bis-Tris 10% protein gel (Invitrogen, NP0301BOX). For staining, the gels were first fixed with Destain I (40% MeOH, 7% AcOH) for 15 minutes, stained with Coomassie (0.025% Brilliant Blue R-250, 40% MeOH, 7% AcOH) for 5 minutes, de-stained with Destain I buffer 2 times for 30 minutes, and left in water overnight. Four band regions were cut for each sample. The bands were further de-stained completely with Destain II (40% MeOH, 50 mM NH4CO3), equilibrated in water, dehydrated with 75% ACN, and incubated in 50 mM Ammonium bicarbonate solution for 1 hr. Then each band was crushed and digested with 22 μl of Trypsin solution (20 μl 50 mM Ammonium Bicarbonate and 2 μl of 100 ng/μl Trypsin (GenDepot: T9600) overnight at 37° C. Next, the digest was acidified by adding 20 μl 2% Formic acid (Thermo, 85178). The peptide from gel was extracted by adding 350 μl of 100% ACN for 15 min and collected by centrifugation at 21,000 rpm for 5 min. The extracted peptides were dried down in a SpeedVac (Thermo Scientific SC210). For MS runs, peptides from all 4 Bands were re-suspended in 20 μl 5% methanol+0.1% FA solution, pooled together, and measured on an Exploris Orbitrap 480 mass spectrometer (Thermo Fisher Scientific, San Jose, Calif.) with online separation with Thermo Scientific™ EASY-nLC™ 1200 Liquid Chromatography system. Online separation was performed on 1 μg with a 20 cm long, 100 m inner diameter packed column with sub-2 μm C18 beads (Reprosil-Pur Basic C18, Catalogue #r119.b9.0003, Dr. Maisch GmbH). A linear reverse phase gradient from 2-30% B (100% ACN) was run for 90 minutes.

Raw mass spectrometry data was processed Proteome Discoverer (PD version 2.0.0.802; Thermo Fisher Scientific). Spectra were matched to peptides from the Human RefSeq protein database (downloaded through RefProtDB on 2020-03-24) within 350-10,000 Da mass range and trypsin/P in silico digestion and up to 2 missed cleavages. Mass error was set to 20 ppm for precursor mass, and 0.02 Da for fragment mass. The following dynamic modifications: Acetyl (Protein N-term), Oxidation (M), Carbamidomethyl (C), DeStreak (C), and Deamidated (NQ). Peptide-Spectral Matches (PSMs) were validated with Percolator (v2.05) (Kall et al 2007, PMID 17952086). The target strict and relaxed FDR levels for Percolator were set at 0.01 and 0.05 (1% and 5%), respectively. Label-free quantification of PSMs was made using Proteome Discoverer's Area Detector Module.

Protein inference and quantitation was performed by gpGrouper (v1.0.040) with shared peptide iBAQ area distribution (Saltzman et al 2018 PMID 30093420). Resulting protein values were median normalized and log transformed for downstream analyses. For statistical assessment, missing value imputation was employed through sampling a normal distribution N(μ-1.8 σ, 0.8σ), where μ, σ are the mean and standard deviation of the quantified values. To assess differences between groups, the moderated t-test was used as implemented in the R package limma (Ritchie et al., 2015). Multiple-hypothesis testing correction was performed with the Benjamini-Hochberg procedure (Benjamini and Hochberg, 1995). Pathway analyses for phenotype associations were examined using Reactome, Kyoto Encyclopedia of Genes and Genome (KEGG), and Gene Set Enrichment Analysis (GSEA).

### 8.8.3. Proteomic Study Finding

The results presented in these examples demonstrate that the methods of producing ex vivo-expanded Tregs result in expanded Treg cell populations with superior characteristics, eg., purity, viability and potency characteristics. In this example, an unbiased single-shot proteomic analysis via mass spectrometry identified gene products from the expanded Treg cell populations before and after cryopreservation, as well as from baseline Tregs (freshly isolated, enriched Tregs pre-expansion, here, from ALS patient cell samples). The following results were obtained:
1. A baseline Treg gene product signature was identified that is resolved following the Treg expansion process. This signature suggests, e.g., dysfunctional epigenetic/methylation mechanisms in the baseline Tregs. A methylation gene product signature was also identified within this baseline signature.
2. A unique proteomic gene product signature of the expanded Tregs was identified, indicating that the Treg cell population produced via the methods presented herein is new and that the expanded Treg cell population represents a robust, potent population. Of importance, this signature is remarkably conserved among the different ALS patient Tregs that went through the expansion process.
3. The enhanced Treg gene product signature following expansion can be further defined by at least the following unique gene product signatures:
   a. Treg-associated gene product signature.
   b. Mitochondria gene product signature.
   c. Cell proliferation gene product signature (cell division, cell cycle, and DNA replication/repair).
   d. Highest protein expression gene product signature following expansion process.
4. Remarkably, the expanded Treg signatures do not significantly change following cryopreservation, as tested following thawing and without additional expansion. Thus, a freeze/thaw cycle that is utilized for storage of expanded Treg cell population batches for therapeutic treatment does not significantly change the Treg expansion signature. Interestingly, as demonstrated herein, the Treg cell populations produced via the methods of the present application also maintain their purity, viability and suppressive functions following thawing, without the need for further expansion.

Representation of significant proteomic signatures of these overall experimental findings can be found in the experimental heat map in FIG. 28.

### 8.8.4. Results

### 8.8.4.1 Dysfunctional Baseline Tregs Display Proteomic Signature that is Ameliorated Following Expansion

Single-shot proteomic profiling identified peptide sequences that map to 82 gene products out of the 3,709 total found that were increased in the baseline samples but were subsequently significantly reduced or lost during the expansion process (Table 12; dysfunctional baseline gene produce signature). Analysis of the signature included gene products that had a p value of p<0.05 after correction for false discovery rate and multiple hypothesis testing while also having a fold change of at least 4 (log 2 FC>2). Pathway analysis of these significant gene product sets reveals a dysfunctional Treg phenotype including dysregulated calcium dynamics (p=0.0278), loss of MECP2 binding ability to 5hmC-DNA (p=6.96e-6), dysregulation of MECP2 expression and activity (p=0.0166), and loss of MECP2 regulation (p=0.0303), phosphorylation (p=0.037), and binding abilities (p=0.0456). It has been previously shown that proper MECP2 expression and function are pivotal for the expression of Treg health and function marker FOXP3 (PMID: 24958888).

Additionally, multiple gene products in this signature are also mapped to histone proteins and other proteins that are modified and play a role in the control of the unwinding of DNA to enable epigenetic changes, particularly methylation of DNA that directly affects transcription. Expression of these dysfunctional epigenetic/methylation-associated gene products is decreased in the population of Tregs after expansion relative to that observed for baseline Tregs. See Table 13 (methylation gene product signature).

TABLE 12
Genes products that were increased in the baseline samples but were
subsequently significantly reduced or lost during the expansion process.

| | | | Log2 of | |
|---|---|---|---|---|
| | | | fold- | |
| | | | change | |
| | | | of | |
| | NCBI | | baseline | |
| Gene | Gene | | vs. | Adjusted |
| Symbol | ID | Gene Description | expanded | p-value |
| HIST1H2BC | 8347 | histone cluster 1 H2B | −14.362 | <0.001 |
| | | family member c | | |
| HIST1H2BE | 8344 | histone cluster 1 H2B | −14.199 | 0.005 |
| | | family member e | | |
| HIST1H2BG | 8339 | histone cluster 1 H2B | −14.091 | 0.002 |
| | | family member g | | |
| HIST1H2BI | 8346 | histone cluster 1 H2B | −13.849 | 0.004 |
| | | family member i | | |
| HIST1H2BF | 8343 | histone cluster 1 H2B | −11.737 | 0.020 |
| | | family member f | | |
| RAB3C | 115827 | RAB3C, member RAS | −9.908 | 0.004 |
| | | oncogene family | | |
| CLC | 1178 | Charcot-Leyden crystal | −8.446 | 0.023 |
| | | galectin | | |
| TUBA1A | 7846 | tubulin alpha 1a | −8.424 | 0.027 |
| HBB | 3043 | hemoglobin subunit beta | −8.120 | <0.001 |
| HBA1 | 3039 | hemoglobin subunit | −8.068 | 0.001 |
| | | alpha 1 | | |
| HBA2 | 3040 | hemoglobin subunit | −7.585 | 0.002 |
| | | alpha 2 | | |
| CDK3 | 1018 | cyclin dependent kinase 3 | −7.110 | 0.007 |
| MECP2 | 4204 | methyl-CpG binding | −6.924 | <0.001 |
| | | protein 2 | | |
| MTHFS | 10588 | methenyltetrahydrofolate | −6.566 | 0.001 |
| | | synthetase | | |
| PLIN3 | 10226 | perilipin 3 | −6.556 | <0.001 |
| H1F0 | 3005 | H1 histone family | −6.342 | 0.006 |
| | | member 0 | | |
| ANXA1 | 301 | annexin A1 | −6.131 | <0.001 |
| RAB3D | 9545 | RAB3D, member RAS | −5.629 | 0.017 |
| | | oncogene family | | |
| GIMAP1 | 170575 | GTPase, IMAP family | −5.611 | 0.009 |
| | | member 1 | | |
| MPO | 4353 | myeloperoxidase | −5.571 | 0.012 |
| LRRD1 | 401387 | leucine rich repeats and | −5.326 | 0.030 |
| | | death domain containing 1 | | |
| HARS2 | 23438 | histidyl-tRNA synthetase | −5.091 | 0.030 |
| | | 2, mitochondrial | | |
| RAB3A | 5864 | RAB3A, member RAS | −5.019 | 0.006 |
| | | oncogene family | | |
| GIMAP1- | 1E+08 | GIMAP1-GIMAP5 | −4.978 | <0.001 |
| GIMAP5 | | readthrough | | |
| PPM1F | 9647 | protein phosphatase, | −4.905 | 0.004 |
| | | Mg2+/Mn2+ dependent | | |
| | | 1F | | |
| PRG2 | 5553 | proteoglycan 2, pro | −4.903 | 0.004 |
| | | eosinophil major basic | | |
| | | protein | | |
| CA5B | 11238 | carbonic anhydrase 5B | −4.877 | 0.007 |
| NAAA | 27163 | N-acylethanolamine acid | −4.764 | 0.023 |
| | | amidase | | |
| ELANE | 1991 | elastase, neutrophil | −4.762 | 0.026 |
| | | expressed | | |
| A1BG | 1 | alpha-1-B glycoprotein | −4.649 | 0.006 |
| PRPF38B | 55119 | pre-mRNA processing | −4.580 | 0.007 |
| | | factor 38B | | |
| CFAP99 | 402160 | cilia and flagella | −4.512 | 0.020 |
| | | associated protein 99 | | |
| AFM | 173 | afamin | −4.357 | 0.003 |
| PCYOX1 | 51449 | prenylcysteine oxidase 1 | −4.301 | 0.042 |
| HK3 | 3101 | hexokinase 3 | −4.219 | 0.020 |
| ARFGAP3 | 26286 | ADP ribosylation factor | −4.168 | 0.048 |
| | | GTPase activating | | |
| | | protein 3 | | |
| CRIP2 | 1397 | cysteine rich protein 2 | −4.156 | 0.009 |
| HMGN4 | 10473 | high mobility group | −4.035 | 0.007 |
| | | nucleosomal binding | | |
| | | domain 4 | | |
| SGSH | 6448 | N-sulfoglucosamine | −4.027 | <0.001 |
| | | sulfohydrolase | | |
| RASSF2 | 9770 | Ras association domain | −3.973 | 0.002 |
| | | family member 2 | | |
| CRLF3 | 51379 | cytokine receptor like | −3.923 | 0.035 |
| | | factor 3 | | |
| HRNR | 388697 | hornerin | −3.801 | 0.001 |
| DPP7 | 29952 | dipeptidyl peptidase 7 | −3.626 | 0.032 |
| WDHD1 | 11169 | WD repeat and HMG-box | −3.497 | 0.049 |
| | | DNA binding protein 1 | | |
| KPRP | 448834 | keratinocyte proline rich | −3.431 | 0.004 |
| | | protein | | |
| SKAP1 | 8631 | src kinase associated | −3.366 | 0.003 |
| | | phosphoprotein 1 | | |
| RIPOR2 | 9750 | RHO family interacting | −3.356 | 0.045 |
| | | cell polarization | | |
| | | regulator 2 | | |
| ATG5 | 9474 | autophagy related 5 | −3.344 | 0.046 |
| SERPINB9 | 5272 | serpin family B member 9 | −3.250 | <0.001 |
| ALOX5AP | 241 | arachidonate | −3.232 | 0.001 |
| | | 5-lipoxygenase activating | | |
| | | protein | | |
| GIMAP4 | 55303 | GTPase, IMAP family | −3.187 | 0.003 |
| | | member 4 | | |
| SIGIRR | 59307 | single Ig and TIR domain | −3.181 | 0.025 |
| | | containing | | |
| WDR37 | 22884 | WD repeat domain 37 | −3.143 | 0.045 |
| HDDC3 | 374659 | HD domain containing 3 | −3.143 | 0.029 |
| HPX | 3263 | hemopexin | −3.094 | 0.001 |
| RASGRP2 | 10235 | RAS guanyl releasing | −3.055 | 0.007 |
| | | protein 2 | | |
| IL16 | 3603 | interleukin 16 | −3.035 | <0.001 |
| VAT1 | 10493 | vesicle amine transport 1 | −3.020 | 0.002 |
| DSG1 | 1828 | desmoglein 1 | −3.003 | 0.031 |
| UBASH3A | 53347 | ubiquitin associated and | −2.984 | 0.010 |
| | | SH3 domain containing A | | |
| AAK1 | 22848 | AP2 associated kinase 1 | −2.966 | 0.008 |
| HRG | 3273 | histidine rich glycoprotein | −2.815 | 0.034 |
| ERGIC1 | 57222 | endoplasmic reticulum- | −2.777 | 0.003 |
| | | golgi intermediate | | |
| | | compartment 1 | | |
| PIK3R1 | 5295 | phosphoinositide-3-kinase | −2.706 | 0.026 |
| | | regulatory subunit 1 | | |
| PGM2 | 55276 | phosphoglucomutase 2 | −2.698 | <0.001 |
| EML4 | 27436 | EMAP like 4 | −2.694 | <0.001 |
| GCA | 25801 | grancalcin | −2.624 | 0.036 |
| SH3KBP1 | 30011 | SH3 domain containing | −2.604 | 0.001 |
| | | kinase binding protein 1 | | |
| DCXR | 51181 | dicarbonyl and L-xylulose | −2.581 | <0.001 |
| | | reductase | | |
| AHNAK | 79026 | AHNAK nucleoprotein | −2.573 | 0.001 |
| FYB1 | 2533 | FYN binding protein 1 | −2.551 | 0.001 |
| HP1BP3 | 50809 | heterochromatin protein 1 | −2.529 | <0.001 |
| | | binding protein 3 | | |
| HP | 3240 | haptoglobin | −2.527 | 0.003 |
| APOH | 350 | apolipoprotein H | −2.468 | <0.001 |
| PDP1 | 54704 | pyruvate dehyrogenase | −2.386 | 0.008 |
| | | phosphatase catalytic | | |
| | | subunit 1 | | |
| KRT5 | 3852 | keratin 5 | −2.384 | 0.023 |
| GRK6 | 2870 | G protein-coupled | −2.333 | 0.009 |
| | | receptor kinase 6 | | |
| CYB5R1 | 51706 | cytochrome b5 reductase 1 | −2.168 | 0.004 |
| FLNA | 2316 | filamin A | −2.121 | <0.001 |
| PIP4K2A | 5305 | phosphatidylinositol-5- | −2.094 | 0.001 |
| | | phosphate 4-kinase type 2 | | |
| | | alpha | | |
| SRSF9 | 8683 | serine and arginine rich | −2.088 | 0.028 |
| | | splicing factor 9 | | |
| ALB | 213 | albumin | −2.001 | <0.001 |

TABLE 13
Dysfunctional epigentic/methylation signature in baseline Tregs

| | | | Log2 of | |
|---|---|---|---|---|
| | | | fold- | |
| | | | change | |
| | | | of | |
| | | | baseline | |
| Gene | NCBI | | vs. | Adjusted |
| Symbol | Gene ID | Gene Description | expanded | p-value |
| HIST1H2BC | 8347 | histone cluster 1 H2B | −14.362 | <0.001 |
| | | family member c | | |
| HIST1H2BE | 8344 | histone cluster 1 H2B | −14.199 | 0.005 |
| | | family member e | | |
| HIST1H2BG | 8339 | histone cluster 1 H2B | −14.091 | 0.002 |
| | | family member g | | |
| HIST1H2BI | 8346 | histone cluster 1 H2B | −13.849 | 0.004 |
| | | family member i | | |
| HIST1H2BF | 8343 | histone cluster 1 H2B | −11.737 | 0.020 |
| | | family member f | | |
| MECP2 | 4204 | methyl-CpG binding | −6.924 | <0.001 |
| | | protein 2 | | |
| H1F0 | 3005 | H1 histone family | −6.342 | 0.006 |
| | | member 0 | | |
| HP1BP3 | 50809 | heterochromatin protein | −2.529 | <0.001 |
| | | 1 binding protein 3 | | |

### 8.8.4.2 Enriched Proteomic Signature Following Expansion of Patient Tregs that is Observed Across Patient Groups

The proteomic analysis of expanded Tregs compared to baseline Tregs identified peptide sequences that mapped back to 391 unique gene products out of 3,709 total that were found that are enriched in the expanded Tregs compared to the baseline patient Tregs (Table 14) These genes are a compilation of all significant gene products that had a p value of p<0.05 after correction for false discovery rate and multiple hypothesis testing while also having a fold change of at least 4 (log 2 FC>2).

TABLE 14
Gene products that were enriched in the expanded Tregs compared to
the baseline patient Tregs.

| | | | Log2 of | |
|---|---|---|---|---|
| | | | fold- | |
| | | | change | |
| | | | of | |
| | | | baseline | |
| Gene | NCBI | | vs. | Adjusted |
| Symbol | Gene ID | Gene Description | expanded | p-value |
| NME1 | 4830 | NME/NM23 nucleoside | 13.947 | 0.006 |
| | | diphosphate kinase 1 | | |
| HIST1H2BJ | 8970 | histone cluster 1 H2B | 13.792 | <0.001 |
| | | family member j | | |
| NQO1 | 1728 | NAD(P)H quinone | 9.019 | <0.001 |
| | | dehydrogenase 1 | | |
| TUBB8 | 347688 | tubulin beta 8 class VIII | 8.661 | 0.028 |
| TUBB4A | 10382 | tubulin beta 4A class | 8.606 | 0.026 |
| | | IVa | | |
| AK4 | 205 | adenylate kinase 4 | 8.292 | 0.001 |
| PTMS | 5763 | parathymosin | 8.181 | 0.005 |
| CD70 | 970 | CD70 molecule | 8.139 | <0.001 |
| IL1RN | 3557 | interleukin 1 receptor | 7.715 | <0.001 |
| | | antagonist | | |
| PGRMC1 | 10857 | progesterone receptor | 7.555 | 0.002 |
| | | membrane component 1 | | |
| FAH | 2184 | fumaryl acetoacetate | 7.513 | <0.001 |
| | | hydrolase | | |
| TFRC | 7037 | transferrin receptor | 7.114 | <0.001 |
| MRPL46 | 26589 | mitochondrial ribosomal | 6.991 | 0.006 |
| | | protein L46 | | |
| BST2 | 684 | bone marrow stromal | 6.904 | <0.001 |
| | | cell antigen 2 | | |
| ARL6IP1 | 23204 | ADP ribosylation factor | 6.827 | <0.001 |
| | | like GTPase 6 | | |
| | | interacting protein 1 | | |
| HLA-DQB1 | 3119 | major histocompatibility | 6.779 | 0.001 |
| | | complex, class II, DQ | | |
| | | beta 1 | | |
| COX17 | 10063 | cytochrome c oxidase | 6.680 | 0.002 |
| | | copper chaperone | | |
| | | COX17 | | |
| MZB1 | 51237 | marginal zone B and B1 | 6.647 | <0.001 |
| | | cell specific protein | | |
| CDK1 | 983 | cyclin dependent kinase 1 | 6.615 | 0.007 |
| MCM5 | 4174 | minichromosome | 6.473 | <0.001 |
| | | maintenance complex | | |
| | | component 5 | | |
| CD38 | 952 | CD38 molecule | 6.393 | 0.007 |
| HMOX1 | 3162 | heme oxygenase 1 | 6.359 | <0.001 |
| CDK6 | 1021 | cyclin dependent | 6.206 | 0.006 |
| | | kinase 6 | | |
| MCM2 | 4171 | minichromosome | 6.203 | 0.010 |
| | | maintenance complex | | |
| | | component 2 | | |
| ENO3 | 2027 | enolase 3 | 6.086 | 0.001 |
| PITRM1 | 10531 | pitrilysin | 6.046 | <0.001 |
| | | metallopeptidase 1 | | |
| MCM4 | 4173 | minichromosome | 6.021 | <0.001 |
| | | maintenance complex | | |
| | | component 4 | | |
| TBL2 | 26608 | transducin beta like 2 | 5.989 | <0.001 |
| CDK5 | 1020 | cyclin dependent | 5.954 | <0.001 |
| | | kinase 5 | | |
| DHRS2 | 10202 | dehydrogenase/ | 5.867 | 0.001 |
| | | reductase 2 | | |
| TOMM34 | 10953 | translocase of outer | 5.836 | 0.007 |
| | | mitochondrial | | |
| | | membrane 34 | | |
| ADI1 | 55256 | acireductone | 5.825 | <0.001 |
| | | dioxygenase 1 | | |
| SLC25A10 | 1468 | solute carrier family 25 | 5.776 | <0.001 |
| | | member 10 | | |
| APOBEC3D | 140564 | apolipoprotein B | 5.672 | 0.001 |
| | | mRNA editing enzyme | | |
| | | catalytic subunit 3D | | |
| GK | 2710 | glycerol kinase | 5.635 | <0.001 |
| MCM3 | 4172 | minichromosome | 5.613 | <0.001 |
| | | maintenance complex | | |
| | | component 3 | | |
| DHFR | 1719 | dihydrofolate reductase | 5.566 | 0.001 |
| HLA-DRB1 | 3123 | major histocompatibility | 5.489 | <0.001 |
| | | complex, class II, DR | | |
| | | beta 1 | | |
| DHCR24 | 1718 | 24-dehydrocholesterol | 5.432 | 0.005 |
| | | reductase | | |
| ITGB7 | 3695 | integrin subunit beta 7 | 5.421 | <0.001 |
| MMGT1 | 93380 | membrane magnesium | 5.355 | 0.002 |
| | | transporter 1 | | |
| ATOX1 | 475 | antioxidant 1 copper | 5.355 | 0.007 |
| | | chaperone | | |
| SELPLG | 6404 | selectin P ligand | 5.317 | 0.019 |
| USP10 | 9100 | ubiquitin specific | 5.286 | <0.001 |
| | | peptidase 10 | | |
| CTSH | 1512 | cathepsin H | 5.282 | 0.012 |
| HM13 | 81502 | histocompatibility minor | 5.277 | 0.001 |
| | | 13 | | |
| MRPL22 | 29093 | mitochondrial ribosomal | 5.268 | 0.007 |
| | | protein L22 | | |
| SPTLC1 | 10558 | serine | 5.205 | 0.001 |
| | | palmitoyltransferase | | |
| | | long chain base | | |
| | | subunit 1 | | |
| TST | 7263 | thiosulfate | 5.170 | <0.001 |
| | | sulfurtransferase | | |
| APOA1 | 335 | apolipoprotein A1 | 5.165 | 0.022 |
| CHP1 | 11261 | calcineurin like EF-hand | 5.113 | 0.001 |
| | | protein 1 | | |
| SLC25A4 | 291 | solute carrier family 25 | 5.089 | 0.030 |
| | | member 4 | | |
| STMN2 | 11075 | stathmin 2 | 5.041 | 0.023 |
| ATP2A2 | 488 | ATPase sarcoplasmic/ | 5.017 | 0.005 |
| | | endoplasmic reticulum | | |
| | | Ca2+ transporting 2 | | |
| CTLA4 | 1493 | cytotoxic T-lymphocyte | 4.993 | 0.002 |
| | | associated protein 4 | | |
| CD59 | 966 | CD59 molecule | 4.979 | 0.007 |
| | | (CD59 blood group) | | |
| GLA | 2717 | galactosidase alpha | 4.978 | 0.005 |
| PYDC1 | 260434 | pyrin domain containing | 4.931 | 0.049 |
| | | 1 | | |
| MYCBP | 26292 | MYC binding protein | 4.922 | 0.005 |
| TNFRSF18 | 8784 | TNF receptor superfamily | 4.896 | 0.001 |
| | | member 18 | | |
| IRF4 | 3662 | interferon regulatory | 4.896 | 0.003 |
| | | factor 4 | | |
| MTHFD2 | 10797 | methylenetetrahydrofolate | 4.894 | 0.022 |
| | | dehydrogenase | | |
| | | (NADP+ dependent) 2, | | |
| | | methenyltetrahydrofolate | | |
| | | cyclohydrolase | | |
| GNA15 | 2769 | G protein subunit alpha | 4.877 | 0.002 |
| | | 15 | | |
| CCDC124 | 115098 | coiled-coil domain | 4.855 | 0.006 |
| | | containing 124 | | |
| TMEM97 | 27346 | transmembrane protein | 4.848 | 0.000 |
| | | 97 | | |
| ACP5 | 54 | acid phosphatase 5, | 4.822 | 0.014 |
| | | tartrate resistant | | |
| TIGAR | 57103 | TP53 induced glycolysis | 4.808 | <0.001 |
| | | regulatory phosphatase | | |
| MED20 | 9477 | mediator complex | 4.788 | 0.004 |
| | | subunit 20 | | |
| SLC3A2 | 6520 | solute carrier family 3 | 4.787 | <0.001 |
| | | member 2 | | |
| ARMC1 | 55156 | armadillo repeat | 4.781 | 0.044 |
| | | containing 1 | | |
| MRPL14 | 64928 | mitochondrial ribosomal | 4.773 | 0.029 |
| | | protein L14 | | |
| PAIP2 | 51247 | poly(A) binding protein | 4.770 | 0.020 |
| | | interacting protein 2 | | |
| MCM7 | 4176 | minichromosome | 4.743 | <0.001 |
| | | maintenance complex | | |
| | | component 7 | | |
| RPL22L1 | 200916 | ribosomal protein L22 | 4.733 | 0.002 |
| | | like 1 | | |
| ITPK1 | 3705 | inositol-tetrakisphosphate | 4.723 | 0.002 |
| | | 1-kinase | | |
| HLA-DRA | 3122 | major histocompatibility | 4.722 | <0.001 |
| | | complex, class II, DR | | |
| | | alpha | | |
| BSG | 682 | basigin (Ok blood group) | 4.721 | 0.001 |
| OCIAD2 | 132299 | OCIA domain containing | 4.678 | <0.001 |
| | | 2 | | |
| CHMP6 | 79643 | charged multivesicular | 4.644 | 0.029 |
| | | body protein 6 | | |
| RALB | 5899 | RAS like proto-oncogene | 4.644 | 0.008 |
| | | B | | |
| MAOA | 4128 | monoamine oxidase A | 4.637 | 0.002 |
| HMBS | 3145 | hydroxymethylbilane | 4.635 | 0.005 |
| | | synthase | | |
| SLC25A19 | 60386 | solute carrier family 25 | 4.627 | 0.001 |
| | | member 19 | | |
| FOXP3 | 50943 | forkhead box P3 | 4.589 | 0.001 |
| SLC16A1 | 6566 | solute carrier family 16 | 4.588 | 0.016 |
| | | member 1 | | |
| ACOT7 | 11332 | acyl-CoA thioesterase 7 | 4.566 | <0.001 |
| RBX1 | 9978 | ring-box 1 | 4.562 | 0.010 |
| DDB2 | 1643 | damage specific DNA | 4.516 | 0.004 |
| | | binding protein 2 | | |
| DYNLL1 | 8655 | dynein light chain | 4.503 | <0.001 |
| | | LC8-type 1 | | |
| RRAS2 | 22800 | RAS related 2 | 4.486 | 0.006 |
| SMC2 | 10592 | structural maintenance | 4.463 | 0.002 |
| | | of chromosomes 2 | | |
| VAMP8 | 8673 | vesicle associated | 4.459 | 0.024 |
| | | membrane protein 8 | | |
| CDK2 | 1017 | cyclin dependent kinase 2 | 4.455 | 0.005 |
| HYPK | 25764 | huntingtin interacting | 4.434 | 0.004 |
| | | protein K | | |
| BPGM | 669 | bisphosphoglycerate | 4.434 | 0.005 |
| | | mutase | | |
| RBM38 | 55544 | RNA binding motif | 4.433 | 0.004 |
| | | protein 38 | | |
| AKR1C3 | 8644 | aldo-keto reductase | 4.415 | 0.014 |
| | | family 1 member C3 | | |
| MCM6 | 4175 | minichromosome | 4.394 | <0.001 |
| | | maintenance | | |
| | | complex component 6 | | |
| AUH | 549 | AU RNA binding | 4.352 | 0.003 |
| | | methylglutaconyl-CoA | | |
| | | hydratase | | |
| SAP30 | 8819 | Sin3A associated protein | 4.350 | 0.016 |
| | | 30 | | |
| EMC7 | 56851 | ER membrane protein | 4.338 | 0.037 |
| | | complex subunit 7 | | |
| NRBP1 | 29959 | nuclear receptor binding | 4.333 | 0.011 |
| | | protein 1 | | |
| ICOS | 29851 | inducible T cell | 4.331 | 0.001 |
| | | costimulator | | |
| HSPH1 | 10808 | heat shock protein family | 4.329 | 0.009 |
| | | H (Hsp110) member 1 | | |
| PPP1R8 | 5511 | protein phosphatase 1 | 4.321 | 0.042 |
| | | regulatory subunit 8 | | |
| TCAF2 | 285966 | TRPM8 channel | 4.305 | <0.001 |
| | | associated factor 2 | | |
| DAP3 | 7818 | death associated protein 3 | 4.303 | 0.002 |
| MRPS27 | 23107 | mitochondrial ribosomal | 4.293 | 0.003 |
| | | protein S27 | | |
| MRPS14 | 63931 | mitochondrial ribosomal | 4.291 | 0.023 |
| | | protein S14 | | |
| CTPS1 | 1503 | CTP synthase 1 | 4.290 | <0.001 |
| LAMP2 | 3920 | lysosomal associated | 4.288 | 0.011 |
| | | membrane protein 2 | | |
| ERI1 | 90459 | exoribonuclease 1 | 4.285 | 0.007 |
| RHEB | 6009 | Ras homolog, mTORC1 | 4.259 | 0.006 |
| | | binding | | |
| MAEA | 10296 | macrophage erythroblast | 4.259 | 0.007 |
| | | attacher | | |
| MRPL17 | 63875 | mitochondrial ribosomal | 4.252 | 0.002 |
| | | protein L17 | | |
| MRPL43 | 84545 | mitochondrial ribosomal | 4.246 | 0.030 |
| | | protein L43 | | |
| REXO2 | 25996 | RNA exonuclease 2 | 4.242 | <0.001 |
| DCTN3 | 11258 | dynactin subunit 3 | 4.232 | 0.001 |
| CASP3 | 836 | caspase 3 | 4.228 | 0.017 |
| APOL2 | 23780 | apolipoprotein L2 | 4.176 | 0.001 |
| ACSL4 | 2182 | acyl-CoA synthetase long | 4.164 | 0.029 |
| | | chain family member 4 | | |
| ERMP1 | 79956 | endoplasmic reticulum | 4.149 | 0.025 |
| | | metallopeptidase 1 | | |
| PPME1 | 51400 | protein phosphatase | 4.122 | 0.001 |
| | | methylesterase 1 | | |
| IWS1 | 55677 | IWS1, SUPT6H | 4.113 | 0.017 |
| | | interacting protein | | |
| BNIP1 | 662 | BCL2 interacting | 4.112 | 0.022 |
| | | protein 1 | | |
| PPID | 5481 | peptidylprolyl isomerase | 4.107 | <0.001 |
| | | D | | |
| MRPS2 | 51116 | mitochondrial ribosomal | 4.105 | 0.023 |
| | | protein S2 | | |
| MAIP1 | 79568 | matrix AAA peptidase | 4.099 | 0.004 |
| | | interacting protein 1 | | |
| RIOX2 | 84864 | ribosomal oxygenase 2 | 4.095 | 0.004 |
| BCL2L1 | 598 | BCL2 like 1 | 4.094 | 0.031 |
| ALDH3A2 | 224 | aldehyde dehydrogenase | 4.083 | 0.001 |
| | | 3 family member A2 | | |
| NAMPT | 10135 | nicotinamide | 4.070 | <0.001 |
| | | phosphoribosyltransferase | | |
| SEC63 | 11231 | SEC63 homolog, protein | 4.052 | 0.010 |
| | | translocation regulator | | |
| UBAP2L | 9898 | ubiquitin associated | 4.024 | <0.001 |
| | | protein 2 like | | |
| GCLM | 2730 | glutamate-cysteine ligase | 4.023 | <0.001 |
| | | modifier subunit | | |
| TMEM14C | 51522 | transmembrane protein | 4.016 | 0.013 |
| | | 14C | | |
| BCCIP | 56647 | BRCA2 and CDKN1A | 4.012 | <0.001 |
| | | interacting protein | | |
| LGMN | 5641 | legumain | 4.004 | 0.001 |
| RFC5 | 5985 | replication factor C | 4.000 | 0.006 |
| | | subunit 5 | | |
| TGFBI | 7045 | transforming growth | 3.982 | 0.003 |
| | | factor beta induced | | |
| APOD | 347 | apolipoprotein D | 3.982 | 0.007 |
| MAD2L1 | 4085 | mitotic arrest deficient 2 | 3.978 | 0.001 |
| | | like 1 | | |
| TXN | 7295 | thioredoxin | 3.977 | <0.001 |
| GGH | 8836 | gamma-glutamyl | 3.970 | 0.008 |
| | | hydrolase | | |
| HLA-DQA1 | 3117 | major histocompatibility | 3.944 | <0.001 |
| | | complex, class II, DQ | | |
| | | alpha l | | |
| EIF2B2 | 8892 | eukaryotic translation | 3.931 | 0.012 |
| | | initiation factor 2B | | |
| | | subunit beta | | |
| TRABD | 80305 | TraB domain containing | 3.922 | <0.001 |
| GGCT | 79017 | gamma- | 3.913 | 0.001 |
| | | glutamylcyclotransferase | | |
| MVD | 4597 | mevalonate diphosphate | 3.908 | 0.037 |
| | | decarboxylase | | |
| LRRC59 | 55379 | leucine rich repeat | 3.907 | <0.001 |
| | | containing 59 | | |
| TM9SF3 | 56889 | transmembrane 9 | 3.905 | 0.007 |
| | | superfamily member 3 | | |
| PTRH2 | 51651 | peptidyl-tRNA | 3.904 | <0.001 |
| | | hydrolase 2 | | |
| CUL4B | 8450 | cullin 4B | 3.896 | 0.034 |
| ACP2 | 53 | acid phosphatase 2, | 3.894 | 0.032 |
| | | lysosomal | | |
| SEC11C | 90701 | SEC11 homolog C, | 3.892 | 0.014 |
| | | signal peptidase complex | | |
| | | subunit | | |
| HPGD | 3248 | 15-hydroxyprostaglandin | 3.890 | 0.022 |
| | | dehydrogenase | | |
| ACOT8 | 10005 | acyl-CoA thioesterase 8 | 3.848 | 0.014 |
| CD82 | 3732 | CD82 molecule | 3.842 | 0.034 |
| L2HGDH | 79944 | L-2-hydroxyglutarate | 3.842 | 0.030 |
| | | dehydrogenase | | |
| HUWE1 | 10075 | HECT, UBA and WWE | 3.829 | 0.005 |
| | | domain containing 1, E3 | | |
| | | ubiquitin protein ligase | | |
| ARG2 | 384 | arginase 2 | 3.827 | 0.010 |
| SLC29A1 | 2030 | solute carrier family 29 | 3.825 | 0.048 |
| | | member 1 | | |
| | | (Augustine blood group) | | |
| SATB1 | 6304 | SATB homeobox 1 | 3.823 | <0.001 |
| FCHO1 | 23149 | FCH domain only 1 | 3.804 | 0.007 |
| MRPL4 | 51073 | mitochondrial ribosomal | 3.799 | 0.014 |
| | | protein L4 | | |
| CD28 | 940 | CD28 molecule | 3.789 | 0.009 |
| MRGBP | 55257 | MRG domain binding | 3.778 | 0.004 |
| | | protein | | |
| TMA16 | 55319 | translation machinery | 3.767 | 0.015 |
| | | associated 16 homolog | | |
| PPIF | 10105 | peptidylprolyl isomerase | 3.731 | 0.020 |
| | | F | | |
| SMS | 6611 | spermine synthase | 3.726 | 0.004 |
| PGP | 283871 | phosphoglycolate | 3.718 | 0.001 |
| | | phosphatase | | |
| WARS | 7453 | tryptophanyl-tRNA | 3.715 | <0.001 |
| | | synthetase | | |
| CPOX | 1371 | coproporphyrinogen | 3.711 | <0.001 |
| | | oxidase | | |
| SCPEP1 | 59342 | serine carboxypeptidase | 3.689 | 0.020 |
| | | 1 | | |
| MFSD10 | 10227 | major facilitator | 3.684 | 0.019 |
| | | superfamily domain | | |
| | | containing 10 | | |
| MCMBP | 79892 | minichromosome | 3.680 | 0.013 |
| | | maintenance complex | | |
| | | binding protein | | |
| GBE1 | 2632 | 1,4-alpha-glucan | 3.672 | 0.012 |
| | | branching enzyme 1 | | |
| RFC3 | 5983 | replication factor C | 3.663 | 0.012 |
| | | subunit 3 | | |
| TRUB1 | 142940 | TruB pseudouridine | 3.653 | 0.047 |
| | | synthase family | | |
| | | member 1 | | |
| BAG6 | 7917 | BCL2 associated | 3.651 | 0.026 |
| | | athanogene 6 | | |
| MRPL48 | 51642 | mitochondrial ribosomal | 3.649 | 0.007 |
| | | protein L48 | | |
| MRPS11 | 64963 | mitochondrial ribosomal | 3.623 | <0.001 |
| | | protein S11 | | |
| RSU1 | 6251 | Ras suppressor protein 1 | 3.606 | 0.001 |
| THOC6 | 79228 | THO complex 6 | 3.593 | 0.004 |
| GTF3C3 | 9330 | general transcription | 3.571 | 0.028 |
| | | factor IIIC subunit 3 | | |
| MRPL44 | 65080 | mitochondrial ribosomal | 3.569 | 0.003 |
| | | protein L44 | | |
| NMI | 9111 | N-myc and STAT | 3.565 | 0.008 |
| | | interactor | | |
| LIG1 | 3978 | DNA ligase 1 | 3.564 | 0.001 |
| RFC4 | 5984 | replication factor C | 3.546 | <0.001 |
| | | subunit 4 | | |
| MANF | 7873 | mesencephalic astrocyte | 3.543 | <0.001 |
| | | derived neurotrophic | | |
| | | factor | | |
| CELF1 | 10658 | CUGBP Elav-like family | 3.543 | 0.048 |
| | | member 1 | | |
| ACY1 | 95 | aminoacylase 1 | 3.530 | 0.003 |
| MRPS31 | 10240 | mitochondrial ribosomal | 3.520 | 0.039 |
| | | protein S31 | | |
| EIF4E2 | 9470 | eukaryotic translation | 3.502 | 0.013 |
| | | initiation factor | | |
| | | 4E family member 2 | | |
| POLD2 | 5425 | DNA polymerase delta 2, | 3.491 | 0.003 |
| | | accessory subunit | | |
| FASN | 2194 | fatty acid synthase | 3.483 | <0.001 |
| NADSYN1 | 55191 | NAD synthetase 1 | 3.462 | 0.010 |
| KPNA2 | 3838 | karyopherin subunit | 3.456 | 0.006 |
| | | alpha 2 | | |
| RNASEH2A | 10535 | ribonuclease H2 | 3.447 | 0.037 |
| | | subunit A | | |
| HAT1 | 8520 | histone | 3.441 | <0.001 |
| | | acetyltransferase 1 | | |
| STAT1 | 6772 | signal transducer | 3.425 | <0.001 |
| | | and activator of | | |
| | | transcription 1 | | |
| UAP1L1 | 91373 | UDP-N- | 3.401 | 0.007 |
| | | acetylglucosamine | | |
| | | pyrophosphorylase 1 | | |
| | | like 1 | | |
| PYCR2 | 29920 | pyrroline-5-carboxylate | 3.399 | 0.021 |
| | | reductase 2 | | |
| PLEKHA2 | 59339 | pleckstrin homology | 3.397 | 0.020 |
| | | domain containing A2 | | |
| NCF4 | 4689 | neutrophil cytosolic | 3.388 | <0.001 |
| | | factor 4 | | |
| RNF213 | 57674 | ring finger protein 213 | 3.383 | <0.001 |
| MAN1A1 | 4121 | mannosidase alpha | 3.356 | 0.018 |
| | | class 1A member 1 | | |
| POFUT1 | 23509 | protein | 3.347 | 0.001 |
| | | O-fucosyltransferase 1 | | |
| CSDE1 | 7812 | cold shock domain | 3.347 | 0.025 |
| | | containing E1 | | |
| IDE | 3416 | insulin degrading | 3.324 | 0.003 |
| | | enzyme | | |
| HELLS | 3070 | helicase, lymphoid | 3.317 | 0.037 |
| | | specific | | |
| ATXN2L | 11273 | ataxin 2 like | 3.315 | 0.004 |
| CALU | 813 | calumenin | 3.310 | 0.048 |
| KTN1 | 3895 | kinectin 1 | 3.306 | <0.001 |
| FAS | 355 | Fas cell surface death | 3.302 | <0.001 |
| | | receptor | | |
| TBCD | 6904 | tubulin folding cofactor | 3.300 | 0.016 |
| | | D | | |
| JPT1 | 51155 | Jupiter microtubule | 3.295 | 0.004 |
| | | associated homolog 1 | | |
| OAT | 4942 | ornithine aminotransferase | 3.292 | 0.010 |
| BRK1 | 55845 | BRICK1, SCAR/WAVE | 3.292 | 0.022 |
| | | actin nucleating complex | | |
| | | subunit | | |
| TAOK3 | 51347 | TAO kinase 3 | 3.285 | 0.026 |
| MSI2 | 124540 | musashi RNA binding | 3.285 | 0.030 |
| | | protein 2 | | |
| VPS28 | 51160 | VPS28, ESCRT-I subunit | 3.279 | 0.041 |
| MRPL12 | 6182 | mitochondrial ribosomal | 3.274 | 0.009 |
| | | protein L12 | | |
| LACTB2 | 51110 | lactamase beta 2 | 3.255 | 0.012 |
| SFXN2 | 118980 | sideroflexin 2 | 3.240 | 0.032 |
| FAM160B1 | 57700 | family with sequence | 3.240 | 0.001 |
| | | similarity 160 member B1 | | |
| EXOSC5 | 56915 | exosome component 5 | 3.237 | 0.011 |
| HMGB3 | 3149 | high mobility group box 3 | 3.236 | 0.002 |
| ZC2HC1A | 51101 | zinc finger C2HC-type | 3.233 | 0.008 |
| | | containing 1A | | |
| PLSCR3 | 57048 | phospholipid scramblase 3 | 3.226 | 0.005 |
| DTD1 | 92675 | D-tyrosyl-tRNA | 3.198 | 0.034 |
| | | deacylase 1 | | |
| NAPG | 8774 | NSF attachment protein | 3.185 | <0.001 |
| | | gamma | | |
| LSM1 | 27257 | LSM1 homolog, mRNA | 3.172 | 0.018 |
| | | degradation associated | | |
| TIMM13 | 26517 | translocase of inner | 3.159 | 0.017 |
| | | mitochondrial membrane | | |
| | | 13 | | |
| S1PR4 | 8698 | sphingosine-1-phosphate | 3.145 | 0.022 |
| | | receptor 4 | | |
| IPO9 | 55705 | importin 9 | 3.122 | 0.029 |
| MFSD1 | 64747 | major facilitator | 3.077 | 0.027 |
| | | superfamily domain | | |
| | | containing 1 | | |
| MSH2 | 4436 | mutS homolog 2 | 3.075 | 0.020 |
| CPT1A | 1374 | carnitine | 3.069 | <0.001 |
| | | palmitoyltransferase 1A | | |
| FAM192A | 80011 | family with sequence | 3.055 | 0.023 |
| | | similarity 192 member A | | |
| AP3D1 | 8943 | adaptor related protein | 3.047 | 0.037 |
| | | complex 3 subunit delta 1 | | |
| RCL1 | 10171 | RNA terminal phosphate | 3.042 | 0.019 |
| | | cyclase like 1 | | |
| PTGES3L- | 1E+08 | PTGES3L-AARSD1 | 3.038 | 0.023 |
| AARSD1 | | readthrough | | |
| GBP5 | 115362 | guanylate binding | 3.034 | <0.001 |
| | | protein 5 | | |
| MRPL13 | 28998 | mitochondrial ribosomal | 3.030 | <0.001 |
| | | protein L13 | | |
| NPM3 | 10360 | nucleophosmin/ | 3.030 | 0.019 |
| | | nucleoplasmin 3 | | |
| PPP2R5D | 5528 | protein phosphatase 2 | 3.022 | 0.003 |
| | | regulatory subunit | | |
| | | B′delta | | |
| CYB5B | 80777 | cytochrome b5 type B | 3.021 | 0.043 |
| MRPS35 | 60488 | mitochondrial ribosomal | 3.003 | 0.040 |
| | | protein S35 | | |
| POLD1 | 5424 | DNA polymerase delta | 2.998 | 0.001 |
| | | 1, catalytic subunit | | |
| AGMAT | 79814 | agmatinase | 2.994 | 0.007 |
| PTDSS1 | 9791 | phosphatidylserine | 2.977 | 0.036 |
| | | synthase 1 | | |
| IPO7 | 10527 | importin 7 | 2.974 | 0.014 |
| ARL3 | 403 | ADP ribosylation factor | 2.973 | <0.001 |
| | | like GTPase 3 | | |
| MRPS9 | 64965 | mitochondrial ribosomal | 2.970 | 0.033 |
| | | protein S9 | | |
| PBXIP1 | 57326 | PBX homeobox | 2.957 | 0.001 |
| | | interacting protein 1 | | |
| SLC16A3 | 9123 | solute carrier family 16 | 2.954 | 0.000 |
| | | member 3 | | |
| EIF2B3 | 8891 | eukaryotic translation | 2.954 | 0.022 |
| | | initiation factor 2B | | |
| | | subunit gamma | | |
| NUDT1 | 4521 | nudix hydrolase 1 | 2.947 | <0.001 |
| WDR61 | 80349 | WD repeat domain 61 | 2.944 | 0.041 |
| MPST | 4357 | mercaptopyruvate | 2.938 | <0.001 |
| | | sulfurtransferase | | |
| ASF1A | 25842 | anti-silencing function | 2.937 | 0.001 |
| | | 1A histone chaperone | | |
| HTRA2 | 27429 | HtrA serine peptidase 2 | 2.929 | 0.017 |
| SLC2A3 | 6515 | solute carrier family 2 | 2.923 | 0.042 |
| | | member 3 | | |
| HSPB1 | 3315 | heat shock protein | 2.921 | 0.004 |
| | | family B (small) | | |
| | | member 1 | | |
| LPXN | 9404 | leupaxin | 2.904 | 0.001 |
| GLRX3 | 10539 | glutaredoxin 3 | 2.885 | <0.001 |
| GCLC | 2729 | glutamate-cysteine ligase | 2.881 | 0.009 |
| | | catalytic subunit | | |
| TF | 7018 | transferrin | 2.873 | 0.001 |
| CARM1 | 10498 | coactivator associated | 2.872 | 0.005 |
| | | arginine | | |
| | | methyltransferase 1 | | |
| RNASEH2B | 79621 | ribonuclease H2 subunit | 2.858 | <0.001 |
| | | B | | |
| AIMP2 | 7965 | aminoacyl tRNA | 2.852 | 0.003 |
| | | synthetase complex | | |
| | | interacting | | |
| | | multifunctional protein 2 | | |
| TOMM40 | 10452 | translocase of outer | 2.848 | 0.005 |
| | | mitochondrial membrane | | |
| | | 40 | | |
| EMC2 | 9694 | ER membrane protein | 2.828 | 0.013 |
| | | complex subunit 2 | | |
| CD2 | 914 | CD2 molecule | 2.826 | 0.001 |
| UROD | 7389 | uroporphyrinogen | 2.825 | 0.007 |
| | | decarboxylase | | |
| ADAM10 | 102 | ADAM metallopeptidase | 2.816 | 0.012 |
| | | domain 10 | | |
| HTATIP2 | 10553 | HIV-1 Tat interactive | 2.804 | <0.001 |
| | | protein 2 | | |
| MTX1 | 4580 | metaxin 1 | 2.803 | 0.014 |
| RPL7L1 | 285855 | ribosomal protein L7 | 2.779 | 0.038 |
| | | like 1 | | |
| ERBIN | 55914 | erbb2 interacting protein | 2.778 | 0.001 |
| CASP6 | 839 | caspase 6 | 2.778 | 0.001 |
| MRPL37 | 51253 | mitochondrial ribosomal | 2.762 | 0.002 |
| | | protein L37 | | |
| EIF4G1 | 1981 | eukaryotic translation | 2.759 | 0.004 |
| | | initiation factor 4 | | |
| | | gamma 1 | | |
| CACYBP | 27101 | calcyclin binding protein | 2.754 | <0.001 |
| DNAJB11 | 51726 | DnaJ heat shock protein | 2.750 | 0.001 |
| | | family (Hsp40) member | | |
| | | B11 | | |
| TRAF1 | 7185 | TNF receptor associated | 2.735 | 0.017 |
| | | factor 1 | | |
| RRP1B | 23076 | ribosomal RNA | 2.734 | 0.023 |
| | | processing 1B | | |
| RFC2 | 5982 | replication factor C | 2.731 | 0.001 |
| | | subunit 2 | | |
| PPP2R5C | 5527 | protein phosphatase 2 | 2.696 | 0.004 |
| | | regulatory subunit | | |
| | | B′gamma | | |
| RAB1A | 5861 | RAB1A, member RAS | 2.680 | 0.001 |
| | | oncogene family | | |
| IFI35 | 3430 | interferon induced | 2.675 | 0.002 |
| | | protein 35 | | |
| RFTN1 | 23180 | raftlin, lipid raft linker 1 | 2.675 | 0.038 |
| RMND1 | 55005 | required for meiotic | 2.669 | 0.019 |
| | | nuclear division 1 | | |
| | | homolog | | |
| RRM1 | 6240 | ribonucleotide reductase | 2.657 | 0.010 |
| | | catalytic subunit M1 | | |
| TBL1XR1 | 79718 | transducin beta like 1 | 2.649 | 0.017 |
| | | X-linked receptor 1 | | |
| RAP2B | 5912 | RAP2B, member of RAS | 2.647 | 0.017 |
| | | oncogene family | | |
| CORO1C | 23603 | coronin 1C | 2.643 | 0.003 |
| DPP4 | 1803 | dipeptidyl peptidase 4 | 2.628 | 0.001 |
| CD74 | 972 | CD74 molecule | 2.625 | <0.001 |
| PCNA | 5111 | proliferating cell nuclear | 2.615 | <0.001 |
| | | antigen | | |
| TXNDC5 | 81567 | thioredoxin domain | 2.612 | 0.001 |
| | | containing 5 | | |
| MRPL39 | 54148 | mitochondrial ribosomal | 2.610 | 0.038 |
| | | protein L39 | | |
| B4GALT5 | 9334 | beta-1,4- | 2.582 | 0.028 |
| | | galactosyltransferase 5 | | |
| FIBP | 9158 | FGF1 intracellular | 2.575 | 0.003 |
| | | binding protein | | |
| ACADVL | 37 | acyl-CoA dehydrogenase | 2.567 | <0.001 |
| | | very long chain | | |
| POLDIP3 | 84271 | DNA polymerase delta | 2.561 | 0.024 |
| | | interacting protein 3 | | |
| LGALS3 | 3958 | galectin 3 | 2.523 | <0.001 |
| KIF5B | 3799 | kinesin family member | 2.519 | <0.001 |
| | | 5B | | |
| ACAA1 | 30 | acetyl-CoA | 2.519 | 0.006 |
| | | acyltransferase 1 | | |
| ATXN10 | 25814 | ataxin 10 | 2.517 | 0.003 |
| GMDS | 2762 | GDP-mannose 4,6- | 2.514 | 0.001 |
| | | dehydratase | | |
| ATP13A1 | 57130 | ATPase 13A1 | 2.506 | 0.042 |
| RBPJ | 3516 | recombination signal | 2.481 | 0.003 |
| | | binding protein for | | |
| | | immunoglobulin kappa J | | |
| | | region | | |
| ECI1 | 1632 | enoyl-CoA delta | 2.477 | 0.012 |
| | | isomerase 1 | | |
| ACSL5 | 51703 | acyl-CoA synthetase long | 2.457 | 0.003 |
| | | chain family member 5 | | |
| BCLAF1 | 9774 | BCL2 associated | 2.455 | 0.032 |
| | | transcription factor 1 | | |
| EIF1 | 10209 | eukaryotic translation | 2.454 | 0.037 |
| | | initiation factor 1 | | |
| DUT | 1854 | deoxyuridine | 2.436 | <0.001 |
| | | triphosphatase | | |
| STMN1 | 3925 | stathmin 1 | 2.411 | <0.001 |
| MTHFD1 | 4522 | methylenetetrahydrofolate | 2.410 | <0.001 |
| | | dehydrogenase, | | |
| | | cyclohydrolase and | | |
| | | formyltetrahydrofolate | | |
| | | synthetase l | | |
| ADPRH | 141 | ADP-ribosylarginine | 2.387 | <0.001 |
| | | hydrolase | | |
| CD84 | 8832 | CD84 molecule | 2.379 | 0.043 |
| TRMT112 | 51504 | tRNA methyltransferase | 2.378 | 0.031 |
| | | subunit 11-2 | | |
| BYSL | 705 | bystin like | 2.375 | 0.040 |
| SNAP23 | 8773 | synaptosome associated | 2.371 | 0.013 |
| | | protein 23 | | |
| ICMT | 23463 | isoprenylcysteine | 2.357 | 0.010 |
| | | carboxyl | | |
| | | methyltransferase | | |
| ZC3H12D | 340152 | zinc finger CCCH-type | 2.357 | 0.007 |
| | | containing 12D | | |
| IPO5 | 3843 | importin 5 | 2.350 | <0.001 |
| ACSL1 | 2180 | acyl-CoA synthetase long | 2.348 | 0.002 |
| | | chain family member 1 | | |
| TUBAL3 | 79861 | tubulin alpha like 3 | 2.336 | <0.001 |
| GET4 | 51608 | golgi to ER traffic | 2.336 | 0.033 |
| | | protein 4 | | |
| LCP2 | 3937 | lymphocyte cytosolic | 2.314 | 0.040 |
| | | protein 2 | | |
| TUBG1 | 7283 | tubulin gamma 1 | 2.304 | 0.001 |
| MRI1 | 84245 | methylthioribose-1- | 2.300 | 0.001 |
| | | phosphate isomerase 1 | | |
| TXNRD1 | 7296 | thioredoxin reductase 1 | 2.299 | 0.000 |
| SERPINH1 | 871 | serpin family H | 2.288 | 0.042 |
| | | member 1 | | |
| TTC1 | 7265 | tetratricopeptide repeat | 2.287 | 0.021 |
| | | domain 1 | | |
| ERG28 | 11161 | ergosterol biosynthesis | 2.280 | 0.042 |
| | | 28 homolog | | |
| LIMS1 | 3987 | LIM zinc finger domain | 2.270 | 0.003 |
| | | containing 1 | | |
| ARL2 | 402 | ADP ribosylation factor | 2.250 | 0.020 |
| | | like GTPase 2 | | |
| YBX1 | 4904 | Y-box binding protein 1 | 2.249 | 0.013 |
| FEN1 | 2237 | flap structure-specific | 2.245 | 0.000 |
| | | endonuclease 1 | | |
| NCSTN | 23385 | nicastrin | 2.234 | 0.001 |
| AGK | 55750 | acylglycerol kinase | 2.232 | 0.001 |
| RNMT | 8731 | RNA guanine-7 | 2.230 | 0.002 |
| | | methyltransferase | | |
| OTULIN | 90268 | OTU deubiquitinase with | 2.226 | 0.007 |
| | | linear linkage specificity | | |
| NDUFA8 | 4702 | NADH:ubiquinone | 2.224 | 0.004 |
| | | oxidoreductase subunit | | |
| | | A8 | | |
| TUBA1B | 10376 | tubulin alpha 1b | 2.224 | 0.001 |
| RMDN1 | 51115 | regulator of microtubule | 2.219 | <0.001 |
| | | dynamics 1 | | |
| ACAA2 | 10449 | acetyl-CoA | 2.217 | <0.001 |
| | | acyltransferase 2 | | |
| TMCO1 | 54499 | transmembrane and | 2.204 | <0.001 |
| | | coiled-coil domains 1 | | |
| LRPPRC | 10128 | leucine rich | 2.193 | <0.001 |
| | | pentatricopeptide repeat | | |
| | | containing | | |
| DTYMK | 1841 | deoxythymidylate kinase | 2.186 | <0.001 |
| FDXR | 2232 | ferredoxin reductase | 2.169 | 0.006 |
| IGBP1 | 3476 | immunoglobulin binding | 2.169 | 0.001 |
| | | protein 1 | | |
| FAHD2A | 51011 | fumarylacetoacetate | 2.146 | 0.029 |
| | | hydrolase domain | | |
| | | containing 2A | | |
| HTATSF1 | 27336 | HIV-1 Tat specific | 2.143 | 0.001 |
| | | factor 1 | | |
| GRSF1 | 2926 | G-rich RNA sequence | 2.142 | 0.031 |
| | | binding factor 1 | | |
| DNMIL | 10059 | dynamin 1 like | 2.141 | 0.010 |
| AP3M1 | 26985 | adaptor related protein | 2.133 | 0.005 |
| | | complex 3 subunit mu 1 | | |
| UBE2L6 | 9246 | ubiquitin conjugating | 2.129 | 0.001 |
| | | enzyme E2 L6 | | |
| CISD2 | 493856 | CDGSH iron sulfur | 2.128 | 0.002 |
| | | domain 2 | | |
| HSPBP1 | 23640 | HSPA (Hsp70) binding | 2.127 | 0.001 |
| | | protein 1 | | |
| MRPL1 | 65008 | mitochondrial ribosomal | 2.126 | <0.001 |
| | | protein L1 | | |
| PMPCA | 23203 | peptidase, mitochondrial | 2.125 | 0.025 |
| | | processing alpha subunit | | |
| ACADM | 34 | acyl-CoA dehydrogenase | 2.116 | 0.001 |
| | | medium chain | | |
| BDH1 | 622 | 3-hydroxybutyrate | 2.108 | 0.002 |
| | | dehydrogenase 1 | | |
| DCAF8 | 50717 | DDB1 and CUL4 | 2.108 | 0.018 |
| | | associated factor 8 | | |
| STRAP | 11171 | serine/threonine kinase | 2.108 | <0.001 |
| | | receptor associated | | |
| | | protein | | |
| TIMM23 | 1E+08 | translocase of inner | 2.107 | 0.027 |
| | | mitochondrial membrane | | |
| | | 23 | | |
| FKBP3 | 2287 | FKBP prolyl isomerase 3 | 2.105 | <0.001 |
| SEC61A1 | 29927 | Sec61 translocon alpha 1 | 2.097 | 0.012 |
| | | subunit | | |
| TM9SF2 | 9375 | transmembrane 9 | 2.096 | 0.011 |
| | | superfamily member 2 | | |
| TMEM65 | 157378 | transmembrane protein | 2.089 | 0.002 |
| | | 65 | | |
| ENOPH1 | 58478 | enolase-phosphatase 1 | 2.087 | 0.026 |
| CPT2 | 1376 | carnitine | 2.079 | 0.019 |
| | | palmitoyltransferase 2 | | |
| CIAPIN1 | 57019 | cytokine induced | 2.074 | 0.003 |
| | | apoptosis inhibitor 1 | | |
| IDI1 | 3422 | isopentenyl-diphosphate | 2.065 | <0.001 |
| | | delta isomerase 1 | | |
| POLDIP2 | 26073 | DNA polymerase delta | 2.050 | 0.011 |
| | | interacting protein 2 | | |
| SUMF2 | 25870 | sulfatase modifying | 2.046 | <0.001 |
| | | factor 2 | | |
| NUDC | 10726 | nuclear distribution C, | 2.046 | <0.001 |
| | | dynein complex regulator | | |
| PKN1 | 5585 | protein kinase N1 | 2.044 | 0.013 |
| NAA50 | 80218 | N(alpha)- | 2.037 | 0.010 |
| | | acetyltransferase 50, | | |
| | | NatE catalytic subunit | | |
| CHDH | 55349 | choline dehydrogenase | 2.029 | 0.029 |
| PPP4R3A | 55671 | protein phosphatase 4 | 2.025 | 0.022 |
| | | regulatory subunit 3 A | | |
| KRR1 | 11103 | KRR1, small subunit | 2.023 | 0.020 |
| | | processome component | | |
| | | homolog | | |
| TOMM22 | 56993 | translocase of outer | 2.022 | 0.002 |
| | | mitochondrial membrane | | |
| | | 22 | | |
| FKBP8 | 23770 | FKBP prolyl isomerase 8 | 2.014 | 0.001 |
| LGALS1 | 3956 | galectin 1 | 2.014 | <0.001 |
| AIMP1 | 9255 | aminoacyl tRNA | 2.012 | 0.002 |
| | | synthetase complex | | |
| | | interacting multifunctional | | |
| | | protein 1 | | |

### 8.8.4.3 Enhanced Treg Proteomic Signatures Following Expansion

The phenotypic analysis reveals that the expanded Treg gene product signature includes gene products from prominent pathways that are associated with functional processes, specifically pathways enriched in Treg immune signatures, mitochondria activation and energetics, and cellular proliferation including cell division, cell cycle, and DNA replication/repair. The proteomics data has also been stratified to present the highest expression signatures found in the patient Tregs following expansion. Each of these gene product signatures of the expandd Treg cell population is described below.

### 8.8.4.3.1 Treg-Associated Gene Product Signature

The proteomic analysis reveals a number of gene products involved in immunological pathways that are enriched in the expanded Treg cell populations, as evidenced by their increased expression relative to that observed in baseline Tregs. These pathways include, for example, adaptive immune pathways (p=0.00726), innate immune pathways (p=0.09), cytokine signaling in the immune system (p=0.0338), MHC class II antigen presentation (p=9.33e-13), PD-1 signaling (p=7.66e-11), costimulation by the CD28 family (p=9.12e-11), generation of second messenger molecules (p=7.69e-13), Interferon signaling (p=1.31e-7), downstream TCR signaling (p=1.31e-7), and RUNX1 and FOXP3 control of the development of regulatory T lymphocytes (p=1.05e-3). Table 15 (Treg-associated gene product signature) lists gene products whose expression is increased relative to baseline Tregs, wherein the gene products are documented in the literature as being important to the proliferation, health, identification, and/or mechanism of Treg cells.

As shown in Table 15, the Treg-associated gene product signature includes, for example: ADAM10, AIMP1, AIMP2, ARG2, BCL2L1, BSG, CD2, CD28, CD38, CD74, CD84, CTLA4, FAS, FOXP3, GCLC, HAT1, HLA-DQA1, HLA-DQB1, HLA-DRA, HLA-DRB1, NPGD, ICOS, IL1RN, IRF4, KPNA2, LGALS7, LGMN, PCNA, POFUT1, SATB1, SELPLG, STAT1, TFRC, and TNFRSF18. PMTD: Pubmed ID.

TABLE 15
Gene products in expanded Tregs that are documented in the literature as being
important to the proliferation, health, identification, and/or mechanism of Treg cells.

| | | | | log2 of | | log2 of | |
|---|---|---|---|---|---|---|---|
| | | | PMID | fold-change | | fold-change | |
| Gene | NCBI | | cross | of baseline | Adjusted | of baseline | Adjusted |
| Symbol | GENE ID | Gene Description | reference | vs. expanded | p-value | vs. freeze-thaw | p-value |
| ILIRN | 3557 | interleukin 1 receptor | 24770649 | 7.715 | <0.001 | 0.218 | 0.911 |
| | | antagonist | | | | | |
| TFRC | 7037 | transferrin receptor | 29311383 | 7.114 | <0.001 | 0.261 | 0.902 |
| HLA-DQB1 | 3119 | major histocompatibility | 16585553 | 6.779 | 0.001 | 0.367 | 0.942 |
| | | complex, class II, DQ beta 1 | | | | | |
| CD38 | 952 | CD38 molecule | 28249894 | 6.393 | 0.007 | −0.897 | 0.873 |
| HLA-DRB1 | 3123 | major histocompatibility | 16585553 | 5.489 | <0.001 | 0.347 | 0.685 |
| | | complex, class II, DR beta 1 | | | | | |
| SELPLG | 6404 | selectin P ligand | 24174617 | 5.317 | 0.019 | −1.004 | 0.848 |
| CTLA4 | 1493 | cytotoxic T-lymphocyte | 23849743 | 4.993 | 0.002 | −0.610 | 0.856 |
| | | associated protein 4 | | | | | |
| TNFRSF18 | 8784 | TNF receptor superfamily | 25961057 | 4.896 | 0.001 | −0.185 | 0.964 |
| | | member 18 | | | | | |
| IRF4 | 3662 | interferon regulatory | 32125291 | 4.896 | 0.003 | 0.478 | 0.907 |
| | | factor 4 | | | | | |
| HLA-DRA | 3122 | major histocompatibility | 16585553 | 4.722 | 0.000 | 0.353 | 0.793 |
| | | complex, class II, DR alpha | | | | | |
| BSG | 682 | basigin (Ok blood group) | 21937704 | 4.721 | 0.001 | 0.042 | 0.993 |
| FOXP3 | 50943 | forkhead box P3 | 25683611 | 4.589 | 0.001 | 0.522 | 0.946 |
| ICOS | 29851 | inducible T cell | 32983168 | 4.331 | 0.001 | 0.629 | 0.798 |
| | | costimulator | | | | | |
| BCL2L1 | 598 | BCL2 like 1 | 31068951 | 4.094 | 0.031 | −0.344 | 0.954 |
| LGMN | 5641 | legumain | 19453521 | 4.004 | 0.001 | 0.528 | 0.819 |
| HLA-DQA1 | 3117 | major histocompatibility | 16585553 | 3.944 | <0.001 | 0.215 | 0.932 |
| | | complex, class II, DQ | | | | | |
| | | alpha 1 | | | | | |
| HPGD | 3248 | 15-hydroxyprostaglandin | 31027998 | 3.890 | 0.022 | −0.067 | 0.993 |
| | | dehydrogenase | | | | | |
| ARG2 | 384 | arginase 2 | 31852848 | 3.827 | 0.010 | 1.012 | 0.722 |
| SATBI | 6304 | SATB homeobox 1 | 27992401 | 3.823 | <0.001 | 0.567 | 0.739 |
| CD28 | 940 | CD28 molecule | 18684917 | 3.789 | 0.009 | 0.770 | 0.805 |
| KPNA2 | 3838 | karyopherin subunit alpha 2 | 31597697 | 3.456 | 0.006 | 0.657 | 0.804 |
| HAT1 | 8520 | histone acetyltransferase 1 | 24315995 | 3.441 | <0.001 | −0.868 | 0.279 |
| STAT1 | 6772 | signal transducer and | 19337996 | 3.425 | <0.001 | −0.104 | 0.882 |
| | | activator of transcription 1 | | | | | |
| POFUT1 | 23509 | protein O-fucosyltransferase 1 | 26437242 | 3.347 | 0.001 | 0.941 | 0.543 |
| FAS | 355 | Fas cell surface death | 32294156 | 3.302 | <0.001 | 0.368 | 0.543 |
| | | receptor | | | | | |
| GCLC | 2729 | glutamate-cysteine ligase | 32213345 | 2.881 | 0.009 | −1.135 | 0.560 |
| | | catalytic subunit | | | | | |
| AIMP2 | 7965 | aminoacyl tRNA synthetase | 32709848 | 2.852 | 0.003 | −0.485 | 0.805 |
| | | complex interacting | | | | | |
| | | multifunctional protein 2 | | | | | |
| CD2 | 914 | CD2 molecule | 22539784 | 2.826 | 0.001 | 1.083 | 0.345 |
| ADAM10 | 102 | ADAM metallopeptidase | 31269441 | 2.816 | 0.012 | 0.725 | 0.743 |
| | | domain 10 | | | | | |
| CD74 | 972 | CD74 molecule | 27760760 | 2.625 | <0.001 | 0.366 | 0.357 |
| PCNA | 5111 | proliferating cell nuclear | 29166588 | 2.615 | <0.001 | 0.183 | 0.655 |
| | | antigen | | | | | |
| CD84 | 8832 | CD84 molecule | 26371251 | 2.379 | 0.043 | 0.538 | 0.848 |
| LGALS1 | 3956 | galectin 1 | 16836768 | 2.014 | <0.001 | 0.096 | 0.877 |
| AIMP1 | 9255 | aminoacyl tRNA synthetase | 31084930 | 2.012 | 0.002 | 0.123 | 0.946 |
| | | complex interacting | | | | | |
| | | multifunctional protein 1 | | | | | |

### 8.8.4.3.2 Mitochondria Gene Product Signature

Mitochondria play a large role in Treg health and function. The proteomic study revealed a large, enriched gene product signature of mitochondria-related genes in the ex vivo-expanded Treg cell populations whose expression is increased relative to that seen in baseline Tregs (p=2.96e-29). See Table 16. The literature describes the importance of mitochondrial fitness and energetics in Treg function and mitochondrial dysfunction inevitably leads to Treg dysfunction (ex. PMID: 30320604). Targeting and restoring mitochondrial function is currently looked at as a way to revive dysfunctional Tregs (PMID: 30473188). This mitochondria gene product signature is, for example, highly enriched with pathways involved in mitochondria replication (p=1.12e-14) and mitochondrial energy metabolism (p=1.83e-2). This gene product signature indicates that mitochondrial activation, function, and restoration is an important product of the Treg expansion processes described herein.

As shown in Table 16, the mitochondria gene product signature includes, for example: ACAA2, ACADM, ACADVL, ACOT7, ACSL1, ACSL4, ACSL5, AGK, AGMAT, AK4, ARG2, ARL2, AUH, BCL2L1, BDH1, BNIP1, CDK1, CHIDH, CIAPIN1, CISD2, COX17, CPOX, CPT1A, CPT2, CYB5B, DAP3, DHIRS2, DNM1L, DUT, DYNLL1, ECI1, FDXR, FEN1, FKBP8, GK, GRSF1, HTRA2, L2HGDH, LACTB2, LRPPRC, MAIP1, MAOA, MIPST, MRPL1, MIRPL12, MIRPL13, MIRPL14, MIRPL17, MRPL22, MRPL37, MIRPL39, MRPL4, MRPL43, MRPL44, MIRPL46, MIRPL48, MVIRPS11, MRPS14, MVIRPS2, MIRPS27, MRPS31, MIRPS35, MRPS9, MTHFD2, MTX1, MYCBP, NDUFA8, NUJDT1, OAT, PITRM1, PLSCR3, PMIPCA, PPIF, PTRH2, PYCR2, REXO2, RM1VND1, SFXN2, SLC25A10, SLC25A19, SLC25A4, TIGAR, TIMM13, TTMM23, TMEM14C, TOMM22, TOMM34, TOMM40, and TST.

TABLE 16
Enriched signature of mitochondria-related genes products

| | | | log2 of | | log2 of | |
|---|---|---|---|---|---|---|
| | | | fold-change | | fold-change | |
| Gene | NCBI | | of baseline | Adjusted | of baseline | Adjusted |
| Symbol | GENE ID | Gene Description | vs. expanded | p-value | vs. freeze-thaw | p-value |
| MRPL46 | 26589 | mitochondrial ribosomal protein L46 | 6.991 | 0.006 | −0.041 | 0.997 |
| COX17 | 10063 | cytochrome c oxidase copper | 6.680 | 0.002 | 0.654 | 0.897 |
| | | chaperone COX 17 | | | | |
| CDK1 | 983 | cyclin dependent kinase 1 | 6.615 | 0.007 | 1.621 | 0.502 |
| PITRM1 | 10531 | pitrilysin metallopeptidase 1 | 6.046 | <0.001 | 0.611 | 0.805 |
| DHRS2 | 10202 | dehydrogenase/reductase 2 | 5.867 | 0.001 | 0.562 | 0.893 |
| TOMM34 | 10953 | translocase of outer mitochondrial | 5.836 | 0.007 | −0.161 | 0.984 |
| | | membrane 34 | | | | |
| SLC25A10 | 1468 | solute carrier family 25 member 10 | 5.776 | <0.001 | 0.960 | 0.568 |
| GK | 2710 | glycerol kinase | 5.635 | <0.001 | 0.636 | 0.826 |
| MRPL22 | 29093 | mitochondrial ribosomal protein L22 | 5.268 | 0.007 | 0.605 | 0.904 |
| TST | 7263 | thiosulfate sulfurtransferase | 5.170 | <0.001 | 1.050 | 0.566 |
| SLC25A4 | 291 | solute carrier family 25 member 4 | 5.089 | 0.030 | 0.730 | 0.903 |
| MYCBP | 26292 | MYC binding protein | 4.922 | 0.005 | −1.834 | 0.526 |
| MTHFD2 | 10797 | methylenetetrahydrofolate | 4.894 | 0.022 | 0.301 | 0.969 |
| | | dehydrogenase (NADP+ | | | | |
| | | dependent) 2, | | | | |
| | | methenyltetrahydrofolate | | | | |
| | | cyclohydrolase | | | | |
| TIGAR | 57103 | TP53 induced glycolysis regulatory | 4.808 | <0.001 | −0.569 | 0.819 |
| | | phosphatase | | | | |
| MRPL14 | 64928 | mitochondrial ribosomal protein L14 | 4.773 | 0.029 | −0.961 | 0.855 |
| MAOA | 4128 | monoamine oxidase A | 4.637 | 0.002 | 0.210 | 0.960 |
| SLC25A19 | 60386 | solute carrier family 25 member 19 | 4.627 | 0.001 | 0.924 | 0.703 |
| ACOT7 | 11332 | acyl-CoA thioesterase 7 | 4.566 | <0.001 | −0.075 | 0.973 |
| DYNLL1 | 8655 | dynein light chain LC8-type 1 | 4.503 | <0.001 | 0.610 | 0.712 |
| AUH | 549 | AU RNA binding | 4.352 | 0.003 | 0.422 | 0.908 |
| | | methylglutaconyl-CoA hydratase | | | | |
| DAP3 | 7818 | death associated protein 3 | 4.303 | 0.002 | 1.091 | 0.655 |
| MRPS27 | 23107 | mitochondrial ribosomal protein S27 | 4.293 | 0.003 | 0.400 | 0.908 |
| MRPS14 | 63931 | mitochondrial ribosomal protein S14 | 4.291 | 0.023 | 1.703 | 0.645 |
| MRPL17 | 63875 | mitochondrial ribosomal protein L17 | 4.252 | 0.002 | 0.756 | 0.769 |
| MRPL43 | 84545 | mitochondrial ribosomal protein L43 | 4.246 | 0.030 | 0.905 | 0.847 |
| REXO2 | 25996 | RNA exonuclease 2 | 4.242 | 0.000 | 0.213 | 0.940 |
| ACSL4 | 2182 | acyl-CoA synthetase long chain | 4.164 | 0.029 | 0.596 | 0.911 |
| | | family member 4 | | | | |
| BNIP1 | 662 | BCL2 interacting protein 1 | 4.112 | 0.022 | −0.296 | 0.960 |
| MRPS2 | 51116 | mitochondrial ribosomal protein S2 | 4.105 | 0.023 | 0.593 | 0.904 |
| MAIP1 | 79568 | matrix AAA peptidase interacting | 4.099 | 0.004 | −0.022 | 0.997 |
| | | protein 1 | | | | |
| BCL2L1 | 598 | BCL2 like 1 | 4.094 | 0.031 | −0.344 | 0.954 |
| TMEM14C | 51522 | transmembrane protein 14C | 4.016 | 0.013 | 0.699 | 0.848 |
| PTRH2 | 51651 | peptidyl-tRNA hydrolase 2 | 3.904 | <0.001 | 0.699 | 0.512 |
| L2HGDH | 79944 | L-2-hydroxy glutarate | 3.842 | 0.030 | −0.072 | 0.993 |
| | | dehydrogenase | | | | |
| ARG2 | 384 | arginase 2 | 3.827 | 0.010 | 1.012 | 0.722 |
| MRPL4 | 51073 | mitochondrial ribosomal protein L4 | 3.799 | 0.014 | 1.653 | 0.552 |
| PPIF | 10105 | peptidylprolyl isomerase F | 3.731 | 0.020 | 0.930 | 0.792 |
| CPOX | 1371 | coproporphyrinogen oxidase | 3.711 | <0.001 | 0.318 | 0.714 |
| MRPL48 | 51642 | mitochondrial ribosomal protein L48 | 3.649 | 0.007 | 1.113 | 0.662 |
| MRPS11 | 64963 | mitochondrial ribosomal protein S11 | 3.623 | <0.001 | 1.272 | 0.301 |
| MRPL44 | 65080 | mitochondrial ribosomal protein L44 | 3.569 | 0.003 | 1.303 | 0.496 |
| MRPS31 | 10240 | mitochondrial ribosomal protein S31 | 3.520 | 0.039 | 0.148 | 0.982 |
| PYCR2 | 29920 | pyrroline-5-carboxylate reductase 2 | 3.399 | 0.021 | 1.046 | 0.722 |
| OAT | 4942 | ornithine aminotransferase | 3.292 | 0.010 | 0.508 | 0.865 |
| MRPL12 | 6182 | mitochondrial ribosomal protein L12 | 3.274 | 0.009 | 0.857 | 0.722 |
| LACTB2 | 51110 | lactamase beta 2 | 3.255 | 0.012 | −0.091 | 0.986 |
| SFXN2 | 118980 | sideroflexin 2 | 3.240 | 0.032 | 0.731 | 0.840 |
| PLSCR3 | 57048 | phospholipid scramblase 3 | 3.226 | 0.005 | 0.094 | 0.982 |
| TIMM13 | 26517 | translocase of inner mitochondrial | 3.159 | 0.017 | 0.257 | 0.950 |
| | | membrane 13 | | | | |
| CPT1A | 1374 | carnitine palmitoyltransferase 1A | 3.069 | <0.001 | 0.247 | 0.834 |
| MRPL13 | 28998 | mitochondrial ribosomal protein L13 | 3.030 | <0.001 | 0.581 | 0.607 |
| CYB5B | 80777 | cytochrome b5 type B | 3.021 | 0.043 | −0.428 | 0.920 |
| MRPS35 | 60488 | mitochondrial ribosomal protein S35 | 3.003 | 0.040 | 1.467 | 0.588 |
| AGMAT | 79814 | agmatinase | 2.994 | 0.007 | 0.588 | 0.805 |
| MRPS9 | 64965 | mitochondrial ribosomal protein S9 | 2.970 | 0.033 | 1.633 | 0.518 |
| NUDT1 | 4521 | nudix hydrolase 1 | 2.947 | <0.001 | −1.222 | 0.227 |
| MPST | 4357 | mercaptopyruvate sulfurtransferase | 2.938 | <0.001 | 0.621 | 0.552 |
| HTRA2 | 27429 | HtrA serine peptidase 2 | 2.929 | 0.017 | 1.077 | 0.652 |
| TOMM40 | 10452 | translocase of outer mitochondrial | 2.848 | 0.005 | 0.692 | 0.710 |
| | | membrane 40 | | | | |
| MTXI | 4580 | metaxin 1 | 2.803 | 0.014 | 0.075 | 0.989 |
| MRPL37 | 51253 | mitochondrial ribosomal protein L37 | 2.762 | 0.002 | 0.667 | 0.657 |
| RMND1 | 55005 | required for meiotic nuclear | 2.669 | 0.019 | −0.033 | 0.994 |
| | | division 1 homolog | | | | |
| MRPL39 | 54148 | mitochondrial ribosomal protein L39 | 2.610 | 0.038 | 0.254 | 0.950 |
| ACADVL | 37 | acyl-CoA dehydrogenase very long | 2.567 | <0.001 | 0.411 | 0.070 |
| | | chain | | | | |
| ECU | 1632 | enoyl-CoA delta isomerase 1 | 2.477 | 0.012 | −2.003 | 0.185 |
| ACSL5 | 51703 | acyl-CoA synthetase long chain | 2.457 | 0.003 | 0.610 | 0.684 |
| | | family member 5 | | | | |
| DUT | 1854 | deoxyuridine triphosphatase | 2.436 | <0.001 | 0.116 | 0.847 |
| ACSL1 | 2180 | acyl-CoA synthetase long chain | 2.348 | 0.002 | 0.254 | 0.873 |
| | | family member 1 | | | | |
| ARL2 | 402 | ADP ribosylation factor like | 2.250 | 0.020 | 0.579 | 0.781 |
| | | GTPase 2 | | | | |
| FEN1 | 2237 | flap structure-specific endonuclease 1 | 2.245 | <0.001 | 0.235 | 0.566 |
| AGK | 55750 | acylglycerol kinase | 2.232 | 0.001 | −0.159 | 0.938 |
| NDUFA8 | 4702 | NADHubiquinone oxidoreductase | 2.224 | 0.004 | 0.276 | 0.873 |
| | | subunit A8 | | | | |
| ACAA2 | 10449 | acetyl-CoA acyltransferase 2 | 2.217 | <0.001 | 0.313 | 0.260 |
| LRPPRC | 10128 | leucine rich pentatricopeptide | 2.193 | <0.001 | 0.207 | 0.810 |
| | | repeat containing | | | | |
| FDXR | 2232 | ferredoxin reductase | 2.169 | 0.006 | 0.407 | 0.806 |
| GRSF1 | 2926 | G-rich RNA sequence binding | 2.142 | 0.031 | 1.055 | 0.566 |
| | | factor 1 | | | | |
| DNMIL | 10059 | dynamin 1 like | 2.141 | 0.010 | −0.193 | 0.938 |
| CISD2 | 493856 | CDGSH iron sulfur domain 2 | 2.128 | 0.002 | −0.021 | 0.993 |
| MRPL1 | 65008 | mitochondrial ribosomal protein LI | 2.126 | 0.000 | 0.537 | 0.497 |
| PMPCA | 23203 | peptidase, mitochondrial processing | 2.125 | 0.025 | 1.008 | 0.565 |
| | | alpha subunit | | | | |
| ACADM | 34 | acyl-CoA dehydrogenase medium | 2.116 | 0.001 | 0.518 | 0.581 |
| | | chain | | | | |
| BDH1 | 622 | 3-hydroxybutyrate dehydrogenase 1 | 2.108 | 0.002 | 0.589 | 0.566 |
| TIMM23 | 1E+08 | translocase of inner mitochondrial | 2.107 | 0.027 | 0.411 | 0.858 |
| | | membrane 23 | | | | |
| CPT2 | 1376 | carnitine palmitoyltransferase 2 | 2.079 | 0.019 | 0.652 | 0.714 |
| CIAPIN1 | 57019 | cytokine induced apoptosis | 2.074 | 0.003 | −0.691 | 0.540 |
| | | inhibitor 1 | | | | |
| CHDH | 55349 | choline dehydrogenase | 2.029 | 0.029 | 1.195 | 0.465 |
| TOMM22 | 56993 | translocase of outer mitochondrial | 2.022 | 0.002 | 0.624 | 0.546 |
| | | membrane 22 | | | | |
| FKBP8 | 23770 | FKBP prolyl isomerase 8 | 2.014 | 0.001 | 1.093 | 0.138 |

### 8.8.4.3.3 Cell Proliferation Gene Product Signature (Cell Division, Cell Cycle, and DNA Replication/Repair)

The methods of producing Tregs presented herein yield ex vivo-expanded Tregs that proliferate at a rapid rate. The proteomics study has revealed that the expression of a number of gene products associated with cell proliferation pathways is increased relative to their expression in baseline Tregs. See Table 17. These enriched gene products include, for example, ones associated with cell cycle (p=0.0014), cell division (p=0.0478), DNA replication (p=5.05e-13), and DNA Repair (p=0.0496) pathways.

As shown in Table 17, the cell proliferation gene product signature includes, for example: ARL2, ARL3, BCCIP, CCDC124, CDK1, CDK2, CDK5, CDK6, CUL4B, DCTN3, FEN1, HELLS, LIG1, MAD2L1, MAEA, MCM2, MCM2, MCM3, MCM4, MCM5, MCM6, MCM7, MCMBP, NUDC, PCNA, POLD1, POLD2, RALB, RBM38, RFC2, RFC3, RFC4, RFC5, RNASEH2A, RNASEH2B, SMC2.

TABLE 17
Cell proliferation gene product signature

| | | | | | log2 of | |
|---|---|---|---|---|---|---|
| | | | log2 of | | fold-change | |
| Gene | NCBI | | fold-change | Adjusted | of baseline | Adjusted |
| Symbol | GENE ID | Gene Description | of [symbol] | p-value | vs. freeze-thaw | p-value |
| CDK1 | 983 | cyclin dependent kinase 1 | 6.615 | 0.007 | −0.077 | 0.993 |
| MCM5 | 4174 | minichromosome maintenance | 6.473 | <0.001 | −0.031 | 0.993 |
| | | complex component 5 | | | | |
| CDK6 | 1021 | cyclin dependent kinase 6 | 6.206 | 0.006 | −1.043 | 0.831 |
| MCM2 | 4171 | minichromosome maintenance | 6.203 | 0.010 | 1.548 | 0.735 |
| | | complex component 2 | | | | |
| MCM4 | 4173 | minichromosome maintenance | 6.021 | <0.001 | 0.213 | 0.839 |
| | | complex component 4 | | | | |
| CDK5 | 1020 | cyclin dependent kinase 5 | 5.954 | <0.001 | 0.197 | 0.949 |
| MCM3 | 4172 | minichromosome maintenance | 5.613 | <0.001 | 0.122 | 0.966 |
| | | complex component 3 | | | | |
| CCDC124 | 115098 | coiled-coil domain containing 124 | 4.855 | 0.006 | 0.277 | 0.960 |
| MCM7 | 4176 | minichromosome maintenance | 4.743 | 0.000 | 0.060 | 0.924 |
| | | complex component 7 | | | | |
| RALB | 5899 | RAS like proto-oncogene B | 4.644 | 0.008 | −0.199 | 0.975 |
| SMC2 | 10592 | structural maintenance of | 4.463 | 0.002 | 0.600 | 0.616 |
| | | chromosomes 2 | | | | |
| CDK2 | 1017 | cyclin dependent kinase 2 | 4.455 | 0.005 | −0.124 | 0.982 |
| RBM38 | 55544 | RNA binding motif protein 38 | 4.433 | 0.004 | −0.435 | 0.912 |
| MCM6 | 4175 | minichromosome maintenance | 4.394 | <0.001 | 0.152 | 0.946 |
| | | complex component 6 | | | | |
| MAEA | 10296 | macrophage erythroblast attacher | 4.259 | 0.007 | −1.045 | 0.722 |
| DCTN3 | 11258 | dynactin subunit 3 | 4.232 | 0.001 | 0.377 | 0.902 |
| BCCIP | 56647 | BRCA2 and CDKN1A interacting | 4.012 | <0.001 | 0.194 | 0.908 |
| | | protein | | | | |
| RFC5 | 5985 | replication factor C subunit 5 | 4.000 | 0.006 | −0.243 | 0.954 |
| MAD2L1 | 4085 | mitotic arrest deficient 2 like 1 | 3.978 | 0.001 | −0.209 | 0.975 |
| CUL4B | 8450 | cullin 4B | 3.896 | 0.034 | −0.871 | 0.844 |
| MCMBP | 79892 | minichromosome maintenance | 3.680 | 0.013 | 0.344 | 0.938 |
| | | complex binding protein | | | | |
| RFC3 | 5983 | replication factor C subunit 3 | 3.663 | 0.012 | −0.308 | 0.946 |
| LIG1 | 3978 | DNA ligase 1 | 3.564 | 0.001 | −0.547 | 0.770 |
| RFC4 | 5984 | replication factor C subunit 4 | 3.546 | <0.001 | 0.130 | 0.960 |
| POLD2 | 5425 | DNA polymerase delta 2, | 3.491 | 0.003 | 0.289 | 0.920 |
| | | accessory subunit | | | | |
| RNASEH2A | 10535 | ribonuclease H2 subunit A | 3.447 | 0.037 | 0.751 | 0.848 |
| HELLS | 3070 | helicase, lymphoid specific | 3.317 | 0.037 | 0.966 | 0.783 |
| POLDI | 5424 | DNA polymerase delta l, catalytic | 2.998 | 0.001 | −0.228 | 0.915 |
| | | subunit | | | | |
| ARL3 | 403 | ADP ribosylation factor like | 2.973 | <0.001 | 0.034 | 0.990 |
| | | GTPase 3 | | | | |
| RNASEH2B | 79621 | ribonuclease H2 subunit B | 2.858 | <0.001 | 0.037 | 0.990 |
| RFC2 | 5982 | replication factor C subunit 2 | 2.731 | 0.001 | −0.203 | 0.911 |
| PCNA | 5111 | proliferating cell nuclear antigen | 2.615 | <0.001 | 0.183 | 0.655 |
| ARL2 | 402 | ADP ribosylation factor like | 2.250 | 0.020 | 0.579 | 0.781 |
| | | GTPase 2 | | | | |
| FEN1 | 2237 | flap structure-specific | 2.245 | <0.001 | 0.235 | 0.566 |
| | | endonuclease 1 | | | | |
| NUDC | 10726 | nuclear distribution C, dynein | 2.046 | <0.001 | −0.212 | 0.742 |
| | | complex regulator | | | | |

[symbol]

indicates data missing or illegible when filed

### 8.8.4.3.4 Highest Protein Expression Gene Product Signature (Following Expansion)

Next, gene products obtained from the proteomics study were stratified based on highest protein expression observed in the ex vivo-expanded Tregs produced by the methods presented herein, as quantified using intensity based absolute quantification (iBAQ) which is a measure of protein abundance in the proteomics assay. The top 40 expressed gene products obtained from the study are compiled at Table 18. As noted in Table 18, the expression of each of the members of this highest protein expression gene product signature is increased relative to the expression seen in baseline Tregs.

The gene products making up the highest expressing protein signatures include, for example: ACAA2, ACADM, ACADVL, ACOT7, BSG, CACYBP, CD74, CDK1, CPOX, DUT, ECI1, ENO3, FEN1, FKBP3, HISTH2BJ, 2LA-DQA1, HLA-DRA, HLA-DRB1, LGALS1, LGALS3, MCM5, MCM6, MCM7, MTHFD1, NAMPT, NME1, NQO1, PCNA, RAB1A, RALB, SLC25A4, STAT1, STMN1, STMN2, TUBA1B, TUBB4A, TUBB8, TXN, TXNRD1, and WARS.

TABLE 18
Highest protein expression gene product signature

| | | | log2 of | | log2 of | |
|---|---|---|---|---|---|---|
| | | | fold-change | | fold-change | |
| Gene | NCBI | | of baseline | Adjusted | of baseline | Adjusted |
| Symbol | GENE ID | Gene Description | vs. expanded | p-value | vs. freeze-thaw | p-value |
| HIST1H2BJ | 8970 | histone cluster 1 H2B family | 13.792 | <0.001 | −2.190 | 0.000 |
| | | member j | | | | |
| TXN | 7295 | thioredoxin | 3.977 | <0.001 | 0.064 | 0.908 |
| TUBAIB | 10376 | tubulin alpha 1b | 2.224 | 0.001 | 0.111 | 0.946 |
| LGALS3 | 3958 | galectin 3 | 2.523 | <0.001 | −0.108 | 0.940 |
| NME1 | 4830 | NME/NM23 nucleoside | 13.947 | 0.006 | −3.549 | 0.693 |
| | | diphosphate kinase 1 | | | | |
| TUBB8 | 347688 | tubulin beta 8 class VIII | 8.661 | 0.028 | −3.253 | 0.672 |
| STMN1 | 3925 | stathmin 1 | 2.411 | <0.001 | −0.029 | 0.975 |
| TUBB4A | 10382 | tubulin beta 4A class IVa | 8.606 | 0.026 | 1.514 | 0.874 |
| STAT1 | 6772 | signal transducer and activator of | 3.425 | <0.001 | −0.104 | 0.882 |
| | | transcription 1 | | | | |
| LGALS1 | 3956 | galectin 1 | 2.014 | <0.001 | 0.096 | 0.877 |
| CACYBP | 27101 | calcyclin binding protein | 2.754 | <0.001 | −0.007 | 0.994 |
| WARS | 7453 | tryptophanyl-tRNA synthetase | 3.715 | <0.001 | 0.025 | 0.982 |
| PCNA | 5111 | proliferating cell nuclear antigen | 2.615 | <0.001 | 0.183 | 0.655 |
| ACAA2 | 10449 | acetyl-CoA acyltransferase 2 | 2.217 | <0.001 | 0.313 | 0.260 |
| CDK1 | 983 | cyclin dependent kinase 1 | 6.615 | 0.007 | 1.621 | 0.502 |
| ECU | 1632 | enoyl-CoA delta isomerase 1 | 2.477 | 0.012 | −2.003 | 0.185 |
| ACADVL | 37 | acyl-CoA dehydrogenase very long | 2.567 | <0.001 | 0.411 | 0.070 |
| | | chain | | | | |
| SLC25A4 | 291 | solute carrier family 25 member 4 | 5.089 | 0.030 | −2.206 | 0.621 |
| RAB1A | 5861 | RABI A, member RAS oncogene | 2.680 | 0.001 | −0.017 | 0.996 |
| | | family | | | | |
| DUT | 1854 | deoxyuridine triphosphatase | 2.436 | <0.001 | 0.116 | 0.847 |
| HLA-DRA | 3122 | major histocompatibility complex, | 4.722 | <0.001 | 0.353 | 0.353 |
| | | class II, DR alpha | | | | |
| FKBP3 | 2287 | FKBP prolyl isomerase 3 | 2.105 | <0.001 | 0.039 | 0.979 |
| NAMPT | 10135 | nicotinamide | 4.070 | <0.001 | 0.255 | 0.385 |
| | | phosphoribosyltransferase | | | | |
| FENI | 2237 | flap structure-specific | 2.245 | <0.001 | 0.235 | 0.566 |
| | | endonuclease 1 | | | | |
| STMN2 | 11075 | stathmin 2 | 5.041 | 0.023 | 0.073 | 0.993 |
| NQOl | 1728 | NAD(P)H quinone dehydrogenase 1 | 9.019 | <0.001 | −0.188 | 0.882 |
| HLA-DRB1 | 3123 | major histocompatibility complex, | 5.489 | <0.001 | 0.347 | 0.685 |
| | | class II, DR beta 1 | | | | |
| TXNRD1 | 7296 | thioredoxin reductase 1 | 2.299 | <0.001 | 0.080 | 0.940 |
| MCM7 | 4176 | minichromosome maintenance | 4.743 | <0.001 | 0.060 | 0.924 |
| | | complex component 7 | | | | |
| HLA-DQA1 | 3117 | major histocompatibility complex, | 3.944 | <0.001 | 0.215 | 0.932 |
| | | class II, DQ alpha l | | | | |
| CD74 | 972 | CD74 molecule | 2.625 | <0.001 | 0.366 | 0.357 |
| MTHFD1 | 4522 | methylenetetrahydrofolate | 2.410 | <0.001 | −0.065 | 0.938 |
| | | dehydrogenase, cyclohydrolase and | | | | |
| | | formyltetrahydrofolate synthetase l | | | | |
| MCM5 | 4174 | minichromosome maintenance | 6.473 | <0.001 | −0.031 | 0.993 |
| | | complex component 5 | | | | |
| CPOX | 1371 | coproporphyrinogen oxidase | 3.711 | <0.001 | 0.318 | 0.714 |
| RALB | 5899 | RAS like proto-oncogene B | 4.644 | 0.008 | −0.199 | 0.975 |
| BSG | 682 | basigin (Ok blood group) | 4.721 | 0.001 | 0.042 | 0.993 |
| ACADM | 34 | acyl-CoA dehydrogenase medium | 2.116 | 0.001 | 0.518 | 0.581 |
| | | chain | | | | |
| MCM6 | 4175 | minichromosome maintenance | 4.394 | <0.001 | 0.152 | 0.946 |
| | | complex component 6 | | | | |
| ACOT7 | 11332 | acyl-CoA thioesterase 7 | 4.566 | <0.001 | −0.075 | 0.973 |
| ENO3 | 2027 | enolase 3 | 6.086 | 0.001 | −0.296 | 0.950 |

### 8.8.4.4 Freeze/Thaw Storage of Expanded Tregs does not Significantly Change their Proteomic Signature

Surprisingly, even after cryopreservation and thawing, the ex vivo-expanded Tregs produced, cryopreserved and thawed by the methods presented herein exhibit minimal proteomic change. In particular, a freeze/thaw cycle utilized for Treg storage following the expansion process shows minimal proteomic change that mapped back to only 29 gene products out of 3,709 total gene products found (Table 19). The cryopreserved Tregs were assessed following thawing without additional expansion, demonstrating that the cryopreserved and then thawed Tregs retained their gene product signatures without the need for post-thaw expansion.

Pathway analysis suggests the changes that are occurring are implicated in pathways involved with mRNA processing such as mRNA splicing (p=5e-15), mRNA 3′-end processing (p=4.91e-10), RNA polymerase II transcription termination (p=1.04e-9), and transport of mature mRNA to the cytoplasm (p=5.76e-9).

The proteomic gene product signature enhanced during the ex vivo-expansion process of the patient Tregs described herein does not significantly change following the freeze/thaw cycle. Interestingly, most of the gene products identified as belonging to the methylation gene product signature (Table 13), Treg-associated gene product signature (Table 15), the mitochondria gene product signature (Table 16), or the cell proliferation gene product signature (Table 17) exhibited did not substantial change pre- vs. post-cryopreservation. Most of the gene products identified as belonging to the methylation gene product signature (Table 13) were lost during the expansion process (i.e., expression of these gene products was decreased in the expanded Tregs compared to baseline).

TABLE 19
Very few proteomic changes are observed following cryopreservation
and Thawing

| | | | Log2 of | |
|---|---|---|---|---|
| | | | fold- | |
| | | | change | |
| | | | of | |
| | NCBI | | baseline | |
| Gene | Gene | | vs. | Adjusted |
| Symbol | ID | Gene Description | expanded | p-value |
| CD247 | 919 | CD247 molecule | 6.610 | 0.022 |
| ATP8 | 4509 | ATP synthase F0 | 6.594 | 0.018 |
| | | subunit 8 | | |
| PRPF38B | 55119 | pre-mRNA | 6.243 | 0.012 |
| | | processing factor | | |
| | | 38B | | |
| FUS | 2521 | FUS RNA binding | 6.227 | 0.033 |
| | | protein | | |
| DDX41 | 51428 | DEAD-box helicase | 5.717 | 0.042 |
| | | 41 | | |
| DDX23 | 9416 | DEAD-box helicase | 5.267 | 0.018 |
| | | 23 | | |
| APOH | 350 | apolipoprotein H | 4.546 | <0.001 |
| AFM | 173 | afamin | 4.533 | 0.020 |
| SRSF1 | 6426 | serine and arginine | 4.064 | 0.030 |
| | | rich splicing factor 1 | | |
| SRSF9 | 8683 | serine and arginine | 3.870 | 0.009 |
| | | rich splicing factor 9 | | |
| CIRBP | 1153 | cold inducible RNA | 3.811 | <0.001 |
| | | binding protein | | |
| SRSF3 | 6428 | serine and arginine | 2.990 | 0.001 |
| | | rich splicing factor 3 | | |
| SRSF7 | 6432 | serine and arginine | 2.749 | 0.002 |
| | | rich splicing factor 7 | | |
| SRRT | 51593 | serrate, RNA effector | 2.683 | 0.025 |
| | | molecule | | |
| HNRNPA3 | 220988 | heterogeneous nuclear | 2.625 | 0.001 |
| | | ribonucleoprotein A3 | | |
| SRSF6 | 6431 | serine and arginine | 2.596 | 0.010 |
| | | rich splicing factor 6 | | |
| ALB | 213 | albumin | 2.527 | <0.001 |
| DDX17 | 10521 | DEAD-box helicase | 2.371 | <0.001 |
| | | 17 | | |
| HNRNPA2B1 | 3181 | heterogeneous nuclear | 2.209 | <0.001 |
| | | ribonucleoprotein | | |
| | | A2/B1 | | |
| SRSF2 | 6427 | serine and arginine | 2.191 | 0.012 |
| | | rich splicing factor 2 | | |
| U2AF2 | 11338 | U2 small nuclear | 2.166 | 0.038 |
| | | RNA auxiliary | | |
| | | factor 2 | | |
| DDX3X | 1654 | DEAD-box helicase | 2.073 | 0.001 |
| | | 3 X-linked | | |
| HNRNPA0 | 10949 | heterogeneous nuclear | 2.070 | 0.020 |
| | | ribonucleoprotein A0 | | |
| RBM3 | 5935 | RNA binding motif | 2.042 | 0.002 |
| | | protein 3 | | |
| HIST2H2BE | 8349 | histone cluster 2 H2B | −2.190 | 0.001 |
| | | family member e | | |
| HIST1H2BJ | 8970 | histone cluster 1 H2B | −2.190 | <0.001 |
| | | family member j | | |
| EIPR1 | 7260 | EARP complex and | −3.820 | 0.010 |
| | | GARP complex | | |
| | | interacting protein 1 | | |
| SLC25A18 | 83733 | solute carrier family | −4.960 | 0.031 |
| | | 25 member 18 | | |
| PECAM1 | 5175 | platelet and | −5.305 | 0.001 |
| | | endothelial cell | | |
| | | adhesion molecule 1 | | |

### 8.9 Example 8: Representative Automated Treg Expansion Protocol

Following isolation and enrichment (e.g., CD25⁺ enrichment/CD8+CD19+ depletion, for example, via CliniMACS), the CD25⁺-enriched cells are incubated in a Quantum Cell Expansion System (Terumo BCT). One day (about 16-24 hours) following isolation (Day 1), the CD25⁺ cells are activated with anti-CD3/anti-CD28 beads at a 4:1 beads-to-cell ratio in the bioreactor. IL-2 and rapamycin are also be added on Day 1. The cells are expanded in the Quantum bioreactor from Day 2 to Day 7. Cell counts and viability are determined each day. Glucose and lactate levels in the culture media are also measured daily and the medium flow rate is adjusted based on the glucose:lactate ratio. On Day 8, if the cell expansion yields the dose of cells required (typically ≥2×10⁹ cells), then the cells are harvested and cryopreserved following bead removal. If the cell expansion process has not reached dose, then the cells are reactivated with CD3/CD28 beads at a 1:1 beads-to-cell ratio in the bioreactor. The expansion process may continue in the bioreactor from Day 9 to Day 15, as necessary. Cell counts and viability are measured each day and glucose:lactate ratios are monitored to adjust the medium flow rate. Once the cell expansion process yields the dose of cells needed (occurring any day between Days 9 and 15), then the cells are immediately harvested and cryopreserved following bead removal.

### 8.10 Example 10: mRNA Therapy for Improved Adoptive T-Cell Transfer

A patients own T-cells may be obtained, genetically modified and administered back to the patient, for example to attack a the patient's tumors as part of a cancer immunotherapy program, or to suppress exuberant immune system activation for treatment of graft versus host disease.

Advancement of atherosclerotic lesions requires cytokines promoting a Th1 response within the plaques leading to macrophage and T cell activation and the release of pro-inflammatory cytokines such as tumor necrosis factor and interleukin-1. Preliminary studies support the use of adoptive T-cell therapy for atherosclerosis as demonstrated by a mouse model wherein Treg depletion was observed in lymphoid tissues and plaques, indicating the important antiatherosclerotic role of Treg.

The studies presented in this Example demonstrate that human telomerase (hTERT) mRNA has great potential to improve the therapeutic benefits of chimeric antigen receptors (CAR-T) therapy as transfection of hTERT mRNA increased CAR-T cell number and telomere length. In addition, other therapeutic mRNAs have been introduced along with hTERT mRNA into Treg to address amyotrophic lateral sclerosis. Tregs transfected with the therapeutic mRNA displays an increased inhibitory function, which is preferred for the Treg activity. T-cell specific mRNA transcripts with optimal codon sequences have also been analyzed. An aim is to develop a novel codon-optimized RNA-based reagent and a biomimetic nanoparticle-based delivery of therapeutic mRNAs including hTERT for improvement of immunotherapies by enhancing the proliferation and function.

### 8.10.1. METHODS

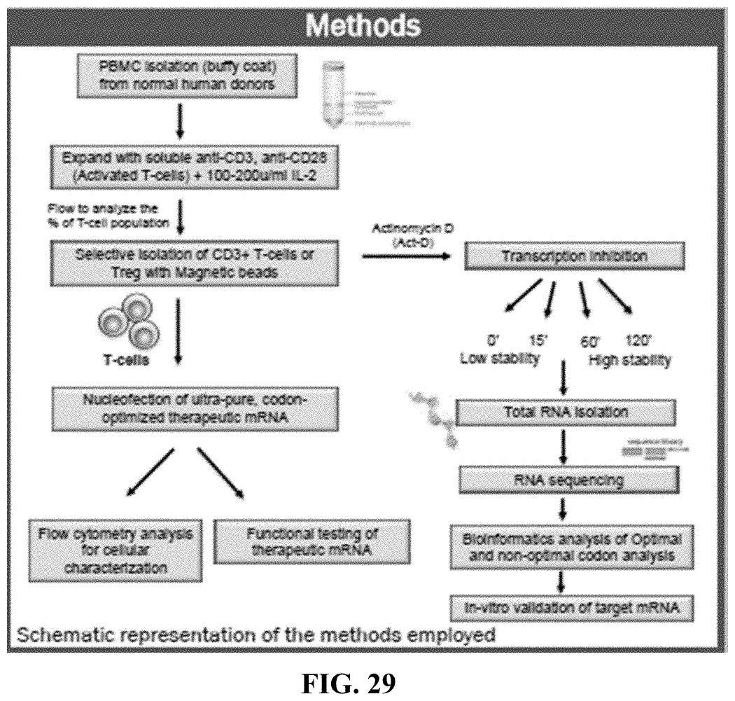

A schematic representation of the methods employed in this example is shown in FIG. 29.

### 8.10.2. RESULTS

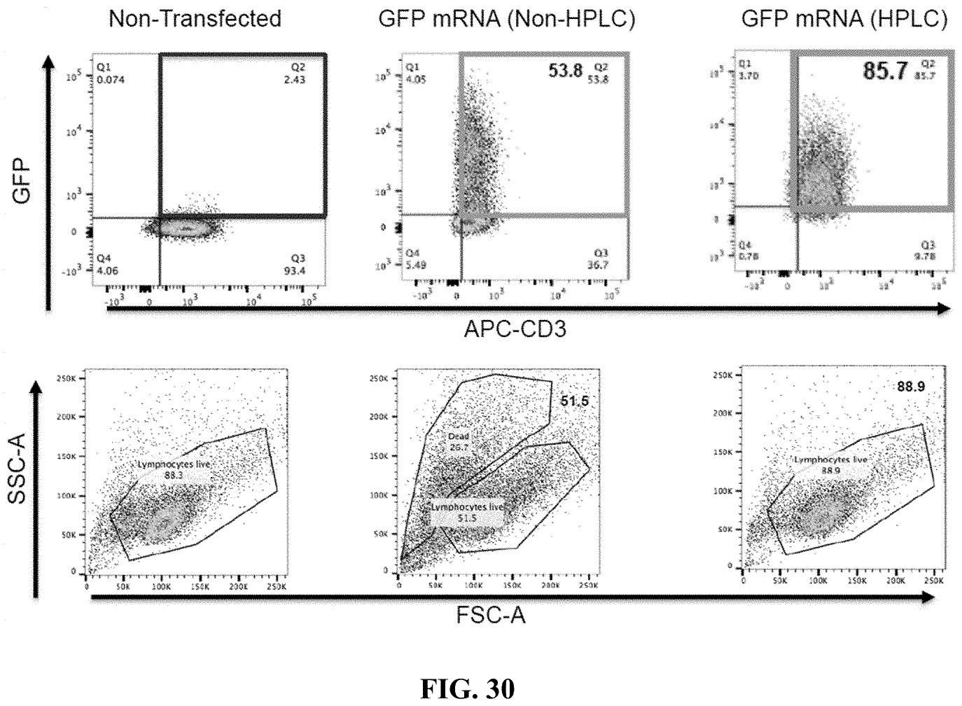

FIG. 30 shows nucleofection of GFP-mRNA in T-cells. Flow cytometry analysis shows improved transfection efficiency (Upper panel) and T-cell viability (Lower panel) with HPLC-grade GFP mRNA compared to non-HPLC mRNA. Nucleofection was performed using Lonza 4D-Nucleofector system and the T-cells were allowed to recover for 24 hrs prior to flow analysis.

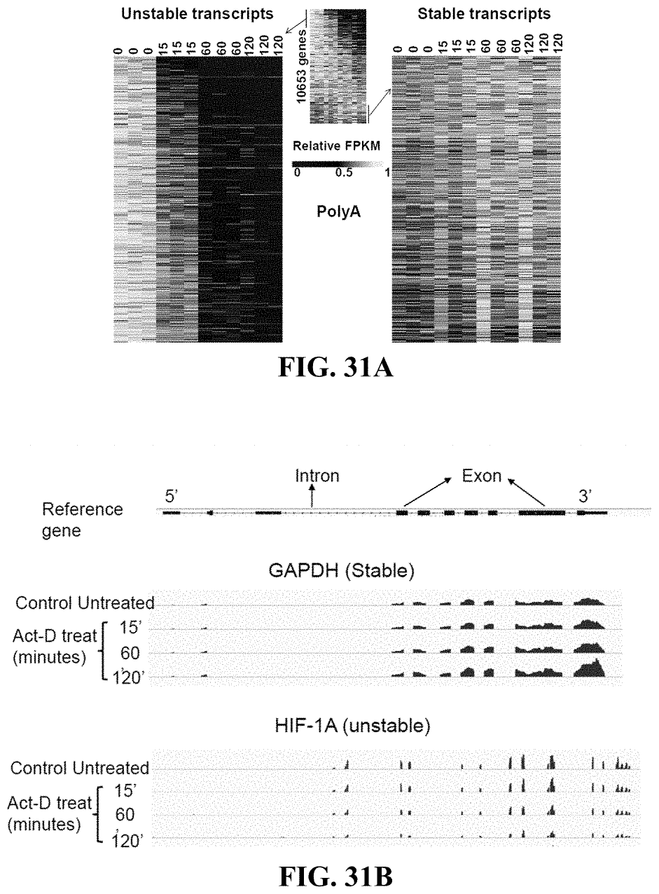

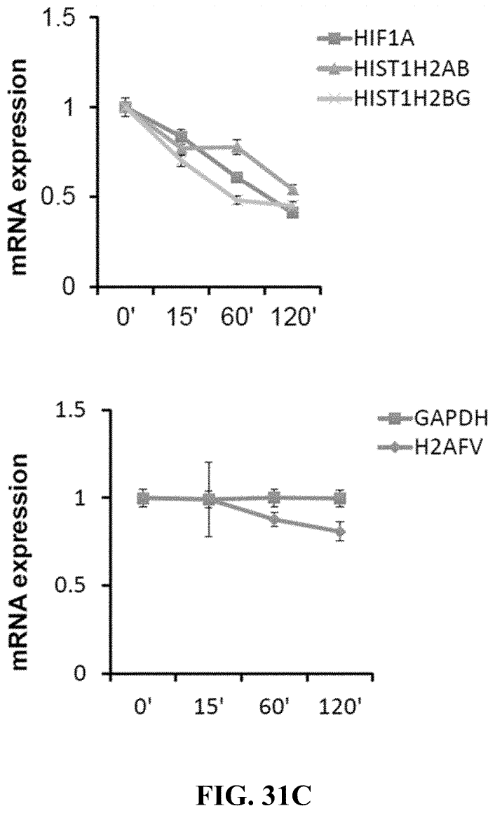

FIGS. 31A-31C show Bioinformatics analysis and in-vitro validation of stable mRNA transcripts in T-cells. FIG. 31A: Heat map of stable and unstable transcript levels at different time points post Actinomycin D treatment. FIG. 31B: RNA track plot showing expression profile of a stable (GAPDH) and an unstable transcript (HIF-1A) analyzed by bioinformatics. FIG. 31C: shows in-vitro validation of mRNA transcripts. FIG. 31C, upper panel is a qRTPCR analysis showing (upper panel) decreased expression of histone transcripts (HIST1H2AB and HIST1H2BG) and HIF1A (Hypoxia inducible Factor 1A). FIG. 31C, lower panel, is a qRTPCR analysis showing stable GAPDH transcript and Histone H2A transcript expression was unaltered after transcription block by Actinomycin D, validating the bioinformatics analysis.

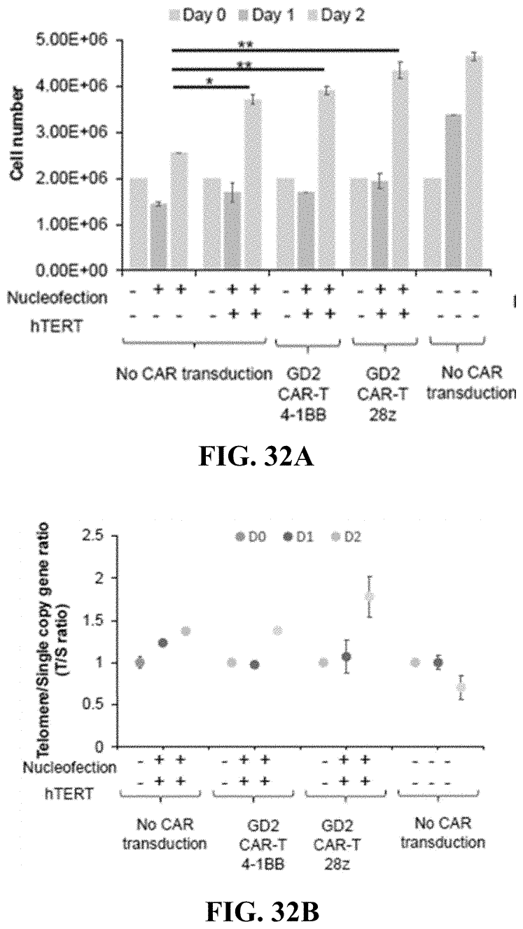

FIGS. 32A and 32B show increased cell number and telomere length with hTERT expression in non-transduced and CAR-transduced Tcells. CAR directed against the disialoganglioside GD2, a surface molecule expressed in neuroblastoma and neuroectoderm-derived neoplasms was considered for transfection of hTERT mRNA. These are second generation CARs with either 28z or 4-1BB co-stimulatory signals. FIG. 32A: Viable cell count was performed using trypan blue in a cell counter. There was a major cell death 24-hour post-transfection. However, the cell number of both the no-CAR and GD2-CAR transduced Tcells was higher with hTERT mRNA transfection post 48 hours compared to controls. FIG. 32B: Relative telomere length was measured using mmQPCR, which determines the ratio of telomeric signals (T) to single copy gene signal telomere (S) (T/S ratios). hTERT mRNA transfected cells displayed an increased telomere length compared to the non-transfected controls in both no-CAR transduction and CAR transduced T-cells.

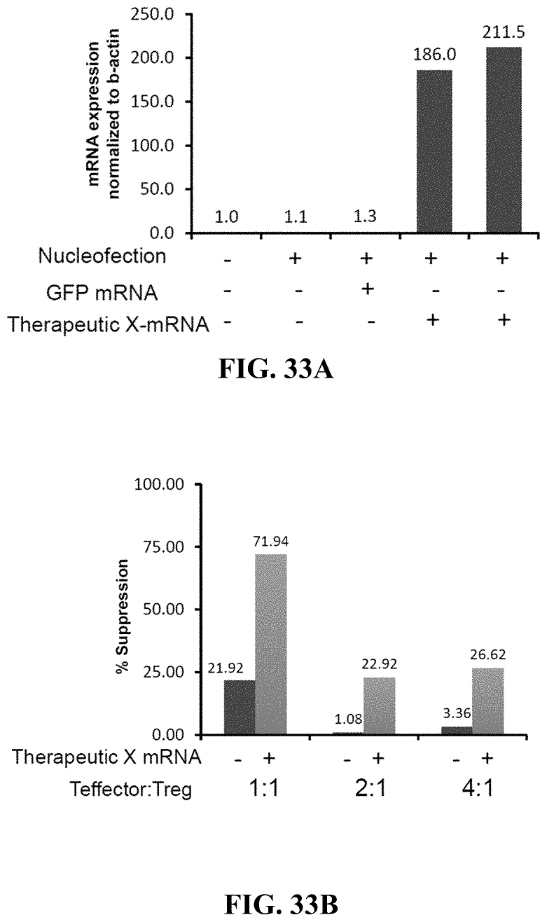

FIGS. 33A and 33B show nucleofection of GFP and X-mRNA in T regulatory cells (Tregs). FIG. 33A: T-regs were selectively isolated from PBMCs and nucleofected with mRNA. qRT-PCR was performed 24 hours post nucleofection and the results show an approximately 200 fold increase in the therapeutic X-mRNA compared to non-transfected and GFP-transfected controls. FIG. 33B: Treg suppression assay was performed to determine its function wherein Tregs were cocultured with effector T cells (Teff) at different ratios. Tregs transfected with mRNA displayed an increased suppressive function at 1:1 ratio compared to non-transfected cells.

### 8.10.3. SUMMARY

These studies demonstrate that mRNA (e.g. hTERT) has great potential to improve the therapeutic benefits of CAR-T and Treg cell therapy. An additional aim is to develop a novel codon-optimized RNA-based reagent for delivery of therapeutic mRNAs, including hTERT, to improve immunotherapies by enhancing the proliferation capacity of CAR-T and Treg cells, an approach which can be applied to other somatic cell therapies. A summary of the mRNA therapy for improved adoptive T-cell transfer described in this example is shown in FIG. 34.

The results presented here demonstrate a 53% transfection efficiency with non-HPLC grade mRNA and an 85% efficiency with HPLC grade mRNA, accompanied by a superior viability using HPLC mRNA.

Bioinformatics analysis of the T-cell mRNAs after transcription inhibition displayed highly stable vs unstable transcripts, which will be further analyzed for the T-cell codon optimality.

The results presented herein represent the first pre-clinical evidence that transfection of hTERT mRNA increases T-cell replicative capacity in vitro and improves cell number of CAR-T cells directed against disialoganglioside (GD2), compared to control. The therapeutic mRNA was also introduced into Tregs resulting in superior expression of the therapeutic mRNA and cell function.

### 8.10.4. REFERENCES

1. Y. Bai, S. Kan, S. Zhou, Y. Wang, J. Xu, J. P. Cooke, J. Wen, H. Deng, Enhancement of the in vivo persistence and antitumor efficacy of CD19 chimeric antigen receptor T cells through the delivery of modified TERT mRNA, Cell Discov 1 (2015) 15040.
2. J. S. Henkel, D. R. Beers, S. Wen, A. L. Rivera, K. M. Toennis, J. E. Appel, W. Zhao, D. H. Moore, S. Z. Powell, S. H. Appel, Regulatory T-lymphocytes mediate amyotrophic lateral sclerosis progression and survival, EMBO Mol Med 5(1) (2013) 64-79.
3. M. Prapa, S. Caldrer, C. Spano, M. Bestagno, G. Golinelli, G. Grisendi, T. Petrachi, P. Conte, E. M. Horwitz, D. Campana, P. Paolucci, M. Dominici, A novel anti-GD2/4-1BB chimeric antigen receptor triggers neuroblastoma cell killing, Oncotarget 6(28) (2015) 24884-94.
4. X. Meng, J. Yang, M. Dong, K. Zhang, E. Tu, Q. Gao, W. Chen, C. Zhang, Y. Zhang, Regulatory T cells in cardiovascular diseases, Nat Rev Cardiol 13(3) (2016) 167-79.
5. L. Huang, Y. Zheng, X. Yuan, Y. Ma, G. Xie, W. Wang, H. Chen, L. Shen, Decreased frequencies and impaired functions of the CD31+ subpopulation in Treg cells associated with decreased FoxP3 expression and enhanced Treg cell defects in patients with coronary heart disease, Clin Exp Immunol 187(3) (2017) 441-454.

### 9. REFERENCES

The following references, to the extent that they provide exemplary procedural or other details supplementary to those set forth herein, are specifically incorporated herein in their entirety by express reference thereto:

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved.

What is claimed is:

1. A method of producing a cryopreserved therapeutic population of regulatory T cells (Tregs), said method comprising the steps of:
a. enriching Tregs from a cell sample suspected of containing Tregs, to produce a baseline Treg cell population;
b. expanding the baseline Treg cell population to produce an expanded Treg cell population, wherein the baseline Treg cell population is not cryopreserved prior to the initiation of step (b); and
c. cryopreserving the expanded Treg cell population to produce a cryopreserved therapeutic population of Tregs.

2. The method of claim 1, wherein the expanded Treg cell population and the cryopreserved therapeutic population of Tregs, following thawing and without t additional expansion, exhibit an ability to suppress inflammatory cells, as measured by pro-inflammatory cytokine production by the inflammatory cells, wherein the inflammatory cells are macrophages or monocytes from human donors or generated from induced pluripotent stem cells.

3. The method of claim 2, wherein the ability of the cryopreserved therapeutic population of Tregs, following thawing and without additional expansion, to suppress inflammatory cells is at least 70% that of the expanded Treg cell population.

4. The method of claim 2 or 3, wherein the ability to suppress inflammatory cells is measured by IL-6, TNFα, IL1β, IL8, and/or Interferon-γ production by the inflammatory cells.

5. The method of claim 4, wherein the ability to suppress inflammatory cells is measured by IL-6 production by the inflammatory cells.

6. The method of any one of claims 1-5, wherein the cryopreserved therapeutic population of Tregs, following thawing and without additional expansion, exhibits a suppressive function, wherein the suppressive function is greater than that of the baseline Treg cell population, as determined by suppression of proliferation of responder T cells.

7. The method of claim 6, wherein the suppressive function of the cryopreserved therapeutic population of Tregs, following thawing and without additional expansion, is at least 25%, at least 50%, at least 75%, at least 100% at least 150%, or at least 300% greater than the suppressive function of the baseline Treg cell population as determined by suppression of proliferation of responder T cells.

8. The method of any one of claims 1-7, wherein the cryopreserved therapeutic population of Tregs, following thawing and without additional expansion, exhibits a suppressive function, wherein the suppressive function is at least 50%, at least 60%, at least 70%, at least 75%, at least 80%, at least 85%, at least 90% or at least 95%, as determined by suppression of proliferation of responder T cells.

9. The method of any one of claims 1-8, wherein the suppressive function of the cryopreserved therapeutic population of Tregs, following thawing and without t additional expansion, is at least 50%, at least 60%, at least 70%, at least 75%, at least 80%, at least 85%, at least 90% or at least 95% of the suppressive function of the expanded Treg cell population before cryopreservation.

10. The method of any one of claims 6-9, wherein the proliferation of responder T cells is determined by flow cytometry or thymidine incorporation.

11. The method of any one of claims 1-10, wherein the viability of the cryopreserved therapeutic population of Tregs, following thawing and without additional expansion, is at least 70%, at least 75%, at least 80%, at least 85%, at least 90% or at least 95%, as determined by trypan blue staining.

12. The method of any one of claims 1-11, wherein the viability of the cryopreserved therapeutic population of Tregs, following thawing and without additional expansion, is at least 70%, at least 75%, at least 80%, at least 85%, at least 90% or at least 95% of the viability of the expanded Treg cell population prior to the expanded Treg cell population being cryopreserved in step (c), as determined by trypan blue staining.

13. The method of any one of claims 1-12, wherein the cryopreserved therapeutic population of Tregs comprises FoxP3+ Tregs wherein the proportion of FoxP3+ Tregs is increased relative to the proportion of FoxP3+ Tregs in the Tregs in the baseline Treg cell population.

14. The method of any one of claims 1-13, wherein the cryopreserved therapeutic population of Tregs comprises at least 40%, at least 50%, at least 60%, at least 70%, or at least 80% or at least 90% FoxP3+ Tregs, as determined by flow cytometry.

15. The method of any one of claims 1-14, wherein the cryopreserved therapeutic population of Tregs comprise FoxP3-expressing Tregs wherein the expression of FoxP3 is increased in the Tregs relative to expression of FoxP3 in the Tregs in the baseline Treg cell population.

16. The method of any one of claims 1-15, wherein the cryopreserved therapeutic population of Tregs comprises at least 70%, at least 75%, at least 80%, at least 85%, at least 90% or at least 95% CD4⁺CD25⁺ cells, as determined by flow cytometry.

17. The method of any one of claims 1-16, wherein the cryopreserved therapeutic population of Tregs comprises fewer than 20% CD8+ cells, as determined by flow cytometry.

18. The method of any one of claims 1-17, wherein the cryopreserved therapeutic population of Tregs comprises at least 70%, at least 80%, or at least 90% CD4⁺CD25^{high}CD127^{low} Tregs, as determined by flow cytometry.

19. The method of any one of claims 1-18, wherein the cell sample is a leukapheresis cell sample.

20. The method of any one of claims 1-19, wherein the method further comprises obtaining the cell sample from a donor by leukapheresis.

21. The method of any one of claims 1-20, wherein the cell sample is not stored overnight or frozen before carrying out the enriching step (a).

22. The method of any one of claims 1-21, wherein the cell sample is obtained within 30 minutes before initiation of enriching step (a).

23. The method of any one of claims 1-22, wherein step (a) comprises depleting CD8+/CD19+ cells then enriching for CD25+ cells.

24. The method of any one of claims 1-23, wherein step (b) is carried out within 30 minutes after step (a).

25. The method of any one of claims 1-24, wherein step (b) comprises culturing the Tregs in a culture medium that comprises beads coated with anti-CD3 antibodies and anti-CD28 antibodies.

26. The method of claim 25, wherein the beads are first added to the culture medium within about 24 hours of the initiation of the culturing.

27. The method of claim 25 or 26, wherein beads coated with anti-CD3 antibodies and anti-CD28 antibodies are added to the culture medium about 14 days after beads coated with anti-CD3 antibodies and anti-CD28 antibodies were first added to the culture medium.

28. The method of any one of claims 1-26, wherein step (b) further comprises adding IL-2 to the culture medium within about 6 days of the initiation of culturing.

29. The method of claim 28, wherein step (b) further comprises replenishing the culture medium with IL-2 about every 2-3 days after IL-2 is first added to the culture medium.

30. The method of any one of claims 1-29, wherein step (b) further comprises adding rapamycin to the culture medium within about 24 hours of the initiation of the culturing.

31. The method of claim 30, wherein step (b) further comprises replenishing the culture medium with rapamycin every 2-3 days after the rapamycin is first added to the culture medium.

32. The method of any one of claims 28-31, wherein the cryopreserving step (c) is carried out at least 6 days following IL-2 addition to or replenishment of the culture medium in step (b).

33. The method of any one of claims 1-32, wherein the cryopreserving step (c) is carried out about 8-25 days after the initiation of the culturing step (b).

34. The method of any one of claims 1-33, wherein step(c) comprises cryopreserving the Tregs in a cryoprotectant comprising DMSO.

35. The method of any one of claims 1-34, wherein the cryopreservation step (c) comprises changing the temperature of the population of Tregs in the following increments: 1° C./min to 4° C., 25° C./min to −40° C., 10° C./min to −12° C., 1° C./min to −40° C., and 10° C./min to −80° C.-−90° C.

36. The method of any one of claims 1-35, wherein the cryopreserved therapeutic population of Tregs is frozen at a Treg density of at least 50 million cells/mL.

37. The method of any one of claims 1-36, wherein the cryopreserved therapeutic population of Tregs is frozen in a total volume of 1-1.5 mL.

38. The method of any one of claim 1-37, wherein the method further comprises thawing the cryopreserved therapeutic population of Tregs after cryopreservation for about 1 week, 1 month, about 3 months, about 6 months, about 9 months, about 12 months, about 18 months or about 24 months.

39. The method of any one of claims 1-38, wherein the cell sample is from a human donor.

40. The method of claim 39, wherein the human donor is a healthy donor.

41. The method of claim 39, wherein the human donor is diagnosed with or suspected of having a neurodegenerative disorder.

42. The method of claim 41, wherein the neurodegenerative disorder is amyotrophic lateral sclerosis, Alzheimer's disease, Parkinson's disease or frontotemporal dementia.

43. The method of any one of claims 1-42, wherein the population of Tregs is subjected to genetic engineering at any point of the method prior to cryopreserving step (c).

44. The method of any one of claims 1-43, wherein step (b) is automated.

45. The method of any one of claims 1-44, wherein step (b) takes place in a bioreactor.

46. The method of any one of claims 1-45, wherein step (b) takes place in a G-REX culture system.

47. The method of any one of claims 1-46, wherein the method is performed in a closed system.

48. The method of any one of claims 1-47, wherein the method further comprises thawing the cryopreserved therapeutic population of Tregs and, without further expansion, placing the population into a pharmaceutical composition comprising a pharmaceutically acceptable carrier, to produce a Treg pharmaceutical composition.

49. The method of claim 48, wherein the Treg pharmaceutical composition comprises normal saline and 5% human serum albumin.

50. The method of claim 48 or 49, wherein the method further comprises administering the Treg pharmaceutical composition to a human subject.

51. The method of any one of claims 48-50, wherein the Tregs in the pharmaceutical composition are autologous to the human subject.

52. The method of claim 50 or 51, wherein the human subject has been diagnosed with or is suspected of having a neurodegenerative disorder.

53. The method of claim 52, wherein the neurodegenerative disorder is amyotrophic lateral sclerosis (ALS), Alzheimer's disease, Parkinson's disease or frontotemporal dementia.

54. A cryopreserved therapeutic population of Tregs produced by the method of any one of claims 1-53.

55. A pharmaceutical composition comprising the cryopreserved therapeutic population of Tregs of claim 54, following thawing and without further expansion, and a pharmaceutically acceptable carrier.

56. An ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells.

57. The ex vivo-expanded Treg cell population of claim 56, wherein the ability to suppress inflammatory cells is measured by IL-6, TNFα, IL1β, IL8, and/or Interferon-γ production by the inflammatory cells.

58. The ex vivo-expanded Treg cell population of claim 57, wherein the ability to suppress inflammatory cells is measured by IL-6 production by the inflammatory cells.

59. The ex vivo-expanded Treg cell population of any one of claims 56-58, wherein the Treg cell population is autologous to a human subject with ALS.

60. The ex vivo-expanded Treg cell population of any one of claims 56-58, wherein the Treg cell population has been expanded from a cell sample from a human subject with ALS.

61. A pharmaceutical composition comprising the ex vivo-expanded Treg cell population of any one of claims 56-60 and a pharmaceutically acceptable carrier.

62. A cryopreserved therapeutic population of ex vivo-expanded Tregs that, following thawing and without additional expansion, exhibits an ability to suppress inflammatory cells, as measured by pro-inflammatory cytokine production by the inflammatory cells, wherein the inflammatory cells are macrophages or monocytes from human donors or generated from induced pluripotent stem cells.

63. The cryopreserved therapeutic population of ex vivo-expanded Tregs of claim 62, wherein the ability of the cryopreserved therapeutic population of ex vivo-expanded Tregs to suppress inflammatory cells, following expansion and without additional expansion, is at least 70%, that of the ex vivo-expanded Tregs prior to cryopreservation.

64. The cryopreserved therapeutic population of ex vivo-expanded Tregs of claim 62 or 63, wherein the ability to suppress inflammatory cells is measured by IL-6, TNFα, IL1β, IL8, and/or Interferon-γ production by the inflammatory cells.

65. The cryopreserved therapeutic population of ex vivo-expanded Tregs of claim 64, wherein the ability to suppress inflammatory cells is measured by IL-6 production by the inflammatory cells.

66. The cryopreserved therapeutic population of ex vivo-expanded Tregs of any one of claims 62-65, wherein the cryopreserved therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% as determined by suppression of proliferation of responder T cells by flow cytometry or thymidine incorporation.

67. The cryopreserved therapeutic population of ex vivo-expanded Tregs of any one of claims 62-66 wherein the cryopreserved therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% of the suppressive function of the ex vivo-expanded Tregs prior to cryopreservation, as determined by suppression of proliferation of responder T cells by flow cytometry or thymidine incorporation.

68. The cryopreserved therapeutic population of ex vivo-expanded Tregs of any one of claims 62-67, wherein the cryopreserved therapeutic population of ex vivo-expanded Tregs, following thawing and without additional expansion, exhibits at least 70%, at least 75%, at least 80%, at least 85%, at least 90% or at least 95% viability, as determined by trypan blue staining.

69. The cryopreserved therapeutic population of ex vivo-expanded Tregs of any one of claims 62-68, wherein the cryopreserved therapeutic population of ex vivo-expanded Tregs, following thawing and without additional expansion, exhibits a viability that is at least 70%, at least 75%, at least 80%, at least 85%, at least 90% or at least 95% of the viability of the Tregs before cryopreservation, as determined by trypan blue staining.

70. The cryopreserved therapeutic population of ex vivo-expanded Tregs of any one of claims 62-69, wherein the cryopreserved therapeutic population of ex vivo-expanded Tregs, following thawing and without additional expansion, comprises at least 40%, at least 50%, at least 60%, at least 70%, or at least 80% or at least 90% FoxP3⁺ Tregs, as determined by flow cytometry.

71. The cryopreserved therapeutic population of ex vivo-expanded Tregs of any one of claims 62-70, wherein the cryopreserved therapeutic population of ex vivo-expanded Tregs, following thawing and without additional expansion, comprises at least 70%, at least 75%, at least 80%, at least 85%, at least 90% or at least 95% CD4⁺CD25⁺ cells, as determined by flow cytometry.

72. The cryopreserved therapeutic population of ex vivo-expanded Tregs of any one of claims 62-71, wherein the cryopreserved therapeutic population of ex vivo-expanded Tregs, following thawing and without additional expansion, comprises fewer than 20% CD8+ cells, as determined by flow cytometry.

73. The cryopreserved therapeutic population of ex vivo-expanded Tregs of any one of claims 62-72, wherein the cryopreserved therapeutic population of ex vivo-expanded Tregs, following thawing and without additional expansion, comprises at least 70%, at least 80%, or at least 90% CD4⁺CD25^{high} CD127^{low} Tregs, as determined by flow cytometry.

74. The cryopreserved therapeutic population of ex vivo-expanded Tregs of any one of claims 62-73, wherein the ex vivo-expanded Tregs are autologous to a human subject with ALS.

75. The cryopreserved therapeutic population of ex vivo-expanded Tregs of any one of claims 62-73, wherein the ex vivo-expanded Tregs have been expanded from a cell sample from a human subject with ALS.

76. A pharmaceutical composition comprising the cryopreserved therapeutic population of Tregs of any one of claims 64-73, following thawing and without further expansion, and a pharmaceutically acceptable carrier.

77. An ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population:
a) expression of one or more dysfunctional baseline signature gene products listed in Table 12 and/or Table 13 is decreased relative to the expression of the one or more gene products in baseline Tregs;
b) expression of one or more dysfunctional baseline signature gene products listed in Table 14 is decreased relative to the expression of the one or more gene products in baseline Tregs;
c) expression of one or more Treg-associated signature gene products listed in Table 15 is increased relative to the expression of the one or more gene products in baseline Tregs;
d) expression of one or more mitochondria signature gene products listed in Table 16 is increased relative to the expression of the one or more gene products in baseline Tregs;
e) expression of one or more cell proliferation signature gene products listed in Table 17 is increased relative to the expression of the one or more gene products in baseline Tregs; or
f) expression of one or more highest protein expression signature gene products listed in Table 18 is increased relative to the expression of the one or more gene products in baseline Tregs.

78. The ex vivo-expanded Treg cell population of claim 77, wherein the ability to suppress inflammatory cells is measured by IL-6, TNFα, IL1β, IL8, and/or Interferon-7 production by the inflammatory cells.

79. The ex vivo-expanded Treg cell population of claim 78, wherein the ability to suppress inflammatory cells is measured by IL-6 production by the inflammatory cells.

80. The ex vivo-expanded Treg cell population of any one of claims 77-79, wherein, in the ex vivo-expanded Treg cell population, expression of one or more of the following gene products is increased relative to expression of the one or more gene products in baseline Tregs: ADAM10, AIMIP1, AIMIP2, ARG2, BCL2L1, BSG, CD2, CD28, CD38, CD74, CD84, CTLA4, FAS, FOXP3, GCLC, HAT1, HLA-DQA1, HLA-DQB1, HLA-DRA, HLA-DRB1, HPGD, ICOS, IL1RN, IRF4, KPNA2, LGALS1, LGMN, PCNA, POFUT1, SATB1, SELPLG, STAT1, TFRC and TNFRSF18.

81. The ex vivo-expanded Treg cell population of any one of claims 77-80, wherein, in the ex vivo-expanded Treg cell population, expression of one or more of the following gene products is increased relative to expression of the one or more gene products in baseline Tregs: ACAA2, ACADM, ACADVL, ACOT7, ACSL1, ACSL4, ACSL5, AGK, AGMAT, AK4, ARG2, ARL2, AUH, BCL2L1, BDH1, BNIP1, CDK1, CHDH, CIAPIN1, CISD2, COX17, CPOX, CPT1A, CPT2, CYB5B, DAP3, DHRS2, DNM1L, DUT, DYNLL1, ECI1, FDXR, FEN1, FKBP8, GK, GRSF1, HTRA2, L2HGDH, LACTB2, LRPPRC, MAIP1, MAOA, MPST, MRPL1, MRPL12, MRPL13, MRPL14, MRPL17, MRPL22, MRPL37, MRPL39, MRPL4, MRPL43, MRPL44, MRPL46, MRPL48, MRPS11, MRPS14, MRPS2, MRPS27, MRPS31, MRPS35, MRPS9, MTHFD2, MTX1, MYCBP, NDUFA8, NUDT1, OAT, PITRM1, PLSCR3, PMPCA, PPIF, PTRH2, PYCR2, REXO2, RMND1, SFXN2, SLC25A10, SLC25A19, SLC25A4, TIGAR, TIMM13, TIMM23, TMEM14C, TOMM22, TOMM34, TOMM40, and TST.

82. The ex vivo-expanded Treg cell population of any one claims 77-81, wherein, in the ex vivo-expanded Treg cell population, expression of one or more of the following gene products is increased relative to expression of the one or more gene products in baseline Tregs: ARL2, ARL3, BCCIP, CCDC124, CDK1, CDK2, CDK5, CDK6, CUL4B, DCTN3, FEN1, HELLS, LIG1, MAD2L1, MAEA, MCM2, MCM2, MCM3, MCM4, MCM5, MCM6, MCM7, MCMBP, NUDC, PCNA, POLD1, POLD2, RALB, RBM38, RFC2, RFC3, RFC4, RFC5, RNASEH2A, RNASEH2B, and SMC2.

83. The ex vivo-expanded Treg cell population of any one of claims 77-82, wherein, in the ex vivo-expanded Treg cell population, expression of one or more of the following gene products is increased relative to expression of the one or more gene products in baseline Tregs: ACAA2, ACADM, ACADVL, ACOT7, BSG, CACYBP, CD74, CDK1, CPOX, DUT, ECI1, ENO3, FEN1, FKBP3, HIST1H2BJ, HLA-DQA1, HLA-DRA, HLA-DRB1, LGALS1, LGALS3, MCM5, MCM6, MCM7, MTHFD1, NAMPT, NME1, NQO1, PCNA, RAB1A, RALB, SLC25A4, STAT1, STMN1, STMN2, TUBAIB, TUBB4A, TUBB8, TXN, TXNRD1, and WARS.

84. The ex vivo-expanded Treg cell population of any one claims 77-83, wherein, in the ex vivo-expanded Treg cell population, expression of one or more dysfunctional baseline signature gene products listed in Table 12 and/or Table 13 is decreased relative to the expression of the one or more gene products in baseline Tregs.

85. An ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population: expression of one or more Treg-associated signature gene products listed in Table 14 is increased relative to the expression of the one or more gene products in baseline Tregs.

86. The ex vivo-expanded Treg cell population of claim 85, wherein, in the ex vivo-expanded Treg cell population expression of one or more dysfunctional baseline signature gene products listed in Table 12 and/or Table 13 is decreased relative to the expression of the one or more gene products in baseline Tregs.

87. The ex vivo-expanded Treg cell population of claim 85 or 86, wherein the ex vivo-expanded Treg cell population exhibits a suppressive function, wherein the suppressive function is at least 50%, at least 60%, at least 70%, at least 75%, at least 80%, at least 85%, at least 90% or at least 95% as determined by suppression of proliferation of responder T cells by flow cytometry or thymidine incorporation.

88. The ex vivo-expanded Treg cell population of any one of claims 85-87, wherein the ex vivo-expanded Treg cell population exhibits a suppressive function, wherein the suppressive function is at least 50%, at least 75%, at least 100%, or at least 150% that of baseline Tregs, as determined by suppression of proliferation of responder T cells by flow cytometry or thymidine incorporation.

89. The ex vivo-expanded Treg cell population of any one of claims 77-88, wherein the ex vivo-expanded Tregs are autologous to a human subject with ALS.

90. The ex vivo-expanded Treg cell population of any one of claims 77-88, wherein the ex vivo-expanded Tregs have been expanded from a cell sample from a human subject with ALS.

91. The ex vivo-expanded Treg cell population of any one of claims 77-90, wherein the expression of the one or more gene products is changed by a log 2 change of at least about 4-fold, at least about 5-fold, at least about 6-fold, at least about 7-fold, at least about 8-fold, at least about 9-fold, or at least about 10-fold.

92. The ex vivo-expanded Treg cell population of any one of claims 77-91, wherein expression is determined by single-shot proteomic analysis.

93. A pharmaceutical composition comprising the ex vivo-expanded Treg cell population of any one of claims 77-88 or 91-92, following thawing and without further expansion, and a pharmaceutically acceptable carrier.

94. A cryopreserved composition comprising a therapeutic population of ex vivo-expanded Tregs, wherein following thawing and without further expansion, the expression of one or more of gene products listed in Table 12-Table 18 is substantially the same in therapeutic population of Tregs as the expression of the one or more gene products in the ex vivo-expanded Tregs prior to cryopreservation.

95. The cryopreserved composition of claim 94, wherein the one or more gene products is not also listed in Table 19.

96. The cryopreserved composition of claim 94 or 95, wherein the one or more gene products is a gene product associated with a dysfunctional Treg phenotype, a methylation- or epigenetics-associated gene product, a mitochondria-related gene product, or a gene products associated with the cell cycle, cell division, DNA replication or DNA repair.

97. The cryopreserved composition of any one of claims 94-96, wherein the one or more gene products is known to be important for the proliferation, health, identification, and/or mechanism of Treg cells.

98. The cryopreserved composition of any one of claims 94-97 wherein the therapeutic population of ex vivo-expanded Tregs, following thawing and without additional expansion, exhibits an ability to suppress inflammatory cells, as measured by pro-inflammatory cytokine production by the inflammatory cells, wherein the inflammatory cells are macrophages or monocytes from human donors or generated from induced pluripotent stem cells.

99. The cryopreserved composition of claim 98, wherein the ability of the ex vivo-expanded Tregs to suppress inflammatory cells, following expansion and without t additional expansion, is at least 70%, that of the ex vivo-expanded Tregs prior to cryopreservation.

100. The cryopreserved composition of claim 98 or 99, wherein the ability of the ex vivo-expanded Tregs to suppress inflammatory cells is measured by IL-6, TNFα, IL1β, IL8, and/or Interferon-γ production by the inflammatory cells.

101. The cryopreserved composition of claim 100, wherein the ability to suppress inflammatory cells is measured by IL-6 production by the inflammatory cells.

102. The cryopreserved composition of any one of claim 94-101 wherein the therapeutic population of ex vivo-expanded Tregs, following thawing and without t additional expansion, exhibits a suppressive function that is at least 50%, at least 60%, at least 70%, at least 75%, at least 80%, at least 85%, at least 90% or at least 95% as determined by suppression of proliferation of responder T cells by flow cytometry or thymidine incorporation.

103. The cryopreserved composition of any one of claim 94-102, wherein the therapeutic population of ex vivo-expanded Tregs, following thawing and without t additional expansion, exhibits a suppressive function that is at least 50%, at least 60%, at least 70%, at least 75%, at least 80%, at least 85%, at least 90% or at least 95% of the suppressive function of the ex vivo-expanded Tregs prior to cryopreservation, as determined by suppression of proliferation of responder T cells by flow cytometry or thymidine incorporation.

104. The cryopreserved composition of any one of claim 94-103, wherein the therapeutic population of ex vivo-expanded Tregs, following thawing and without t additional expansion, exhibits at least 70%, at least 75%, at least 80%, at least 85%, at least 90% or at least 95% viability, as determined by trypan blue staining.

105. The cryopreserved composition of any one of claim 94-104, wherein the therapeutic population of ex vivo-expanded Tregs, following thawing and without t additional expansion, exhibits a viability that is at least 70%, at least 75%, at least 80%, at least 85%, at least 90% or at least 95% of the viability of the Tregs before cryopreservation, as determined by trypan blue staining.

106. The cryopreserved composition of any one of claim 94-105, wherein the therapeutic population of ex vivo-expanded Tregs, following thawing and without t additional expansion, comprises at least 40%, at least 50%, at least 60%, at least 70%, or at least 80% or at least 90% FoxP3⁺ Tregs, as determined by flow cytometry.

107. The cryopreserved composition of any one of claim 94-106, wherein the therapeutic population of ex vivo-expanded Tregs, following thawing and without t additional expansion, comprises at least 70%, at least 75%, at least 80%, at least 85%, at least 90% or at least 95% CD4⁺CD25⁺ cells, as determined by flow cytometry.

108. The cryopreserved composition of any one of claim 94-107, wherein the therapeutic population of ex vivo-expanded Tregs, following thawing and without t additional expansion, comprises fewer than 20% CD8+ cells, as determined by flow cytometry.

109. The cryopreserved composition of any one of claim 94-108, wherein the therapeutic population of ex vivo-expanded Tregs, following thawing and without t additional expansion, comprises at least 70%, at least 80%, or at least 90% CD4⁺CD25^{high}CD127^{low} Tregs, as determined by flow cytometry.

110. The cryopreserved composition of any one of claims 94-109, wherein the ex vivo-expanded Tregs are autologous to a human subject with ALS.

111. The cryopreserved composition of any one of claims 94-109, wherein the ex vivo-expanded Tregs have been expanded from a cell sample from a human subject with ALS.

112. The cryopreserved composition of any one of claims 94-111, wherein gene product expression is determined by single-shot proteomic analysis.

113. A pharmaceutical composition comprising the cryopreserved composition comprising a therapeutic population of ex vivo-expanded Tregs of any one of claims 94-106 or 109-112 following thawing and without further expansion, and a pharmaceutically acceptable carrier.

114. A method of treating a disorder associated with Treg dysfunction, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

115. A method of treating a disorder associated with Treg deficiency, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

116. A method of treating a disorder associated with overactivation of the immune system, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

117. A method of treating an inflammatory condition driven by a T cell response, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

118. A method of treating an inflammatory condition driven by a myeloid cell response, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

119. The method of claim 118, wherein the myeloid cell is a monocyte, macrophage or microglia.

120. A method of treating a neurodegenerative disorder in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering.

121. The method of claim 120, wherein the neurodegenerative disease is Amyotrophic Lateral Sclerosis (ALS), Alzheimer's disease, Parkinson's disease, frontotemporal dementia or Huntington's disease.

122. A method of treating an autoimmune disorder in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering.

123. The method of claim 122, wherein the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, systemic lupus erythematosus, cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

124. A method of treating graft-versus-host disease in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering.

125. The method of claim 124, wherein the subject has received a bone marrow transplant, kidney transplant or liver transplant.

126. A method of improving islet graft survival in a subject in need thereof, comprising: combining islet transplantation with administering to the subject a pharmaceutical composition comprising a therapeutic population of Tregs, wherein the Tregs had been ex vivo expanded and cryopreserved, and wherein the Tregs are not further expanded prior to the administering.

127. A method of treating cardio-inflammation in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering.

128. The method of claim 127, wherein the cardio-inflammation is associated with atherosclerosis, myocardial infarction, ischemic cardiomyopathy or heart failure.

129. A method of treating neuroinflammation in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering.

130. The method of claim 129, wherein the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

131. A method of treating a Tregopathy in a subject in need thereof, comprising: administering to the subject a pharmaceutical composition comprising a therapeutic population of Tregs, wherein the Tregs had been ex vivo expanded and cryopreserved, and wherein the Tregs are not further expanded prior to the administering.

132. The method of claim 131, wherein the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

133. The method of any one of claims 112-132, wherein the Tregs are autologous to the subject.

134. The method of any one of claims 112-132, wherein the Tregs are allogeneic to the subject.

135. The method of any one of claims 112-134, wherein the pharmaceutical composition is the pharmaceutical composition of claim 55.

136. The method of any one of claims 112-134, wherein the pharmaceutical composition is the pharmaceutical composition of claim 61.

137. The method of any one of claims 112-134, wherein the pharmaceutical composition is the pharmaceutical composition of claim 76.

138. The method of any one of claims 112-134, wherein the pharmaceutical composition is the pharmaceutical composition of claim 93.

139. The method of any one of claims 112-134, wherein the pharmaceutical composition is the pharmaceutical composition of claim 113.
